(19) United States
(12) Patent Application Publication
(10) Pub. No.: US 2011/0269201 A1
(43) Pub. Date: Nov. 3, 2011

(54) REDIRECTED BIOENERGETICS IN RECOMBINANT CELLULOLYTIC CLOSTRIDIUM MICROORGANISMS

(75) Inventors: Kevin Gray, Northborough, MA (US); Patrick O'Mullan, Westborough, MA (US)

(73) Assignee: Qteros, Inc., Marlborough, MA (US)

(21) Appl. No.: 13/098,264

(22) Filed: Apr. 29, 2011

Related U.S. Application Data
(60) Provisional application No. 61/330,138, filed on Apr. 30, 2010.

(51) Int. Cl. C12P 7/06; C12P 7/02; C12P 1/00; C12P 1/04; C12N 1/21; C12M 1/00

(52) U.S. Cl. 435/161; 435/252.3; 435/289.1; 435/41; 435/170; 435/155

(57) ABSTRACT

Compositions and methods are provided for redirecting metabolic solventogenesis pathways to enhance the product yield from fermentation of biomass. Clostridium microorganism pathways are modified to extend the growth phase and prevent inhibition of acetaldehyde while bypassing the synthesis of acetyl CoA.

### CROSS-REFERENCE

This application claims the benefit of U.S. Provisional Application No. 61/330,138, filed Apr. 30, 2010, which application is incorporated herein by reference in its entirety.

### BACKGROUND OF THE INVENTION

Biomass is a renewable source of energy, which can be biologically fermented to produce an end-product such as a fuel or other useful compound (e.g. alcohol, ethanol, organic acid, acetic acid, lactic acid, methane, or hydrogen). Biomass includes agricultural residues (corn stalks, grass, straw, grain hulls, bagasse, etc.), animal waste (manure from cattle, poultry, and hogs), Distillers Dried Solubles (DDS), Distillers Dried Grains (DDG), Condensed Distillers Solubles (CDS), Distillers Wet Grains (DWG), Distillers Dried Grains with Solubles (DDGS), woody materials (wood or bark, sawdust, timber slash, and mill scrap), municipal waste (waste paper, recycled toilet papers, yard clippings, etc.), and energy crops (poplars, willows, switch grass, alfalfa, prairie bluestem, algae etc.). Lignocellulosic biomass has cellulose and hemicellulose as two major components.

There is a growing consensus that fermenting chemicals from renewable resources such as cellulosic and lignocellulosic plant materials has great potential and can replace chemical synthesis that use petroleum reserves as energy sources, thus, reducing greenhouse gases while supporting agriculture. However, microbial fermentation requires adapting strains of microorganisms to industrial fermentation parameters to be economically feasible. Unfortunately, many organisms used for fermentation of carbonaceous substrates cannot generate enough product yield to make the fermentation process cost effective. Progress in bioproduct fermentation has been hampered by lack of suitable microorganisms that can effectively hydrolyze and metabolize all of the sugars present in a biomass and generate ethanol or other preferred chemicals with 90% or better theoretical yield. There is great need for organisms that can efficiently utilize polysaccharides such as cellulose and hemicellulose without diverting energy to the conversion of undesirable products.

Clostridia species are well known as natural synthesizers of chemical products and several can adapt to commercial fermentation systems. However, few Clostridia species can saccharify and ferment biomass to commercially desirable biofuels and other chemical end products, and most of these end products are produced in low amounts. Although it is ecologically desirable to develop renewable organic substances, it is not yet economically feasible. There remains a strong need for microbial species that can consolidate the process of saccharification and fermentation in an efficient and cost-effective manner.

To obtain a high fermentation efficiency of lignocellulosic biomass to end-product (yield) it is important to provide an appropriate fermentation microorganism that directs metabolism to increase yields of preferred end-products. Under anaerobic conditions, ethanolic Clostridia sp. carry out alcoholic fermentation by the decarboxylation of pyruvate into acetaldehyde, catalysed by pyruvate dehydrogenase (PDH) and the subsequent reduction of acetaldehyde into ethanol by NADH, catalysed by alcohol dehydrogenase (ADH). In some organisms, pyruvate is also converted to lactic acid through catalysis by lactate dehydrogenase (LDH). Inactivation of LDH can result in improved ethanol yields in these organisms by directing the conversion of pyruvate to ethanol rather than lactic acid. More importantly, modification of metabolic pathways to increase glycolytic flux can improve end-product yields.

### SUMMARY OF THE INVENTION

Disclosed herein are genetically modified Clostridium bacteria that express a pyruvate decarboxylase protein, wherein the genetically modified Clostridium bacteria produce an increased yield of a fermentation end-product as compared to non-genetically modified Clostridium bacteria. Also disclosed herein are genetically modified Clostridium bacteria that express a pyruvate decarboxylase protein, wherein the Clostridium bacteria produce a fermentation end-product at a greater rate as compared to non-genetically modified Clostridium bacteria. In some embodiments, the pyruvate decarboxylase protein is endogenous or heterologous. In some embodiments, the pyruvate decarboxylase gene has greater than 90% identity to SEQ ID NO: 19. In some embodiments, a genetically modified Clostridium bacterium further comprises a genetic modification that expresses a heterologous alcohol dehydrogenase gene. In some embodiments, the heterologous alcohol dehydrogenase gene has greater than 90% identity to SEQ ID NO: 17. In some embodiments, a genetically modified Clostridium bacterium further comprises a genetic modification that expresses a heterologous acetyl-CoA synthetase protein. In some embodiments, the heterologous acetyl-CoA synthetase gene has greater than 90% identity to SEQ ID NO: 21. In some embodiments, a genetically modified Clostridium bacterium further comprises a genetic modification that inactivates an endogenous lactate dehydrogenase gene. In some embodiments, the fermentation end-product is an alcohol. In some embodiments, the alcohol is ethanol. In some embodiments, the genetically modified Clostridium bacterium is genetically modified C. phytofermentans or Clostridium sp Q.D. In some embodiments, the genetically modified Clostridium bacterium produces the fermentation end-product at a yield that is at least 1.5 times greater than the non-genetically modified Clostridium bacterium. In some embodiments, the genetically modified microorganism produces the fermentation end-product at a rate at least 1.5 times greater than the non-genetically modified Clostridium bacterium. In some embodiments, the genetically modified Clostridium bacterium can hydrolyze hexose or pentose sugars. In some embodiments, the genetically modified Clostridium bacterium can hydrolyze and ferment hexose or pentose sugars. In some embodiments, the genetically modified Clostridium bacterium can hydrolyze and ferment cellulosic and/or lignocellulosic material.

Disclosed herein are genetically modified Clostridium bacteria that express a heterologous alcohol dehydrogenase protein, wherein the genetically modified Clostridium bacteria produce an increased yield of a fermentation end-product as compared to non-genetically modified Clostridium bacteria. Also disclosed herein are genetically modified Clostridium bacteria that express a heterologous alcohol dehydrogenase protein, wherein the genetically modified Clostridium bacteria produce a fermentation end-product at a greater rate as compared to non-genetically modified Clostridium bacteria. In some embodiments, the heterologous alcohol dehydrogenase gene has greater than 90% identity to SEQ ID NO: 17. In some embodiments, a genetically modified Clostridium bacterium further comprises a genetic modification that expresses a pyruvate decarboxylase gene. In some embodiments, the pyruvate decarboxylase gene is endogenous or heterologous. In some embodiments, the pyruvate decarboxylase gene has greater than 90% identity to SEQ ID NO: 19. In some embodiments, a genetically modified Clostridium bacterium further comprises a genetic modification that expresses a heterologous acetyl-CoA synthetase protein. In some embodiments, the heterologous acetyl-CoA synthetase gene has greater than 90% identity to SEQ ID NO: 21. In some embodiments, a genetically modified Clostridium bacterium further comprises a genetic modification that inactivates an endogenous lactate dehydrogenase gene. In some embodiments, the fermentation end-product is an alcohol. In some embodiments, the alcohol is ethanol. In some embodiments, the genetically modified Clostridium bacterium is genetically modified C. phytofermentans or Clostridium sp Q.D. In some embodiments, the genetically modified Clostridium bacterium produces the fermentation end-product at a yield that is at least 1.5 times greater than the non-genetically modified Clostridium bacterium. In some embodiments, the genetically modified microorganism produces the fermentation end-product at a rate at least 1.5 times greater than the non-genetically modified Clostridium bacterium. In some embodiments, the genetically modified Clostridium bacterium can hydrolyze hexose or pentose sugars. In some embodiments, the genetically modified Clostridium bacterium can hydrolyze and ferment hexose or pentose sugars. In some embodiments, the genetically modified Clostridium bacterium can hydrolyze and ferment cellulosic and/or lignocellulosic material.

Disclosed herein are methods of producing a fermentation end-product, comprising: contacting a carbonaceous biomass with a genetically modified Clostridium bacterium that expresses a pyruvate decarboxylase protein in a medium, wherein the genetically modified Clostridium bacterium produces an increased yield of the fermentation end-product as compared to a non-genetically modified Clostridium bacterium; and, incubating the carbonaceous biomass, medium, and genetically modified Clostridium bacterium for a sufficient amount of time to produce the fermentation end-product. Also disclosed herein are methods of producing a fermentation end-product, comprising: contacting a carbonaceous biomass with a genetically modified Clostridium bacterium that expresses a pyruvate decarboxylase protein in a medium, wherein the genetically modified Clostridium bacterium produces the fermentation end-product at an increased rate as compared to a non-genetically modified Clostridium bacterium; and, incubating the carbonaceous biomass, medium, and genetically modified Clostridium bacterium for a sufficient amount of time to produce the fermentation end-product. In some embodiments, the pyruvate decarboxylase protein is endogenous or heterologous. In some embodiments, the pyruvate decarboxylase gene has greater than 90% identity to SEQ ID NO: 19. In some embodiments, the genetically modified Clostridium bacterium further comprises a genetic modification that expresses a heterologous alcohol dehydrogenase protein. In some embodiments, the heterologous alcohol dehydrogenase gene has greater than 90% identity to SEQ ID NO: 17. In some embodiments, the genetically modified Clostridium bacterium further comprises a genetic modification that expresses a heterologous acetyl-CoA synthetase protein. In some embodiments, the heterologous acetyl-CoA synthetase gene has greater than 90% identity to SEQ ID NO: 21. In some embodiments, the genetically modified Clostridium bacterium further comprises a genetic modification that inactivates an endogenous lactate dehydrogenase gene. In some embodiments, the fermentation end-product is an alcohol. In some embodiments, the alcohol is ethanol. In some embodiments, the genetically modified Clostridium bacterium is genetically modified C. phytofermentans. In some embodiments, the genetically modified Clostridium bacterium is genetically modified Clostridium sp Q.D. In some embodiments, the genetically modified Clostridium bacterium produces the fermentation end-product at a yield that is at least 1.5 times greater than the non-genetically modified Clostridium bacterium. In some embodiments, the genetically modified Clostridium bacterium produces the fermentation end-product at a rate at least 1.5 times greater than the non-genetically modified Clostridium bacterium. In some embodiments, the genetically modified Clostridium bacterium can hydrolyze hexose or pentose sugars. In some embodiments, the genetically modified Clostridium bacterium can hydrolyze and ferment hexose or pentose sugars. In some embodiments, the genetically modified Clostridium bacterium can hydrolyze and ferment cellulosic and/or lignocellulosic material. In some embodiments, the carbonaceous biomass comprises woody plant matter, non-woody plant matter, cellulosic material, lignocellulosic material, hemicellulosic material, carbohydrates, pectin, starch, inulin, fructans, glucans, corn, corn stover, sugar cane, grasses, switch grass, sorghum, bamboo, distillers grains, Distillers Dried Solubles (DDS), Distillers Dried Grains (DDG), Condensed Distillers Solubles (CDS), Distillers Wet Grains (DWG), Distillers Dried Grains with Solubles (DDGS), peels, citrus peels, bagasse, poplar, or algae. In some embodiments, the carbonaceous biomass comprises cellulosic or lignocellulosic materials. In some embodiments, the carbonaceous biomass is pretreated to make the polysaccharides more available to the bacterium.

Disclosed herein are methods of producing a fermentation end-product, comprising: contacting a carbonaceous biomass with a genetically modified Clostridium bacterium that expresses a heterologous alcohol dehydrogenase protein in a medium, wherein the genetically modified Clostridium bacterium produces an increased yield of the fermentation end-product as compared to a non-genetically modified Clostridium bacterium; and, incubating the carbonaceous biomass, medium, and genetically modified Clostridium bacterium for a sufficient amount of time to produce the fermentation end-product. Also disclosed herein are methods of producing a fermentation end-product, comprising: contacting a carbonaceous biomass with a genetically modified Clostridium bacterium that expresses a heterologous alcohol dehydrogenase protein in a medium, wherein the genetically modified Clostridium bacterium produces the fermentation end-product at an increased rate as compared to a non-genetically modified Clostridium bacterium; and, incubating the carbonaceous biomass, medium, and genetically modified Clostridium bacterium for a sufficient amount of time to produce the fermentation end-product. In some embodiments, the heterologous alcohol dehydrogenase gene has greater than 90% identity to SEQ ID NO: 17. In some embodiments, the genetically modified Clostridium bacterium further comprises a genetic modification that expresses a pyruvate decarboxylase protein. In some embodiments, the pyruvate decarboxylase protein is endogenous or heterologous. In some embodiments, the pyruvate decarboxylase gene has greater than 90% identity to SEQ ID NO: 19. In some embodiments, the genetically modified Clostridium bacterium further comprises a genetic modification that expresses a heterologous acetyl-CoA synthetase protein. In some embodiments, the heterologous acetyl-CoA synthetase gene has greater than 90% identity to SEQ ID NO: 21. In some embodiments, the genetically modified Clostridium bacterium further comprises a genetic modification that inactivates an endogenous lactate dehydrogenase gene. In some embodiments, the fermentation end-product is an alcohol. In some embodiments, the alcohol is ethanol. In some embodiments, the genetically modified Clostridium bacterium is genetically modified C. phytofermentans. In some embodiments, the genetically modified Clostridium bacterium is genetically modified Clostridium sp Q.D. In some embodiments, the genetically modified Clostridium bacterium produces the fermentation end-product at a yield that is at least 1.5 times greater than the non-genetically modified Clostridium bacterium. In some embodiments, the genetically modified Clostridium bacterium produces the fermentation end-product at a rate at least 1.5 times greater than the non-genetically modified Clostridium bacterium. In some embodiments, the genetically modified Clostridium bacterium can hydrolyze hexose or pentose sugars. In some embodiments, the genetically modified Clostridium bacterium can hydrolyze and ferment hexose or pentose sugars. In some embodiments, the genetically modified Clostridium bacterium can hydrolyze and ferment cellulosic and/or lignocellulosic material. In some embodiments, the carbonaceous biomass comprises woody plant matter, non-woody plant matter, cellulosic material, lignocellulosic material, hemicellulosic material, carbohydrates, pectin, starch, inulin, fructans, glucans, corn, corn stover, sugar cane, grasses, switch grass, sorghum, bamboo, distillers grains, Distillers Dried Solubles (DDS), Distillers Dried Grains (DDG), Condensed Distillers Solubles (CDS), Distillers Wet Grains (DWG), Distillers Dried Grains with Solubles (DDGS), peels, citrus peels, bagasse, poplar, or algae. In some embodiments, the carbonaceous biomass comprises cellulosic or lignocellulosic materials. In some embodiments, the carbonaceous biomass is pretreated to make the polysaccharides more available to the bacterium.

Disclosed herein are systems for producing a fermentation end-product comprising: a fermentation vessel; a carbonaceous biomass; a genetically modified Clostridium bacterium that expresses a pyruvate decarboxylase protein, wherein the genetically modified Clostridium bacterium produces an increased yield of the fermentation end-product as compared to a non-genetically modified Clostridium bacterium; and, a medium. Also disclosed herein are systems for producing a fermentation end-product comprising: a fermentation vessel; a carbonaceous biomass; a genetically modified Clostridium bacterium that expresses a pyruvate decarboxylase protein, wherein the genetically modified Clostridium bacterium produces the fermentation end-product at an increased rate as compared to a non-genetically modified Clostridium bacterium; and, a medium. In some embodiments, the fermentation vessel is configured to house the medium and the microorganism, and wherein the carbonaceous biomass comprises a cellulosic and/or lignocellulosic material. In some embodiments, the pyruvate decarboxylase protein is endogenous or heterologous. In some embodiments, the pyruvate decarboxylase gene has greater than 90% identity to SEQ ID NO: 19. In some embodiments, the genetically modified Clostridium bacterium further comprises a genetic modification that expresses a heterologous alcohol dehydrogenase protein. In some embodiments, the heterologous alcohol dehydrogenase gene has greater than 90% identity to SEQ ID NO: 17. In some embodiments, the genetically modified Clostridium bacterium further comprises a genetic modification that expresses a heterologous acetyl-CoA synthetase protein. In some embodiments, the heterologous acetyl-CoA synthetase gene has greater than 90% identity to SEQ ID NO: 21. In some embodiments, the genetically modified Clostridium bacterium further comprises a genetic modification that inactivates an endogenous lactate dehydrogenase gene. In some embodiments, the fermentation end-product is an alcohol. In some embodiments, the alcohol is ethanol. In some embodiments, the genetically modified Clostridium bacterium is genetically modified C. phytofermentans. In some embodiments, the genetically modified Clostridium bacterium is genetically modified Clostridium sp Q.D. In some embodiments, the genetically modified Clostridium bacterium produces the fermentation end-product at a yield that is at least 1.5 times greater than the non-genetically modified Clostridium bacterium. In some embodiments, the genetically modified Clostridium bacterium produces the fermentation end-product at a rate at least 1.5 times greater than the non-genetically modified Clostridium bacterium. In some embodiments, the genetically modified Clostridium bacterium can hydrolyze hexose or pentose sugars. In some embodiments, the genetically modified Clostridium bacterium can hydrolyze and ferment hexose or pentose sugars. In some embodiments, the genetically modified Clostridium bacterium can hydrolyze and ferment cellulosic and/or lignocellulosic material. In some embodiments, the carbonaceous biomass comprises woody plant matter, non-woody plant matter, cellulosic material, lignocellulosic material, hemicellulosic material, carbohydrates, pectin, starch, inulin, fructans, glucans, corn, corn stover, sugar cane, grasses, switch grass, sorghum, bamboo, distillers grains, Distillers Dried Solubles (DDS), Distillers Dried Grains (DDG), Condensed Distillers Solubles (CDS), Distillers Wet Grains (DWG), Distillers Dried Grains with Solubles (DDGS), peels, citrus peels, bagasse, poplar, or algae. In some embodiments, the carbonaceous biomass comprises cellulosic or lignocellulosic materials. In some embodiments, the carbonaceous biomass is pretreated to make the polysaccharides more available to the bacterium.

Disclosed herein are systems for producing a fermentation end-product comprising: a fermentation vessel; a carbonaceous biomass; a genetically modified Clostridium bacterium that expresses a heterologous alcohol dehydrogenase protein, wherein the genetically modified Clostridium bacterium produces an increased yield of the fermentation end-product as compared to a non-genetically modified Clostridium bacterium; and, a medium. Also disclosed herein are systems for producing a fermentation end-product comprising: a fermentation vessel; a carbonaceous biomass; a genetically modified Clostridium bacterium that expresses a heterologous alcohol dehydrogenase protein, wherein the genetically modified Clostridium bacterium produces the fermentation end-product at an increased rate as compared to a non-genetically modified Clostridium bacterium; and, a medium. In some embodiments, the fermentation vessel is configured to house the medium and the microorganism, and wherein the carbonaceous biomass comprises a cellulosic and/or lignocellulosic material. In some embodiments, the heterologous alcohol dehydrogenase gene has greater than 90% identity to SEQ ID NO: 17. In some embodiments, the genetically modified Clostridium bacterium further comprises a genetic modification that expresses a pyruvate decarboxylase protein. In some embodiments, the pyruvate decarboxylase gene has greater than 90% identity to SEQ ID NO: 19. In some embodiments, the genetically modified Clostridium bacterium further comprises a genetic modification that expresses a heterologous acetyl-CoA synthetase protein. In some embodiments, the heterologous acetyl-CoA synthetase gene has greater than 90% identity to SEQ ID NO: 21. In some embodiments, the genetically modified Clostridium bacterium further comprises a genetic modification that inactivates an endogenous lactate dehydrogenase gene. In some embodiments, the fermentation end-product is an alcohol. In some embodiments, the alcohol is ethanol. In some embodiments, the genetically modified Clostridium bacterium is genetically modified C. phytofermentans. In some embodiments, the genetically modified Clostridium bacterium is genetically modified Clostridium sp Q.D. In some embodiments, the genetically modified Clostridium bacterium produces the fermentation end-product at a yield that is at least 1.5 times greater than the non-genetically modified Clostridium bacterium. In some embodiments, the genetically modified Clostridium bacterium produces the fermentation end-product at a rate at least 1.5 times greater than the non-genetically modified Clostridium bacterium. In some embodiments, the genetically modified Clostridium bacterium can hydrolyze hexose or pentose sugars. In some embodiments, the genetically modified Clostridium bacterium can hydrolyze and ferment hexose or pentose sugars. In some embodiments, the genetically modified Clostridium bacterium can hydrolyze and ferment cellulosic and/or lignocellulosic material. In some embodiments, the carbonaceous biomass comprises woody plant matter, non-woody plant matter, cellulosic material, lignocellulosic material, hemicellulosic material, carbohydrates, pectin, starch, inulin, fructans, glucans, corn, corn stover, sugar cane, grasses, switch grass, sorghum, bamboo, distillers grains, Distillers Dried Solubles (DDS), Distillers Dried Grains (DDG), Condensed Distillers Solubles (CDS), Distillers Wet Grains (DWG), Distillers Dried Grains with Solubles (DDGS), peels, citrus peels, bagasse, poplar, or algae. In some embodiments, the carbonaceous biomass comprises cellulosic or lignocellulosic materials. In some embodiments, the carbonaceous biomass is pretreated to make the polysaccharides more available to the bacterium.

Disclosed herein are fuel plants comprising a fermentation vessel configured to house a medium and a genetically modified Clostridium bacterium that expresses a heterologous pyruvate decarboxylase and/or a heterologous alcohol dehydrogenase, wherein the fermentation vessel comprises a cellulosic and/or lignocellulosic material, wherein the genetically modified Clostridium bacterium produces an increased yield of a fermentation end-product as compared to a non-genetically modified Clostridium bacterium. Also disclosed herein are fuel plants comprising a fermentation vessel configured to house a medium and a genetically modified Clostridium bacterium that expresses a heterologous pyruvate decarboxylase and/or a heterologous alcohol dehydrogenase, wherein the fermentation vessel comprises a cellulosic and/or lignocellulosic material, wherein the genetically modified Clostridium bacterium produces a fermentation end-product at an increased rate as compared to a non-genetically modified Clostridium bacterium. In some embodiments, the genetically modified Clostridium bacterium expresses a pyruvate decarboxylase and a heterologous alcohol dehydrogenase. In some embodiments, the cellulosic and/or lignocellulosic material is pretreated.

Further aspects of the disclosure are fermentation end-products produced by any of the methods disclosed herein.

Disclosed herein are genetically modified microorganisms that express a pyruvate decarboxylase protein, wherein the microorganisms produce an increased yield of a fermentation end-product as compared to non-genetically modified microorganisms. Also disclosed herein genetically modified microorganisms that express a pyruvate decarboxylase protein, wherein the genetically modified microorganisms produce a fermentation end-product at an increased rate as compared to non-genetically modified microorganisms. In some embodiments, a genetically modified microorganism further comprises a genetic modification that expresses a heterologous alcohol dehydrogenase protein. Also disclosed herein are genetically modified microorganisms that express a heterologous alcohol dehydrogenase protein, wherein the genetically modified microorganisms produce an increased yield of a fermentation end-product as compared to non-genetically modified microorganisms. Also disclosed herein are genetically modified microorganisms that express a heterologous alcohol dehydrogenase protein, wherein the genetically modified microorganisms produce a fermentation end-product at a greater rate as compared to non-genetically modified microorganisms. In some embodiments, the pyruvate decarboxylase protein is endogenous or heterologous. In some embodiments, the pyruvate decarboxylase gene has greater than 90% identity to SEQ ID NO: 19. In some embodiments, the heterologous alcohol dehydrogenase gene has greater than 90% identity to SEQ ID NO: 17. In some embodiments, a genetically modified microorganism further comprises a genetic modification that expresses a heterologous acetyl-CoA synthetase protein. In some embodiments, the heterologous acetyl-CoA synthetase gene has greater than 90% identity to SEQ ID NO: 21. In some embodiments, the genetically modified microorganism can hydrolyze and ferment hemicellulose and lignocellulose. In some embodiments, the genetically modified microorganism is mesophilic. In some embodiments, a genetically modified microorganism further comprises a genetic modification that inactivates an endogenous lactate dehydrogenase gene. In some embodiments, the fermentation end-product is an alcohol. In some embodiments, the alcohol is ethanol. In some embodiments, the genetically modified microorganism is a genetically modified Clostridium bacterium. In some embodiments, the genetically modified microorganism is genetically modified C. phytofermentans or Clostridium sp Q.D. In some embodiments, the genetically modified microorganism produces the fermentation end-product at a yield that is at least 1.5 times greater than the non-genetically modified microorganism. In some embodiments, the genetically modified microorganism produces the fermentation end-product at a rate at least 1.5 times greater than the non-genetically modified microorganism. In some embodiments, the genetically modified microorganism can hydrolyze hexose or pentose sugars. In some embodiments, the genetically modified microorganism can hydrolyze and ferment hexose or pentose sugars.

Disclosed herein are microorganisms from NRRL Accession No. NRRL B-50361, NRRL B-50362, NRRL B-50363, NRRL B-50364, NRRL B-50436, or NRRL B-50437, genetically modified to express a heterologous alcohol dehydrogenase protein and or a pyruvate decarboxylase protein, wherein the microorganisms produce an increased yield of an alcohol as compared to non-genetically modified microorganisms. In one embodiment, the microorganism is genetically modified to express a heterologous alcohol dehydrogenase protein and a pyruvate decarboxylase protein.

Disclosed herein are processes for producing a fermentation end-product comprising: contacting a carbonaceous biomass with a microorganism genetically modified to express a heterologous alcohol dehydrogenase protein and/or a pyruvate decarboxylase protein; and, allowing sufficient time for hydrolysis and fermentation to produce the fermentation end-product. In one embodiment, the microorganism is genetically modified to express a heterologous alcohol dehydrogenase protein and a pyruvate decarboxylase protein. In some embodiments, the genetically modified microorganism produces an increased yield of the fermentation end-product as compared to a non-genetically modified microorganism. In some embodiments, the genetically modified microorganism produces the fermentation end-product at a greater rate as compared to a non-genetically modified microorganism. In some embodiments, the genetically modified microorganism further comprises a genetic modification that inactivates an endogenous lactate dehydrogenase gene. In some embodiments, the genetically modified microorganism further comprises a genetic modification that expresses an acetyl-CoA synthetase protein. In some embodiments, the genetically modified microorganism is gram negative. In some embodiments, the genetically modified microorganism is gram positive. In some embodiments, the genetically modified microorganism is mesophilic. In some embodiments, the genetically modified microorganism is a Clostridium species. In some embodiments, the Clostridium species is C. phytofermentans. In some embodiments, the Clostridium species is Clostridium sp Q.D. In some embodiments, the fermentation end-product is produced at a yield that is at least 1.5 times greater than a process using a non-genetically modified microorganism. In some embodiments, the fermentation end-product is produced at a rate at least 1.5 times greater than a process using a non-genetically modified microorganism. In some embodiments, the biomass comprises cellulosic or lignocellulosic materials. In some embodiments, the biomass comprises woody plant matter, non-woody plant matter, cellulosic material, lignocellulosic material, hemicellulosic material, carbohydrates, pectin, starch, inulin, fructans, glucans, corn, corn stover, sugar cane, grasses, switch grass, sorghum, bamboo, distillers grains, Distillers Dried Solubles (DDS), Distillers Dried Grains (DDG), Condensed Distillers Solubles (CDS), Distillers Wet Grains (DWG), Distillers Dried Grains with Solubles (DDGS), peels, citrus peels, bagasse, poplar, or algae. In some embodiments, the process occurs at a temperature between 10° C. and 35° C. In some embodiments, the fermentation end-product is an alcohol. In some embodiments, the alcohol is ethanol.

Disclosed herein are Clostridium bacteria that convert pyruvate directly to acetaldehyde. Also disclosed herein are Clostridium bacteria that: convert pyruvate directly to acetaldehyde; and, convert acetaldehyde directly to ethanol.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of these embodiments are set forth with particularity in the appended claims. A better understanding of the features and advantages of the embodiments will be obtained by reference to the following detailed description that sets forth illustrative embodiments, in which the principles of the invention are utilized, and the accompanying drawings of which:

FIG. 1 illustrates a representation of several end-products synthesized from pyruvate in the glycolysis metabolic pathway.

FIG. 2 illustrates an ethanol production pathway of an anaerobic organism.

FIG. 3 illustrates an ethanol production pathway of an anaerobic organism that expresses an endogenous alcohol dehydrogenase and a heterologous alcohol dehydrogenase such as the alcohol dehydrogenase gene adhB, from Zymomonas mobilis.

FIG. 4 illustrates an ethanol production pathway of an anaerobic organism that expresses an endogenous alcohol dehydrogenase and a pyruvate decarboxylase to allow direct conversion of pyruvate to acetaldehyde; optionally a heterologous alcohol dehydrogenase is also expressed.

FIG. 5 illustrates an ethanol production pathway of an anaerobic organism that expresses an acetyl-CoA synthetase.

FIG. 6 illustrates a method for producing fermentation end products from biomass by first treating biomass with an acid at elevated temperature and pressure in a hydrolysis unit.

FIG. 7 illustrates a method for producing fermentation end products from biomass by using solvent extraction or separation methods.

FIG. 8 illustrates a method for producing fermentation end products from biomass by charging biomass to a fermentation vessel.

FIG. 9 A-C illustrates pretreatments that produce hexose or pentose saccharides or oligomers that are then unprocessed or processed further and either fermented separately or together.

FIG. 10 illustrates the primers designed for inactivating LDH genes.

FIG. 11 illustrates plasmids containing Cphy_{—}1232 and Cphy_{—}1117 cloned fragments.

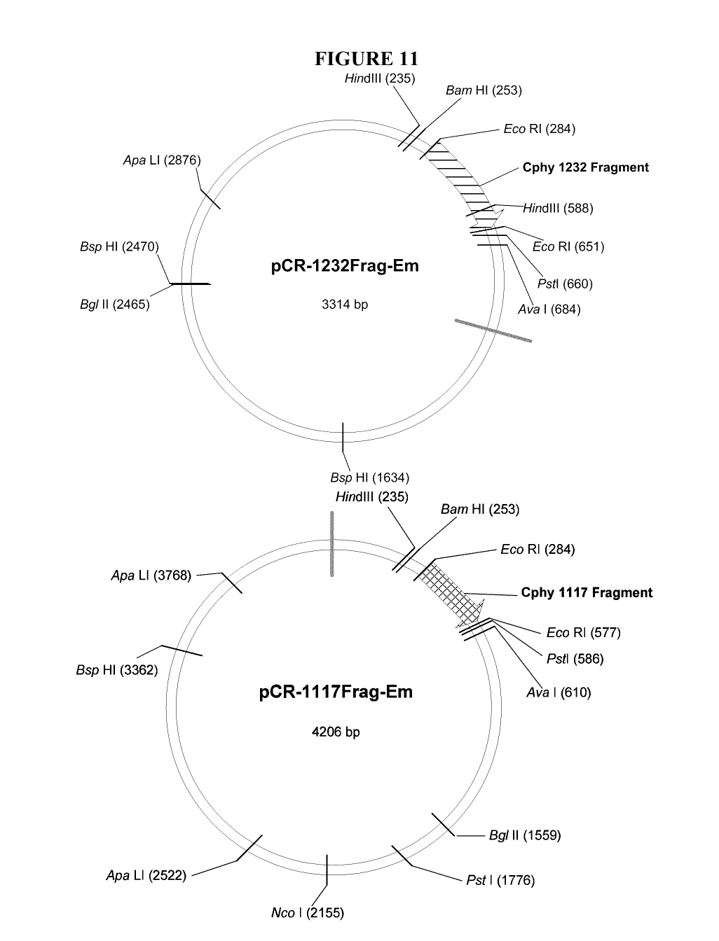

FIG. 12 illustrates the pQSeq plasmid.

FIG. 13 illustrates the pQSeq plasmid comprising Cphy_{—}1232 and Cphy_{—}1117 cloned fragments.

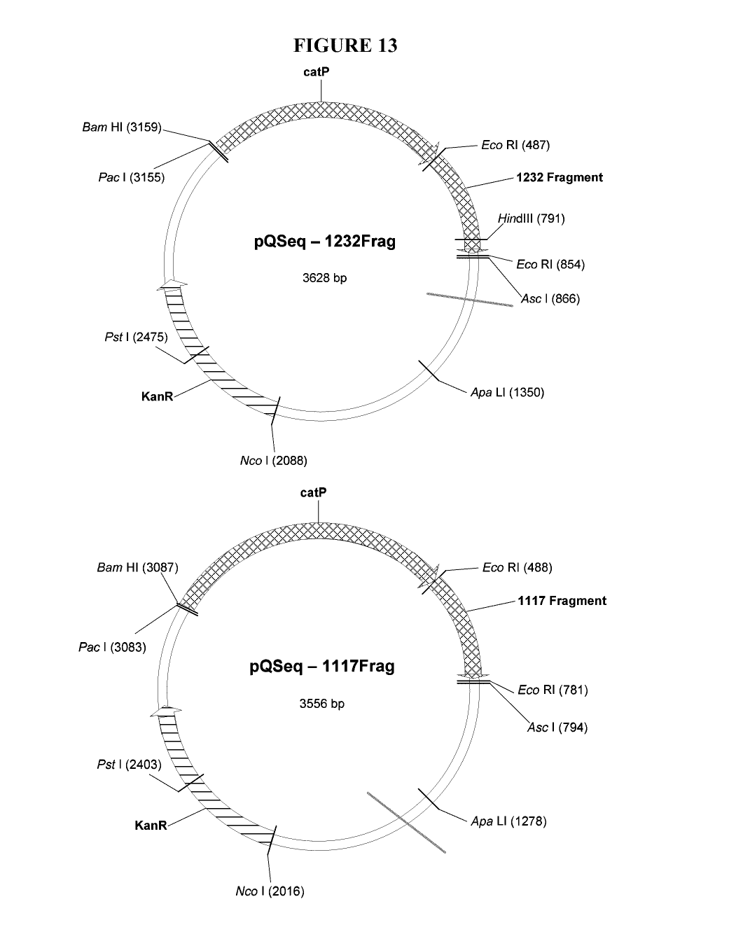

FIG. 14 illustrates the plasmid pQInt.

FIG. 15 illustrates the plasmid pQInt1.

FIG. 16 illustrates the plasmid pQInt2.

FIG. 17 illustrates CMC-congo red plate and Cellazyme Y assays.

FIG. 18 illustrates a plasmid map for pIMP.1, a non-conjugal shuttle vector that can replicate in

Escherichia coli and C. phytofermentans.

FIG. 19 illustrates a plasmid map of pIMPCphy.

FIG. 20 illustrates a plasmid map for pCphyP3510.

FIG. 21 illustrates a plasmid map for pCphyP3510-1163.

FIG. 22 illustrates the plasmid pQInt.

FIG. 23 illustrates the plasmid pQP3558-PDC/AdhB.

FIG. 24 illustrates operon construction for pQP3558-PDC/AdhB.

FIG. 25: illustrates ethanol production of recombinant C. phytofermentans.

### DETAILED DESCRIPTION OF THE INVENTION

The following description and examples illustrate embodiments of the invention in detail. It is to be understood that this invention is not limited to the particular methodology, protocols, cell lines, constructs and reagents described herein and as such can vary. Those of skill in the art will recognize that there are numerous variations and modifications of this invention that are encompassed within its scope.

The invention comprises methods and compositions directed to saccharification and fermentation of various biomass substrates to desired products.

In one embodiment, products include modified strains of microorganisms, including algae, fungi, gram-positive and gram-negative bacteria, including species of Clostridium, including C. phytofermentans that can be used in production of chemicals from lignocellulosic, cellulosic, hemicellulosic, algal, and other plant-based feedstocks or plant polysaccharides. Products further include the chemical compounds, fermentive-end products, biofuels and the like from the processes using these modified organisms. Described herein are also methods of producing chemical compounds, fermentive-end products, biofuels and the like using these referenced microorganisms.

In another embodiment, organisms are genetically-modified strains of bacteria, including Clostridium sp., including C. phytofermentans. Bacteria comprising altered expression or structure of a gene or genes relative to the original organisms strain, wherein such genetic modifications result in increased efficiency of chemical production. In some embodiments, the genetic modifications are introduced by genetic recombination. In some embodiments, the genetic modifications are introduced by nucleic acid transformation. In further embodiments, the genetic modifications encompass inactivation of one or more genes of Clostridium sp., including C. phytofermentans through any number of genetic methods, including but not limited to single-crossover or double-crossover gene replacement, transposable element insertion, integrational plasmid technology (e.g., using non-replicative or replicative integrative plasmids), targeted gene inactivation using group II intron-based Targetron technology (Chen Y. et al. (2005) Appl Environ Microbial 71:7542-7547), or targeted gene inactivation using ClosTron Group II intron directed mutagenesis (Heap J T et al. (2010) J. Microbiol Methods 80:49-55. The restriction and modification system of a Clostridium sp. can be modified to increase the efficiency of transformation with unmethylated DNA (Dong H. et al. (2010) PLOS One 5(2): e9038). Interspecific conjugation (for example, with E. coli), can be used to transfer nucleic acid into a Clostridium sp. (Tolonen A C et al. (2009) Molecular Microbiology, 74: 1300-1313). In some strains, genetic modification can comprise inactivation of one or more endogenous nucleic acid sequence(s) and also comprise introduction and activation of heterologous or exogenous nucleic acid sequence(s) and promoters.

In some variations, the recombinant C. phytofermentans organisms described herein comprise a heterologous nucleic acid sequence. In some variations, the recombinant C. phytofermentans comprise one or more introduced heterologous nucleic acid(s). In some embodiments, the heterologous nucleic acid sequence is controlled by an inducible promoter. In some variations, expression of the heterologous nucleic acid sequence is controlled by a constitutive promoter.

The discovery that C. phytofermentans microorganisms can produce a variety of chemical products is a great advantage over other fermenting organisms. C. phytofermentans is capable of simultaneous hydrolysis and fermentation of a variety of feedstocks comprised of cellulosic, hemicellulosic or lignocellulosic materials, thus eliminating or drastically reducing the need for hydrolysis of polysaccharides prior to fermentation of sugars. Further, C. phytofermentans utilizes both hexose and pentose polysaccharides and sugars, producing a highly efficient yield from feedstocks.

Another advantage of C. phytofermentans is its ability to ferment oligomers, resulting in a great cost savings for processors that have to pretreat biomass prior to fermentation. To produce a stream of monosaccharides for most fermenting organisms such as yeasts, that cannot ferment oligomers or polymeric saccharides, harsh prolonged pretreatment is required. This results in higher costs due to the chemical and energy requirements and to the loss of sugars during the pretreatment, as well as the increased production of breakdown products and inhibitors. Because C. phytofermentans can hydrolyze polysaccharides and ferment oligomers, it does not require severe biomass pretreatment resulting in a higher conversion efficiency of carbohydrate in biomass and increased yields at reduced costs.

### DEFINITIONS

Unless characterized differently, technical and scientific terms used herein have the same meaning as commonly understood by one of ordinary skill in the art to which this invention belongs.

The term “about” as used herein refers to a range that is 15% plus or minus from a stated numerical value within the context of the particular usage. For example, about 10 would include a range from 8.5 to 11.5.

“Optional” or “optionally” means that the subsequently described event or circumstance may or may not occur, and that the description includes instances where said event or circumstance occurs and instances where it does not. For example, the phrase “the medium can optionally contain glucose” means that the medium may or may not contain glucose as an ingredient and that the description includes both media containing glucose and media not containing glucose.

The term “enzyme reactive conditions” as used herein refers to environmental conditions (i.e., such factors as temperature, pH, or lack of inhibiting substances) which will permit the enzyme to function. Enzyme reactive conditions can be either in vitro, such as in a test tube, or in vivo, such as within a cell.

The terms “function” and “functional” and the like as used herein refer to a biological or enzymatic function.

The term “gene” as used herein, refers to a unit of inheritance that occupies a specific locus on a chromosome and consists of transcriptional and/or translational regulatory sequences and/or a coding region and/or non-translated sequences (i.e., introns, 5′ and 3′ untranslated sequences).

The term “host cell” includes an individual cell or cell culture which can be or has been a recipient of any recombinant vector(s) or isolated polynucleotide. Host cells include progeny of a single host cell, and the progeny can not necessarily be completely identical (in morphology or in total DNA complement) to the original parent cell due to natural, accidental, or deliberate mutation and/or change. A host cell includes cells transfected, transformed, or infected in vivo or in vitro with a recombinant vector or a polynucleotide. A host cell which comprises a recombinant vector is a recombinant host cell, recombinant cell, or recombinant microorganism.

The term “isolated” as used herein, refers to material that is substantially or essentially free from components that normally accompany it in its native state. For example, an “isolated polynucleotide”, as used herein, refers to a polynucleotide, which has been purified from the sequences which flank it in a naturally-occurring state, e.g., a DNA fragment which has been removed from the sequences that are normally adjacent to the fragment. Alternatively, an “isolated peptide” or an “isolated polypeptide” and the like, as used herein, refer to in vitro isolation and/or purification of a peptide or polypeptide molecule from its natural cellular environment, and from association with other components of the cell, i.e., it is not associated with in vivo substances.

The terms “increased” or “increasing” as used herein, refers to the ability of one or more recombinant microorganisms to produce a greater amount of a given product or molecule (e.g., commodity chemical, biofuel, or intermediate product thereof) as compared to a control microorganism, such as an unmodified microorganism or a differently modified microorganism. An “increased” amount is typically a “statistically significant” amount, and can include an increase that is 1, 2, 3, 4, 5, 6, 7, 8, 9, 10, 15, 20, 30 or more times (including all integers and decimal points in between, e.g., 1.5, 1.6, 1.7. 1.8, etc.) the amount produced by an unmodified microorganism or a differently modified microorganism.

The term “operably linked” as used herein means placing a gene under the regulatory control of a promoter, which then controls the transcription and optionally the translation of the gene. In one example for the construction of promoter/structural gene combinations, the genetic sequence or promoter is positioned at a distance from the gene transcription start site that is approximately the same as the distance between that genetic sequence or promoter and the gene it controls in its natural setting; i.e. the gene from which the genetic sequence or promoter is derived. As is known in the art, some variation in this distance can be accommodated without loss of function Similarly, a regulatory sequence element can be positioned with respect to a gene to be placed under its control in the same position as the element is situated in its in its natural setting with respect to the native gene it controls.

The term “constitutive promoter” refers to a polynucleotide sequence that induces transcription or is typically active, (i.e., promotes transcription), under most conditions, such as those that occur in a host cell. A constitutive promoter is generally active in a host cell through a variety of different environmental conditions.

The term “inducible promoter” refers to a polynucleotide sequence that induces transcription or is typically active only under certain conditions, such as in the presence of a specific transcription factor or transcription factor complex, a given molecule factor (e.g., IPTG) or a given environmental condition (e.g., CO₂ concentration, nutrient levels, light, heat). In the absence of that condition, inducible promoters typically do not allow significant or measurable levels of transcriptional activity.

The term “low temperature-adapted” refers to an enzyme that has been adapted to have optimal activity at a temperature below about 20° C., such as 19° C., 18° C., 17° C., 16° C., 15° C., 14° C., 13° C., 12° C., 11° C., 10° C., 9° C., 8° C., 7° C., 6° C., 5° C., 4° C., 3° C., 2° C., 1° C.-1° C., −2° C., −3° C., −4° C., −5° C., −6° C., −7° C., −8° C., −9° C., −10° C., −11° C., −12° C., −13° C., −14° C., or −15° C.

The terms “polynucleotide” or “nucleic acid” as used herein designates RNA, mRNA, cRNA, rRNA, DNA, or cDNA. The term typically refers to polymeric form of nucleotides of at least 10 bases in length, either ribonucleotides or deoxyribonucleotides or a modified form of either type of nucleotide. The term includes single and double stranded forms of DNA.

As will be understood by those skilled in the art, a polynucleotide sequence can include genomic sequences, extra-genomic and plasmid-encoded sequences and smaller engineered gene segments that express, or can be adapted to express, proteins, polypeptides, peptides and the like. Such segments can be naturally isolated, or modified synthetically by the hand of man.

Polynucleotides can be single-stranded (coding or antisense) or double-stranded, and can be DNA (genomic, cDNA or synthetic) or RNA molecules. In one embodiment, additional coding or non-coding sequences can, but need not, be present within a polynucleotide, and a polynucleotide can, but need not, be linked to other molecules and/or support materials.

Polynucleotides can comprise a native sequence (i.e., an endogenous sequence) or can comprise a variant, or a biological functional equivalent of such a sequence. Polynucleotide variants can contain one or more base substitutions, additions, deletions and/or insertions, as further described below. In one embodiment a polynucleotide variant encodes a polypeptide with the same sequence as the native protein. In another embodiment a polynucleotide variant encodes a polypeptide with substantially similar enzymatic activity as the native protein. In another embodiment a polynucleotide variant encodes a protein with increased enzymatic activity relative to the native polypeptide. The effect on the enzymatic activity of the encoded polypeptide can generally be assessed as described herein.

A polynucleotide, can be combined with other DNA sequences, such as promoters, polyadenylation signals, additional restriction enzyme sites, multiple cloning sites, other coding segments, and the like, such that their overall length can vary considerably. In one embodiment, the maximum length of a polynucleotide sequence which can be used to transform a microorganism is governed only by the nature of the recombinant protocol employed.

The terms “polynucleotide variant” and “variant” and the like refer to polynucleotides that display substantial sequence identity with any of the reference polynucleotide sequences or genes described herein, and to polynucleotides that hybridize with any polynucleotide reference sequence described herein, or any polynucleotide coding sequence of any gene or protein referred to herein, under low stringency, medium stringency, high stringency, or very high stringency conditions that are defined hereinafter and known in the art. These terms also encompass polynucleotides that are distinguished from a reference polynucleotide by the addition, deletion or substitution of at least one nucleotide. Accordingly, the terms “polynucleotide variant” and “variant” include polynucleotides in which one or more nucleotides have been added or deleted, or replaced with different nucleotides. In this regard, it is well understood in the art that certain alterations inclusive of mutations, additions, deletions and substitutions can be made to a reference polynucleotide whereby the altered polynucleotide retains the biological function or activity of the reference polynucleotide, or has increased activity in relation to the reference polynucleotide (i.e., optimized). Polynucleotide variants include, for example, polynucleotides having at least 50% (and at least 51% to at least 99% and all integer percentages in between) sequence identity with a reference polynucleotide described herein.

The terms “polynucleotide variant” and “variant” also include naturally-occurring allelic variants that encode these enzymes. Examples of naturally-occurring variants include allelic variants (same locus), homologs (different locus), and orthologs (different organism). Naturally occurring variants such as these can be identified and isolated using well-known molecular biology techniques including, for example, various polymerase chain reaction (PCR) and hybridization-based techniques as known in the art. Naturally occurring variants can be isolated from any organism that encodes one or more genes having a suitable enzymatic activity described herein (e.g., C≡C ligase, diol dehydrogenase, pectate lyase, alginate lyase, diol dehydratase, transporter, etc.).

Non-naturally occurring variants can be made by mutagenesis techniques, including those applied to polynucleotides, cells, or microorganisms. The variants can contain nucleotide substitutions, deletions, inversions and insertions. Variation can occur in either or both the coding and non-coding regions. In certain aspects, non-naturally occurring variants can have been optimized for use in a given microorganism (e.g., E. coli), such as by engineering and screening the enzymes for increased activity, stability, or any other desirable feature. The variations can produce both conservative and non-conservative amino acid substitutions (as compared to the originally encoded product). For polynucleotide sequences, conservative variants include those sequences that, because of the degeneracy of the genetic code, encode the amino acid sequence of a reference polypeptide. Variant polynucleotide sequences also include synthetically derived nucleotide sequences, such as those generated, for example, by using site-directed mutagenesis but which still encode a biologically active polypeptide. Generally, variants of a reference polynucleotide sequence will have at least about 30%, 40% 50%, 55%, 60%, 65%, 70%, generally at least about 75%, 80%, 85%, 90% to 95% or more, and even about 97% or 98% or more sequence identity to that particular nucleotide sequence as determined by sequence alignment programs described elsewhere herein using default parameters. In one embodiment a variant polynucleotide sequence encodes a protein with substantially similar activity compared to a protein encoded by the respective reference polynucleotide sequence. Substantially similar activity means variant protein activity that is within +/−15% of the activity of a protein encoded by the respective reference polynucleotide sequence. In another embodiment a variant polynucleotide sequence encodes a protein with greater activity compared to a protein encoded by the respective reference polynucleotide sequence.

“Stringent conditions” refers to the washing conditions used in a hybridization protocol. In general, the washing conditions should be a combination of temperature and salt concentration chosen so that the denaturation temperature is approximately 5° C. to 20° C. below the calculated melting temperature (Tₘ) of the nucleic acid hybrid under study. In one embodiment, the denaturation temperature is approximately 5° C., 6° C., 7° C., 8° C., 9° C., 10° C., 11° C., 12° C., 13° C., 14° C., 15° C., 16° C., 17° C., 18° C., 19° C., or 20° C. below the calculated Tₘ of the nucleic acid hybrid under study. The temperature and salt conditions are readily determined empirically in preliminary experiments in which samples of reference DNA immobilized on filters are hybridized to the probe or polypeptide-coding nucleic acid of interest and then washed under conditions of different stringencies. The Tₘ of such an oligonucleotide can be estimated by allowing 2° C. for each A or T nucleotide, and 4° C. for each G or C. For example, an 18 nucleotide probe of 50% G+C would, therefore, have an approximate Tₘ of 54° C. Stringent conditions are known to one of skill in the art. See, for example, Sambrook et al. (2001). The following is an exemplary set of hybridization conditions and is not limiting:

### Very High Stringency

Hybridization: 5× saline-sodium citrate buffer (SSC; 1×SSC: 0.1 M sodium chloride, 15 mM trisodium citrate, pH 7.0) at 65° C. for 16 hours. Wash twice: 2×SSC at room temperature (RT) for 15 minutes each. Wash twice: 0.5×SSC at 65° C. for 20 minutes each.

### High Stringency

Hybridization: 5×-6×SSC at 65° C.-70° C. for 16-20 hours. Wash twice: 2×SSC at RT for 5-20 minutes each. Wash twice: 1×SSC at 55° C.-70° C. for 30 minutes each.

### Low Stringency

Hybridization: 6×SSC at RT to 55° C. for 16-20 hours. Wash at least twice: 2×-3×SSC at RT to 55° C. for 20-30 minutes each.

The genetic code is redundant in that it contains 64 different codons (triplet nucleotide sequence) but only codes for 22 standard amino acids and a stop signal (Table 1). Due to the degeneracy of the genetic code, nucleotides within a protein-coding polynucleotide sequence can be substituted without altering the encoded amino acid sequence. These changes (e.g. substitutions, mutations, optimizations, etc.) are therefore “silent”. It is thus contemplated that various changes can be made within a disclosed nucleic acid sequence without any loss of biological activity relating to either the polynucleotide sequence or the encoded peptide sequence.

In one embodiment, a polynucleotide comprises codons, within a coding sequence, that are optimized to increase the thermostability of an mRNA transcribed from the polynucleotide. In one embodiment, this optimization does not change the amino acid sequence encoded by the polynucleotide (i.e. they are “silent”). In another embodiment, a polynucleotide comprises codons, within a protein coding sequence, that are optimized to increase translation efficiency of an mRNA transcribed from the polynucleotide in a host cell. In one embodiment, this optimization is silent (does not change the amino acid sequence encoded by the polynucleotide).

The RNA codon table below (Table 1) shows the 64 codons and the encoded amino acid for each.

The direction of the mRNA is 5′ to 3′.

TABLE 1

| 1st | 2nd base | | | |
|---|---|---|---|---|
| base | U | C | A | G |
| U | UUU (Phe/F) | UCU (Ser/S) Serine | UAU (Tyr/Y) Tyrosine | UGU (Cys/C) Cysteine |
| | Phenylalanine | | | |
| | UUC (Phe/F) | UCC (Ser/S) Serine | UAC (Tyr/Y) Tyrosine | UGC (Cys/C) Cysteine |
| | Phenylalanine | | | |
| | UUA (Leu/L) Leucine | UCA (Ser/S) Serine | UAA Ochre (Stop) | UGA Opal (Stop) |
| | UUG (Leu/L) Leucine | UCG (Ser/S) Serine | UAG Amber (Stop) | UGG (Trp/W) |
| | | | | Tryptophan |

| | | | | |
|---|---|---|---|---|
| C | CUU (Leu/L) Leucine | CCU (Pro/P) Proline | CAU (His/H) Histidine | CGU (Arg/R) Arginine |
| | CUC (Leu/L) Leucine | CCC (Pro/P) Proline | CAC (His/H) Histidine | CGC (Arg/R) Arginine |
| | CUA (Leu/L) Leucine | CCA (Pro/P) Proline | CAA (Gln/Q) Glutamine | CGA (Arg/R) Arginine |
| | CUG (Leu/L) Leucine | CCG (Pro/P) Proline | CAG (Gln/Q) Glutamine | CGG (Arg/R) Arginine |
| A | AUU (Ile/I) Isoleucine | ACU (Thr/T) | AAU (Asn/N) | AGU (Ser/S) Serine |
| | | Threonine | Asparagine | |
| | AUC (Ile/I) Isoleucine | ACC (Thr/T) | AAC (Asn/N) | AGC (Ser/S) Serine |
| | | Threonine | Asparagine | |
| | AUA (Ile/I) Isoleucine | ACA (Thr/T) | AAA (Lys/K) Lysine | AGA (Arg/R) Arginine |
| | | Threonine | | |
| | AUG^{[A]} (Met/M) | ACG (Thr/T) | AAG (Lys/K) Lysine | AGG (Arg/R) Arginine |
| | Methionine | Threonine | | |
| G | GUU (Val/V) Valine | GCU (Ala/A) | GAU (Asp/D) Aspartic | GGU (Gly/G) Glycine |
| | | Alanine | acid | |
| | GUC (Val/V) Valine | GCC (Ala/A) | GAC (Asp/D) Aspartic | GGC (Gly/G) Glycine |
| | | Alanine | acid | |
| | GUA (Val/V) Valine | GCA (Ala/A) | GAA (Glu/E) Glutamic | GGA (Gly/G) Glycine |
| | | Alanine | acid | |
| | GUG (Val/V) Valine | GCG (Ala/A) | GAG (Glu/E) Glutamic | GGG (Gly/G) Glycine |
| | | Alanine | acid | |

^{A}The codon AUG both codes for methionine and serves as an initiation site: the first
AUG in an mRNA's coding region is where translation into protein begins.

It will be appreciated by one of skill in the art that amino acids can be substituted for other amino acids in a protein sequence without appreciable loss of the desired activity. It is thus contemplated that various changes can be made in the peptide sequences of the disclosed protein sequences, or their corresponding nucleic acid sequences without appreciable loss of the biological activity.

In making such changes, the hydropathic index of amino acids can be considered. The importance of the hydropathic amino acid index in conferring interactive biological function on a protein is generally understood in the art (Kyte and Doolittle, J. Mol. Biol., 157: 105-132, 1982). It is accepted that the relative hydropathic character of the amino acid contributes to the secondary structure of the resultant protein, which in turn defines the interaction of the protein with other molecules, for example, enzymes, substrates, receptors, DNA, antibodies, antigens, and the like.

Amino acids have been assigned a hydropathic index on the basis of their hydrophobicity and charge characteristics. These are: isoleucine (+4.5); valine (+4.2); leucine (+3.8); phenylalanine (+2.8); cysteine (+2.5); methionine (+1.9); alanine (+1.8); glycine (−0.4); threonine (−0.7); serine (−0.8); tryptophan (−0.9); tyrosine (−1.3); proline (−1.6); histidine (−3.2); glutamate/glutamine/aspartate/asparagine (−3.5); lysine (−3.9); and arginine (−4.5).

It is known in the art that certain amino acids can be substituted by other amino acids having a similar hydropathic index or score and result in a protein with similar biological activity, i.e., still obtain a biologically-functional protein. In one embodiment, the substitution of amino acids whose hydropathic indices are within +/−0.2 is preferred, those within +/−0.1 are more preferred, and those within +/−0.5 are most preferred.

It is also understood in the art that the substitution of like amino acids can be made effectively on the basis of hydrophilicity. U.S. Pat. No. 4,554,101 (Hopp, which is herein incorporated by reference in its entirety) states that the greatest local average hydrophilicity of a protein, as governed by the hydrophilicity of its adjacent amino acids, correlates with a biological property of the protein. The following hydrophilicity values have been assigned to amino acids: arginine/lysine (+3.0); aspartate/glutamate (+3.0.+−0.1); serine (+0.3); asparagine/glutamine (+0.2); glycine (0); threonine (−0.4); proline (−0.5.+-0.1); alanine/histidine (−0.5); cysteine (−1.0); methionine (−1.3); valine (−1.5); leucine/isoleucine (−1.8); tyrosine (−2.3); phenylalanine (−2.5); and tryptophan (−3.4).

It is understood that an amino acid can be substituted by another amino acid having a similar hydrophilicity score and still result in a protein with similar biological activity, i.e., still obtain a biologically functional protein. In one embodiment the substitution of amino acids whose hydropathic indices are within +/−0.2 is preferred, those within +/−0.1 are more preferred, and those within. +/−.0.5 are most preferred.

As outlined above, amino acid substitutions can be based on the relative similarity of the amino acid side-chain substituents, for example, their hydrophobicity, hydrophilicity, charge, size, and the like. Exemplary substitutions which take any of the foregoing characteristics into consideration are well known to those of skill in the art and include: arginine and lysine; glutamate and aspartate; serine and threonine; glutamine and asparagine; and valine, leucine, and isoleucine. Changes which are not expected to be advantageous can also be used if these resulting proteins have the same or improved characteristics, relative to the unmodified polypeptide from which they are engineered.

In one embodiment, a method is provided for that uses variants of full-length polypeptides having any of the enzymatic activities described herein, truncated fragments of these full-length polypeptides, variants of truncated fragments, as well as their related biologically active fragments. Typically, biologically active fragments of a polypeptide can participate in an interaction, for example, an intra-molecular or an inter-molecular interaction. An inter-molecular interaction can be a specific binding interaction or an enzymatic interaction (e.g., the interaction can be transient and a covalent bond is formed or broken). Biologically active fragments of a polypeptide/enzyme an enzymatic activity described herein include peptides comprising amino acid sequences sufficiently similar to, or derived from, the amino acid sequences of a (putative) full-length reference polypeptide sequence. Typically, biologically active fragments comprise a domain or motif with at least one enzymatic activity, and can include one or more (and in some cases all) of the various active domains. A biologically active fragment of a an enzyme can be a polypeptide fragment which is, for example, 10, 11, 12, 13, 14, 15, 16, 17, 18, 19, 20, 21, 22, 23, 24, 25, 26, 27, 28, 29, 30, 40, 50, 60, 70, 80, 90, 100, 110, 120, 130, 140, 150, 160, 170, 180, 190, 200, 220, 240, 260, 280, 300, 320, 340, 360, 380, 400, 450, 500, 600 or more contiguous amino acids, including all integers in between, of a reference polypeptide sequence. In certain embodiments, a biologically active fragment comprises a conserved enzymatic sequence, domain, or motif, as described elsewhere herein and known in the art. Suitably, the biologically-active fragment has no less than about 1%, 10%, 25%, or 50% of an activity of the wild-type polypeptide from which it is derived.

The term “exogenous” as used herein, refers to a polynucleotide sequence or polypeptide that does not naturally occur in a given wild-type cell or microorganism, but is typically introduced into the cell by a molecular biological technique, i.e., engineering to produce a recombinant microorganism. Examples of “exogenous” polynucleotides include vectors, plasmids, and/or man-made nucleic acid constructs encoding a desired protein or enzyme.

The term “endogenous” as used herein, refers to naturally-occurring polynucleotide sequences or polypeptides that can be found in a given wild-type cell or microorganism. For example, certain naturally-occurring bacterial or yeast species do not typically contain a benzaldehyde lyase gene, and, therefore, do not comprise an “endogenous” polynucleotide sequence that encodes a benzaldehyde lyase. In this regard, it is also noted that even though a microorganism can comprise an endogenous copy of a given polynucleotide sequence or gene, the introduction of a plasmid or vector encoding that sequence, such as to over-express or otherwise regulate the expression of the encoded protein, represents an “exogenous” copy of that gene or polynucleotide sequence. Any of the of pathways, genes, or enzymes described herein can utilize or rely on an “endogenous” sequence, or can be provided as one or more “exogenous” polynucleotide sequences, and/or can be used according to the endogenous sequences already contained within a given microorganism.

The term “sequence identity” for example, comprising a “sequence 50% identical to,” as used herein, refers to the extent that sequences are identical on a nucleotide-by-nucleotide basis or an amino acid-by-amino acid basis over a window of comparison. Thus, a “percentage of sequence identity” can be calculated by comparing two optimally aligned sequences over the window of comparison, determining the number of positions at which the identical nucleic acid base (e.g., A, T, C, G, I) or the identical amino acid residue (e.g., Ala, Pro, Ser, Thr, Gly, Val, Leu, Ile, Phe, Tyr, Trp, Lys, Arg, His, Asp, Glu, Asn, Gln, Cys and Met) occurs in both sequences to yield the number of matched positions, dividing the number of matched positions by the total number of positions in the window of comparison (i.e., the window size), and multiplying the result by 100 to yield the percentage of sequence identity.

The terms used to describe sequence relationships between two or more polynucleotides or polypeptides include “reference sequence”, “comparison window”, “sequence identity”, “percentage of sequence identity” and “substantial identity”. A “reference sequence” is at least 12 but frequently 15 to 18 and often at least 25 monomer units, inclusive of nucleotides and amino acid residues, in length. Because two polynucleotides can each comprise (1) a sequence (i.e., only a portion of the complete polynucleotide sequence) that is similar between the two polynucleotides, and (2) a sequence that is divergent between the two polynucleotides, sequence comparisons between two (or more) polynucleotides are typically performed by comparing sequences of the two polynucleotides over a “comparison window” to identify and compare local regions of sequence similarity. A “comparison window” refers to a conceptual segment of at least 6 contiguous positions, usually about 50 to about 100, more usually about 100 to about 150 in which a sequence is compared to a reference sequence of the same number of contiguous positions after the two sequences are optimally aligned. The comparison window can comprise additions or deletions (i.e., gaps) of about 20% or less as compared to the reference sequence (which does not comprise additions or deletions) for optimal alignment of the two sequences. Optimal alignment of sequences for aligning a comparison window can be conducted by computerized implementations of algorithms (GAP, BESTFIT, FASTA, and TFASTA in the Wisconsin Genetics Software Package Release 7.0, Genetics Computer Group, 575 Science Drive Madison, Wis., USA) or by inspection and the best alignment (i.e., resulting in the highest percentage homology over the comparison window) generated by any of the various methods selected. Reference also can be made to the BLAST family of programs as for example disclosed by Altschul et al., 1997, Nucl. Acids Res. 25:3389, which is herein incorporated by reference in its entirety. A detailed discussion of sequence analysis can be found in Unit 19.3 of Ausubel et al., “Current Protocols in Molecular Biology”, John Wiley & Sons Inc, 1994-1998, Chapter 15, which is herein incorporated by reference in its entirety.

The term “transformation” as used herein, refers to the permanent, heritable alteration in a cell resulting from the uptake and incorporation of foreign DNA into the host-cell genome. This includes the transfer of an exogenous gene from one microorganism into the genome of another microorganism as well as the transfer of additional copies of an endogenous gene into a microorganism.

The term “recombinant” as used herein, refers to an organism that is genetically modified to comprise one or more heterologous or endogenous nucleic acid molecules, such as in a plasmid or vector. Such nucleic acid molecules can be comprised extra-chromosomally or integrated into the chromosome of an organism. The term “non-recombinant” means an organism is not genetically modified. For example, a recombinant organism can be modified to overexpress an endogenous gene encoding an enzyme through modification of promoter elements (e.g., replacing an endogenous promoter element with a constitutive or highly active promoter). Alternatively, a recombinant organism can be modified by introducing a heterologous nucleic acid molecule encoding a protein that is not otherwise expressed in the host organism.

The term “vector” as used herein, refers to a polynucleotide molecule, such as a DNA molecule. It can be derived from a plasmid, bacteriophage, yeast or virus into which a polynucleotide can be inserted or cloned. A vector can contain one or more unique restriction sites and can be capable of autonomous replication in a defined host cell including a target cell or tissue or a progenitor cell or tissue thereof, or be integrable with the genome of the defined host such that the cloned sequence is reproducible. Accordingly, the vector can be an autonomously replicating vector, i.e., a vector that exists as an extra-chromosomal entity, the replication of which is independent of chromosomal replication, e.g., a linear or closed circular plasmid, an extra-chromosomal element, a mini-chromosome, or an artificial chromosome. The vector can contain any means for assuring self-replication. Alternatively, the vector can be one which, when introduced into the host cell, is integrated into the genome and replicated together with the chromosome(s) into which it has been integrated. Such a vector can comprise specific sequences that allow recombination into a particular, desired site of the host chromosome. A vector system can comprise a single vector or plasmid, two or more vectors or plasmids, which together contain the total DNA to be introduced into the genome of the host cell, or a transposon. The choice of the vector will typically depend on the compatibility of the vector with the host cell into which the vector is to be introduced. A vector can be one which is operably functional in a bacterial cell, such as a cyanobacterial cell. The vector can include a reporter gene, such as a green fluorescent protein (GFP), which can be either fused in frame to one or more of the encoded polypeptides, or expressed separately. The vector can also include a selection marker, such as an antibiotic resistance gene, that can be used for selection of suitable transformants.

The terms “inactivate” or “inactivating” as used herein for a gene, refer to a reduction in expression and/or activity of the gene. The terms “inactivate” or “inactivating” as used herein for a biological pathway, refer to a reduction in the activity of an enzyme in a the pathway. For example, inactivating an enzyme of the lactic acid pathway would lead to the production of less lactic acid.

The terms “wild-type” and “naturally-occurring” as used herein are used interchangeably to refer to a gene or gene product that has the characteristics of that gene or gene product when isolated from a naturally occurring source. A wild type gene or gene product (e.g., a polypeptide) is that which is most frequently observed in a population and is thus arbitrarily designed the “normal” or “wild-type” form of the gene.

The term “fuel” or “biofuel” as used herein has its ordinary meaning as known to those skilled in the art and can include one or more compounds suitable as liquid fuels, gaseous fuels, biodiesel fuels (long-chain alkyl (methyl, propyl, or ethyl) esters), heating oil (hydrocarbons in the 14-20 carbon range), reagents, chemical feedstocks and includes, but is not limited to, hydrocarbons (both light and heavy), hydrogen, methane, hydroxy compounds such as alcohols (e.g. ethanol, butanol, propanol, methanol, etc.), and carbonyl compounds such as aldehydes and ketones (e.g. acetone, formaldehyde, 1-propanal, etc.).

The terms “fermentation end-product” or “end-product” as used herein has its ordinary meaning as known to those skilled in the art and can include one or more biofuels, chemical additives, processing aids, food additives, organic acids (e.g. acetic, lactic, formic, citric acid etc.), derivatives of organic acids such as esters (e.g. wax esters, glycerides, etc.) or other functional compounds. These end-products include, but are not limited to, alcohols (e.g. ethanol, butanol, methanol, 1,2-propanediol, 1,3-propanediol, etc.), acids (e.g. lactic acid, formic acid, acetic acid, succinic acid, pyruvic acid, etc.), and enzymes (e.g. cellulases, polysaccharases, lipases, proteases, ligninases, hemicellulases, etc.). End-products can be present as a pure compound, a mixture, or an impure or diluted form.

Various end-products can be produced through saccharification and fermentation using enzyme-enhancing products and processes. These end-products include, but are not limited to, alcohols (e.g. ethanol, butanol, methanol, 1,2-propanediol, 1,3-propanediol), acids (e.g. lactic acid, formic acid, acetic acid, succinic acid, pyruvic acid), and enzymes (e.g. cellulases, polysaccharases, lipases, proteases, ligninases, and hemicellulases) and can be present as a pure compound, a mixture, or an impure or diluted form.

The term “external source”, as it relates to a quantity of an enzyme or enzymes provided to a product or a process, means that the quantity of the enzyme or enzymes is not produced by a microorganism in the product or process. An external source of an enzyme can include, but is not limited to, an enzyme provided in purified form, cell extracts, culture medium or an enzyme obtained from a commercially available source.

The term “plant polysaccharide” as used herein has its ordinary meaning as known to those skilled in the art and can comprise one or more carbohydrate polymers of sugars and sugar derivatives as well as derivatives of sugar polymers and/or other polymeric materials that occur in plant matter. Exemplary plant polysaccharides include lignin, cellulose, starch, pectin, and hemicellulose. Others are chitin, sulfonated polysaccharides such as alginic acid, agarose, carrageenan, porphyran, furcelleran and funoran. Generally, the polysaccharide can have two or more sugar units or derivatives of sugar units. The sugar units and/or derivatives of sugar units can repeat in a regular pattern, or non-regular pattern. The sugar units can be hexose units or pentose units, or combinations of these. The derivatives of sugar units can be sugar alcohols, sugar acids, amino sugars, etc. The polysaccharides can be linear, branched, cross-linked, or a mixture thereof. One type or class of polysaccharide can be cross-linked to another type or class of polysaccharide.

The term “fermentable sugars” as used herein has its ordinary meaning as known to those skilled in the art and can include one or more sugars and/or sugar derivatives that can be used as a carbon source by the microorganism, including monomers, dimers, and polymers of these compounds including two or more of these compounds. In some cases, the microorganism can break down these polymers, such as by hydrolysis, prior to incorporating the broken down material. Exemplary fermentable sugars include, but are not limited to glucose, xylose, arabinose, galactose, mannose, rhamnose, cellobiose, lactose, sucrose, maltose, and fructose.

The term “saccharification” as used herein has its ordinary meaning as known to those skilled in the art and can include conversion of plant polysaccharides to lower molecular weight species that can be used by the microorganism at hand. For some microorganisms, this would include conversion to monosaccharides, disaccharides, trisaccharides, and oligosaccharides of up to about seven monomer units, as well as similar sized chains of sugar derivatives and combinations of sugars and sugar derivatives. For some microorganisms, the allowable chain-length can be longer (e.g. 8, 9, 10, 11, 12, 13, 14, 15, 16, 17, 18, 19, or 20 monomer units or more) and for some microorganisms the allowable chain-length can be shorter (e.g. 1, 2, 3, 4, 5, 6, or 7 monomer units).

The term “biomass” comprises organic material derived from living organisms, including any member from the kingdoms: Monera, Protista, Fungi, Plantae, or Animalia. Organic material that comprises oligosaccharides (e.g., pentose saccharides, hexose saccharides, or longer saccharides) is of particular use in the processes disclosed herein. Organic material includes organisms or material derived therefrom. Organic material includes cellulosic, hemicellulosic, and/or lignocellulosic material. In one embodiment biomass comprises genetically-modified organisms or parts of organisms, such as genetically-modified plant matter, algal matter, or animal matter. In another embodiment biomass comprises non-genetically modified organisms or parts of organisms, such as non-genetically modified plant matter, algal matter, or animal matter. The term “feedstock” is also used to refer to biomass being used in a process, such as those described herein.

Plant matter comprises members of the kingdom Plantae, such as terrestrial plants and aquatic or marine plants. In one embodiment terrestrial plants comprise crop plants (such as fruit, vegetable or grain plants). In one embodiment aquatic or marine plants include, but are not limited to, sea grass, salt marsh grasses (such as Spartina sp. or Phragmites sp.) or the like. In one embodiment a crop plant comprises a plant that is cultivated or harvested for oral consumption, or for utilization in an industrial, pharmaceutical, or commercial process. In one embodiment, crop plants include but are not limited to corn, wheat, rice, barley, soybeans, bamboo, cotton, crambe, jute, sorghum, high biomass sorghum, oats, tobacco, grasses, (e.g., Miscanthus grass or switch grass), trees (softwoods and hardwoods) or tree leaves, beans rape/canola, alfalfa, flax, sunflowers, safflowers, millet, rye, sugarcane, sugar beets, cocoa, tea, Brassica sp., cotton, coffee, sweet potatoes, flax, peanuts, clover; lettuce, tomatoes, cucurbits, cassaya, potatoes, carrots, radishes, peas, lentils, cabbages, cauliflower, broccoli, Brussels sprouts, grapes, peppers, or pineapples; tree fruits or nuts such as citrus, apples, pears, peaches, apricots, walnuts, almonds, olives, avocadoes, bananas, or coconuts; flowers such as orchids, carnations and roses; nonvascular plants such as ferns; oil producing plants (such as castor beans, jatropha, or olives); or gymnosperms such as palms. Plant matter also comprises material derived from a member of the kingdom Plantae, such as woody plant matter, non-woody plant matter, cellulosic material, lignocellulosic material, or hemicellulosic material. Plant matter includes carbohydrates (such as pectin, starch, inulin, fructans, glucans, lignin, cellulose, or xylan). Plant matter also includes sugar alcohols, such as glycerol. In one embodiment plant matter comprises a corn product, (e.g. corn stover, corn cobs, corn grain, corn steep liquor, corn steep solids, or corn grind), stillage, bagasse, leaves, pomace, or material derived therefrom. In another embodiment plant matter comprises distillers grains, Distillers Dried Solubles (DDS), Distillers Dried Grains (DDG), Condensed Distillers Solubles (CDS), Distillers Wet Grains (DWG), Distillers Dried Grains with Solubles (DDGS), peels, pits, fermentation waste, skins, straw, seeds, shells, beancake, sawdust, wood flour, wood pulp, paper pulp, paper pulp waste streams, rice or oat hulls, bagasse, grass clippings, lumber, or food leftovers. These materials can come from farms, forestry, industrial sources, households, etc. In another embodiment plant matter comprises an agricultural waste byproduct or side stream. In another embodiment plant matter comprises a source of pectin such as citrus fruit (e.g., orange, grapefruit, lemon, or limes), potato, tomato, grape, mango, gooseberry, carrot, sugar-beet, and apple, among others. In another embodiment plant matter comprises plant peel (e.g., citrus peels) and/or pomace (e.g., grape pomace). In one embodiment plant matter is characterized by the chemical species present, such as proteins, polysaccharides or oils. In one embodiment plant matter is from a genetically modified plant. In one embodiment a genetically-modified plant produces hydrolytic enzymes (such as a cellulase, hemicellulase, or pectinase etc.) at or near the end of its life cycles. In another embodiment a genetically-modified plant encompasses a mutated species or a species that can initiate the breakdown of cell wall components. In another embodiment plant matter is from a non-genetically modified plant.

Animal matter comprises material derived from a member of the kingdom Animaliae (e.g., bone meal, hair, heads, tails, beaks, eyes, feathers, entrails, skin, shells, scales, meat trimmings, hooves or feet) or animal excrement (e.g., manure). In one embodiment animal matter comprises animal carcasses, milk, meat, fat, animal processing waste, or animal waste (manure from cattle, poultry, and hogs).

Algal matter comprises material derived from a member of the kingdoms Monera (e.g. Cyanobacteria) or Protista (e.g. algae (such as green algae, red algae, glaucophytes, cyanobacteria,) or fungus-like members of Protista (such as slime molds, water molds, etc). Algal matter includes seaweed (such as kelp or red macroalgae), or marine microflora, including plankton.

Organic material comprises waste from farms, forestry, industrial sources, households or municipalities. In one embodiment organic material comprises sewage, garbage, food waste (e.g., restaurant waste), waste paper, toilet paper, yard clippings, or cardboard.

The term “carbonaceous biomass” as used herein has its ordinary meaning as known to those skilled in the art and can include one or more biological materials that can be converted into a biofuel, chemical or other product. Carbonaceous biomass can comprise municipal waste (waste paper, recycled toilet papers, yard clippings, etc.), wood, plant material, plant matter, plant extract, bacterial matter (e.g. bacterial cellulose), distillers' grains, a natural or synthetic polymer, or a combination thereof.

In one embodiment, biomass does not include fossilized sources of carbon, such as hydrocarbons that are typically found within the top layer of the Earth's crust (e.g., natural gas, nonvolatile materials composed of almost pure carbon, like anthracite coal, etc.).

Examples of polysaccharides, oligosaccharides, monosaccharides or other sugar components of biomass include, but are not limited to, alginate, agar, carrageenan, fucoidan, floridean starch, pectin, gluronate, mannuronate, mannitol, lyxose, cellulose, hemicellulose, glycerol, xylitol, glucose, mannose, galactose, xylose, xylan, mannan, arabinan, arabinose, glucuronate, galacturonate (including di- and tri-galacturonates), rhamnose, and the like.

The term “broth” as used herein has its ordinary meaning as known to those skilled in the art and can include the entire contents of the combination of soluble and insoluble matter, suspended matter, cells and medium, such as for example the entire contents of a fermentation reaction can be referred to as a fermentation broth.

The term “productivity” as used herein has its ordinary meaning as known to those skilled in the art and can include the mass of a material of interest produced in a given time in a given volume. Units can be, for example, grams per liter-hour, or some other combination of mass, volume, and time. In fermentation, productivity is frequently used to characterize how fast a product can be made within a given fermentation volume. The volume can be referenced to the total volume of the fermentation vessel, the working volume of the fermentation vessel, or the actual volume of broth being fermented. The context of the phrase will indicate the meaning intended to one of skill in the art. Productivity (e.g. g/L/d) is different from “titer” (e.g. g/L) in that productivity includes a time term, and titer is analogous to concentration.

The terms “conversion efficiency” or “yield” as used herein have their ordinary meaning as known to those skilled in the art and can include the mass of product made from a mass of substrate. The term can be expressed as a percentage yield of the product from a starting mass of substrate. For the production of ethanol from glucose, the net reaction is generally accepted as:

C₆H₁₂O₆→2C₂H₅OH+2CO₂

and the theoretical maximum conversion efficiency or yield is 51% (wt.). Frequently, the conversion efficiency will be referenced to the theoretical maximum, for example, “80% of the theoretical maximum.” In the case of conversion of glucose to ethanol, this statement would indicate a conversion efficiency of 41% (wt.). The context of the phrase will indicate the substrate and product intended to one of skill in the art. For substrates comprising a mixture of different carbon sources such as found in biomass (xylan, xylose, glucose, cellobiose, arabinose cellulose, hemicellulose etc.), the theoretical maximum conversion efficiency of the biomass to ethanol is an average of the maximum conversion efficiencies of the individual carbon source constituents weighted by the relative concentration of each carbon source. In some cases, the theoretical maximum conversion efficiency is calculated based on an assumed saccharification yield. In one embodiment, given carbon source comprising 10 g of cellulose, the theoretical maximum conversion efficiency can be calculated by assuming saccharification of the cellulose to the assimilable carbon source glucose of about 75% by weight. In this embodiment, 10 g of cellulose can provide 7.5 g of glucose which can provide a maximum theoretical conversion efficiency of about 7.5 g·51% or 3.8 g of ethanol. In other cases, the efficiency of the saccharification step can be calculated or determined, i.e., saccharification yield. Saccharification yields can include between about 10-100%, about 20-90%, about 30-80%, about 40-70% or about 50-60%, such as about 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or about 100% for any carbohydrate carbon sources larger than a single monosaccharide subunit.

The saccharification yield takes into account the amount of ethanol and acidic products produced plus the amount of residual monomeric sugars detected in the media. The ethanol figures resulting from media components may not be adjusted. These can account for up to 3 g/L ethanol production or equivalent of up to 6 g/L sugar as much as +/−10%-15% saccharification yield (or saccharification efficiency). For this reason the saccharification yield % can be greater than 100% for some plots. The terms “fed-batch” or “fed-batch fermentation” as used herein has its ordinary meaning as known to those skilled in the art and can include a method of culturing microorganisms where nutrients, other medium components, or biocatalysts (including, for example, enzymes, fresh microorganisms, extracellular broth, etc.) are supplied to the fermentor during cultivation, but culture broth is not harvested from the fermentor until the end of the fermentation, although it can also include “self seeding” or “partial harvest” techniques where a portion of the fermentor volume is harvested and then fresh medium is added to the remaining broth in the fermentor, with at least a portion of the inoculum being the broth that was left in the fermentor. In some embodiments, a fed-batch process might be referred to with a phrase such as, “fed-batch with cell augmentation.” This phrase can include an operation where nutrients and microbial cells are added or one where microbial cells with no substantial amount of nutrients are added. The more general phrase “fed-batch” encompasses these operations as well. The context where any of these phrases is used will indicate to one of skill in the art the techniques being considered.

A term “phytate” as used herein has its ordinary meaning as known to those skilled in the art can be include phytic acid, its salts, and its combined forms as well as combinations of these.

The terms “pretreatment” or “pretreated” as used herein refer to any mechanical, chemical, thermal, biochemical process or combination of these processes whether in a combined step or performed sequentially, that achieves disruption or expansion of a biomass so as to render the biomass more susceptible to attack by enzymes and/or microorganisms. In some embodiments, pretreatment can include removal or disruption of lignin so is to make the cellulose and hemicellulose polymers in the plant biomass more available to cellulolytic enzymes and/or microorganisms, for example, by treatment with acid or base. In some embodiments, pretreatment can include the use of a microorganism of one type to render plant polysaccharides more accessible to microorganisms of another type. In some embodiments, pretreatment can also include disruption or expansion of cellulosic and/or hemicellulosic material. Steam explosion, and ammonia fiber expansion (or explosion) (AFEX) are well known thermal/chemical techniques. Hydrolysis, including methods that utilize acids and/or enzymes can be used. Other thermal, chemical, biochemical, enzymatic techniques can also be used.

The terms “fed-batch” or “fed-batch fermentation” as used herein has its ordinary meaning as known to those skilled in the art and can include a method of culturing microorganisms where nutrients, other medium components, or biocatalysts (including, for example, enzymes, fresh microorganisms, extracellular broth, etc.) are supplied to the fermentor during cultivation, but culture broth is not harvested from the fermentor until the end of the fermentation, although it can also include “self seeding” or “partial harvest” techniques where a portion of the fermentor volume is harvested and then fresh medium is added to the remaining broth in the fermentor, with at least a portion of the inoculum being the broth that was left in the fermentor. In some embodiments, a fed-batch process might be referred to with a phrase such as, “fed-batch with cell augmentation.” This phrase can include an operation where nutrients and microbial cells are added or one where microbial cells with no substantial amount of nutrients are added. The more general phrase “fed-batch” encompasses these operations as well. The context where any of these phrases is used will indicate to one of skill in the art the techniques being considered.

The term “sugar compounds” as used herein has its ordinary meaning as known to those skilled in the art and can include monosaccharide sugars, including but not limited to hexoses and pentoses; sugar alcohols; sugar acids; sugar amines; compounds containing two or more of these linked together directly or indirectly through covalent or ionic bonds; and mixtures thereof. Included within this description are disaccharides; trisaccharides; oligosaccharides; polysaccharides; and sugar chains, branched and/or linear, of any length.

The term “xylanolytic” as used herein refers to any substance capable of breaking down xylan. The term “cellulolytic” as used herein refers to any substance capable of breaking down cellulose.

Generally, compositions and methods are provided for enzyme conditioning of feedstock or biomass to allow saccharification and fermentation to one or more industrially useful fermentation end-products.

The term “biocatalyst” as used herein has its ordinary meaning as known to those skilled in the art and can include one or more enzymes and microorganisms, including solutions, suspensions, and mixtures of enzymes and microorganisms. In some contexts this word will refer to the possible use of either enzymes or microorganisms to serve a particular function, in other contexts the word will refer to the combined use of the two, and in other contexts the word will refer to only one of the two. The context of the phrase will indicate the meaning intended to one of skill in the art.

Generally, compositions and methods are provided for enzyme conditioning of feedstock or biomass to allow saccharification and fermentation to one or more industrially useful fermentive end-products.

### Microorganisms

Microorganisms useful in these compositions and methods include, but are not limited to bacteria, or yeast. Examples of bacteria include, but are not limited to, any bacterium found in the genus of Clostridium, such as C. acetobutylicum, C. aerotolerans, C. beijerinckii, C. bifermentans, C. botulinum, C. butyricum, C. cadaveric, C. chauvoei, C. clostridioforme, C. colicanis, C. difficile, C. fallax, C. formicaceticum, C. histolyticum, C. innocuum, C. ljungdahlii, C. laramie, C. lavalense, C. novyi, C. oedematiens, C. paraputrificum, C. perfringens, C. phytofermentans (including NRRL B-50364 or NRRL B-50351), C. piliforme, C. ramosum, C. scatologenes, C. septicum, C. sordellii, C. sporogenes, C. sp. Q.D (such as NRRL B-50361, NRRL B-50362, or NRRL B-50363), C. tertium, C. tetani, C. tyrobutyricum, or variants thereof (e.g. C. phytofermentans Q.12 or C. phytofermentans Q.13).

Examples of yeast that can be utilized in co-culture methods described herein include but are not limited to, species found in Cryptococcaceae, Sporobolomycetaceae with the genera Cryptococcus, Torulopsis, Pityrosporum, Brettanomyces, Candida, Kloeckera, Trigonopsis, Trichosporon, Rhodotorula and Sporobolomyces and Bullera, the families Endo- and Saccharomycetaceae, with the genera Saccharomyces, Debaromyces, Lipomyces, Hansenula, Endomycopsis, Pichia, Hanseniaspora, Saccharomyces cerevisiae, Pichia pastoris, Hansenula polymorpha, Schizosaccharomyces pombe, Kluyveromyces lactis, Zygosaccharomyces rouxii, Yarrowia lipolitica, Emericella nidulans, Aspergillus nidulans, Deparymyces hansenii and Torulaspora hansenii.

In another embodiment a microorganism can be wild type, or a genetically modified strain. In one embodiment a microorganism can be genetically modified to express one or more polypeptides capable of neutralizing a toxic by-product or inhibitor, which can result in enhanced end-product production in yield and/or rate of production. Examples of modifications include chemical or physical mutagenesis, directed evolution, or genetic alteration to enhance enzyme activity of endogenous proteins, introducing one or more heterogeneous nucleic acid molecules into a host microorganism to express a polypeptide not otherwise expressed in the host, modifying physical and chemical conditions to enhance enzyme function (e.g., modifying and/or maintaining a certain temperature, pH, nutrient concentration, or biomass concentration), or a combination of one or more such modifications.

### Pretreatment of Biomass

Described herein are also methods and compositions for pre-treating biomass prior to extraction of industrially useful end-products. In some embodiments, more complete saccharification of biomass and fermentation of the saccharification products results in higher fuel yields.

In some embodiments, a Clostridium species, for example Clostridium phytofermentans, Clostridium sp. Q.D or a variant thereof, is contacted with pretreated or non-pretreated feedstock containing cellulosic, hemicellulosic, and/or lignocellulosic material. Additional nutrients can be present or added to the biomass material to be processed by the microorganism including nitrogen-containing compounds such as amino acids, proteins, hydrolyzed proteins, ammonia, urea, nitrate, nitrite, soy, soy derivatives, casein, casein derivatives, milk powder, milk derivatives, whey, yeast extract, hydrolyze yeast, autolyzed yeast, corn steep liquor, corn steep solids, monosodium glutamate, and/or other fermentation nitrogen sources, vitamins, and/or mineral supplements. In some embodiments, one or more additional lower molecular weight carbon sources can be added or be present such as glucose, sucrose, maltose, corn syrup, lactic acid, etc. Such lower molecular weight carbon sources can serve multiple functions including providing an initial carbon source at the start of the fermentation period, help build cell count, control the carbon/nitrogen ratio, remove excess nitrogen, or some other function.

In some embodiments aerobic/anaerobic cycling is employed for the bioconversion of cellulosic/lignocellulosic material to fuels and chemicals. In some embodiments, the anaerobic microorganism can ferment biomass directly without the need of a pretreatment. In some embodiments, the anaerobic microorganism can hydrolyze and ferment a biomass without the need of a pretreatment. In certain embodiments, feedstocks are contacted with biocatalysts capable of breaking down plant-derived polymeric material into lower molecular weight products that can subsequently be transformed by biocatalysts to fuels and/or other desirable chemicals. In some embodiments pretreatment methods can include treatment under conditions of high or low pH. High or low pH treatment includes, but is not limited to, treatment using concentrated acids or concentrated alkali, or treatment using dilute acids or dilute alkali. Alkaline compositions useful for treatment of biomass in the methods of the present invention include, but are not limited to, caustic, such as caustic lime, caustic soda, caustic potash, sodium, potassium, or calcium hydroxide, or calcium oxide. In some embodiments suitable amounts of alkaline useful for the treatment of biomass ranges from 0.01 g to 3 g of alkaline (e.g. caustic) for every gram of biomass to be treated. In some embodiments suitable amounts of alkaline useful for the treatment of biomass include, but are not limited to, about 0.01 g of alkaline (e.g. caustic), 0.02 g, 0.03 g, 0.04 g, 0.05 g, 0.075 g, 0.1 g, 0.2 g, 0.3 g, 0.4 g, 0.5 g, 0.75 g, 1 g, 2 g, or about 3 g of alkaline (e.g. caustic) for every gram of biomass to be treated.

In another embodiment, pretreatment of biomass comprises dilute acid hydrolysis. Example of dilute acid hydrolysis treatment are disclosed in T. A. Lloyd and C. E Wyman, Bioresource Technology, (2005) 96, 1967), incorporated by reference herein in its entirety. In other embodiments, pretreatment of biomass comprises pH controlled liquid hot water treatment. Examples of pH controlled liquid hot water treatments are disclosed in N. Mosier et al., Bioresource Technology, (2005) 96, 1986, incorporated by reference herein in its entirety. In other embodiments, pretreatment of biomass comprises aqueous ammonia recycle process (ARP). Examples of aqueous ammonia recycle process are described in T. H. Kim and Y. Y. Lee, Bioresource Technology, (2005)₉₆, incorporated by reference herein in its entirety.

In another embodiment, the above-mentioned methods have two steps: a pretreatment step that leads to a wash stream, and an enzymatic hydrolysis step of pretreated-biomass that produces a hydrolysate stream. In the above methods, the pH at which the pretreatment step is carried out increases progressively from dilute acid hydrolysis to hot water pretreatment to alkaline reagent based methods (AFEX, ARP, and lime pretreatments). Dilute acid and hot water treatment methods solubilize mostly hemicellulose, whereas methods employing alkaline reagents remove most lignin during the pretreatment step. As a result, the wash stream from the pretreatment step in the former methods contains mostly hemicellulose-based sugars, whereas this stream has mostly lignin for the high-pH methods. The subsequent enzymatic hydrolysis of the residual feedstock leads to mixed carbohydrates (C5 and C6) in the alkali-based pretreatment methods, while glucose is the major product in the hydrolysate from the low and neutral pH methods. The enzymatic digestibility of the residual biomass is somewhat better for the high-pH methods due to the removal of lignin that can interfere with the accessibility of cellulase enzyme to cellulose. In some embodiments, pretreatment results in removal of about 20%, 30%, 40%, 50%, 60%, 70% or more of the lignin component of the feedstock. In other embodiments, more than 40%, 50%, 60%, 70%, 80% or more of the hemicellulose component of the feedstock remains after pretreatment. In some embodiments, the microorganism (e.g., Clostridium phytofermentans, Clostridium. sp. Q.D or a variant thereof) is capable of fermenting both five-carbon and six-carbon sugars, which can be present in the feedstock, or can result from the enzymatic degradation of components of the feedstock.

In another embodiment, a two-step pretreatment is used to partially or entirely remove C5 polysaccharides and other components. After washing, the second step consists of an alkali treatment to remove lignin components. The pretreated biomass is then washed prior to saccharification and fermentation. One such pretreatment consists of a dilute acid treatment at room temperature or an elevated temperature, followed by a washing or neutralization step, and then an alkaline contact to remove lignin. For example, one such pretreatment can consist of a mild acid treatment with an acid that is organic (such as acetic acid, citric acid, malic acid, or oxalic acid) or inorganic (such as nitric, hydrochloric, or sulfuric acid), followed by washing and an alkaline treatment in 0.5 to 2.0% NaOH. This type of pretreatment results in a higher percentage of oligomeric to monomeric saccharides, is preferentially fermented by an microorganism such as Clostridium phytofermentans, Clostridium. sp. Q.D or a variant thereof.

In another embodiment, pretreatment of biomass comprises ionic liquid pretreatment. Biomass can be pretreated by incubation with an ionic liquid, followed by extraction with a wash solvent such as alcohol or water. The treated biomass can then be separated from the ionic liquid/wash-solvent solution by centrifugation or filtration, and sent to the saccharification reactor or vessel. Examples of ionic liquid pretreatment are disclosed in US publication No. 2008/0227162, incorporated herein by reference in its entirety.

Examples of pretreatment methods are disclosed in U.S. Pat. No. 4,600,590 to Dale, U.S. Pat. No. 4,644,060 to Chou, U.S. Pat. No. 5,037,663 to Dale. U.S. Pat. No. 5,171,592 to Holtzapple, et al., et al., U.S. Pat. No. 5,939,544 to Karstens, et al., U.S. Pat. No. 5,473,061 to Bredereck, et al., U.S. Pat. No. 6,416,621 to Karstens., U.S. Pat. No. 6,106,888 to Dale, et al., U.S. Pat. No. 6,176,176 to Dale, et al., PCT publication WO2008/020901 to Dale, et al., Felix, A., et al., Anim Prod. 51, 47-61 (1990)., Wais, A. C., Jr., et al., Journal of Animal Science, 35, No. 1, 109-112 (1972), which are incorporated herein by reference in their entireties.

In some embodiments, after pretreatment by any of the above methods the feedstock contains cellulose, hemicellulose, soluble oligomers, simple sugars, lignins, volatiles and/or ash. The parameters of the pretreatment can be changed to vary the concentration of the components of the pretreated feedstock. For example, in some embodiments a pretreatment is chosen so that the concentration of hemicellulose and/or soluble oligomers is high and the concentration of lignins is low after pretreatment. Examples of parameters of the pretreatment include temperature, pressure, time, and pH.

In some embodiments, the parameters of the pretreatment are changed to vary the concentration of the components of the pretreated feedstock such that concentration of the components in the pretreated stock is optimal for fermentation with a microorganism such as C. phytofermentans, Clostridium sp. Q.D, Clostridium phytofermentans Q.12, Clostridium phytofermentans Q.13, or a variant thereof.

In some embodiments, the parameters of the pretreatment are changed such that concentration of accessible cellulose in the pretreated feedstock is about 1%-99%, such as about 1-10%, 1-20%, 1-30%, 1-40%, 1-50%, 1-60%, 1-70%, 1-80%, 1-90% 1-99%, 5-10%, 5-20%, 5-30%, 5-40%, 5-50%, 5-60%, 5-70%, 5-80%, 5-90% 5-99%, 10-10%, 10-20%, 10-30%, 10-40%, 10-50%, 10-60%, 10-70%, 10-80%, 10-90% 10-99%, 15-10%, 15-20%, 15-30%, 15-40%, 15-50%, 15-60%, 15-70%, 15-80%, 15-90% 15-99%, 20-10%, 20-20%, 20-30%, 20-40%, 20-50%, 20-60%, 20-70%, 20-80%, 20-90% 20-99%, 25-10%, 25-20%, 25-30%, 25-40%, 25-50%, 25-60%, 25-70%, 25-80%, 25-90% 25-99%, 30-10%, 30-20%, 30-30%, 30-40%, 30-50%, 30-60%, 30-70%, 30-80%, 30-90% 30-99%, 35-10%, 35-20%, 35-30%, 35-40%, 35-50%, 35-60%, 35-70%, 35-80%, 35-90% 35-99%, 40-10%, 40-20%, 40-30%, 40-40%, 40-50%, 40-60%, 40-70%, 40-80%, 40-90% 40-99%, 45-10%, 45-20%, 45-30%, 45-40%, 45-50%, 45-60%, 45-70%, 45-80%, 45-90% 45-99%, 50-10%, 50-20%, 50-30%, 50-40%, 50-50%, 50-60%, 50-70%, 50-80%, 50-90% 50-99%, 55-10%, 55-20%, 55-30%, 55-40%, 55-50%, 55-60%, 55-70%, 55-80%, 55-90% 55-99%, 60-10%, 60-20%, 60-30%, 60-40%, 60-50%, 60-60%, 60-70%, 60-80%, 60-90% 60-99%, 65-10%, 65-20%, 65-30%, 65-40%, 65-50%, 65-60%, 65-70%, 65-80%, 65-90% 65-99%, 70-10%, 70-20%, 70-30%, 70-40%, 70-50%, 70-60%, 70-70%, 70-80%, 70-90% 70-99%, 75-10%, 75-20%, 75-30%, 75-40%, 75-50%, 75-60%, 75-70%, 75-80%, 75-90% 75-99%, 80-10%, 80-20%, 80-30%, 80-40%, 80-50%, 80-60%, 80-70%, 80-80%, 80-90% 80-99%, 85-10%, 85-20%, 85-30%, 85-40%, 85-50%, 85-60%, 85-70%, 85-80%, 85-90% 85-99%, 90-10%, 90-20%, 90-30%, 90-40%, 90-50%, 90-60%, 90-70%, 90-80%, 90-90% 90-99%, 95-10%, 95-20%, 95-30%, 95-40%, 95-50%, 95-60%, 95-70%, 95-80%, 95-90% 95-99%30%, 20-40%, 20-50%, 30-40% or 30-50%. In some embodiments, the parameters of the pretreatment are changed such that concentration of accessible cellulose in the pretreated feedstock is about 1%, 10%, 15%, 20%, 25%, 30%, 35%, 40%, 45%, 50%, 55%, 60%, 65%, 70%, 75%, 80%, 85%, 90%, 95%, or 99%. In some embodiments, the parameters of the pretreatment are changed such that concentration of accessible cellulose in the pretreated feedstock is 5% to 30%. In some embodiments, the parameters of the pretreatment are changed such that concentration of accessible cellulose in the pretreated feedstock is 10% to 20%.

In some embodiments, the parameters of the pretreatment are changed such that concentration of hemicellulose in the pretreated feedstock is about 1%-99%, such as about 1-10%, 1-20%, 1-30%, 1-40%, 1-50%, 1-60%, 1-70%, 1-80%, 1-90% 1-99%, 5-10%, 5-20%, 5-30%, 5-40%, 5-50%, 5-60%, 5-70%, 5-80%, 5-90% 5-99%, 10-10%, 10-20%, 10-30%, 10-40%, 10-50%, 10-60%, 10-70%, 10-80%, 10-90% 10-99%, 15-10%, 15-20%, 15-30%, 15-40%, 15-50%, 15-60%, 15-70%, 15-80%, 15-90% 15-99%, 20-10%, 20-20%, 20-30%, 20-40%, 20-50%, 20-60%, 20-70%, 20-80%, 20-90% 20-99%, 25-10%, 25-20%, 25-30%, 25-40%, 25-50%, 25-60%, 25-70%, 25-80%, 25-90% 25-99%, 30-10%, 30-20%, 30-30%, 30-40%, 30-50%, 30-60%, 30-70%, 30-80%, 30-90% 30-99%, 35-10%, 35-20%, 35-30%, 35-40%, 35-50%, 35-60%, 35-70%, 35-80%, 35-90% 35-99%, 40-10%, 40-20%, 40-30%, 40-40%, 40-50%, 40-60%, 40-70%, 40-80%, 40-90% 40-99%, 45-10%, 45-20%, 45-30%, 45-40%, 45-50%, 45-60%, 45-70%, 45-80%, 45-90% 45-99%, 50-10%, 50-20%, 50-30%, 50-40%, 50-50%, 50-60%, 50-70%, 50-80%, 50-90% 50-99%, 55-10%, 55-20%, 55-30%, 55-40%, 55-50%, 55-60%, 55-70%, 55-80%, 55-90% 55-99%, 60-10%, 60-20%, 60-30%, 60-40%, 60-50%, 60-60%, 60-70%, 60-80%, 60-90% 60-99%, 65-10%, 65-20%, 65-30%, 65-40%, 65-50%, 65-60%, 65-70%, 65-80%, 65-90% 65-99%, 70-10%, 70-20%, 70-30%, 70-40%, 70-50%, 70-60%, 70-70%, 70-80%, 70-90% 70-99%, 75-10%, 75-20%, 75-30%, 75-40%, 75-50%, 75-60%, 75-70%, 75-80%, 75-90% 75-99%, 80-10%, 80-20%, 80-30%, 80-40%, 80-50%, 80-60%, 80-70%, 80-80%, 80-90% 80-99%, 85-10%, 85-20%, 85-30%, 85-40%, 85-50%, 85-60%, 85-70%, 85-80%, 85-90% 85-99%, 90-10%, 90-20%, 90-30%, 90-40%, 90-50%, 90-60%, 90-70%, 90-80%, 90-90% 90-99%, 95-10%, 95-20%, 95-30%, 95-40%, 95-50%, 95-60%, 95-70%, 95-80%, 95-90% 95-99%. In some embodiments, the parameters of the pretreatment are changed such that concentration of hemicellulose in the pretreated feedstock is about 1%, 10%, 15%, 20%, 25%, 30%, 35%, 40%, 45%, 50%, 55%, 60%, 65%, 70%, 75%, 80%, 85%, 90%, 95%, or 99%. In some embodiments, the parameters of the pretreatment are changed such that concentration of hemicellulose in the pretreated feedstock is 5% to 40%. In some embodiments, the parameters of the pretreatment are changed such that concentration of hemicellulose in the pretreated feedstock is 10% to 30%.

In some embodiments, the parameters of the pretreatment are changed such that concentration of soluble oligomers in the pretreated feedstock is about 1%-99%, such as about 1-10%, 1-20%, 1-30%, 1-40%, 1-50%, 1-60%, 1-70%, 1-80%, 1-90% 1-99%, 5-10%, 5-20%, 5-30%, 5-40%, 5-50%, 5-60%, 5-70%, 5-80%, 5-90% 5-99%, 10-10%, 10-20%, 10-30%, 10-40%, 10-50%, 10-60%, 10-70%, 10-80%, 10-90% 10-99%, 15-10%, 15-20%, 15-30%, 15-40%, 15-50%, 15-60%, 15-70%, 15-80%, 15-90% 15-99%, 20-10%, 20-20%, 20-30%, 20-40%, 20-50%, 20-60%, 20-70%, 20-80%, 20-90% 20-99%, 25-10%, 25-20%, 25-30%, 25-40%, 25-50%, 25-60%, 25-70%, 25-80%, 25-90% 25-99%, 30-10%, 30-20%, 30-30%, 30-40%, 30-50%, 30-60%, 30-70%, 30-80%, 30-90% 30-99%, 35-10%, 35-20%, 35-30%, 35-40%, 35-50%, 35-60%, 35-70%, 35-80%, 35-90% 35-99%, 40-10%, 40-20%, 40-30%, 40-40%, 40-50%, 40-60%, 40-70%, 40-80%, 40-90% 40-99%, 45-10%, 45-20%, 45-30%, 45-40%, 45-50%, 45-60%, 45-70%, 45-80%, 45-90% 45-99%, 50-10%, 50-20%, 50-30%, 50-40%, 50-50%, 50-60%, 50-70%, 50-80%, 50-90% 50-99%, 55-10%, 55-20%, 55-30%, 55-40%, 55-50%, 55-60%, 55-70%, 55-80%, 55-90% 55-99%, 60-10%, 60-20%, 60-30%, 60-40%, 60-50%, 60-60%, 60-70%, 60-80%, 60-90% 60-99%, 65-10%, 65-20%, 65-30%, 65-40%, 65-50%, 65-60%, 65-70%, 65-80%, 65-90% 65-99%, 70-10%, 70-20%, 70-30%, 70-40%, 70-50%, 70-60%, 70-70%, 70-80%, 70-90% 70-99%, 75-10%, 75-20%, 75-30%, 75-40%, 75-50%, 75-60%, 75-70%, 75-80%, 75-90% 75-99%, 80-10%, 80-20%, 80-30%, 80-40%, 80-50%, 80-60%, 80-70%, 80-80%, 80-90% 80-99%, 85-10%, 85-20%, 85-30%, 85-40%, 85-50%, 85-60%, 85-70%, 85-80%, 85-90% 85-99%, 90-10%, 90-20%, 90-30%, 90-40%, 90-50%, 90-60%, 90-70%, 90-80%, 90-90% 90-99%, 95-10%, 95-20%, 95-30%, 95-40%, 95-50%, 95-60%, 95-70%, 95-80%, 95-90% 95-99%. In some embodiments, the parameters of the pretreatment are changed such that concentration of soluble oligomers in the pretreated feedstock is about 1%, 10%, 15%, 20%, 25%, 30%, 35%, 40%, 45%, 50%, 55%, 60%, 65%, 70%, 75%, 80%, 85%, 90%, 95%, or 99%. Examples of soluble oligomers include, but are not limited to, cellobiose and xylobiose. In some embodiments, the parameters of the pretreatment are changed such that concentration of soluble oligomers in the pretreated feedstock is 30% to 90%. In some embodiments, the parameters of the pretreatment are changed such that concentration of soluble oligomers in the pretreated feedstock is 45% to 80%. In some embodiments, the parameters of the pretreatment are changed such that concentration of soluble oligomers in the pretreated feedstock is 45% to 80% and the soluble oligomers are primarily cellobiose and xylobiose.

In some embodiments, the parameters of the pretreatment are changed such that concentration of simple sugars in the pretreated feedstock is about 1%-99%, such as about 1-10%, 1-20%, 1-30%, 1-40%, 1-50%, 1-60%, 1-70%, 1-80%, 1-90% 1-99%, 5-10%, 5-20%, 5-30%, 5-40%, 5-50%, 5-60%, 5-70%, 5-80%, 5-90% 5-99%, 10-10%, 10-20%, 10-30%, 10-40%, 10-50%, 10-60%, 10-70%, 10-80%, 10-90% 10-99%, 15-10%, 15-20%, 15-30%, 15-40%, 15-50%, 15-60%, 15-70%, 15-80%, 15-90% 15-99%, 20-10%, 20-20%, 20-30%, 20-40%, 20-50%, 20-60%, 20-70%, 20-80%, 20-90% 20-99%, 25-10%, 25-20%, 25-30%, 25-40%, 25-50%, 25-60%, 25-70%, 25-80%, 25-90% 25-99%, 30-10%, 30-20%, 30-30%, 30-40%, 30-50%, 30-60%, 30-70%, 30-80%, 30-90% 30-99%, 35-10%, 35-20%, 35-30%, 35-40%, 35-50%, 35-60%, 35-70%, 35-80%, 35-90% 35-99%, 40-10%, 40-20%, 40-30%, 40-40%, 40-50%, 40-60%, 40-70%, 40-80%, 40-90% 40-99%, 45-10%, 45-20%, 45-30%, 45-40%, 45-50%, 45-60%, 45-70%, 45-80%, 45-90% 45-99%, 50-10%, 50-20%, 50-30%, 50-40%, 50-50%, 50-60%, 50-70%, 50-80%, 50-90% 50-99%, 55-10%, 55-20%, 55-30%, 55-40%, 55-50%, 55-60%, 55-70%, 55-80%, 55-90% 55-99%, 60-10%, 60-20%, 60-30%, 60-40%, 60-50%, 60-60%, 60-70%, 60-80%, 60-90% 60-99%, 65-10%, 65-20%, 65-30%, 65-40%, 65-50%, 65-60%, 65-70%, 65-80%, 65-90% 65-99%, 70-10%, 70-20%, 70-30%, 70-40%, 70-50%, 70-60%, 70-70%, 70-80%, 70-90% 70-99%, 75-10%, 75-20%, 75-30%, 75-40%, 75-50%, 75-60%, 75-70%, 75-80%, 75-90% 75-99%, 80-10%, 80-20%, 80-30%, 80-40%, 80-50%, 80-60%, 80-70%, 80-80%, 80-90% 80-99%, 85-10%, 85-20%, 85-30%, 85-40%, 85-50%, 85-60%, 85-70%, 85-80%, 85-90% 85-99%, 90-10%, 90-20%, 90-30%, 90-40%, 90-50%, 90-60%, 90-70%, 90-80%, 90-90% 90-99%, 95-10%, 95-20%, 95-30%, 95-40%, 95-50%, 95-60%, 95-70%, 95-80%, 95-90% 95-99%. In some embodiments, the parameters of the pretreatment are changed such that concentration of simple sugars in the pretreated feedstock is about 1%, 10%, 15%, 20%, 25%, 30%, 35%, 40%, 45%, 50%, 55%, 60%, 65%, 70%, 75%, 80%, 85%, 90%, 95%, or 99%. In some embodiments, the parameters of the pretreatment are changed such that concentration of simple sugars in the pretreated feedstock is 0% to 20%. In some embodiments, the parameters of the pretreatment are changed such that concentration of simple sugars in the pretreated feedstock is 0% to 5%. Examples of simple sugars include, but are not limited to monomers and dimers.

In some embodiments, the parameters of the pretreatment are changed such that concentration of lignins in the pretreated feedstock is about 1%, 10%, 15%, 20%, 25%, 30%, 35%, 40%, 45%, 50%, 55%, 60%, 65%, 70%, 75%, 80%, 85%, 90%, 95%, or 99%. In some embodiments, the parameters of the pretreatment are changed such that concentration of lignins in the pretreated feedstock is 0% to 20%. In some embodiments, the parameters of the pretreatment are changed such that concentration of lignins in the pretreated feedstock is 0% to 5%. In some embodiments, the parameters of the pretreatment are changed such that concentration of lignins in the pretreated feedstock is less than 1% to 2%. In some embodiments, the parameters of the pretreatment are changed such that the concentration of phenolics is minimized.

In some embodiments, the parameters of the pretreatment are changed such that concentration of furfural and low molecular weight lignins in the pretreated feedstock is less than 10%, 9%, 8%, 7%, 6%, 5%, 4%, 3%, 2%, or 1%. In some embodiments, the parameters of the pretreatment are changed such that concentration of furfural and low molecular weight lignins in the pretreated feedstock is less than 1% to 2%.

In some embodiments, the parameters of the pretreatment are changed such that concentration of accessible cellulose is 10% to 20%, the concentration of hemicellulose is 10% to 30%, the concentration of soluble oligomers is 45% to 80%, the concentration of simple sugars is 0% to 5%, and the concentration of lignins is 0% to 5% and the concentration of furfural and low molecular weight lignins in the pretreated feedstock is less than 1% to 2%.

In some embodiments, the parameters of the pretreatment are changed to obtain a high concentration of hemicellulose (e.g., 30%, 35%, 40%, 45%, 50%, 55%, 60%, 65%, 70% or higher) and a low concentration of lignins (e.g., 1%, 2%, 3%, 4%, 5%, 10%, 15%, 20%, 25%, or 30%). In some embodiments, the parameters of the pretreatment are changed to obtain a high concentration of hemicellulose and a low concentration of lignins such that concentration of the components in the pretreated stock is optimal for fermentation with a microorganism such as a member of the genus Clostridium, for example Clostridium phytofermentans, Clostridium sp. Q.D, Clostridium phytofermentans Q.12, Clostridium phytofermentans Q.13 or variants thereof.

Certain conditions of pretreatment can be modified prior to, or concurrently with, introduction of a fermentative microorganism into the feedstock. For example, pretreated feedstock can be cooled to a temperature which allows for growth of the microorganism(s). As another example, pH can be altered prior to, or concurrently with, addition of one or more microorganisms.

Alteration of the pH of a pretreated feedstock can be accomplished by washing the feedstock (e.g., with water) one or more times to remove an alkaline or acidic substance, or other substance used or produced during pretreatment. Washing can comprise exposing the pretreated feedstock to an equal volume of water 2, 3, 4, 5, 6, 7, 8, 9, 10, 11, 12, 13, 14, 15, 16, 17, 18, 19, 20, 21, 22, 23, 24, 25 or more times. In another embodiment, a pH modifier can be added. For example, an acid, a buffer, or a material that reacts with other materials present can be added to modulate the pH of the feedstock. In some embodiments, more than one pH modifier can be used, such as one or more bases, one or more bases with one or more buffers, one or more acids, one or more acids with one or more buffers, or one or more buffers. When more than one pH modifiers are utilized, they can be added at the same time or at different times. Other non-limiting exemplary methods for neutralizing feedstocks treated with alkaline substances have been described, for example in U.S. Pat. Nos. 4,048,341; 4,182,780; and 5,693,296.

In some embodiments, one or more acids can be combined, resulting in a buffer. Suitable acids and buffers that can be used as pH modifiers include any liquid or gaseous acid that is compatible with the microorganism. Non-limiting examples include peroxyacetic acid, sulfuric acid, lactic acid, citric acid, phosphoric acid, and hydrochloric acid. In some instances, the pH can be lowered to neutral pH or acidic pH, for example a pH of 7.0, 6.5, 6.0, 5.5, 5.0, 4.5, 4.0, or lower. In some embodiments, the pH is lowered and/or maintained within a range of about pH 4.5 to about 7.1, or about 4.5 to about 6.9, or about pH 5.0 to about 6.3, or about pH 5.5 to about 6.3, or about pH 6.0 to about 6.5, or about pH 5.5 to about 6.9 or about pH 6.2 to about 6.7.

In another embodiment, biomass can be pre-treated at an elevated temperature and/or pressure. In one embodiment biomass is pre treated at a temperature range of 20° C. to 400° C. In another embodiment biomass is pretreated at a temperature of about 20° C., 25° C., 30° C., 35° C., 40° C., 45° C., 50° C., 55° C., 60° C., 65° C., 80° C., 90° C., 100° C., 120° C., 150° C., 200° C., 250° C., 300° C., 350° C., 400° C. or higher. In another embodiment, elevated temperatures are provided by the use of steam, hot water, or hot gases. In one embodiment steam can be injected into a biomass containing vessel. In another embodiment the steam, hot water, or hot gas can be injected into a vessel jacket such that it heats, but does not directly contact the biomass.

In another embodiment, a biomass can be treated at an elevated pressure. In one embodiment biomass is pre treated at a pressure range of about 1 psi to about 30 psi. In another embodiment biomass is pre treated at a pressure or about 1 psi, 2 psi, 3 psi, 4 psi, 5 psi, 6 psi, 7 psi, 8 psi, 9 psi, 10 psi, 12 psi, 15 psi, 18 psi, 20 psi, 22 psi, 24 psi, 26 psi, 28 psi, 30 psi or more. In some embodiments, biomass can be treated with elevated pressures by the injection of steam into a biomass containing vessel. In other embodiments, the biomass can be treated to vacuum conditions prior or subsequent to alkaline or acid treatment or any other treatment methods provided herein.

In one embodiment alkaline or acid pretreated biomass is washed (e.g. with water (hot or cold) or other solvent such as alcohol (e.g. ethanol)), pH neutralized with an acid, base, or buffering agent (e.g. phosphate, citrate, borate, or carbonate salt) or dried prior to fermentation. In one embodiment, the drying step can be performed under vacuum to increase the rate of evaporation of water or other solvents. Alternatively, or additionally, the drying step can be performed at elevated temperatures such as about 20° C., 25° C., 30° C., 35° C., 40° C., 45° C., 50° C., 55° C., 60° C., 65° C., 80° C., 90° C., 100° C., 120° C., 150° C., 200° C., 250° C., 300° C. or more.

In some embodiments, the pretreatment step includes a step of solids recovery. The solids recovery step can be during or after pretreatment (e.g., acid or alkali pretreatment), or before the drying step. In some embodiments, the solids recovery step provided by the methods described herein includes the use of a sieve, filter, screen, or a membrane for separating the liquid and solids fractions. In one embodiment a suitable sieve pore diameter size ranges from about 0.001 microns to 8 mm, such as about 0.005 microns to 3 mm or about 0.01 microns to 1 mm. In one embodiment a sieve pore size has a pore diameter of about 0.01 microns, 0.02 microns, 0.05 microns, 0.1 microns, 0.5 microns, 1 micron, 2 microns, 4 microns, 5 microns, 10 microns, 20 microns, 25 microns, 50 microns, 75 microns, 100 microns, 125 microns, 150 microns, 200 microns, 250 microns, 300 microns, 400 microns, 500 microns, 750 microns, 1 mm or more.

In some embodiments, biomass (e.g. corn stover) is processed or pretreated prior to fermentation. In one embodiment a method of pre-treatment includes but is not limited to, biomass particle size reduction, such as for example shredding, milling, chipping, crushing, grinding, or pulverizing. In some embodiments, biomass particle size reduction can include size separation methods such as sieving, or other suitable methods known in the art to separate materials based on size. In one embodiment size separation can provide for enhanced yields. In some embodiments, separation of finely shredded biomass (e.g. particles smaller than about 8 mm in diameter, such as, 8, 7.9, 7.7, 7.5, 7.3, 7, 6.9, 6.7, 6.5, 6.3, 6, 5.9, 5.7, 5.5, 5.3, 5, 4.9, 4.7, 4.5, 4.3, 4, 3.9, 3.7, 3.5, 3.3, 3, 2.9, 2.7, 2.5, 2.3, 2, 1.9, 1.7, 1.5, 1.3, 1, 0.9, 0.8, 0.7, 0.6, 0.5, 0.4, 0.3, 0.2, or 0.1 mm) from larger particles allows the recycling of the larger particles back into the size reduction process, thereby increasing the final yield of processed biomass. In one embodiment, a fermentative mixture is provided which comprises a pretreated lignocellulosic feedstock comprising less than about 50% of a lignin component present in the feedstock prior to pretreatment and comprising more than about 60% of a hemicellulose component present in the feedstock prior to pretreatment; and a microorganism capable of fermenting a five-carbon sugar, such as xylose, arabinose or a combination thereof, and a six-carbon sugar, such as glucose, galactose, mannose or a combination thereof. In some instances, pretreatment of the lignocellulosic feedstock comprises adding an alkaline substance which raises the pH to an alkaline level, for example NaOH. In some embodiments, NaOH is added at a concentration of about 0.5% to about 2% by weight of the feedstock. In other embodiments, pretreatment also comprises addition of a chelating agent. In some embodiments, the microorganism is a bacterium, such as a member of the genus Clostridium, for example Clostridium phytofermentans, Clostridium sp. Q.D, Clostridium phytofermentans Q.12, Clostridium phytofermentans Q.13 or variant thereof.

The present disclosure also provides a fermentative mixture comprising: a cellulosic feedstock pre-treated with an alkaline substance which maintains an alkaline pH, and at a temperature of from about 80° C. to about 120° C.; and a microorganism capable of fermenting a five-carbon sugar and a six-carbon sugar. In some instances, the five-carbon sugar is xylose, arabinose, or a combination thereof. In other instances, the six-carbon sugar is glucose, galactose, mannose, or a combination thereof. In some embodiments, the alkaline substance is NaOH. In some embodiments, NaOH is added at a concentration of about 0.5% to about 2% by weight of the feedstock. In some embodiments, the microorganism is a bacterium, such as a member of the genus Clostridium, for example Clostridium phytofermentans, Clostridium sp. Q.D, Clostridium phytofermentans Q.12 or Clostridium phytofermentans Q.13 or variants thereof. In still other embodiments, the microorganism is genetically modified to enhance activity of one or more hydrolytic enzymes.

Further provided herein is a fermentative mixture comprising a cellulosic feedstock pre-treated with an alkaline substance which increases the pH to an alkaline level, at a temperature of from about 80° C. to about 120° C.; and a microorganism capable of uptake and fermentation of an oligosaccharide. In some embodiments the alkaline substance is NaOH. In some embodiments, NaOH is added at a concentration of about 0.5% to about 2% by weight of the feedstock. In some embodiments, the microorganism is a bacterium, such as a member of the genus Clostridium, for example Clostridium phytofermentans, Clostridium sp. Q.D, Clostridium phytofermentans Q.12, Clostridium phytofermentans Q.13, or variants thereof. In other embodiments, the microorganism is genetically modified to express or increase expression of an enzyme capable of hydrolyzing the oligosaccharide, a transporter capable of transporting the oligosaccharide, or a combination thereof.

Another aspect of the present disclosure provides a fermentative mixture comprising a cellulosic feedstock comprising cellulosic material from one or more sources, wherein the feedstock is pre-treated with a substance which increases the pH to an alkaline level, at a temperature of from about 80° C. to about 120° C.; and a microorganism capable of fermenting the cellulosic material from at least two different sources to produce a fermentation end-product at substantially a same yield coefficient. In some instances, the sources of cellulosic material are corn stover, bagasse, switchgrass or poplar. In some embodiments the alkaline substance is NaOH. In some embodiments, NaOH is added at a concentration of about 0.5% to about 2% by weight of the feedstock. In some embodiments, the microorganism is a bacterium, such as a member of the genus Clostridium, for example Clostridium phytofermentans, Clostridium sp. Q.D, Clostridium phytofermentans Q.12 or Clostridium phytofermentans Q.13 or variants thereof.

In some embodiments, a process for simultaneous saccharification and fermentation of cellulosic solids from biomass into biofuel or another end-product is provided. In one embodiment the process comprises treating the biomass in a closed container with a microorganism under conditions where the microorganism produces saccharolytic enzymes sufficient to substantially convert the biomass into oligomers, monosaccharides and disaccharides. In one embodiment the microorganism subsequently converts the oligomers, monosaccharides and disaccharides into ethanol and/or another biofuel or product.

In an another embodiment, a process for saccharification and fermentation comprises treating the biomass in a container with the microorganism, and adding one or more enzymes before, concurrent or after contacting the biomass with the microorganism, wherein the enzymes added aid in the breakdown or detoxification of carbohydrates or lignocellulosic material.

In one embodiment, the bioconversion process comprises a separate hydrolysis and fermentation (SHF) process. In an SHF embodiment, the enzymes can be used under their optimal conditions regardless of the fermentation conditions and the microorganism is only required to ferment released sugars. In this embodiment, hydrolysis enzymes are externally added.

In another embodiment, the bioconversion process comprises a saccharification and fermentation (SSF) process. In an SSF embodiment, hydrolysis and fermentation take place in the same reactor under the same conditions.

In another embodiment, the bioconversion process comprises a consolidated bioprocess (CBP). In essence, CBP is a variation of SSF in which the enzymes are produced by the microorganism that carries out the fermentation. In this embodiment, enzymes can be both externally added enzymes and enzymes produced by the fermentative microorganism. In this embodiment, biomass is partially hydrolyzed with externally added enzymes at their optimal condition, the slurry is then transferred to a separate tank in which the fermentative microorganism (e.g. Clostridium phytofermentans, Clostridium sp. Q.D, Clostridium phytofermentans Q.12 or Clostridium phytofermentans Q.13 or variants thereof) converts the hydrolyzed sugar into the desired product (e.g. fuel or chemical) and completes the hydrolysis of the residual cellulose and hemicellulose.

In one embodiment, pretreated biomass is partially hydrolyzed by externally added enzymes to reduce the viscosity. Hydrolysis occurs at the optimal pH and temperature conditions (e.g. pH 5.5, 50° C. for fungal cellulases). Hydrolysis time and enzyme loading can be adjusted such that conversion is limited to cellodextrins (soluble and insoluble) and hemicellulose oligomers. At the conclusion of the hydrolysis time, the resultant mixture can be subjected to fermentation conditions. For example, the resultant mixture can be pumped over time (fed batch) into a reactor containing a microorganism (e.g. Clostridium phytofermentans, Clostridium sp. Q.D, Clostridium phytofermentans Q.12 or Clostridium phytofermentans Q.13 or variants thereof) and media. The microorganism can then produce endogenous enzymes to complete the hydrolysis into fermentable sugars (soluble oligomers) and convert those sugars into ethanol and/or other products in a production tank. The production tank can then be operated under fermentation optimal conditions (e.g. pH 6.5, 35° C.). In this way externally added enzyme is minimized due to operation under the enzyme's optimal conditions and due to a portion of the enzyme coming from the microorganism (e.g. Clostridium phytofermentans, Clostridium sp. Q.D, Clostridium phytofermentans Q.12 or Clostridium phytofermentans Q.13 or variants thereof).

In some embodiments, exogenous enzymes added include a xylanase, a hemicellulase, a glucanase or a glucosidase. In some embodiments, exogenous enzymes added do not include a xylanase, a hemicellulase, a glucanase or a glucosidase. In other embodiments, the amount of exogenous cellulase is greatly reduced, one-quarter or less of the amount normally added to a fermentation by a microorganism that cannot saccharify the biomass.

In one embodiment a second microorganism can be used to convert residual carbohydrates into a fermentation end-product. In one embodiment the second microorganism is a yeast such as Saccharomyces cerevisiae; a Clostridia species such as C. thermocellum, C. acetobutylicum, or C. cellovorans; or Zymomonas mobilis.

In one embodiment, a process of producing a biofuel or chemical product from a lignin-containing biomass is provided. In one embodiment the process comprises: 1) contacting the lignin-containing biomass with an aqueous alkaline solution at a concentration sufficient to hydrolyze at least a portion of the lignin-containing biomass; 2) neutralizing the treated biomass to a pH between 5 to 9 (e.g. 5.5, 6, 6.5, 7, 7.5, 8, 8.5, or 9); 3) treating the biomass in a closed container with a Clostridium microorganism, (such as Clostridium phytofermentans, a Clostridium sp. Q.D, a Clostridium phytofermentans Q.13 or a Clostridium phytofermentans Q.12 or variants thereof) under conditions wherein the Clostridium microorganism, optionally with the addition of one or more hydrolytic enzymes to the container, substantially converts the treated biomass into oligomers, monosaccharides and disaccharides, and/or biofuel or other fermentation end-product; and 4) optionally, introducing a culture of a second microorganism wherein the second microorganism is capable of substantially converting the oligomers, monosaccharides and disaccharides into biofuel.

Of various molecules typically found in biomass, cellulose is useful as a starting material for the production of fermentation end-products in methods and compositions described herein. Cellulose is one of the major components in plant cell wall. Cellulose is a linear condensation polymer consisting of D-anhydro glucopyranose joined together by β-1,4-linkage. The degree of polymerization ranges from 100 to 20,000. Adjacent cellulose molecules are coupled by extensive hydrogen bonds and van der Waals forces, resulting in a parallel alignment. The parallel sheet-like structure renders cellulose very stable.

Pretreatment can also include utilization of one or more strong cellulose swelling agents that facilitate disruption of the fiber structure and thus rendering the cellulosic material more amendable to saccharification and fermentation. Some considerations have been given in selecting an efficient method of swelling for various cellulosic material: 1) the hydrogen bonding fraction; 2) solvent molar volume; 3) the cellulose structure. The width and distribution of voids (between the chains of linear cellulosic polymer) are important as well. It is known that the swelling is more pronounced in the presence of electrostatic repulsion, provided by alkali solution or ionic surfactants. Of course, with respect to utilization of any of the methods disclosed herein, conditioning of a biomass can be concurrent to contact with a microorganism that is capable of saccharification and fermentation. In addition, other examples describing the pretreatment of lignocellulosic biomass have been published as U.S. Pat. Nos. 4,304,649, 5,366,558, 5,411,603, and 5,705,369.

### Biomass Processing

Described herein are compositions and methods allowing saccharification and fermentation to one or more industrially useful fermentation end-products. Saccharification includes conversion of long-chain sugar polymers, such as cellulose, to monosaccharides, disaccharides, trisaccharides, and oligosaccharides of up to about seven monomer units, as well as similar sized chains of sugar derivatives and combinations of sugars and sugar derivatives. The chain-length for saccharides can be longer (e.g. 8, 9, 10, 11, 12, 13, 14, 15, 16, 17, 18, 19, or 20 monomer units or more) and or shorter (e.g. 1, 2, 3, 4, 5, 6 monomer units). As used herein, “directly processing” means that a microorganism is capable of both hydrolyzing biomass and fermenting without the need for conditioning the biomass, such as subjecting the biomass to chemical, heat, enzymatic treatment or combinations thereof.

Methods and compositions described herein contemplate utilizing fermentation process for extracting industrially useful fermentation end-products from biomass. The term “fermentation” as used herein has its ordinary meaning as known to those skilled in the art and can include culturing of a microorganism or group of microorganisms in or on a suitable medium for the microorganisms. The microorganisms can be aerobes, anaerobes, facultative anaerobes, heterotrophs, autotrophs, photoautotrophs, photoheterotrophs, chemoautotrophs, and/or chemoheterotrophs. The cellular activity, including cell growth can be growing aerobic, microaerophilic, or anaerobic. The cells can be in any phase of growth, including lag (or conduction), exponential, transition, stationary, death, dormant, vegetative, sporulating, etc.

Organisms disclosed herein can be incorporated into methods and compositions so as to enhance fermentation end-product yield and/or rate of production. One example of such a microorganism is Clostridium phytofermentans (“C. phytofermentans”), which can simultaneously hydrolyze and ferment lignocellulosic biomass. Furthermore, C. phytofermentans is capable of hydrolyzing and fermenting hexose (C6) and pentose (C5) polysaccharides (e.g. carbohydrates). In addition, C. phytofermentans is capable of acting directly on lignocellulosic biomass without any pretreatment. Other examples of microorganisms that can hydrolyze and ferment hexose (C6) and pentose (C5) polysaccharides include Clostridium sp. Q.D, or variants of Clostridium phytofermentans (e.g. mutagenized or recombinant), such as Clostridium Q.8, Clostridium Q.12, or Clostridium phytofermentans Q.13. Additionally, these organisms can produce hemicellulases, pectinases, xylansases, or chitinases.

In one embodiment, modified microorganisms are provided which ferment hexose and pentose polysaccharides which are part of a biomass. In some embodiments, a Clostridium hydrolyzes and ferment hexose and pentose polysaccharides which are part of a biomass. In a further embodiment, C. phytofermentans or variants thereof hydrolyze and ferment hexose and pentose polysaccharides which are part of a biomass. In some embodiments, the biomass comprises lignocellulose. In some embodiments, the biomass comprises hemicellulose.

### Co-Culture Methods and Compositions

Methods can also include co-culture with a microorganism that naturally produces or is genetically modified to produce one or more enzymes, such as hydrolytic enzymes (such as cellulase(s), hemicellulase(s), or pectinases etc.) or antioxidants (such as catalase, superoxide dismutase or glutathione peroxidase). A culture medium containing such a microorganism can be contacted with biomass (e.g., in a bioreactor) prior to, concurrent with, or subsequent to contact with a second microorganism. In one embodiment a first microorganism produces saccharifying enzyme while a second microorganism ferments C5 and C6 sugars. In one embodiment, the first microorganism is C. phytofermentans or Clostridium sp. Q.D. Mixtures of microorganisms can be provided as solid mixtures (e.g., freeze-dried mixtures), or as liquid dispersions of the microorganisms, and grown in co-culture with a second microorganism. Co-culture methods capable of use are known, such as those disclosed in U.S. Patent Application Publication No. 20070178569, which is hereby incorporated by reference in its entirety.

### Fermentation End-Product

The term “fuel” or “biofuel” as used herein has its ordinary meaning as known to those skilled in the art and can include one or more compounds suitable as liquid fuels, gaseous fuels, biodiesel fuels (long-chain alkyl (methyl, propyl or ethyl) esters), heating oils (hydrocarbons in the 14-20 carbon range), reagents, chemical feedstocks and includes, but is not limited to, hydrocarbons (both light and heavy), hydrogen, methane, hydroxy compounds such as alcohols (e.g. ethanol, butanol, propanol, methanol, etc.), and carbonyl compounds such as aldehydes and ketones (e.g. acetone, formaldehyde, 1-propanal, etc.).

The term “fermentation end-product” or “end-product” as used herein has its ordinary meaning as known to those skilled in the art and can include one or more biofuels, or chemicals, (such as additives, processing aids, food additives, organic acids (e.g. acetic, lactic, formic, citric acid etc.), derivatives of organic acids such as esters (e.g. wax esters, glycerides, etc.) or other compounds). These end-products include, but are not limited to, an alcohol (such as ethanol, butanol, methanol, 1,2-propanediol, or 1,3-propanediol), an acid (such as lactic acid, formic acid, acetic acid, succinic acid, or pyruvic acid), enzymes such as cellulases, polysaccharases, lipases, proteases, ligninases, and hemicellulases and can be present as a pure compound, a mixture, or an impure or diluted form. In one embodiment a fermentation end-product is made using a process or microorganism disclosed herein. In another embodiment production of a fermentation end-product is enhanced through saccharification and fermentation using enzyme-enhancing products or processes.

In one embodiment a fermentation end-product is a 1,4 diacid (succinic, fumaric and malic), 2,5 furan dicarboxylic acid, 3-hydroxy propionic acid, aspartic acid, glucaric acid, glutamic acid, itaconic acid, levulinic acid, 3-hydroxybutyrolactone, glycerol, sorbitol, xylitol/arabitol, butanediol, butanol, isopentenyl diphosphate, methane, methanol, ethane, ethene, ethanol, n-propane, 1-propene, 1-propanol, propanal, acetone, propionate, n-butane, 1-butene, 1-butanol, butanal, butanoate, isobutanal, isobutanol, 2-methylbutanal, 2-methylbutanol, 3-methylbutanal, 3-methylbutanol, 2-butene, 2-butanol, 2-butanone, 2,3-butanediol, 3-hydroxy-2-butanone, 2,3-butanedione, ethylbenzene, ethenylbenzene, 2-phenylethanol, phenylacetaldehyde, 1-phenylbutane, 4-phenyl-1-butene, 4-phenyl-2-butene, 1-phenyl-2-butene, 1-phenyl-2-butanol, 4-phenyl-2-butanol, 1-phenyl-2-butanone, 4-phenyl-2-butanone, 1-phenyl-2,3-butandiol, 1-phenyl-3-hydroxy-2-butanone, 4-phenyl-3-hydroxy-2-butanone, 1-phenyl-2,3-butanedione, n-pentane, ethylphenol, ethenylphenol, 2-(4-hydroxyphenyl)ethanol, 4-hydroxyphenylacetaldehyde, 1-(4-hydroxyphenyl)butane, 4-(4-hydroxyphenyl)-1-butene, 4-(4-hydroxyphenyl)-2-butene, 1-(4-hydroxyphenyl)-1-butene, 1-(4-hydroxyphenyl)-2-butanol, 4-(4-hydroxyphenyl)-2-butanol, 1-(4-hydroxyphenyl)-2-butanone, 4-(4-hydroxyphenyl)-2-butanone, 1-(4-hydroxyphenyl)-2,3-butandiol, 1-(4-hydroxyphenyl)-3-hydroxy-2-butanone, 4-(4-hydroxyphenyl)-3-hydroxy-2-butanone, 1-(4-hydroxyphenyl)-2,3-butanonedione, indolylethane, indolylethene, 2-(indole-3-)ethanol, n-pentane, 1-pentene, 1-pentanol, pentanal, pentanoate, 2-pentene, 2-pentanol, 3-pentanol, 2-pentanone, 3-pentanone, 4-methylpentanal, 4-methylpentanol, 2,3-pentanediol, 2-hydroxy-3-pentanone, 3-hydroxy-2-pentanone, 2,3-pentanedione, 2-methylpentane, 4-methyl-1-pentene, 4-methyl-2-pentene, 4-methyl-3-pentene, 4-methyl-2-pentanol, 2-methyl-3-pentanol, 4-methyl-2-pentanone, 2-methyl-3-pentanone, 4-methyl-2,3-pentanediol, 4-methyl-2-hydroxy-3-pentanone, 4-methyl-3-hydroxy-2-pentanone, 4-methyl-2,3-pentanedione, 1-phenylpentane, 1-phenyl-1-pentene, 1-phenyl-2-pentene, 1-phenyl-3-pentene, 1-phenyl-2-pentanol, 1-phenyl-3-pentanol, 1-phenyl-2-pentanone, 1-phenyl-3-pentanone, 1-phenyl-2,3-pentanediol, 1-phenyl-2-hydroxy-3-pentanone, 1-phenyl-3-hydroxy-2-pentanone, 1-phenyl-2,3-pentanedione, 4-methyl-1-phenylpentane, 4-methyl-1-phenyl-1-pentene, 4-methyl-1-phenyl-2-pentene, 4-methyl-1-phenyl-3-pentene, 4-methyl-1-phenyl-3-pentanol, 4-methyl-1-phenyl-2-pentanol, 4-methyl-1-phenyl-3-pentanone, 4-methyl-1-phenyl-2-pentanone, 4-methyl-1-phenyl-2,3-pentanediol, 4-methyl-1-phenyl-2,3-pentanedione, 4-methyl-1-phenyl-3-hydroxy-2-pentanone, 4-methyl-1-phenyl-2-hydroxy-3-pentanone, 1-(4-hydroxyphenyl)pentane, 1-(4-hydroxyphenyl)-1-pentene, 1-(4-hydroxyphenyl)-2-pentene, 1-(4-hydroxyphenyl)-3-pentene, 1-(4-hydroxyphenyl)-2-pentanol, 1-(4-hydroxyphenyl)-3-pentanol, 1-(4-hydroxyphenyl)-2-pentanone, 1-(4-hydroxyphenyl)-3-pentanone, 1-(4-hydroxyphenyl)-2,3-pentanediol, 1-(4-hydroxyphenyl)-2-hydroxy-3-pentanone, 1-(4-hydroxyphenyl)-3-hydroxy-2-pentanone, 1-(4-hydroxyphenyl)-2,3-pentanedione, 4-methyl-1-(4-hydroxyphenyl) pentane, 4-methyl-1-(4-hydroxyphenyl)-2-pentene, 4-methyl-1-(4-hydroxyphenyl)-3-pentene, 4-methyl-1-(4-hydroxyphenyl)-1-pentene, 4-methyl-1-(4-hydroxyphenyl)-3-pentanol, 4-methyl-1-(4-hydroxyphenyl)-2-pentanol, 4-methyl-1-(4-hydroxyphenyl)-3-pentanone, 4-methyl-1-(4-hydroxyphenyl)-2-pentanone, 4-methyl-1-(4-hydroxyphenyl)-2,3-pentanediol, 4-methyl-1-(4-hydroxyphenyl)-2,3-pentanedione, 4-methyl-1-(4-hydroxyphenyl)-3-hydroxy-2-pentanone, 4-methyl-1-(4-hydroxyphenyl)-2-hydroxy-3-pentanone, 1-indole-3-pentane, 1-(indole-3)-1-pentene, 1-(indole-3)-2-pentene, 1-(indole-3)-3-pentene, 1-(indole-3)-2-pentanol, 1-(indole-3)-3-pentanol, 1-(indole-3)-2-pentanone, 1-(indole-3)-3-pentanone, 1-(indole-3)-2,3-pentanediol, 1-(indole-3)-2-hydroxy-3-pentanone, 1-(indole-3)-3-hydroxy-2-pentanone, 1-(indole-3)-2,3-pentanedione, 4-methyl-1-(indole-3-)pentane, 4-methyl-1-(indole-3)-2-pentene, 4-methyl-1-(indole-3)-3-pentene, 4-methyl-1-(indole-3)-1-pentene, 4-methyl-2-(indole-3)-3-pentanol, 4-methyl-1-(indole-3)-2-pentanol, 4-methyl-1-(indole-3)-3-pentanone, 4-methyl-1-(indole-3)-2-pentanone, 4-methyl-1-(indole-3)-2,3-pentanediol, 4-methyl-1-(indole-3)-2,3-pentanedione, 4-methyl-1-(indole-3)-3-hydroxy-2-pentanone, 4-methyl-1-(indole-3)-2-hydroxy-3-pentanone, n-hexane, 1-hexene, 1-hexanol, hexanal, hexanoate, 2-hexene, 3-hexene, 2-hexanol, 3-hexanol, 2-hexanone, 3-hexanone, 2,3-hexanediol, 2,3-hexanedione, 3,4-hexanediol, 3,4-hexanedione, 2-hydroxy-3-hexanone, 3-hydroxy-2-hexanone, 3-hydroxy-4-hexanone, 4-hydroxy-3-hexanone, 2-methylhexane, 3-methylhexane, 2-methyl-2-hexene, 2-methyl-3-hexene, 5-methyl-1-hexene, 5-methyl-2-hexene, 4-methyl-1-hexene, 4-methyl-2-hexene, 3-methyl-3-hexene, 3-methyl-2-hexene, 3-methyl-1-hexene, 2-methyl-3-hexanol, 5-methyl-2-hexanol, 5-methyl-3-hexanol, 2-methyl-3-hexanone, 5-methyl-2-hexanone, 5-methyl-3-hexanone, 2-methyl-3,4-hexanediol, 2-methyl-3,4-hexanedione, 5-methyl-2,3-hexanediol, 5-methyl-2,3-hexanedione, 4-methyl-2,3-hexanediol, 4-methyl-2,3-hexanedione, 2-methyl-3-hydroxy-4-hexanone, 2-methyl-4-hydroxy-3-hexanone, 5-methyl-2-hydroxy-3-hexanone, 5-methyl-3-hydroxy-2-hexanone, 4-methyl-2-hydroxy-3-hexanone, 4-methyl-3-hydroxy-2-hexanone, 2,5-dimethylhexane, 2,5-dimethyl-2-hexene, 2,5-dimethyl-3-hexene, 2,5-dimethyl-3-hexanol, 2,5-dimethyl-3-hexanone, 2,5-dimethyl-3,4-hexanediol, 2,5-dimethyl-3,4-hexanedione, 2,5-dimethyl-3-hydroxy-4-hexanone, 5-methyl-1-phenylhexane, 4-methyl-1-phenylhexane, 5-methyl-1-phenyl-1-hexene, 5-methyl-1-phenyl-2-hexene, 5-methyl-1-phenyl-3-hexene, 4-methyl-1-phenyl-1-hexene, 4-methyl-1-phenyl-2-hexene, 4-methyl-1-phenyl-3-hexene, 5-methyl-1-phenyl-2-hexanol, 5-methyl-1-phenyl-3-hexanol, 4-methyl-1-phenyl-2-hexanol, 4-methyl-1-phenyl-3-hexanol, 5-methyl-1-phenyl-2-hexanone, 5-methyl-1-phenyl-3-hexanone, 4-methyl-1-phenyl-2-hexanone, 4-methyl-1-phenyl-3-hexanone, 5-methyl-1-phenyl-2,3-hexanediol, 4-methyl-1-phenyl-2,3-hexanediol, 5-methyl-1-phenyl-3-hydroxy-2-hexanone, 5-methyl-1-phenyl-2-hydroxy-3-hexanone, 4-methyl-1-phenyl-3-hydroxy-2-hexanone, 4-methyl-1-phenyl-2-hydroxy-3-hexanone, 5-methyl-1-phenyl-2,3-hexanedione, 4-methyl-1-phenyl-2,3-hexanedione, 4-methyl-1-(4-hydroxyphenyl)hexane, 5-methyl-1-(4-hydroxyphenyl)-1-hexene, 5-methyl-1-(4-hydroxyphenyl)-2-hexene, 5-methyl-1-(4-hydroxyphenyl)-3-hexene, 4-methyl-1-(4-hydroxyphenyl)-1-hexene, 4-methyl-1-(4-hydroxyphenyl)-2-hexene, 4-methyl-1-(4-hydroxyphenyl)-3-hexene, 5-methyl-1-(4-hydroxyphenyl)-2-hexanol, 5-methyl-1-(4-hydroxyphenyl)-3-hexanol, 4-methyl-1-(4-hydroxyphenyl)-2-hexanol, 4-methyl-1-(4-hydroxyphenyl)-3-hexanol, 5-methyl-1-(4-hydroxyphenyl)-2-hexanone, 5-methyl-1-(4-hydroxyphenyl)-3-hexanone, 4-methyl-1-(4-hydroxyphenyl)-2-hexanone, 4-methyl-1-(4-hydroxyphenyl)-3-hexanone, 5-methyl-1-(4-hydroxyphenyl)-2,3-hexanediol, 4-methyl-1-(4-hydroxyphenyl)-2,3-hexanediol, 5-methyl-1-(4-hydroxyphenyl)-3-hydroxy-2-hexanone, 5-methyl-1-(4-hydroxyphenyl)-2-hydroxy-3-hexanone, 4-methyl-1-(4-hydroxyphenyl)-3-hydroxy-2-hexanone, 4-methyl-1-(4-hydroxyphenyl)-2-hydroxy-3-hexanone, 5-methyl-1-(4-hydroxyphenyl)-2,3-hexanedione, 4-methyl-1-(4-hydroxyphenyl)-2,3-hexanedione, 4-methyl-1-(indole-3-)hexane, 5-methyl-1-(indole-3)-1-hexene, 5-methyl-1-(indole-3)-2-hexene, 5-methyl-1-(indole-3)-3-hexene, 4-methyl-1-(indole-3)-1-hexene, 4-methyl-1-(indole-3)-2-hexene, 4-methyl-1-(indole-3)-3-hexene, 5-methyl-1-(indole-3)-2-hexanol, 5-methyl-1-(indole-3)-3-hexanol, 4-methyl-1-(indole-3)-2-hexanol, 4-methyl-1-(indole-3)-3-hexanol, 5-methyl-1-(indole-3)-2-hexanone, 5-methyl-1-(indole-3)-3-hexanone, 4-methyl-1-(indole-3)-2-hexanone, 4-methyl-1-(indole-3)-3-hexanone, 5-methyl-1-(indole-3)-2,3-hexanediol, 4-methyl-1-(indole-3)-2,3-hexanediol, 5-methyl-1-(indole-3)-3-hydroxy-2-hexanone, 5-methyl-1-(indole-3)-2-hydroxy-3-hexanone, 4-methyl-1-(indole-3)-3-hydroxy-2-hexanone, 4-methyl-1-(indole-3)-2-hydroxy-3-hexanone, 5-methyl-1-(indole-3)-2,3-hexanedione, 4-methyl-1-(indole-3)-2,3-hexanedione, n-heptane, 1-heptene, 1-heptanol, heptanal, heptanoate, 2-heptene, 3-heptene, 2-heptanol, 3-heptanol, 4-heptanol, 2-heptanone, 3-heptanone, 4-heptanone, 2,3-heptanediol, 2,3-heptanedione, 3,4-heptanediol, 3,4-heptanedione, 2-hydroxy-3-heptanone, 3-hydroxy-2-heptanone, 3-hydroxy-4-heptanone, 4-hydroxy-3-heptanone, 2-methylheptane, 3-methylheptane, 6-methyl-2-heptene, 6-methyl-3-heptene, 2-methyl-3-heptene, 2-methyl-2-heptene, 5-methyl-2-heptene, 5-methyl-3-heptene, 3-methyl-3-heptene, 2-methyl-3-heptanol, 2-methyl-4-heptanol, 6-methyl-3-heptanol, 5-methyl-3-heptanol, 3-methyl-4-heptanol, 2-methyl-3-heptanone, 2-methyl-4-heptanone, 6-methyl-3-heptanone, 5-methyl-3-heptanone, 3-methyl-4-heptanone, 2-methyl-3,4-heptanediol, 2-methyl-3,4-heptanedione, 6-methyl-3,4-heptanediol, 6-methyl-3,4-heptanedione, 5-methyl-3,4-heptanediol, 5-methyl-3,4-heptanedione, 2-methyl-3-hydroxy-4-heptanone, 2-methyl-4-hydroxy-3-heptanone, 6-methyl-3-hydroxy-4-heptanone, 6-methyl-4-hydroxy-3-heptanone, 5-methyl-3-hydroxy-4-heptanone, 5-methyl-4-hydroxy-3-heptanone, 2,6-dimethylheptane, 2,5-dimethylheptane, 2,6-dimethyl-2-heptene, 2,6-dimethyl-3-heptene, 2,5-dimethyl-2-heptene, 2,5-dimethyl-3-heptene, 3,6-dimethyl-3-heptene, 2,6-dimethyl-3-heptanol, 2,6-dimethyl-4-heptanol, 2,5-dimethyl-3-heptanol, 2,5-dimethyl-4-heptanol, 2,6-dimethyl-3,4-heptanediol, 2,6-dimethyl-3,4-heptanedione, 2,5-dimethyl-3,4-heptanediol, 2,5-dimethyl-3,4-heptanedione, 2,6-dimethyl-3-hydroxy-4-heptanone, 2,6-dimethyl-4-hydroxy-3-heptanone, 2,5-dimethyl-3-hydroxy-4-heptanone, 2,5-dimethyl-4-hydroxy-3-heptanone, n-octane, 1-octene, 2-octene, 1-octanol, octanal, octanoate, 3-octene, 4-octene, 4-octanol, 4-octanone, 4,5-octanediol, 4,5-octanedione, 4-hydroxy-5-octanone, 2-methyloctane, 2-methyl-3-octene, 2-methyl-4-octene, 7-methyl-3-octene, 3-methyl-3-octene, 3-methyl-4-octene, 6-methyl-3-octene, 2-methyl-4-octanol, 7-methyl-4-octanol, 3-methyl-4-octanol, 6-methyl-4-octanol, 2-methyl-4-octanone, 7-methyl-4-octanone, 3-methyl-4-octanone, 6-methyl-4-octanone, 2-methyl-4,5-octanediol, 2-methyl-4,5-octanedione, 3-methyl-4,5-octanediol, 3-methyl-4,5-octanedione, 2-methyl-4-hydroxy-5-octanone, 2-methyl-5-hydroxy-4-octanone, 3-methyl-4-hydroxy-5-octanone, 3-methyl-5-hydroxy-4-octanone, 2,7-dimethyloctane, 2,7-dimethyl-3-octene, 2,7-dimethyl-4-octene, 2,7-dimethyl-4-octanol, 2,7-dimethyl-4-octanone, 2,7-dimethyl-4,5-octanediol, 2,7-dimethyl-4,5-octanedione, 2,7-dimethyl-4-hydroxy-5-octanone, 2,6-dimethyloctane, 2,6-dimethyl-3-octene, 2,6-dimethyl-4-octene, 3,7-dimethyl-3-octene, 2,6-dimethyl-4-octanol, 3,7-dimethyl-4-octanol, 2,6-dimethyl-4-octanone, 3,7-dimethyl-4-octanone, 2,6-dimethyl-4,5-octanediol, 2,6-dimethyl-4,5-octanedione, 2,6-dimethyl-4-hydroxy-5-octanone, 2,6-dimethyl-5-hydroxy-4-octanone, 3,6-dimethyloctane, 3,6-dimethyl-3-octene, 3,6-dimethyl-4-octene, 3,6-dimethyl-4-octanol, 3,6-dimethyl-4-octanone, 3,6-dimethyl-4,5-octanediol, 3,6-dimethyl-4,5-octanedione, 3,6-dimethyl-4-hydroxy-5-octanone, n-nonane, 1-nonene, 1-nonanol, nonanal, nonanoate, 2-methylnonane, 2-methyl-4-nonene, 2-methyl-5-nonene, 8-methyl-4-nonene, 2-methyl-5-nonanol, 8-methyl-4-nonanol, 2-methyl-5-nonanone, 8-methyl-4-nonanone, 8-methyl-4,5-nonanediol, 8-methyl-4,5-nonanedione, 8-methyl-4-hydroxy-5-nonanone, 8-methyl-5-hydroxy-4-nonanone, 2,8-dimethylnonane, 2,8-dimethyl-3-nonene, 2,8-dimethyl-4-nonene, 2,8-dimethyl-5-nonene, 2,8-dimethyl-4-nonanol, 2,8-dimethyl-5-nonanol, 2,8-dimethyl-4-nonanone, 2,8-dimethyl-5-nonanone, 2,8-dimethyl-4,5-nonanediol, 2,8-dimethyl-4,5-nonanedione, 2,8-dimethyl-4-hydroxy-5-nonanone, 2,8-dimethyl-5-hydroxy-4-nonanone, 2,7-dimethylnonane, 3,8-dimethyl-3-nonene, 3,8-dimethyl-4-nonene, 3,8-dimethyl-5-nonene, 3,8-dimethyl-4-nonanol, 3,8-dimethyl-5-nonanol, 3,8-dimethyl-4-nonanone, 3,8-dimethyl-5-nonanone, 3,8-dimethyl-4,5-nonanediol, 3,8-dimethyl-4,5-nonanedione, 3,8-dimethyl-4-hydroxy-5-nonanone, 3,8-dimethyl-5-hydroxy-4-nonanone, n-decane, 1-decene, 1-decanol, decanoate, 2,9-dimethyldecane, 2,9-dimethyl-3-decene, 2,9-dimethyl-4-decene, 2,9-dimethyl-5-decanol, 2,9-dimethyl-5-decanone, 2,9-dimethyl-5,6-decanediol, 2,9-dimethyl-6-hydroxy-5-decanone, 2,9-dimethyl-5,6-decanedionen-undecane, 1-undecene, 1-undecanol, undecanal. undecanoate, n-dodecane, 1-dodecene, 1-dodecanol, dodecanal, dodecanoate, n-dodecane, 1-decadecene, 1-dodecanol, ddodecanal, dodecanoate, n-tridecane, 1-tridecene, 1-tridecanol, tridecanal, tridecanoate, n-tetradecane, 1-tetradecene, 1-tetradecanol, tetradecanal, tetradecanoate, n-pentadecane, 1-pentadecene, 1-pentadecanol, pentadecanal, pentadecanoate, n-hexadecane, 1-hexadecene, 1-hexadecanol, hexadecanal, hexadecanoate, n-heptadecane, 1-heptadecene, 1-heptadecanol, heptadecanal, heptadecanoate, n-octadecane, 1-octadecene, 1-octadecanol, octadecanal, octadecanoate, n-nonadecane, 1-nonadecene, 1-nonadecanol, nonadecanal, nonadecanoate, eicosane, 1-eicosene, 1-eicosanol, eicosanal, eicosanoate, 3-hydroxy propanal, 1,3-propanediol, 4-hydroxybutanal, 1,4-butanediol, 3-hydroxy-2-butanone, 2,3-butandiol, 1,5-pentane diol, homocitrate, homoisocitorate, b-hydroxy adipate, glutarate, glutarsemialdehyde, glutaraldehyde, 2-hydroxy-1-cyclopentanone, 1,2-cyclopentanediol, cyclopentanone, cyclopentanol, (S)-2-acetolactate, (R)-2,3-Dihydroxy-isovalerate, 2-oxoisovalerate, isobutyryl-CoA, isobutyrate, isobutyraldehyde, 5-amino pentaldehyde, 1,10-diaminodecane, 1,10-diamino-5-decene, 1,10-diamino-5-hydroxydecane, 1,10-diamino-5-decanone, 1,10-diamino-5,6-decanediol, 1,10-diamino-6-hydroxy-5-decanone, phenylacetoaldehyde, 1,4-diphenylbutane, 1,4-diphenyl-1-butene, 1,4-diphenyl-2-butene, 1,4-diphenyl-2-butanol, 1,4-diphenyl-2-butanone, 1,4-diphenyl-2,3-butanediol, 1,4-diphenyl-3-hydroxy-2-butanone, 1-(4-hydeoxyphenyl)-4-phenylbutane, 1-(4-hydeoxyphenyl)-4-phenyl-1-butene, 1-(4-hydeoxyphenyl)-4-phenyl-2-butene, 1-(4-hydeoxyphenyl)-4-phenyl-2-butanol, 1-(4-hydeoxyphenyl)-4-phenyl-2-butanone, 1-(4-hydeoxyphenyl)-4-phenyl-2,3-butanediol, 1-(4-hydeoxyphenyl)-4-phenyl-3-hydroxy-2-butanone, 1-(indole-3)-4-phenylbutane, 1-(indole-3)-4-phenyl-1-butene, 1-(indole-3)-4-phenyl-2-butene, 1-(indole-3)-4-phenyl-2-butanol, 1-(indole-3)-4-phenyl-2-butanone, 1-(indole-3)-4-phenyl-2,3-butanediol, 1-(indole-3)-4-phenyl-3-hydroxy-2-butanone, 4-hydroxyphenylacetoaldehyde, 1,4-di(4-hydroxyphenyl)butane, 1,4-di(4-hydroxyphenyl)-1-butene, 1,4-di(4-hydroxyphenyl)-2-butene, 1,4-di(4-hydroxyphenyl)-2-butanol, 1,4-di(4-hydroxyphenyl)-2-butanone, 1,4-di(4-hydroxyphenyl)-2,3-butanediol, 1,4-di(4-hydroxyphenyl)-3-hydroxy-2-butanone, 1-(4-hydroxyphenyl)-4-(indole-3-)butane, 1-(4-hydroxyphenyl)-4-(indole-3)-1-butene, 1-di(4-hydroxyphenyl)-4-(indole-3)-2-butene, 1-(4-hydroxyphenyl)-4-(indole-3)-2-butanol, 1-(4-hydroxyphenyl)-4-(indole-3)-2-butanone, 1-(4-hydroxyphenyl)-4-(indole-3)-2,3-butanediol, 1-(4-hydroxyphenyl-4-(indole-3)-3-hydroxy-2-butanone, indole-3-acetoaldehyde, 1,4-di(indole-3-)butane, 1,4-di(indole-3)-1-butene, 1,4-di(indole-3)-2-butene, 1,4-di(indole-3)-2-butanol, 1,4-di(indole-3)-2-butanone, 1,4-di(indole-3)-2,3-butanediol, 1,4-di(indole-3)-3-hydroxy-2-butanone, succinate semialdehyde, hexane-1,8-dicarboxylic acid, 3-hexene-1,8-dicarboxylic acid, 3-hydroxy-hexane-1,8-dicarboxylic acid, 3-hexanone-1,8-dicarboxylic acid, 3,4-hexanediol-1,8-dicarboxylic acid, 4-hydroxy-3-hexanone-1,8-dicarboxylic acid, fucoidan, iodine, chlorophyll, carotenoid, calcium, magnesium, iron, sodium, potassium, phosphate, lactic acid, acetic acid, formic acid, or isoprenoids and terpenes. Additional fermentation end products, and methods of production thereof, can be found in U.S. patent application Ser. No. 12/969,582, which is herein incorporated by reference in its entirety.

### Modification to Alter Enzyme Activity

In various embodiments, one or more modification of conditions for hydrolysis and/or fermentation is implemented to enhance end-product production. Examples of such modifications include genetic modification to enhance enzyme activity in a microorganism that already comprises genes for encoding one or more target enzymes, introducing one or more heterogeneous nucleic acid molecules into a host microorganism to express and enhance activity of an enzyme not otherwise expressed in the host, genetic modifications to disrupt the expression of one or more metabolic pathway genes to direct, modifying physical and chemical conditions to enhance enzyme function (e.g., modifying and/or maintaining a certain temperature, pH, nutrient concentration, temporal), or a combination of one or more such modifications. Other embodiments include overexpression of an endogenous nucleic acid molecule into the host microorganism to express and enhance activity of an enzyme already expressed in the host or to express activity of an enzyme in the host when the enzyme would not normally be expressed in the naturally-occurring host microorganism.

### Genetic Modification

### Genetic Modification to Enhance Enzymatic Activity

In one embodiment, a microorganism can be genetically modified to enhance enzyme activity of one or more enzymes, including but not limited to hydrolytic enzymes (such as cellulase(s), hemicellulase(s), or pectinase(s) etc.), decarboxylases (e.g. pyruvate decarboxylase), dehydrogenases (e.g. alcohol dehydrogenase), and synthetases (e.g. Acetyl CoA synthetase). In one embodiment a method is used to genetically modify a microorganism (such as a Clostridium species) that is disclosed in US 20100086981 or PCT/US2010/40494, which are herein incorporated by reference in their entirety. In another embodiment, an enzyme can be selected from the annotated genome of C. phytofermentans, another bacterial species, such as B. subtilis, E. coli, various Clostridium species, or yeasts such as S. cerevisiae for utilization in products and processes described herein. Examples include enzymes such as L-butanediol dehydrogenase, acetoin reductase, 3-hydroxyacyl-CoA dehydrogenase, cis-aconitate decarboxylase or the like, to create pathways for new products from biomass.

Examples of such modifications include modifying endogenous nucleic acid regulatory elements to increase expression of one or more enzymes (e.g., operably linking a gene encoding a target enzyme to a strong promoter), introducing into a microorganism additional copies of endogenous nucleic acid molecules to provide enhanced activity of an enzyme by increasing its production, and operably linking genes encoding one or more enzymes to an inducible promoter or a combination thereof.

A variety of promoters (e.g., constitutive promoters, inducible promoters) can be used to drive expression of the heterologous genes in a recombinant host microorganism.

Promoters typically used in recombinant technology, such as E. coli lac and trp operons, the tac promoter, the bacteriophage pL promoter, bacteriophage T7 and SP6 promoters, beta-actin promoter, insulin promoter, baculoviral polyhedrin and p10 promoter, can be used to initiate transcription.

In one embodiment a constitutive promoter can be used including, but not limited to the int promoter of bacteriophage lamda, the bla promoter of the beta-lactamase gene sequence of pBR322, hydA or thlA in Clostridium, S. coelicolor hrdB, or whiE, the CAT promoter of the chloramphenicol acetyl transferase gene sequence of pPR325, Staphylococcal constitutive promoter blaZ and the like.

In another embodiment an inducible promoter can be used that regulates the expression of downstream gene in a controlled manner, such as under a specific condition of a cell culture. Examples of inducible prokaryotic promoters include, but are not limited to, the major right and left promoters of bacteriophage, the trp, reca, lacZ, AraC and gal promoters of E. coli, the alpha-amylase (Ulmanen Ett at., J. Bacteriol. 162:176-182, 1985, which is herein incorporated by reference in its entirety) and the sigma-28-specific promoters of B. subtilis (Gilman et al., Gene sequence 32:11-20 (1984), which is herein incorporated by reference in its entirety), the promoters of the bacteriophages of Bacillus (Gryczan, In: The Molecular Biology of the Bacilli, Academic Press, Inc., NY (1982), which is herein incorporated by reference in its entirety), Streptomyces promoters (Ward et at., Mol. Gen. Genet. 203:468-478, 1986, which is herein incorporated by reference in its entirety), and the like. Exemplary prokaryotic promoters are reviewed by Glick (J. Ind. Microtiot. 1:277-282, 1987, which is herein incorporated by reference in its entirety); Cenatiempo (Biochimie 68:505-516, 1986, which is herein incorporated by reference in its entirety); and Gottesman (Ann. Rev. Genet. 18:415-442, 1984, which is herein incorporated by reference in its entirety).

A promoter that is constitutively active under certain culture conditions, can be inactive in other conditions. For example, the promoter of the hydA gene from Clostridium acetobutylicum, wherein expression is known to be regulated by the environmental pH. Furthermore, temperature-regulated promoters are also known and can be used. In some embodiments, depending on the desired host cell, a pH-regulated or temperature-regulated promoter can be used with an expression constructs to initiate transcription. Other pH-regulatable promoters are known, such as P170 functioning in lactic acid bacteria, as disclosed in US Patent Application No. 20020137140, which is herein incorporated by reference in its entirety.

In general, to express the desired gene/nucleotide sequence efficiently, various promoters can be used; e.g., the original promoter of the gene, promoters of antibiotic resistance genes such as for instance kanamycin resistant gene of Tn5, ampicillin resistant gene of pBR322, and promoters of lambda phage and any promoters which can be functional in the host cell. For expression, other regulatory elements, such as for instance a Shine-Dalgarno (SD) sequence (e.g., AGGAGG and so on including natural and synthetic sequences operable in a host cell) and a transcriptional terminator (inverted repeat structure including any natural and synthetic sequence) which are operable in a host cell (into which a coding sequence is introduced to provide a recombinant cell) can be used with the above described promoters.

Examples of promoters that can be used with a product or process disclosed herein include those disclosed in the following patent documents: US20040171824, U.S. Pat. No. 6,410,317, WO 2005/024019, which are herein incorporated by reference in their entirety. Several promoter-operator systems, such as lac, (D. V. Goeddel et al., “Expression in Escherichia coli of Chemically Synthesized Genes for Human Insulin”, Proc. Nat. Acad. Sci. U.S.A., 76:106-110 (1979), which is herein incorporated by reference in its entirety); tip (J. D. Windass et al. “The Construction of a Synthetic Escherichia coli Trp Promoter and Its Use in the Expression of a Synthetic Interferon Gene”, Nucl. Acids. Res., 10:6639-57 (1982), which is herein incorporated by reference in its entirety) and λ PL operons (R. Crowl et al., “Versatile Expression Vectors for High-Level Synthesis of Cloned Gene Products in Escherichia coli”, Gene, 38:31-38 (1985), which is herein incorporated by reference in its entirety) in E. coli and have been used for the regulation of gene expression in recombinant cells. The corresponding repressors are the lac repressor, trpR and cI, respectively.

Repressors are protein molecules that bind specifically to particular operators. For example, the lac repressor molecule binds to the operator of the lac promoter-operator system, while the cro repressor binds to the operator of the lambda pR promoter. Other combinations of repressor and operator are known in the art. See, e.g., J. D. Watson et al., Molecular Biology Of The Gene, p. 373 (4th ed. 1987), which is herein incorporated by reference in its entirety. The structure formed by the repressor and operator blocks the productive interaction of the associated promoter with RNA polymerase, thereby preventing transcription. Other molecules, termed inducers, bind to repressors, thereby preventing the repressor from binding to its operator. Thus, the suppression of protein expression by repressor molecules can be reversed by reducing the concentration of repressor (depression) or by neutralizing the repressor with an inducer.

Analogous promoter-operator systems and inducers are known in other microorganisms. In yeast, the GAL10 and GAL1 promoters are repressed by extracellular glucose, and activated by addition of galactose, an inducer. Protein GAL80 is a repressor for the system, and GAL4 is a transcriptional activator. Binding of GAL80 to galactose prevents GAL80 from binding GAL4. Then, GAL4 can bind to an upstream activation sequence (UAS) activating transcription. See Y. Oshima, “Regulatory Circuits For Gene Expression: The Metabolisms Of Galactose And Phosphate” in The Molecular Biology Of The Yeast Sacharomyces, Metabolism And Gene Expression, J. N. Strathern et al. eds. (1982), which are herein incorporated by reference in their entirety.

Transcription under the control of the PHO5 promoter is repressed by extracellular inorganic phosphate, and induced to a high level when phosphate is depleted. R. A. Kramer and N. Andersen, “Isolation of Yeast Genes With mRNA Levels Controlled By Phosphate Concentration”, Proc. Nat. Acad. Sci. U.S.A., 77:6451-6545 (1980), which is herein incorporated by reference in its entirety. A number of regulatory genes for PHO5 expression have been identified, including some involved in phosphate regulation.

Matα2 is a temperature-regulated promoter system in yeast. A repressor protein, operator and promoter sites have been identified in this system. A. Z. Sledziewski et al., “Construction Of Temperature-Regulated Yeast Promoters Using The Matα2 Repression System”, Bio/Technology, 6:411-16 (1988), which is herein incorporated by reference in its entirety.

Another example of a repressor system in yeast is the CUP1 promoter, which can be induced by Cu⁺2 ions. The CUP1 promoter is regulated by a metallothionine protein. J. A. Gorman et al., “Regulation Of The Yeast Metallothionine Gene”, Gene, 48:13-22 (1986), which is herein incorporated by reference in its entirety.

Promoter elements can be selected and mobilized in a vector (e.g., pIMPCphy). For example, a transcription regulatory sequence is operably linked to gene(s) of interest (e.g., in a expression construct). The promoter can be any array of DNA sequences that interact specifically with cellular transcription factors to regulate transcription of the downstream gene. The selection of a particular promoter depends on what cell type is to be used to express the protein of interest. In one embodiment a transcription regulatory sequences can be derived from the host microorganism. In various embodiments, constitutive or inducible promoters are selected for use in a host cell. Depending on the host cell, there are potentially hundreds of constitutive and inducible promoters which are known and that can be engineered to function in the host cell.

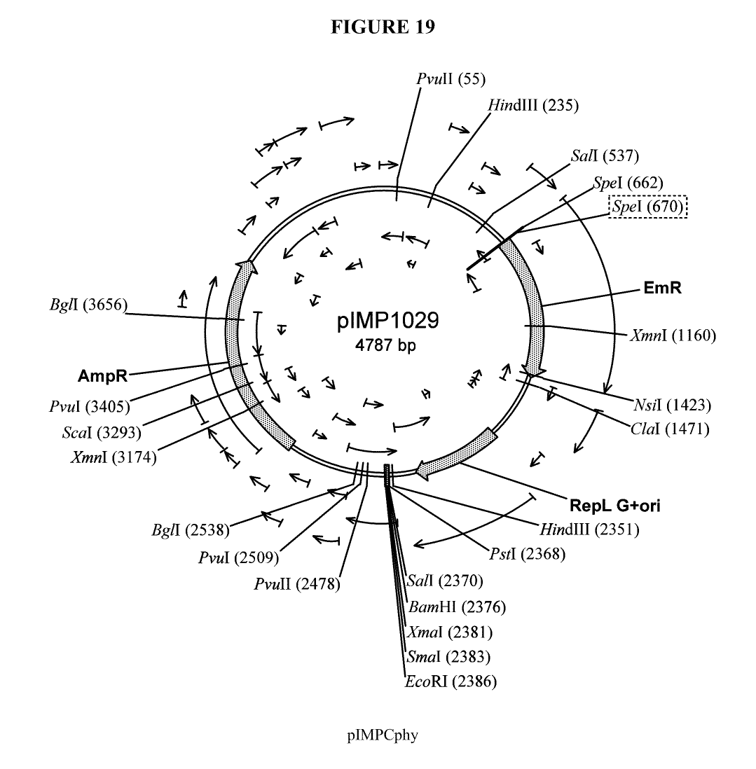

A map of the plasmid pIMPCphy is shown in FIG. 19, and the DNA sequence of this plasmid is provided as SEQ ID NO: 1.

SEQ ID NO: 1:
gcgcccaatacgcaaaccgcctctccccgcgcgttggccgattcatta
atgcagctggcacgacaggtttcccgactggaaagcgggcagtgagcg
caacgcaattaatgtgagttagctcactcattaggcaccccaggcttt
acactttatgcttccggctcgtatgttgtgtggaattgtgagcggata
acaatttcacacaggaaacagctatgaccatgattacgccaaagcttt
ggctaacacacacgccattccaaccaataggttctcggcataaagcca
tgctctgacgataaatgcactaatgccttaaaaaaacattaaagtcta
acacactagacttatttacttcgtaattaagtcgttaaaccgtgtgct
ctacgaccaaaagtataaaacctttaagaactttcttttttcttgtaa
aaaaagaaactagataaatctctcatatcttttattcaataatcgcat
cagattgcagtataaatttaacgatcactcatcatgttcatatttatc
agagctccttatattttatttcgatttatttgttatttatttaacatt
tttctattgacctcatcttttctatgtgttattcttttgttaattgtt
tacaaataatctacgatacatagaaggaggaaaaactagtatactagt
atgaacgagaaaaatataaaacacagtcaaaactttattacttcaaaa
cataatatagataaaataatgacaaatataagattaaatgaacatgat
aatatctttgaaatcggctcaggaaaagggcattttacccttgaatta
gtacagaggtgtaatttcgtaactgccattgaaatagaccataaatta
tgcaaaactacagaaaataaacttgttgatcacgataatttccaagtt
ttaaacaaggatatattgcagtttaaatttcctaaaaaccaatcctat
aaaatatttggtaatataccttataacataagtacggatataatacgc
aaaattgtttttgatagtatagctgatgagatttatttaatcgtggaa
tacgggtttgctaaaagattattaaatacaaaacgctcattggcatta
tttttaatggcagaagttgatatttctatattaagtatggttccaaga
gaatattttcatcctaaacctaaagtgaatagctcacttatcagatta
aatagaaaaaaatcaagaatatcacacaaagataaacagaagtataat
tatttcgttatgaaatgggttaacaaagaatacaagaaaatatttaca
aaaaatcaatttaacaattccttaaaacatgcaggaattgacgattta
aacaatattagctttgaacaattatatctatttcaatagctataaatt
atttaataagtaagttaagggatgcataaactgcatcccttaacttgt
ttttcgtgtacctattttttgtgaatcgatccggccagcctcgcagag
caggattcccgttgagcaccgccaggtgcgaataagggacagtgaaga
aggaacacccgctcgcgggtgggcctacttcacctatcctgcccggat
cgattatgtcttttgcgcattcacttatttctatataaatatgagcga
agcgaataagcgtcggaaaagcagcaaaaagtttcctttttgctgttg
gagcatgggggttcagggggtgcagtatctgacgtcaatgccgagcga
aagcgagccgaagggtagcatttacgttagataaccccctgatatgct
ccgacgctttatatagaaaagaagattcaactaggtaaaatcttaata
taggttgagatgataaggtttataaggaatttgtttgttctaattttt
cactcattttgttctaatttcttttaacaaatgttcttttttttttag
aacagttatgatatagttagaatagtttaaaataaggagtgagaaaaa
gatgaaagaaagatatggaacagtctataaaggctdcagaggctcata
acgaagaaagtggagaagtcatagaggtagacaagttataccgtaaac
aaacgtctggtaacttcgtaaaggcatatatagtgcaattaataagta
tgttagatatgattggcggaaaaaaacttaaaatcgttaactatatcc
tagataatgtccacttaagtaacaatacaatgatagctacaacaagag
aaatagcaaaagctacaggaacaagtctacaaacagtaataacaacac
ttaaaatcttagaagaaggaaatattataaaaagaaaaactggagtat
taatgttaaaccctgaactactaatgagaggcgacgaccaaaaacaaa
aatacctcttactcgaatttgggaactttgagcaagaggcaaatgaaa
tagattgacctcccaataacaccacgtagttattgggaggtcaatcta
tgaaatgcgattaagcttagcttggctgcaggtcgacggatccccggg
aattcactggccgtcgttttacaacgtcgtgactgggaaaaccctggc
gttacccaacttaatcgccttgcagcacatccccctttcgccagctgg
cgtaatagcgaagaggcccgcaccgatcgccatcccaacagttgcgca
gcctgaatggcgaatggcgcctgatgcggtattttctccttacgcatc
tgtgcggtatttcacaccgcatatggtgcactctcagtacaatctgct
ctgatgccgcatagttaagccagccccgacacccgccaacacccgctg
acgcgccctgacgggcttgtctgctcccggcatccgcttacagacaag
ctgtgaccgtctccgggagctgcatgtgtcagaggttttcaccgtcat
caccgaaacgcgcgagacgaaagggcctcgtgatacgcctatttttat
aggttaatgtcatgataataatggtttcttagacgtcaggtggcactt
ttcggggaaatgtgcgcggaacccctatttgtttatttttctaaatac
attcaaatatgtatccgctcatgagacaataaccctgataaatgcttc
aataatattgaaaaaggaagagtatgagtattcaacatttccgtgtcg
cccttattcccttttttgcggcattttgccttcctgtttttgctcacc
cagaaacgctggtgaaagtaaaagatgctgaagatcagttgggtgcac
gagtgggttacatcgaactggatctcaacagcggtaagatccttgaga
gttttcgccccgaagaacgttttccaatgatgagcacttttaaagttc
tgctatgtggcgcggtattatcccgtattgacgccgggcaagagcaac
tcggtcgccgcatacactattctcagaatgacttggttgagtactcac
cagtcacagaaaagcatcttacggatggcatgacagtaagagaattat
gcagtgctgccataaccatgagtgataacactgcggccaacttacttc
tgacaacgatcggaggaccgaaggagctaaccgcttttttgcacaaca
tgggggatcatgtaactcgccttgatcgttgggaaccggagctgaatg
aagccataccaaacgacgagcgtgacaccacgatgcctgtagcaatgg
caacaacgttgcgcaaactattaactggcgaactacttactctagatc
ccggcaacaattaatagactggatggaggcggataaagttgcaggacc
acttctgcgctcggcccttccggctggctggtttattgctgataaatc
tggagccggtgagcgtgggtctcgcggtatcattgcagcactggggcc
agatggtaagccctcccgtatcgtagttatctacacgacggggagtca
ggcaactatggatgaacgaaatagacagatcgctgagataggtgcctc
actgattaagcattggtaactgtcagaccaagtttactcatatatact
ttagattgatttaaaacttcatttttaatttaaaaggatctaggtgaa
gatcctttttgataatctcatgaccaaaatcccttaacgtgagttttc
gttccactgagcgtcagaccccgtagaaaagatcaaaggatcttatga
gatcctttttttctgcgcgtaatctgctgcttgcaaacaaaaaaacca
ccgctaccagcggtggtttgtttgccggatcaagagctaccaactatt
ttccgaaggtaactggcttcagcagagcgcagataccaaatactgtcc
ttctagtgtagccgtagttaggccaccacttcaagaactctgtagcac
cgcctacatacctcgctctgctaatcctgttaccagtggctgctgcca
gtggcgataagtcgtgtcttaccgggttggactcaagacgatagttac
cggataaggcgcagcggtcgggctgaacggggggttcgtgcacacagc
ccagcttggagcgaacgacctacaccgaactgagatacctacagcgtg
agctatgagaaagcgccacgcttcccgaagggagaaaggcggacaggt
atccggtaagcggcagggtcggaacaggagagcgcacgagggagcttc
cagggggaaacgcctggtatctttatagtcctgtcgggtttcgccacc
tctgacttgagcgtcgatttttgtgatgctcgtcaggggggcggagcc
tatggaaaaacgccagcaacgcggcctttttacggttcctggcctttt
gctggccttttgctcacatgttctttcctgcgttatcccctgattctg
tggataaccgtattaccgcctttgagtgagctgataccgctcgccgca
gccgaacgccgagcgcagcgagtcagtgagcgaggaagcggaaga

The vector pIMPCphy was constructed as a shuttle vector for C. phytofermentans and is further described in U.S. Patent Application Publication US20100086981, which is herein incorporated by reference in its entirety. It has an Ampicillin-resistance cassette and an Origin of Replication (ori) for selection and replication in E. coli. It contains a Gram-positive origin of replication that allows the replication of the plasmid in C. phytofermentans. In order to select for the presence of the plasmid, the pIMPCphy carries an erythromycin resistance gene under the control of the C. phytofermentans promoter of the gene Cphyl 029. This plasmid can be transferred to C. phytofermentans by electroporation or by transconjugation with an E. coli strain that has a mobilizing plasmid, for example pRK2030. A plasmid map of pIMPCphy is depicted in FIG. 19. pIMPCphy is an effective replicative vector system for all microorganisms, including all gram⁺ and gram⁻ bacteria, and fungi (including yeasts). A further discussion of promoters, regulation of gene expression products, and additional genetic modifications can be found in U.S. Patent Application Publication US 20100086981A1, which is herein incorporated by reference in its entirety.

Due to inherent cellular mechanisms, it is a challenge to express many forms of heterolgous genetic material in Clostridium due to the presence of the restriction and modification (RM) systems. RM systems in bacteria serve as a defense mechanism against foreign nucleic acids. In order to prevent genetic manipulation, bacterial RM systems are capable of attacking heterologous DNA through the use of enzymes such as DNA methyltransferase (MTase) and restriction endonuclease (REase). For example, bacterial MTases methylate DNA, creating a “self” signal, whereas bacterial REases are restriction enzyme that enymatically cleave DNA that is not methylated, “foreign” DNA. (Dong H. et al. (2010) PLOS One 5(2): e9038). Therefore, one method to achieve effective gene transfer to Clostridium, and avoid Clostridium RM systems, is to methylate a vector comprising heterologous DNA (Mermelstein and Papoutsakis. Appl. Environ. Microbiol. 59: 1077-1081 (1993); Mermelstein et al., Biotechnol. 10: 190-195 (1992)). In some embodiments, a vector comprising a heterologous DNA sequence is methylated prior to transformation into C. phytofermentans. In some embodiments, methylation can be accomplished by the phi3TI methyltransferase. In further embodiments, plasmid DNA can be transformed into DH10β E. coli harboring vector pDHKM (Zhao, et al. Appl. Environ. Microbiol. 69: 2831-41 (2003)) carrying an active copy of the phi3TI methyltransferase gene.

Additionally, variance exists amongst RM systems between different bacterial species. Therefore, another means to enhance heterologous DNA survival is to modify a vector to comprise enzyme restriction sites that are not recognized by a microorganism. In some embodiments, a DNA sequence comprising genetic material from a first microorganism is provided, wherein the DNA sequence comprises restriction enzyme sites that are not recognized by a second microorganism. In further embodiments, the DNA sequence encodes for a gene, or genetically modified variant of the gene, from C. phytofermentans. In further embodiments, the DNA sequence encodes for an expression product that is a protein, or fragment thereof, from C. phytofermentans. In further embodiments, the first microorganism is a Clostridium species and the second microorganism is bacteria or yeast, e.g. E. coli.

### Genetic Modification to Disrupt Enzymatic Activity

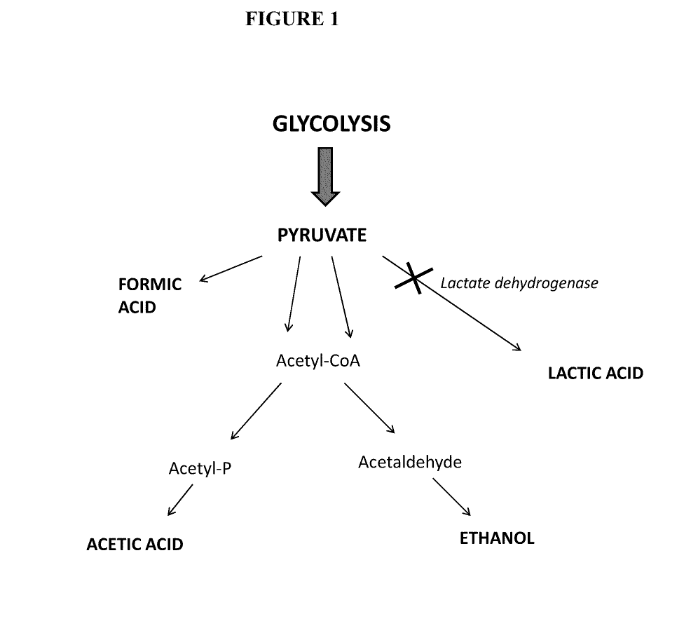

In one embodiment, a mesophilic microorganism is modified to disrupt the expression of one or more metabolic pathway genes (e.g. lactate dehydrogenase). The organism can be a naturally-occurring mesophilic organism or a mutated or recombinant organism. The term “wild-type” refers to any of these organisms with metabolic pathway gene activity that is normal for that organism. A non “wild-type” knockout is the wild-type organism that has been modified to reduce or eliminate activity of a metabolic pathway gene, e.g. lactate dehydrogenase activity or genes encoding for other enzymes listed in FIG. 1, compared to the wild-type activity level of that enzyme.

The nucleic acid sequence for a gene of interest (e.g. lactate dehydrogenase) can be used to target the gene for inactivation through different mechanisms. In one embodiment, a target gene (e.g. lactate dehydrogenase) is inactivated by the insertion of a transposon, or by the deletion of the gene sequence or a portion of the gene sequence. In one embodiment, the lactate dehydrogenase gene is inactivated by the integration of a plasmid that achieves natural homologous recombination or integration between the plasmid and the microorganism's chromosome. Chromosomal integrants can be selected for on the basis of their resistance to an antibacterial agent (for example, kanamycin). The integration into the lactate dehydrogenase gene may occur by a single cross-over recombination event or by a double (or more) cross-over recombination event.

For all DNA constructs in the described embodiments, an effective form is an expression vector. In one embodiment, the DNA construct is a plasmid or vector. In another embodiment, the plasmid comprises the nucleic acid sequence of SEQ ID NO: 2. In another embodiment, the plasmid comprises a nucleic acid with 70-99.9% similarity to the sequence of SEQ ID NO: 2. In another embodiment, the plasmid comprises a nucleic acid with 70% similarity to the sequence of SEQ ID NO: 2. In another embodiment, the plasmid comprises a nucleic acid with 75% similarity to the sequence of SEQ ID NO: 2. In another embodiment, the plasmid comprises a nucleic acid with 80% similarity to the sequence of SEQ ID NO: 2. In another embodiment, the plasmid comprises a nucleic acid with 85% similarity to the sequence of SEQ ID NO: 2. In another embodiment, the plasmid comprises a nucleic acid with 90% similarity to the sequence of SEQ ID NO:2. In another embodiment, the plasmid comprises a nucleic acid with 95% similarity to the sequence of SEQ ID NO: 2. In another embodiment, the plasmid comprises a nucleic acid with 99% similarity to the sequence of SEQ ID NO: 2. In a further embodiment, the DNA construct can only replicate in the host microorganism through recombination with the genome of the host microorganism.

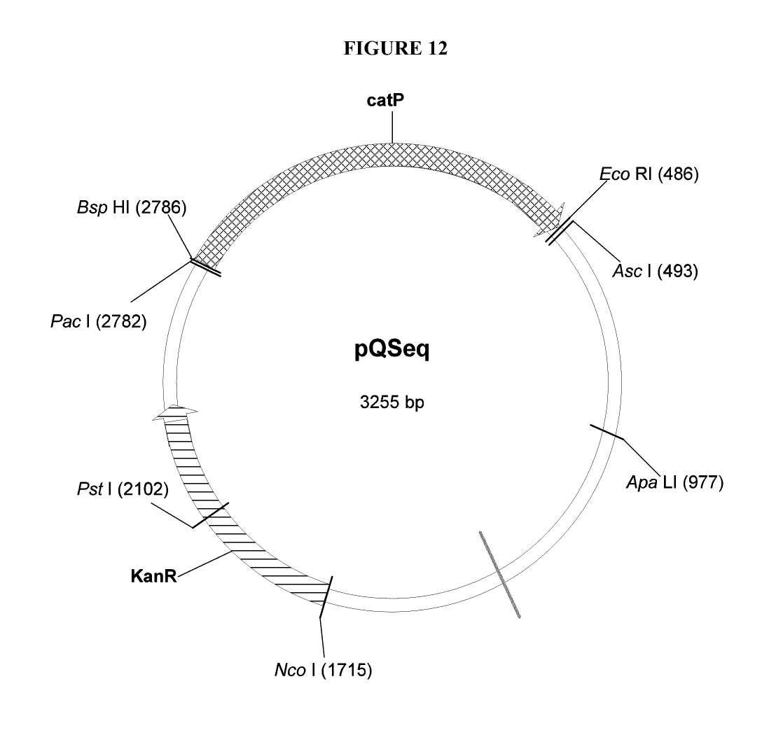

The pMA-0923071 plasmid lacks a gram positive origin of replication, and contains chloramphenicol acetyltransferase (catP) and kanamycin acetyltransferase sites, conferring chloramphenicol and kanamycin resistance, respectively. The fully sequenced version of the plasmid is shown in FIG. 12 (pQSeq) and below.

pQSeq plasmid sequence (SEQ ID NO: 2):
accaagctatacaatatttcacaatgatactgaaacattttccagcct
ttggactgagtgtaagtctgactttaaatcatttttagcagattatga
aagtgatacgcaacggtatggaaacaatcatagaatggaaggaaagcc
aaatgctccggaaaacatttttaatgtatctatgataccgtggtcaac
cttcgatggctttaatctgaatttgcagaaaggatatgattatttgat
tcctatttttactatggggaaatattataaagaagataacaaaattat
acttcctttggcaattcaagttcatcacgcagtatgtgacggatttca
catttgccgttttgtaaacgaattgcaggaattgataaatagttaact
tcaggtttgtctgtaactaaaaacaagtatttaagcaaaaacatcgta
gaaatacggtgttttttgttaccctaaaatctacaattttatacataa
ccacgaattcggcgcgccctgggcctcatgggccttcctttcactgcc
cgctttccagtcgggaaacctgtcgtgccagctgcattaacatggtca
tagctgtttccttgcgtattgggcgctctccgcttcctcgctcactga
ctcgctgcgctcggtcgttcgggtaaagcctggggtgcctaatgagca
aaaggccagcaaaaggccaggaaccgtaaaaaggccgcgttgctggcg
tttttccataggctccgcccccctgacgagcatcacaaaaatcgacgc
tcaagtcagaggtggcgaaacccgacaggactataaagataccaggcg
tttccccctggaagctccctcgtgcgctctcctgttccgaccctgccg
cttaccggatacctgtccgcctttctcccttcgggaagcgtggcgctt
tctcatagctcacgctgtaggtatctcagttcggtgtaggtcgttcgc
tccaagctgggctgtgtgcacgaaccccccgttcagcccgaccgctgc
gccttatccggtaactatcgtcttgagtccaacccggtaagacacgac
ttatcgccactggcagcagccactggtaacaggattagcagagcgagg
tatgtaggcggtgctacagagttatgaagtggtggcctaactacggct
acactagaagaacagtatttggtatctgcgctctgctgaagccagtta
ccttcggaaaaagagttggtagctcttgatccggcaaacaaaccaccg
ctggtagcggtggtttttttgtttgcaagcagcagattacgcgcagaa
aaaaaggatctcaagaagatcctttgatcttttctacggggtctgacg
ctcagtggaacgaaaactcacgttaagggattttggtcatgagattat
caaaaaggatatcacctagatccttttaaattaaaaatgaagttttaa
atcaatctaaagtatatatgagtaaacttggtctgacagttattagaa
aaattcatccagcagacgataaaacgcaatacgctggctatccggtgc
cgcaatgccatacagcaccagaaaacgatccgcccattcgccgcccag
ttcttccgcaatatcacgggtggccagcgcaatatcctgataacgatc
cgccacgcccagacggccgcaatcaataaagccgctaaaacggccatt
ttccaccataatgttcggcaggcacgcatcaccatgggtcaccaccag
atcttcgccatccggcatgctcgctttcagacgcgcaaacagctctgc
cggtgccaggccctgatgttcttcatccagatcatcctgtccaccagg
cccgcttccatacgggtacgcgcacgttcaatacgatgtttcgcctga
tgatcaaacggacaggtcgccgggtccagggtatgcagacgacgcatg
gcatccgccataatgctcactttttctgccggcgccagatggctagac
agcagatcctgacccggcacttcgcccagcagcagccaatcacggccc
gcttcggtcaccacatccagcaccgccgcacacggaacaccggtggtg
gccagccagctcagacgcgccgcttcatcctgcagctcgttcagcgca
ccgctcagatcggttttcacaaacagcaccggacgaccctgcgcgctc
agacgaaacaccgccgcatcagagcagccaatggtctgctgcgcccaa
tcatagccaaacagacgttccacccacgctgccgggctacccgcatgc
aggccatcctgttcaatcatactcttcctttttcaatattattgaagc
atttatcagggttattgtctcatgagcggatacatatttgaatgtatt
tagaaaaataaacaaataggggttccgcgcacatttccccgaaaagtg
ccacctaaattgtaagcgttaatattttgttaaaattcgcgttaaatt
tttgttaaatcagctcattttttaaccaataggccgaaatcggcaaaa
tcccttataaatcaaaagaatagaccgagatagggttgagtggccgct
acagggcgctcccattcgccattcaggctgcgcaactgttgggaaggg
cgtttcggtgcgggcctcttcgctattacgccagctggcgaaaggggg
atgtgctgcaaggcgattaagttgggtaacgccagggttttcccagtc
acgacgttgtaaaacgacggccagtgagcgcgacgtaatacgactcac
tatagggcgaattgaaggaaggccgtcaaggccgcatttaattaagga
tccggcagtttttctttttcggcaagtgttcaagaagttattaagtcg
ggagtgcagtcgaagtgggcaagttgaaaaattcacaaaaatgtggta
taatatctttgttcattagagcgataaacttgaatttgagagggaact
tagatggtatttgaaaaaattgataaaaatagttggaacagaaaagag
tattttgaccactactttgcaagtgtaccttgtacatacagcatgacc
gttaaagtggatatcacacaaataaaggaaaagggaatgaaactatat
cctgcaatgattattatattgcaatgattgtaaaccgccattcagagt
ttaggacggcaatcaatcaagatggtgaattggggatatatgatgaga
tgataccaagctatacaatatttcacaatgatactgaaacattttcca
gcctttggactgagtgtaagtctgactttaaatca

The DNA constructs in these embodiments can also incorporate a suitable reporter gene as an indicator of successful transformation. In one embodiment, the reporter gene is an antibiotic resistance gene, such as a kanamycin, ampicillin or chloramphenicol resistance gene. The DNA constructs can also incorporate multiple reporter genes, as appropriate.

Methods for the preparation and incorporation of these genes into microorganisms are known, for example in Ingram et al, Biotech & BioEng, 1998; 58 (2+3): 204-214 and U.S. Pat. No. 5,916,787, the content of each being incorporated herein by reference in their entirety. The genes may be introduced in a plasmid or integrated into the chromosome, as will be appreciated by a person skilled in the art.

The microorganisms described herein may be cultured under conventional culture conditions, depending on the mesophilic microorganism chosen. The choice of substrates, temperature, pH and other growth conditions can be selected based on known culture requirements, for example see WO01/49865 and WO01/85966, the content of each being incorporated herein by reference in their entirety.

### Non-Recombinant Genetic Modification

In other embodiments, a microorganism can be obtained without the use of recombinant DNA techniques that exhibit desirable properties such as increased productivity, increased yield, or increased titer. For example, mutagenesis, or random mutagenesis can be performed by chemical means or by irradiation of the microorganism. The population of mutagenized microorganisms can then be screened for beneficial mutations that exhibit one or more desirable properties. Screening can be performed by growing the mutagenized microorganisms on substrates that comprise carbon sources that will be utilized during the generation of end-products by fermentation. Screening can also include measuring the production of end-products during growth of the microorganism, or measuring the digestion or assimilation of the carbon source(s). The isolates so obtained can further be transformed with recombinant polynucleotides or used in combination with any of the methods and compositions provided herein to further enhance biofuel production.

Various methods can be used to produce and select mutants that differ from wild-type cells. In some instances, bacterial populations are treated with a mutagenic agent, for example, nitrosoguanidine (N-methyl-N′-nitro-N-nitrosoguanidine) or the like, to increase the mutation frequency above that of spontaneous mutagenesis. This is induced mutagenesis. Techniques for inducing mutagenesis include, but are not limited to, exposure of the bacteria to a mutagenic agent, such as x-rays or chemical mutagenic agents. More sophisticated procedures involve isolating the gene of interest and making a change in the desired location, then reinserting the gene into bacterial cells. This is site-directed mutagenesis.

Directed evolution is usually performed as three steps which can be repeated more than once. First, the gene encoding a protein of interest is mutated and/or recombined at random to create a large library of gene variants. The library is then screened or selected for the presence of mutants or variants that show the desired property. Screens enable the identification and isolation of high-performing mutants by hand; selections automatically eliminate all non functional mutants. Then the variants identified in the selection or screen are replicated, enabling DNA sequencing to determine what mutations occurred. Directed evolution can be carried out in vivo or in vitro. See, for example, Otten, L. G.; Quax, W. J. (2005). Biomolecular Engineering 22 (1-3): 1-9; Yuan, L., et al. (2005) Microbiol. Mol. Biol. Rev. 69 (3): 373-392.

Microorganisms with Enhanced Hydrolytic Enzyme Activity

In one embodiment, a microorganism can be modified to enhance an activity of one or more hydrolytic enzymes (such as cellulase(s), hemicellulase(s), or pectinases etc.) or antioxidants (such as catalase), or other enzymes associated with cellulose processing. For example, in the case of cellulases, various microorganisms described herein can be modified to enhance activity of one or more cellulases, or enzymes associated with cellulose processing.

In one embodiment a hydrolytic enzyme is selected from the annotated genome of C. phytofermentans for utilization in a product or process disclosed herein. In another embodiment the hydrolytic enzyme is an endoglucanase, chitinase, cellobiohydrolase or endo-processive cellulases (either on reducing or non-reducing end).

In another embodiment a microorganism, such as C. phytofermentans, can be modified to enhance production of one or more hydrolytic enzymes (such as cellulase(s), hemicellulase(s), or pectinases etc.) or antioxidants (such as catalase), or other enzymes associated with cellulose processing such as one disclosed in U.S. patent application Ser. No. 12/510,994, which is herein incorporated by reference in its entirety. In another embodiment one or more enzymes can be heterologous expressed in a host (e.g., a bacteria or yeast). For heterologous expression bacteria or yeast can be modified through recombinant technology (e.g., Brat et al. Appl. Env. Microbio. 2009; 75(8):2304-2311, disclosing expression of xylose isomerase in S. cerevisiae and which is herein incorporated by reference in its entirety).

In another embodiment, a microorganism can be modified to enhance an activity of one or more cellulases, or enzymes associated with cellulose processing. The classification of cellulases is usually based on grouping enzymes together that forms a family with similar or identical activity, but not necessary the same substrate specificity. One of these classifications is the CAZy system (CAZy stands for Carbohydrate-Active enzymes), for example, where there are 115 different Glycoside Hydrolases (GH) listed, named GH1 to GH155. Each of the different protein families usually has a corresponding enzyme activity. This database includes both cellulose and hemicellulase active enzymes. Furthermore, the entire annotated genome of C. phytofermentans is available on the worldwideweb at www.ncbi.nlm.nih.gov/sites/entrez.

Several examples of cellulase enzymes whose function can be enhanced for expression endogenously or for expression heterologously in a microorganism include one or more of the genes disclosed in Table 2.

TABLE 2

| Cellulase | |
|---|---|
| Protein ID | Description (on www.ncbi.nlm.nih.gov/sites/entrez) |
| ABX43556 | Cellulase [Clostridium phytofermentans ISDg] |
| | gi\|160429993\|gb\|ABX43556.1\|[160429993] Cphy_3202 |
| ABX42426 | Cellulase [Clostridium phytofermentans ISDg] |
| | gi\|160428863\|gb\|ABX42426.1\|[160428863] Cphy_2058 |
| ABX41541 | Cellulase [Clostridium phytofermentans ISDg] |
| | gi\|160427978\|gb\|ABX41541.1\|[160427978] Cphy_1163 |
| ABX43720 | Cellulose 1,4-beta-cellobiosidase [Clostridium |
| | phytofermentans ISDg] |
| | gi\|160430157\|gb\|ABX43720.1\|[160430157] Cphy_3367 |
| ABX41478 | Cellulase M Cphy_1100 |
| ABX41884 | Endo-1,4-beta-xylanase Cphy_1510 |
| ABX43721 | Cellulase 1,4-beta-cellobiosidase Cphy_3368 |
| ABX42494 | Mannan endo-1,4-beta-mannosidase, Cellulase 1,4-beta- |
| | cellobiosidase Cphy_2128 |

Microorganisms with Reduced Lactic Acid Synthesis

In one embodiment, a mesophilic microorganism is modified to disrupt the expression of one or more lactic acid synthesis pathway genes. Inactivating the lactate dehydrogenase gene helps prevent the breakdown of pyruvate into lactate, and therefore promotes, under appropriate conditions, the breakdown of pyruvate into ethanol using pyruvate decarboxylase and alcohol dehydrogenase. In one embodiment, one or more naturally-occurring lactate dehydrogenase genes are disrupted by a deletion within or of the gene. In another embodiment, lactate dehydrogenase is reduced or eliminated by a chemically-induced or naturally-occurring mutation. In one embodiment, a mesophilic microorganism is modified to disrupt the expression of one or more lactate dehydrogenase pathway genes. In one embodiment, a mesophilic microorganism is modified to disrupt the expression of one or more lactate dehydrogenase genes.

The nucleic acid sequence for a lactate dehydrogenase can be used to target the lactate dehydrogenase gene to inactivate the gene through different mechanisms. In one embodiment, a lactate dehydrogenase gene is inactivated by the insertion of a transposon, or by the deletion of the gene sequence or a portion of the gene sequence. In one embodiment, the lactate dehydrogenase gene is inactivated by the integration of a plasmid that achieves natural homologous recombination or integration between the plasmid and the microorganism's chromosome. Chromosomal integrants can be selected for on the basis of their resistance to an antibacterial agent (for example, kanamycin). The integration into the lactate dehydrogenase gene may occur by a single cross-over recombination event or by a double (or more) cross-over recombination event.

In one embodiment, a recombinant organism wherein the organism lacks expression of LDH or demonstrates reduced synthesis of lactate is useful for the biofuel processes disclosed herein. In one embodiment, the recombinant microorganism used for the biofuel processes is C. phytofermentans demonstrating little or no expression of LDH. In another embodiment, a recombinant microorganism used for the biofuel processes is C. phytofermentans showing lactic acid synthesis of 100-90%, 90-80%, 80-70%, 70-60%, 60-50%, 50-40%, 40-30%, 30-20%, 20%-10%, or lower, compared to the wild-type organism. In another embodiment, a recombinant microorganism used for the generation of a fermentation end-product is a C5/C6 hydrolyzing and fermenting microorganism (e.g., Clostridium phytofermentans, Clostridium sp. Q.D, Clostridium phytofermentans Q.8, Clostridium phytofermentans Q.12, Clostridium phytofermentans Q.13, or genetically-modified cells thereof) lacking LDH activity. In a further embodiment, the microorganism is capable of enhanced production of biofuel(s) or chemical(s) as compared to a wild-type microorganism.

In one embodiment a microorganism engineered to knockout or reduce naturally-occurring lactate dehydrogenase is useful for producing ethanol and other chemical products, fermentive end products and/or biofuels at a higher yield than that of natural, wild-type microorganism. In one embodiment, a genetically modified microorganism such as a Clostridium species expressing reduced yields of lactic acid produces ethanol at a rate measurably faster than a corresponding wild-type microorganism, such as a Clostridium species that does not incorporate LDH knockout DNA construct. In one embodiment, a genetically modified microorganism such as a Clostridium species expressing reduced yields of lactic acid produces more of a fermentation end-product from a biomass in a given amount of time than a corresponding wild-type microorganism, such as a Clostridium species that does not incorporate LDH knockout DNA construct. In one embodiment the given amount of time is between 1 and 500 hrs (e.g., about 1-24 hrs, 1-48 hrs, 1-72 hrs, 1-96 hrs, 1-120 hrs, 1-144 hrs, 1-168 hrs, 1-192 hrs, 1-50 hrs, 1-100 hrs, 1-150 hrs, 1-200 hrs, 1-250 hrs, 1-300 hrs, 1-350 hrs, 1-400 hrs, 1-450 hrs, 25-100 hrs, 25-150 hrs, 25-200 hrs, 25-250 hrs, 25-300 hrs, 25-350 hrs, 25-400 hrs, 25-450 hrs, 25-500 hrs, 50-100 hrs, 50-150 hrs, 50-200 hrs, 50-250 hrs, 50-300 hrs, 50-350 hrs, 50-400 hrs, 50-450 hrs, 50-500 hrs, 100-300 hrs, 100-400 hrs, 100-500 hrs, 200-300 hrs, 200-400 hrs, 200-500 hrs, 300-400 hrs, 300-500 hrs, or 400-500 hrs). In one embodiment, a genetically modified Clostridium expressing an LDH knockout DNA construct ferments cellulose to a fermentation end-product more efficiently. In one embodiment, a Clostridium is engineered to express an LDH knockout DNA construct, where the LDH knockout comprises a modified version of Clostridium LDH gene. For example, a gene of sequences in Table 3 may be modified.

TABLE 3

| SEQ | | |
|---|---|---|
| ID | | |
| NO: | Description | Sequence |
| 3 | Cphy_1232 | ATGGCAAAACCAAGAAAAGTCATTATTATCGGAGCAGGTCACG |
| | L-lactate | TAGGATCTCATGCTGGATATGCACTGGCAGAGCAGGGGCTTGC |
| | dehydrogenase | AGAAGAAATTATCTTTATTGATATTGATAGAGAAAAAGCGAAA |
| | [Clostridium | GCACAAGCACTGGATATCTACGATGCTACAGTATACCTACCAC |
| | phytofermentans | ACAGAGTTAAGGTAAAATCGGGTGATTATAGTGATGCAGCTGA |
| | ISDg] | TGCAGATCTCATGGTGATTGCAGTAGGAACCAATCCAGATAAA |
| | | AATAAGGGTGAAACAAGAATGAGTACCCTTACGAATACTGCTC |
| | | TAATTATTAAAGAGGTAGCTTGGCATATCAAAAATTCAGGTTT |
| | | TGATGGTATGATTGTTAGCATTTCAAATCCAGCAGATGTAATA |
| | | ACACATTATTTACAGCATTTACTTCAGTACTCATCCAATAAAA |
| | | TTATTTCAACAAGTACGGTACTAGACTCTGCCAGACTTAGAAG |
| | | AGCAATTGCAGATGCTGTTGAAATTGATCAAAAATCAATCTAT |
| | | GGATTTGTTCTTGGAGAACACGGAGAAAGCCAGATGGTTGCAT |
| | | GGTCAACGGTATCTATAGCTGGAAAACCAATTTTGGAACTAAT |
| | | CAAGGAAAAACCTGAAAAATATGGGCAGATTGATCTTTCTAAG |
| | | CTTTCTGATGAAGCTAGAGCAGGGGGATGGCATATCCTAACTG |
| | | GAAAAGGCTCAACGGAATTTGGTATTGGTGCATCACTAGCTGA |
| | | GGTTACACGAGCCATTTTCTCAGATGAGAAGAAGGTATTACCA |
| | | GTATCTACTCTCTTAAATGGTGAGTATGGCCAGCATGATGTCT |
| | | ATGCATCTGTTCCTACGGTACTTGGAATTCATGGTGTAGAAGA |
| | | AATCATTGAGCTAAATTTGACACCTGAAGAAAAGGGAAAATTC |
| | | GATGCTTCTTGTAGAACAATGAAAGAAAATTTTCAGTATGCAT |
| | | TGACGCTATCATAA |
| 4 | Cphy_1232 | MAKPRKVIIIGAGHVGSHAGYALAEQGLAEEIIFIDIDREKAK |
| | Protein Sequence | AQALDIYDATVYLPHRVKVKSGDYSDAADADLMVIAVGTNPDK |
| | L-lactate | NKGETRMSTLTNTALIIKEVAWHIKNSGFDGMIVSISNPADVI |
| | dehydrogenase | THYLQHLLQYSSNKIISTSTVLDSARLRRAIADAVEIDQKSIY |
| | [Clostridium | GFVLGEHGESQMVAWSTVSIAGKPILELIKEKPEKYGQIDLSK |
| | phytofermentans | LSDEARAGGWHILTGKGSTEFGIGASLAEVTRAIFSDEKKVLP |
| | ISDg] | VSTLLNGEYGQHDVYASVPTVLGIHGVEEIIELNLTPEEKGKF |
| | GenBank Accession | DASCRTMKENFQYALTLS |
| | No.: NC_010001.1 | |
| | GI:160879381 | |
| 5 | Cphy_1117 | ATGGCGATTACAATAAACCGAAGTAAAGTTATTGTTGTGGGTG |
| | L-lactate | CAGGTTTAGTTGGTACTTCAACGGCGTTTAGTCTAATTACGCA |
| | dehydrogenase | AAGTGTTTGTGATGAGGTTATGTTGATAGATATCAATCGTGCT |
| | [Clostridium | AAGGCGCATGGGGAAGTAATGGATTTGTGTCATAGTATCGAGT |
| | phytofermentans | ATTTAAATCGAAATGTTTTGGTAACGGAAGGAGATTATACAGA |
| | ISDg] | CTGTAAGGACGCTGATATTGTTGTAATAACTGCAGGGCCTCCG |
| | | CCAAAACCAGGACAGTCGCGGCTTGATACTCTTGGGTTATCCG |
| | | CAGATATTGTGAGCACGATTGTGGAACCTGTCATGAAGAGTGG |
| | | GTTCAATGGAATATTCTTAGTCGTGACGAATCCGGTGGATTCG |
| | | ATTGCTCAATATGTTTATCAATTATCGGGGCTTCCAAAGCAAC |
| | | AAGTTCTTGGAACTGGAACAGCGATTGACTCTGCAAGATTAAA |
| | | ACACTTTATTGGAGATATTTTACATGTAGATCCTAGAAGCATA |
| | | CAGGCTTATACGATGGGAGAGCATGGAGATTCTCAAATGTGTC |
| | | CTTGGTCGCTTGTTACGGTTGGCGGTAAAAATATTATGGACAT |
| | | CGTACGGGATAACAAAGAGTATTCCGATATTGACTTTAATGAA |
| | | ATCTTATATAAGGTTACCAGGGTAGGTTTTGATATTTTATCAG |
| | | TGAAGGGTACTACTTGTTATGGAATAGCGTCAGCAGCTGTGGG |
| | | GATTATAAAAGCAATTCTTTATGATGAGAATTCCATCCTTCCG |
| | | GTCTCTACCTTATTGGAGGGGGAATATGGTGAGTTTGATGTAT |
| | | ATGCAGGGGTACCATGCATTCTAAATCGTTTCGGCGTGAAGGA |
| | | TGTAGTGGAAGTAAATATGACAGAAGTAGAGTTAAATCAATTC |
| | | CGAGCCTCTGTTCACGTTGTGAGGGAAGCTATTGAAAACTTAA |
| | | AAGACAGAGATAAAAAGGCATTATTTTTATAA |
| 6 | Cphy_1117 | MAITINRSKVIVVGAGLVGTSTAFSLITQSVCDEVMLIDINRA |
| | L-lactate | KAHGEVMDLCHSIEYLNRNVLVTEGDYTDCKDADIVVITAGPP |
| | dehydrogenase | PKPGQSRLDTLGLSADIVSTIVEPVMKSGFNGIFLVVTNPVDS |
| | [Clostridium | IAQYVYQLSGLPKQQVLGTGTAIDSARLKHFIGDILHVDPRSI |
| | phytofermentans | QAYTMGEHGDSQMCPWSLVTVGGKNIMDIVRDNKEYSDIDFNE |
| | ISDg] | ILYKVTRVGFDILSVKGTTCYGIASAAVGIIKAILYDENSILP |
| | GenBank Accession | VSTLLEGEYGEFDVYAGVPCILNRFGVKDVVEVNMTEVELNQF |
| | No.: NC_010001.1 | RASVHVVREAIENLKDRDKKALFL |
| | GI:160879266 | |

*Sequences 3 and 5 correspond to cDNA sequence whereas sequences
4 and 6 correspond to protein sequence.

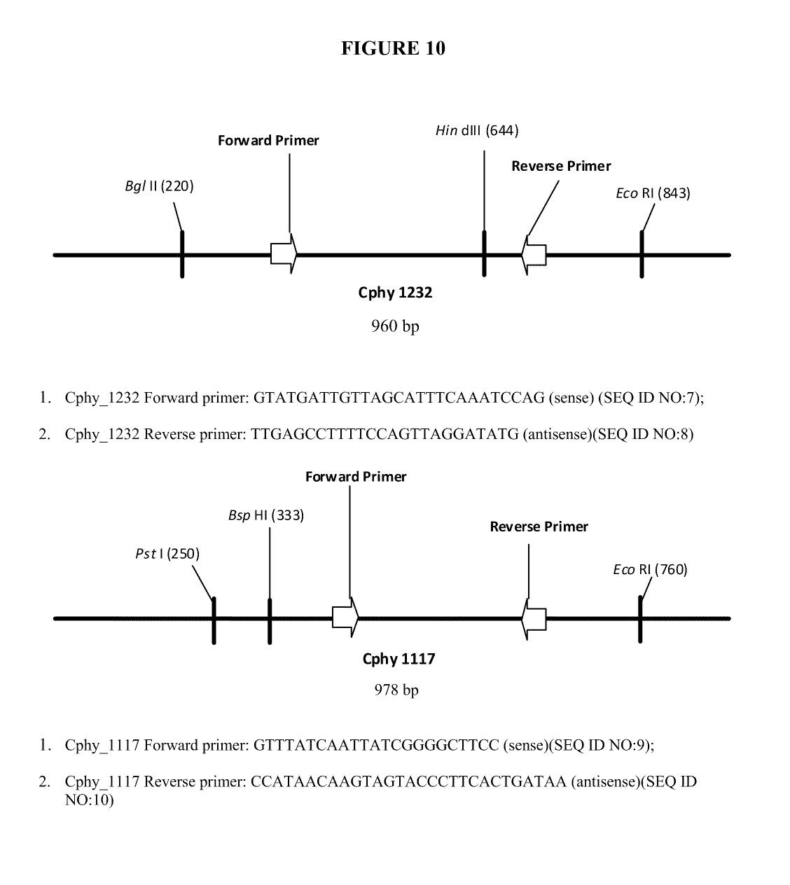

In one embodiment, primers specific to an LDH genomic sequence are generated for design of a plasmid encoding for a LDH knockout gene. In a further embodiment, the LDH gene is SEQ ID NOS: 4 and 6, or an LDG gene from another microorganism. In a further embodiment, the primers are SEQ ID NO: 7, SEQ ID NO: 8 SEQ ID NO: 9, SEQ ID NO: 10 (see FIG. 10), or another DNA construct capable of binding an LDH gene, e.g. the gene of SEQ ID NOS: 3 or 5. In another embodiment, the LDH knockout gene is expressed in a microorganism to provide for a genetically modified microorganism capable of enhanced production of a fermentation end-product. In one embodiment, the fermentation end-product is a fuel or chemical product. In a further embodiment, the chemical product is ethanol. In one embodiment, the genetically modified microorganism is a Clostridium. In another embodiment, the genetically modified microorganism is C. phytofermentans, Clostridium sp. Q.D, Clostridium phytofermentans Q.8, Clostridium phytofermentans Q.12, Clostridium phytofermentans Q.13, or genetically-modified cells thereof.

In one embodiment, a genetically modified microorganism comprises one or more heterologous genes in addition to an LDH knockout DNA construct. In one embodiment, the heterologous gene is a cellulase, a xylanase, a hemicellulase, an endoglucanase, an exoglucanase, a cellobiohydrolase (CBH), a beta-glycosidase, a glycoside hydrolase, a glycosyltransferase, a lysase, an esterase, a chitinase, or a pectinase. In another embodiment, the genetically modified microorganism that is further transformed is a Clostridium strain. In one embodiment the Clostridium strain is C. phytofermentans, Clostridium sp. Q.D, Clostridium phytofermentans Q.8. Clostridium phytofermentans Q.12, Clostridium phytofermentans Q.13, or genetically-modified cells thereof.

In another embodiment, the heterologous gene is an acetic acid or formic acid knockout DNA construct. In a further embodiment, the acetic acid knockout DNA construct comprises all or part of: a phosphotransacetylase (PTA) gene, such as Cphy_{—}1326, an acetyl kinase gene, such as Cphy_{—}1327, and/or a pyruvate formate lyase gene such as Cphy_{—}1174. (See Table 4.) In another embodiment, the genetically modified microorganism that is further transformed is a Clostridium strain. In one embodiment the Clostridium strain is C. phytofermentans, Clostridium sp. Q.D, Clostridium phytofermentans Q.8, Clostridium phytofermentans Q.12, Clostridium phytofermentans Q.13, or genetically-modified cells thereof

TABLE 4

| SEQ | | |
|---|---|---|
| ID | | |
| NO: | Description | Sequence |
| 11 | Cphy_1326; | ATGGGATTTATTGATGACATCAAGGCAAGAGCTAAACAAAGTA |
| | Phosphotransacetylase | TTAAGACTATTGTTTTACCTGAGAGTATGGACAGAAGAACAAT |
| | (PTA) gene; | TGAGGCAGCTGCTAAGACTTTAGAAGAGGGCAATGCTAACGTA |
| | [Clostridium | ATTATTATCGGTAGTGAGGAAGAAGTTAAGAAGAATTCAGAAG |
| | phytofermentans | GTCTTGACATTTCGGGAGCTACAATCGTTGACCCTAAGACATC |
| | ISDg];; | GGACAAGCTTCCAGCTTACATTAACAAGCTTGTAGAACTTAGA |
| | Accession No.: | CAGGCAAAAGGCATGACCCCTGAAAAAGCAAAAGAGCTTTTAA |
| | NC_010001; | CAACAGACTACATTACATACGGTGTAATGATGGTTAAGATGGG |
| | GI:160878162 | CGATGCAGATGGTTTAGTATCTGGTGCTTGTCACTCTACAGCA |
| | | GATACCTTAAGACCATGTCTTCAGATTTTAAAAACTGCTCCAA |
| | | ATACTAAGTTAGTTTCTGCTTTCTTCGTAATGGTAGTACCTAA |
| | | TTGTGATATGGGCGCAAATGGAACTTTCCTTTTCTCTGATGCT |
| | | GGTTTAAATCAGAATCCAAATGCTGAAGAGTTAGCAGCAATCG |
| | | CTGGTTCCACAGCGAAGAGTTTTGAACAATTAGTTGGCTCTGA |
| | | ACCTATCGTAGCTATGCTTTCTCATTCAACAAAGGGAAGCGCA |
| | | AAGCATGCAGATGTTGATAAGGTTGTAGAAGCAACTAAGATTG |
| | | CAAATGAATTATACCCAGAATATAAGATCGACGGCGAGTTCCA |
| | | GTTAGATGCAGCAATCGTTCCTAGTGTAGGTGCTTCAAAAGCT |
| | | CCTGGTAGTGATATTGCTGGAAAAGCTAACGTATTAATCTTCC |
| | | CAGACCTTGATGCTGGTAACATTGGATATAAGTTAACACAGCG |
| | | TCTTGCAAAGGCAGAAGCTTATGGACCATTAACTCAGGGTATT |
| | | GCAGCTCCAGTAAATGATTTATCAAGAGGTTGTTCTTCTGATG |
| | | ATATCGTTGGTGTTGTTGCAATCACTGCTGTTCAGGCACAGAG |
| | | TAAATAA |
| 12 | Cphy_1326; | MGFIDDIKARAKQSIKTIVLPESMDRRTIEAAAKTLEEGNANV |
| | Phosphotransacetylase | IIIGSEEEVKKNSEGLDISGATIVDPKTSDKLPAYINKLVELR |
| | (PTA); | QAKGMTPEKAKELLTTDYITYGVMMVKMGDADGLVSGACHSTA |
| | Clostridium | DTLRPCLQILKTAPNTKLVSAFFVMVVPNCDMGANGTFLFSDA |
| | phytofermentans ISDg; | GLNQNPNAEELAAIAGSTAKSFEQLVGSEPIVAMLSHSTKGSA |
| | Accession No.: | KHADVDKVVEATKIANELYPEYKIDGEFQLDAAIVPSVGASKA |
| | YP_001558442.1; | PGSDIAGKANVLIFPDLDAGNIGYKLTQRLAKAEAYGPLTQGI |
| | GI:160879474 | AAPVNDLSRGCSSDDIVGVVAITAVQAQSK |
| 13 | Cphy_1327 acetate | MKVLVINCGSSSLKYQLIDSVTEQALAVGLCERIGIDGRLTHK |
| | kinase [Clostridium | SADGEKVVLEDALPNHEVAIKNVIAALMNENYGVIKSLDEINA |
| | phytofermentans ISDg]; | VGHRVVHGGEKFAHSVVINDEVLNAIEECNDLAPLHNPANLIG |
| | Accession No.: | INACKSIMPNVPMVAVFDTAFHQTMPKEAYLYGIPFEYYDKYK |
| | YP_001558443; | VRRYGFHGTSHSYVSKRATTLAGLDVNNSKVIVCHLGNGASIS |
| | GI:160879475 | AVKNGESVDTSMGLTPLEGLIMGTRSGDLDPAIIDFVAKKENL |
| | | SLDEVMNILNKKSGVLGMSGVSSDFRDIEAAANEGNEHAKEAL |
| | | AVFAYRVAKYVGSYIVAMNGVDAVVFTAGLGENDKNIRAAVSS |
| | | HLEFLGVSLDAEKNSQRGKELIISNPDSKVKIMVIPTNEELAI |
| | | CREVVELV |
| 14 | Cphy_1327 acetate | ATGAAAGTTTTAGTTATTAATTGCGGAAGTTCTTCCCTTAAAT |
| | kinase [Clostridium | ATCAGTTAATCGACTCTGTGACAGAGCAAGCATTAGCAGTAGG |
| | phytofermentans | TCTTTGTGAAAGAATCGGTATTGATGGCCGTCTTACTCACAAG |
| | ISDg]; | TCAGCTGACGGTGAGAAGGTAGTTCTTGAGGATGCACTTCCAA |
| | GI:160879475 | ACCATGAGGTTGCTATTAAAAATGTAATCGCTGCTCTTATGAA |
| | | TGAAAATTATGGTGTGATTAAGTCCTTAGATGAAATCAACGCT |
| | | GTTGGACATAGAGTAGTACATGGTGGTGAGAAATTTGCTCATT |
| | | CCGTAGTAATCAATGATGAAGTCTTAAATGCAATTGAAGAGTG |
| | | TAATGATCTTGCACCTTTACACAACCCAGCAAACCTTATTGGT |
| | | ATCAACGCTTGTAAATCAATTATGCCAAATGTACCAATGGTAG |
| | | CTGTTTTTGATACTGCATTCCATCAGACAATGCCAAAAGAAGC |
| | | TTACCTTTATGGTATTCCATTTGAGTACTATGATAAATATAAG |
| | | GTAAGAAGATATGGTTTCCACGGAACAAGTCACAGCTATGTTT |
| | | CTAAAAGAGCAACCACGCTTGCTGGCTTAGATGTAAATAACTC |
| | | AAAAGTTATCGTTTGTCACCTTGGTAATGGCGCATCCATTTCC |
| | | GCAGTTAAAAACGGTGAGTCTGTAGATACAAGTATGGGTCTTA |
| | | CACCACTTGAAGGTTTAATCATGGGAACAAGAAGTGGTGATCT |
| | | TGATCCAGCAATCATTGATTTCGTTGCTAAGAAAGAAAACTTA |
| | | TCCTTAGATGAAGTAATGAATATCTTAAATAAGAAATCTGGTG |
| | | TATTAGGTATGTCCGGAGTATCTTCTGACTTTAGAGATATCGA |
| | | AGCAGCAGCAAACGAAGGCAATGAGCATGCAAAAGAAGCTTTA |
| | | GCAGTTTTTGCATACCGTGTTGCTAAATATGTAGGTTCTTATA |
| | | TCGTAGCTATGAATGGTGTAGATGCTGTTGTATTTACAGCAGG |
| | | ACTTGGTGAGAATGATAAGAACATCAGAGCAGCAGTAAGTTCA |
| | | CACCTTGAGTTCCTTGGTGTATCTTTAGATGCTGAGAAGAATT |
| | | CTCAAAGAGGTAAAGAATTAATCATCTCTAACCCAGATTCTAA |
| | | GGTTAAGATTATGGTTATCCCAACTAACGAAGAGCTTGCAATC |
| | | TGTAGAGAAGTTGTTGAATTAGTGTAG |
| 15 | Cphy_1174; pyruvate | MMAEPKKGYEKSPRIQKLMDALYEKMPEIESKRAVLITESYQQ |
| | formate-lyase | TEGEPIISRRSKAFEHIVKNLPVVIRENELIVGSATVAERGCQ |
| | [Clostridium | TFPEFSFDWLIAELDTVATRTADPFYISEEAKKELRKVHSYWK |
| | phytofermentans | GKTTSELADYYMAPETKLAMEHNVFTPGNYFYNGVGHITVQYD |
| | ISDg]; | AILYAKRYAAEAKVIAIGYEGIKDEVLSRKKELHLGDADYASR |
| | Accession No.: | LTFYDAVIRSCDSKRLALSCQDEKRRQELLMISSNCERVPAKG |
| | YP_001558291; | ANTFYEACQAFWFVQLLLQIEASGHSISPGRFDQYLYSYYKAD |
| | GI:160879323 | REAGRITGEQAQEIIDCIFVKLNDINKCRDAASAEGFAGYGMF |
| | | QNMIVGGQDSNGRDATNELSFMILEASIHTMLPQPSLSIRVWN |
| | | GSPHDLLIKAAEVTRTGIGLPAYYNDEVIIPAMMNKGATLEEA |
| | | RNYNIIGCVEPQVPGKTDGWHDAAFFNMCRPLEMVFSSGYENG |
| | | KLVGAPTGSVENFTTFEAFYDAYKTQMEYFISLLVNADNSIDI |
| | | AHAKLCPLPFESSMVEDCIGRGLCVQEGGAKYNFTGPQGFGIA |
| | | NMTDSLYAIKKLVYEEGKVSITELKEALLHNFGMTTKNAGLKE |
| | | SSHLSIDIILAQQITVQIVKELKERGKEPSEKEIEQILKTVLE |
| | | AKKENTESPISTRVSENTSNHSRYQEILQMIEVLPKYGNDILE |
| | | IDEFAREIAYTYTKPLQKYKNPRGGVFQAGLYPVSANVPLGEQ |
| | | TGATPDGRLANTPIADGVGPAPGRDTKGPTAAANSVARLDHMD |
| | | ATNGTLYNQKFHPSALQGRGGLEKFVALIRAFFDQKGMHVQFN |
| | | VVSRETLLDAQKHPENYKHLVVRVAGYSALFTTLSRSLQDDII |
| | | NRTTQGF |
| 16 | Cphy_1174; pyruvate | ATGATGGCTGAACCCAAAAAAGGATATGAAAAATCACCTCGTA |
| | formate-lyase | TACAAAAGCTTATGGATGCTTTATACGAGAAAATGCCAGAGAT |
| | [Clostridium | TGAATCAAAACGTGCAGTTTTAATCACGGAATCGTATCAGCAG |
| | phytofermentans | ACGGAAGGAGAGCCTATCATTAGTAGACGCTCCAAGGCTTTTG |
| | ISDg]; | AACATATAGTAAAGAATCTTCCAGTAGTAATTCGAGAGAATGA |
| | GI:160879323 | ATTAATTGTAGGAAGCGCAACCGTTGCAGAAAGAGGATGTCAA |
| | | ACCTTTCCGGAATTCTCTTTTGATTGGTTAATTGCTGAACTTG |
| | | ATACCGTAGCAACTAGAACTGCTGATCCGTTTTATATCTCAGA |
| | | GGAAGCAAAAAAAGAGTTAAGAAAAGTACATAGCTATTGGAAG |
| | | GGAAAAACAACAAGTGAATTAGCAGATTATTACATGGCTCCAG |
| | | AAACGAAACTTGCGATGGAGCACAATGTATTTACACCAGGTAA |
| | | CTATTTTTATAACGGTGTAGGGCACATTACAGTGCAGTATGAT |
| | | AAGGTAATTGCGATCGGTTATGAAGGAATTAAAGATGAAGTCT |
| | | TAAGCAGAAAAAAAGAATTACATCTAGGTGATGCTGATTATGC |
| | | AAGTCGCCTTACTTTCTATGACGCTGTAATCAGAAGTTGTGAC |
| | | TCGGCTATTTTGTATGCTAAGAGATATGCAGCGGAAGCAAAAA |
| | | GACTTGCACTTTCTTGTCAGGATGAGAAGAGAAGACAAGAACT |
| | | TTTAATGATTTCATCTAATTGTGAGAGAGTCCCAGCAAAGGGT |
| | | GCGAATACATTTTATGAAGCATGTCAGGCATTTTGGTTTGTAC |
| | | AACTTTTATTACAGATTGAAGCTAGTGGACATTCGATTTCACC |
| | | AGGTAGATTTGACCAATATTTATATTCATATTATAAAGCAGAT |
| | | CGTGAAGCAGGCAGAATCACTGGTGAACAGGCACAAGAAATCA |
| | | TCGATTGTATTTTTGTGAAATTAAATGATATTAACAAATGCCG |
| | | TGATGCTGCTTCTGCGGAAGGTTTTGCAGGCTATGGTATGTTC |
| | | CAGAACATGATTGTTGGCGGACAGGATAGTAACGGAAGGGATG |
| | | CTACGAATGAACTTAGTTTTATGATATTAGAGGCATCCATACA |
| | | CACCATGCTTCCACAGCCTTCCTTAAGTATCCGTGTATGGAAT |
| | | GGTTCTCCGCATGATTTACTAATTAAAGCTGCGGAAGTTACCA |
| | | GAACTGGTATCGGTTTACCTGCTTATTACAACGATGAAGTTAT |
| | | TATCCCAGCTATGATGAATAAGGGTGCAACTTTAGAGGAAGCG |
| | | AGAAACTATAATATTATCGGTTGCGTGGAACCTCAAGTACCTG |
| | | GTAAGACCGACGGATGGCATGACGCAGCATTCTTTAATATGTG |
| | | TCGCCCATTGGAAATGGTATTTTCTAGTGGATATGAAAATGGA |
| | | AAATTAGTTGGTGCTCCAACAGGTTCGGTTGAAAACTTCACTA |
| | | CATTTGAGGCATTTTATGATGCTTATAAAACTCAGATGGAATA |
| | | CTTTATCTCTTTACTAGTCAATGCGGATAATTCAATCGATATT |
| | | GCGCATGCAAAACTTTGCCCATTACCATTTGAATCCTCTATGG |
| | | TAGAAGATTGTATCGGACGTGGGTTATGTGTTCAAGAAGGTGG |
| | | AGCAAAATATAATTTTACCGGACCACAAGGGTTTGGTATCGCC |
| | | AATATGACAGACTCCTTATATGCGATTAAGAAACTTGTATACG |
| | | AAGAAGGCAAGGTTTCTATTACTGAATTAAAAGAAGCACTTCT |
| | | ACATAATTTCGGAATGACAACGAAGAACGCTGGCTTAAAGGAA |
| | | AGCTCTCATCTGTCCATAGATATCATATTAGCGCAGCAAATCA |
| | | CAGTGCAGATTGTAAAAGAATTGAAAGAGCGTGGAAAAGAGCC |
| | | TTCAGAGAAGGAAATAGAACAAATATTAAAGACAGTTCTTGAA |
| | | GCAAAGAAAGAAAACACAGAGAGTCCAATATCTACAAGAGTGT |
| | | CAGAGAACACAAGTAATCATTCAAGATATCAAGAAATTCTACA |
| | | GATGATTGAAGTGTTACCAAAGTACGGAAATGATATCCTAGAG |
| | | ATTGATGAATTCGCCAGGGAGATTGCTTATACCTATACAAAGC |
| | | CATTACAAAAATATAAAAATCCAAGAGGTGGTGTATTCCAAGC |
| | | TGGTTTATATCCGGTTTCCGCAAATGTACCGTTAGGTGAACAA |
| | | ACAGGGGCTACTCCAGATGGAAGACTTGCGAATACCCCAATTG |
| | | CAGATGGTGTTGGCCCAGCGCCAGGACGTGATACCAAAGGACC |
| | | AACAGCGGCAGCTAATTCCGTAGCACGCCTTGATCATATGGAT |
| | | GCAACAAATGGTACCTTATACAATCAAAAATTCCATCCATCTG |
| | | CGTTACAGGGTCGTGGTGGACTAGAGAAGTTTGTAGCGTTAAT |
| | | CCGTGCCTTCTTTGATCAAAAGGGTATGCATGTACAGTTTAAT |
| | | GTAGTAAGTAGAGAAACTTTATTAGACGCACAAAAGCACCCAG |
| | | AAAACTATAAACATTTGGTGGTACGTGTTGCTGGTTACAGTGC |
| | | CCTATTTACTACATTATCCAGGTCCTTACAGGATGATATTATT |
| | | AATCGAACAACACAAGGGTTCTAG |

*Sequences 11, 14, and 16 correspond to cDNA sequence
whereas sequences 12 and 13, and 15 correspond to
protein sequence.

Microorganisms with Enhanced Ethanol Production

In another embodiment other modifications can be made to enhance end-product (e.g., ethanol) production in a recombinant microorganism. For example, the host microorganism can further comprise an additional heterologous DNA segment, the expression product of which is a protein involved in the transport of mono- and/or oligosaccharides into the recombinant host. Likewise, additional genes from the glycolytic pathway can be incorporated into the host. In such ways, an enhanced rate of ethanol production can be achieved.

In one embodiment, a redirection of glycolytic or solventogenic pathways can be used to alter the yield of end products such as ethanol or used to reduce ethanol inhibition. In one embodiment, a heterologous alcohol dehydrogenase, for example, the adhB enzyme from Zymomonas mobilis, can be overexpressed in a microorganism, for example a Clostridium species (e.g. Clostridium phytofermentans, Clostridium sp. Q.D or a variant thereof), to ensure that acetaldehyde is reduced to ethanol even when ethanol titers are high in the fermentation medium. In this manner, the overexpression of an alcohol dehydrogenase tolerant to high ethanol titers can boost the ethanol production to 50, 55, 60, 65, 70, and even 75 g/L, thus generating higher overall yields.

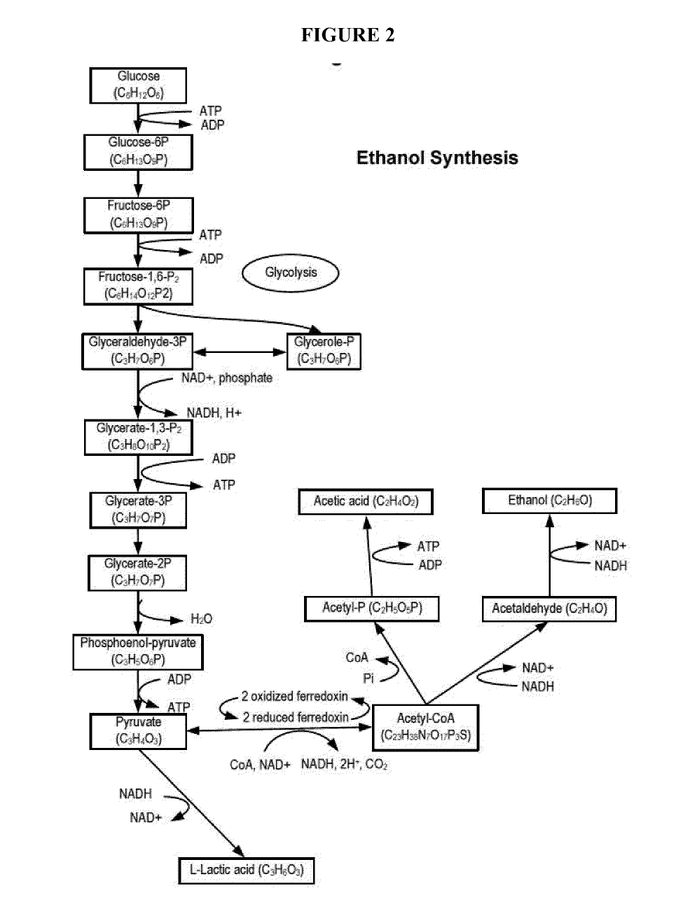

In another embodiment a microorganism can be modified to enhance an activity of one or more decarboxylases (e.g. pyruvate decarboxylase), dehydrogenases (e.g. alcohol dehydrogenase), synthetases (e.g. Acetyl CoA synthetase) or other enzymes associated with glycolic processing e.g. FIG. 2). Through recombinant methodology, for example, incorporation of a pyruvate decarboxylase into an organism such as C. phytofermentans or Q.D can redirect most of the conversion of pyruvate from glycolysis directly into acetaldehyde and subsequently to ethanol, reducing substantially the amount of acetic acid synthesized to practically nothing. The oxidized NAD can enter back into glycolysis. In one embodiment, no acetic acid is synthesized and the small amount of Acetyl-CoA produced is utilized in essential pathways, such as fatty acid synthesis. In a further embodiment, acetyl-CoA synthetase is overexpressed to recycle the acetic acid synthesized so that additional ATP is generated and there is no buildup of acetic acid product.

In another embodiment, one or more genes found in Table 5 are heterologously expressed in a microorganism, for example a Clostridium species (e.g. Clostridium phytofermentans, Clostridium sp. Q.D or a variant thereof). In one embodiment, Zymomonas mobilis pyruvate decarboxylase (pdc) is expressed in a microorganism. In another embodiment, Z. mobilis alcohol dehydrogenase II (adhB) is expressed in a microorganism. In another embodiment, both pdc and adhB from Z. mobilis are expressed in a microorganism. In some embodiments, the microorganism is a Clostridium species (e.g. Clostridium phytofermentans, Clostridium sp. Q.D or a variant thereof). In another embodiment, acetyl-CoA synthetase (acs) from Escherichia coli is heterologously expressed in a microorganism with or without the expression of pdc and/or adhB from Z. mobilus. In another embodiment, a recombinant organism disclosed herein can be further genetically modified to reduce or eliminate the expression of lactate dehydrogenase (ldh).

In one embodiment, a genetically modified microorganism (e.g. a Clostridium bacterium, e.g. Clostridium phytofermentans, Clostridium sp. Q.D or a variant thereof) expressing a gene from a glycolytic or solventogenic pathway (e.g. a gene from Table 5, e.g. pyruvate decarboxylase) produces an increased yield of a fermentation end-product (e.g. an alcohol, e.g. ethanol) as compared to a control strain. The increase in production can be 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 g/L, or more. This increase can be, for example, at least a 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 100%, 110%, 120%, 130%, 140%, 150%, 160%, 170%, 180%, 190%, 200%, or higher percentage increase in fermentation end-product production. An increase in yield from a genetically modified microorganism can be 1.1, 1.2, 1.3, 1.4, 1.5, 1.6, 1.7, 1.8, 1.9, 2.0 or more times the yield of a non-genetically modified microorganism. In another embodiment, a species of C. phytofermentans expressing a heterologous pdc gene from Z. mobilis produces 8-10 g/L more ethanol than a control strain under conditions detailed in Example 5.

TABLE 5
SEQ ID no: 17
Description: Zymomonas mobilis
Alcohol dehydrogenase II (adhB)
GenBank: X17065.1
DNA sequence
gatctgataaaactgatagacatattgcttttgcgctgcccgattgct
gaaaatgcgtaaaattggtgattttactcgttttcaggaaaaactttg
agaaaacgtctcgaaaacgggattaaaacgcaaaaacaatagaaagcg
atttcgcgaaaatggttgttttcgggttgttgctttaaactagtatgt
agggtgaggttatagctatggcttcttcaactttttatattcctttcg
tcaacgaaatgggcgaaggttcgcttgaaaaagcaatcaaggatctta
acggcagcggctttaaaaatgccctgatcgtttctgatgctttcatga
acaaatccggtgttgtgaagcaggttgctgacctgttgaaaacacagg
gtattaattctgctgtttatgatggcgttatgccgaacccgactgtta
ccgcagttctggaaggccttaagatcctgaaggataacaattcagact
tcgtcatctccctcggtggtggttctccccatgactgcgccaaagcca
tcgctctggtcgcaaccaatggtggtgaagtcaaagactacgaaggta
tcgacaaatctaagaaacctgccctgcctttgatgtcaatcaacacga
cggctggtacggcttctgaaatgacgcgtttctgcatcatcactgatg
aagtccgtcacgttaagatggccattgttgaccgtcacgttaccccga
tggtttccgtcaacgatcctctgttgatggttggtatgccaaaaggcc
tgaccgccgccaccggtatggatgctctgacccacgcatttgaagctt
attcttcaacggcagctactccgatcaccgatgcttgcgctttgaaag
cagcttccatgatcgctaagaatctgaagaccgcttgcgacaacggta
aggatatgccagctcgtgaagctatggcttatgcccaattcctcgctg
gtatggccttcaacaacgcttcgcttggttatgtccatgctatggctc
accagttgggcggttactacaacctgccgcatggtgtctgcaacgctg
ttctgcttccgcatgttctggcttataacgcctctgtcgttgctggtc
gtctgaaagacgttggtgttgctatgggtctcgatatcgccaatctcg
gcgataaagaaggcgcagaagccaccattcaggctgttcgcgatctgg
ctgcttccattggtattccagcaaatctgaccgagctgggtgctaaga
aagaagatgtgccgcttcttgctgaccacgctctgaaagatgcttgtg
ctctgaccaacccgcgtcagggtgatcagaaagaagttgaagaactct
tcctgagcgctttctaatttcaaaacaggaaaacggttttccgtcctg
tcttgattttcaagcaaacaatgcctccgatttctaatcggaggcatt
tgtttttgtttattgcaaaaacaaaaaatattgttacaaatttttaca
ggctattaagcctaccgtcataaataatttgccatttaaagcctatta
tcaggattttcgccccgatttcagccatggcagaaatcttttcggttt
aatagcgggaaattctttgatagctggccttttgctcgcttgctttat
tatttttacatccaggcggtgaaagtgtacagaaaagccgcgtttgcc
ttatgaaggcgacgaaatatttttcagataaagtctttaccttgttaa
aaccgcttttcgttttatcgggtaaatgcctaatgcagagtttgattt
caggcctatgtttccgaataaaaagacgccgttgttagacaagatc
SEQ ID no: 18
Description: Zymomonas mobilis
Alcohol dehydrogenase II (adhB)
GenBank: BAF76066.1
Protein sequence
MASSTFYIPFVNEMGEGSLEKAIKDLNGSGFKNALIVSDAFMNKSGVV
KQVADLLKTQGINSAVYDGVMPNPTVTAVLEGLKILKDNNSDFVISLG
GGSPHDCAKAIALVATNGGEVKDYEGIDKSKKPALPLMSINTTAGTAS
EMTRFCIITDEVRHVKMAIVDRHVTPMVSVNDPLLMVGMPKGLTAATG
MDALTHAFEAYSSTAATPITDACALKAASMIAKNLKTACDNGKDMPAR
EAMAYAQFLAGMAFNNASLGYVHAMAHQLGGYYNLPHGVCNAVLLPHV
LAYNASVVAGRLKDVGVAMGLDIANLGDKEGAEATIQAVRDLAASIGI
PANLTELGAKKEDVPLLADHALKDACALTNPRQGDQKEVEELFLSAF
SEQ ID no: 19
Description: Zymomonas mobilis
pyruvate decarboxylase (pdc)
GenBank: HM235920.1
DNA sequence
ggatcctgtaacagctcattgataaagccggtcgctcgcctcgggcag
ttttggattgatcctgccctgtcttgtttggaattgatgaggccgttc
atgacaacagccggaaaaattttaaaacaggcgtcttcggctgcttta
ggtctcggctacgtttctacatctggttctgattcccggtttaccttt
ttcaaggtgtcccgttcctttttcccctttttggaggttggttatgtc
ctataatcacttaatccagaaacgggcgtttagctttgtccatcatgg
ttgtttatcgctcatgatcgcggcatgttctgatatttttcctctaaa
aaagataaaaagtcttttcgcttcggcagaagaggttcatcatgaaca
aaaattcggcatttttaaaaatgcctatagctaaatccggaacgacac
tttagaggtttctgggtcatcctgattcagacatagtgttttgaatat
atggagtaagcaatgagttatactgtcggtacctatttagcggagcgg
cttgtccaaattggtctcaagcatcacttcgcagtcgcgggcgactac
aacctcgtccttcttgacaacctgcttttaaacaaaaacatggagcag
gtttattgctgtaacgaactgaactgcggtttcagtgcagaaggttat
gctcgtgccaaaggcgcagcagcagccgtcgttacctacagcgtcggt
gcgctttccgcattcgatgctatcggtggcgcctatgcagaaaacctt
ccggttatcctgatctccggtgctccgaacaacaatgaccacgctgct
ggtcacgtgttgcatcatgctcttggcaaaaccgactatcactatcag
ttggaaatggccaagaacatcacggccgccgctgaagcgatttatacc
ccggaagaagctccggctaaaatcgatcacgtgattaaaactgctctt
cgtgagaagaagccggtttatctcgaaatcgcttgcaacattgcttcc
atgccctgcgccgctcctggaccggcaagcgcattgttcaatgacgaa
gccagcgacgaagcttctttgaatgcagcggttgaagaaaccctgaaa
ttcatcgccgaccgcgacaaagttgccgtcctcgtcggcagcaagctg
cgcgcagctggtgctgaagaagctgctgtcaaatttgctgatgctctt
ggtggcgcagttgctaccatggctgctgcaaaaagcttcttcccagaa
gaaaacccgcattacatcggtacctcatggggtgaagtcagctatccg
ggcgttgaaaagacgatgaaagaagccgatgcggttatcgctctggct
cctgtctttaacgactactccaccactggttggacggatattcctgat
cctaagaaactggttctcgctgaaccgcgttctgtcgtcgttaacggc
attcgcttccccagcgtccacctgaaagactatctgacccgtttggct
cagaaagtttccaagaaaaccggtgctttggacttcttcaaatccctc
aatgcaggtgaactgaagaaagccgctccggctgatccgagtgctccg
ttggtcaacgcagaaatcgcccgtcaggtcgaagctcttctgaccccg
aacacgacggttattgctgaaaccggtgactcttggttcaatgctcag
cgcataaagctcccgaacggtgctcgcgttgaatatgaaatgcagtgg
ggtcacattggttggtccgttcctgccgccttcggttatgccgtcggt
gctccggaacgtcgcaacatcctcatggttggtgatggttccttccag
ctgacggctcaggaagtcgctcagatggttcgcctgaaaccgccggtt
atcatcttcttgatcaataactatggttacaccatcgaagttatgatc
catgatggtccgtacaacaacatcaagaactgggattatgccggtctg
atggaagtgttcaacggtaacggtggttatgacagcggtgctggtaaa
ggccttaaagctaaaaccggtggcgaactggcagaagctatcaaggtt
gctctggcaaacaccgacggcccaaccctgatcgaatgcttcatcggt
cgggaagactgcactgaagaattggtcaaatggggtaagcgcgttgct
gccgccaacagccgtaagcctgttaacaagctcctctagtttttaaat
aaacttagagaattc
SEQ ID no: 20
Description: Zymomonas mobilis
pyruvate decarboxylase (pdc)
GenBank: CAA42157.1
Protein sequence
MSYTVGTYLAERLVQIGLKHHFAVAGDYNLVLLDNLLLNKNMEQVYCC
NELNCGFSAEGYARAKGAAAAVVTYSVGALSAFDAIGGAYAENLPVIL
ISGAPNNNDHAAGHVLHHALGKTDYHYQLEMAKNITAAAEAIYTPEEA
PAKIDHVIKTALREKKPVYLEIACNIASMPCAAPGPASALFNDEASDE
ASLNAAVEETLKFIADRDKVAVLVGSKLRAAGAEEAAVKFADALGGAV
ATMAAAKSFFPEENPHYIGTSWGEVSYPGVEKTMKEADAVIALAPVFN
DYSTTGWTDIPDPKKLVLAEPRSVVVNGIRFPSVHLKDYLTRLAQKVS
KKTGALDFFKSLNAGELKKAAPADPSAPLVNAEIARQVEALLTPNTTV
IAETGDSWFNAQRIKLPNGARVEYEMQWGHIGWSVPAAFGYAVGAPER
RNILMVGDGSFQLTAQEVAQMVRLKPPVIIFLINNYGYTIEVMIHDGP
YNNIKNWDYAGLMEVFNGNGGYDSGAGKGLKAKTGGELAEAIKVALAN
TDGPTLIECFIGREDCTEELVKWGKRVAAANSRKPVNKLL
SEQ ID no: 21
Description: Escherichia coli
acetyl-CoA synthetase (acs)
GenBank: EU891279.1
DNA sequence
atgagtcaaattcacaaacacaccattcctgccaacatcgcagaccgt
tgcctgataaaccctcagcagtacgaggcgatgtatcaacaatctatt
aacgcacctgataccttctggggcgaacagggaaaaattctcgactgg
atcaaaccgtaccagaaggtgaaaaacacctcctttgcccccggtaat
gtgtccattaaatggtacgaggacggcacgctgaatctggcggcaaac
tgccttgaccgccatctgcaagaaaacggcgatcgtaccgccatcatc
tgggaaggcgacgacgccagccagagcaaacatatcagctataaagag
ctgcaccgcgacgtctgccgcttcgccaataccctgctcaagctgggc
attaaaaaaggtgatgtggtggcgatttatatgccgatggtgccggaa
gccgcggttgcgatgctggcctgcgcccgtattggcgcggtgcattcg
gtaattttcggtggcttctcgccggaagcggttgccgggcgcattatc
gattccaactcacgactggtgatcacttccgacgaaggcgtgcgcgcc
gggcgtagtattccgctgaagaaaaacgttgatgacgcactaaaaaac
ccgaacgtcaccagcgtagagcatgtggtggtactgaagcgtactggc
gggaaaattgactggcaggaagggcgcgacctgtggtggcacgaccag
gttgagcaagccagcgatcagcaccaggcggaagagatgaacgccgaa
gatccgctgtttattctctatacctccggttctaccggaaaaccaaaa
ggcgtactgcacactaccggcggttatctggtgtacgcggcgctgacc
tttaaatatgtctttgattatcatccgggcgatatctactggtgcacc
gccgatgtgggctgggtgaccggacacagttatttgctgtacggcccg
ctggcctgcggcgcgaccacgctgatgtttgaaggcgtaccgaactgg
ccgacgcctgcccgtatggcacaggtggtggacaagcatcaggtcaat
attctctataccgcgcccacggcgattcgcgcgctgatggcggaaggc
gataaagcgatcgaaggcaccgaccgttcgtcgctgcgcattctcggt
tccgtgggcgagccaattaacccggaagcgtgggagtggtactggaaa
aaaatcggcaacgagaaatgtccggtggtcgatacctggtggcagacc
gaaaccggcggtttcatgatcaccccgctgcctggcgctaccgagctg
aaagccggttcggcaacacgtccgttcttcggcgtgcaaccggcgctg
gtcgataacgaaggtaacccgctggaaggggctaccgaaggtagcctg
gtgatcaccgactcctggccgggtcaggcgcgtacgctgtttggcgat
cacgaacgttttgagcagacctatttttccaccttcaaaaatatgtat
ttcagcggcgacggcgcgcgtcgtgatgaagatagctattactggatc
accgggcgtgtggacgatgtgctgaacgtctccggtcaccgtctggga
acggcggagattgagtcggcgctggtggcgcatccgaaaatcgccgaa
gccgctgtcgtcggtattccgcacaatattaaaggtcaggcgatctac
gcctacgtcacgcttaatcacggggaggaaccgtcaccagaactgtac
gcagaagtccgcaactgggtgcgtaaagagattggcccgctggcgacg
ccagacgtgctgcactggaccgactccctgcctaaaacccgctccggc
aaaattatgcgccgtattctgcgcaaaattgcggcgggcgataccagc
aacctgggcgatacctcgacgcttgccgatcctggcgtagtcgagaag
ctgcttgaagagaagcaggctatcgcgatgccatcgtaa
SEQ ID no: 22
Description: Escherichia coli
acetyl-CoA synthetase (acs)
GenBank: ACI73860.1
Protein sequence
MSQIHKHTIPANIADRCLINPQQYEAMYQQSINAPDTFWGEQGKILDW
IKPYQKVKNTSFAPGNVSIKWYEDGTLNLAANCLDRHLQENGDRTAII
WEGDDASQSKHISYKELHRDVCRFANTLLKLGIKKGDVVAIYMPMVPE
AAVAMLACARIGAVHSVIFGGFSPEAVAGRIIDSNSRLVITSDEGVRA
GRSIPLKKNVDDALKNPNVTSVEHVVVLKRTGGKIDWQEGRDLWWHDQ
VEQASDQHQAEEMNAEDPLFILYTSGSTGKPKGVLHTTGGYLVYAALT
FKYVFDYHPGDIYWCTADVGWVTGHSYLLYGPLACGATTLMFEGVPNW
PTPARMAQVVDKHQVNILYTAPTAIRALMAEGDKAIEGTDRSSLRILG
SVGEPINPEAWEWYWKKIGNEKCPVVDTWWQTETGGFMITPLPGATEL
KAGSATRPFFGVQPALVDNEGNPLEGATEGSLVITDSWPGQARTLFGD
HERFEQTYFSTFKNMYFSGDGARRDEDSYYWITGRVDDVLNVSGHRLG
TAEIESALVAHPKIAEAAVVGIPHNIKGQAIYAYVTLNHGEEPSPELY
AEVRNWVRKEIGPLATPDVLHWTDSLPKTRSGKIMRRILRKIAAGDTS
NLGDTSTLADPGVVEKLLEEKQAIAMPS

In some embodiments host cells (e.g., microorganisms) can be transformed with multiple genes encoding one or more enzymes. For example, a single transformed cell can contain exogenous nucleic acids encoding an entire glycolytic or solventogenic pathway. One example of a pathway can include genes encoding a pyruvate decarboxylase, a heterologous alcohol dehydrogenase, and/or a synthetase. Such cells transformed with entire pathways and/or enzymes extracted from them, can ferment certain components of biomass more efficiently than the naturally-occurring organism. Constructs can contain multiple copies of the same gene, and/or multiple genes encoding the same enzyme from different organisms, and/or multiple genes with mutations in one or more parts of the coding sequences. Other constructs can contain plasmids to disrupt the activity of certain enzymes, such as lactate dehydrogenase (See, for example, U.S. application Ser. No. 12/729,037). In some embodiments, the nucleic acid sequences encoding the genes can be similar or identical to the endogenous gene. In other embodiments, the gene inserted into the microbe's genome may not have an endogenous counterpart. There can be a percent similarity of 70% or more in comparing the base pairs of the sequences. Examples of genes that can be used in the methods described supra are shown in Table 5 (supra) and Table 6.

TABLE 6
SEQ ID no: 23
Description: Zymomonas mobilis
glucokinase (glk)
NCBI Ref.: NC_013355.1
Comp.(994156 . . . 995130)
DNA sequence
atggaaattgttgcgattgacatcggtggaacgcatgcgcgtttctct
attgcggaagtaagcaatggtcgggttctttctcttggagaagaaacg
acttttaaaacggcagaacatgctagcttacagttagcttgggaacgt
ttcggtgaaaaactgggtcgtcctctgccacgtgccgcagctattgca
tgggctggcccggttcatggtgaagttttaaaacttaccaataaccct
tgggtattaagaccagctactctgaatgaaaagctggacatcgatacg
catgttctgatcaatgacttcggtgcggttgcccacgcggttgcgcat
atggattcttcttatctggatcatatttgtggtcctgatgaagcgctt
cctagcgatggtgttatcactattcttggtccgggaacgggcttgggt
gttgcccatctgttgcggactgaaggccgttatttcgtcatcgaaact
gaaggcggtcatatcgactttgctccgcttgacagacttgaagacaaa
attctggcacgtttacgtgaacgtttccgccgcgtttctatcgaacgc
attatttctggcccgggtcttggtaatatctacgaagcactggctgcc
attgaaggcgttccgttcagcttgctggatgatattaaattatggcag
atggctttggaaggtaaagacaaccttgctgaagccgctttggatcgc
ttctgcttgagccttggcgctatcgctggtgatcttgctttggcacag
ggtgcaaccagtgttgttattggcggtggtgtcggtcttcgtatcgct
tcccatttgccggaatctggcttccgtcagcgctttgtttcaaaagga
cgctttgaacgcgtcatgtccaagattccggttaagttgattacttat
ccgcagcctggactgctgggtgcggcagctgcctatgccaacaaatat
tctgaagttgaataa
SEQ ID no: 24
Description: Zymomonas mobilis
glucokinase (glk)
NCBI Ref: YP_003226001.1
Protein sequence
MEIVAIDIGGTHARFSIAEVSNGRVLSLGEETTFKTAEHASLQLAWER
FGEKLGRPLPRAAAIAWAGPVHGEVLKLTNNPWVLRPATLNEKLDIDT
HVLINDFGAVAHAVAHMDSSYLDHICGPDEALPSDGVITILGPGTGLG
VAHLLRTEGRYFVIETEGGHIDFAPLDRLEDKILARLRERFRRVSIER
IISGPGLGNIYEALAAIEGVPFSLLDDIKLWQMALEGKDNLAEAALDR
FCLSLGAIAGDLALAQGATSVVIGGGVGLRIASHLPESGFRQRFVSKG
RFERVMSKIPVKLITYPQPGLLGAAAAYANKYSEVE
SEQ ID no: 25
Description: Zymomonas mobilis
glucose transport (facilitator) (glf)
GenBank: M60615.1
(185 . . . 1606)
DNA sequence
cgccatgagttctgaaagtagtcagggtctagtcacgcgactagccct
aatcgctgctataggcggcttgcttttcggttacgattcagcggttat
cgctgcaatcggtacaccggttgatatccattttattgcccctcgtca
cctgtctgctacggctgcggcttccctttctgggatggtcgttgttgc
tgttttggtcggttgtgttaccggttctttgctgtctggctggattgg
tattcgcttcggtcgtcgcggcggattgttgatgagttccatttgttt
cgtcgccgccggttttggtgctgcgttaaccgaaaaattatttggaac
cggtggttcggctttacaaattttttgctttttccggtttcttgccgg
tttaggtatcggtgtcgtttcaaccttgaccccaacctatattgctga
aattcgtccgccagacaaacgtggtcagatggtttctggtcagcagat
ggccattgtgacgggtgctttaaccggttatatctttacctggttact
ggctcatttcggttctatcgattgggttaatgccagtggttggtgctg
gtctccggcttcagaaggcctgatcggtattgccttcttattgctgct
gttaaccgcaccggatacgccgcattggttggtgatgaagggacgtca
ttccgaggctagcaaaatccttgctcgtctggaaccgcaagccgatcc
taatctgacgattcaaaagattaaagctggctttgataaagccatgga
caaaagcagcgcaggtttgtttgcttttggtatcaccgttgtttttgc
cggtgtatccgttgctgccttccagcagttagtcggtattaacgccgt
gctgtattatgcaccgcagatgttccagaatttaggttttggagctga
tacggcattattgcagaccatctctatcggtgttgtgaacttcatctt
caccatgattgcttcccgtgttgttgaccgcttcggccgtaaacctct
gcttatttggggtgctctcggtatggctgcaatgatggctgttttagg
ctgctgtttctggttcaaagtcggtggtgttttgcctttggcttctgt
gcttctttatattgcagtctttggtatgtcatggggccctgtctgctg
ggttgttctgtcagaaatgttcccgagttccatcaagggcgcagctat
gcctatcgctgttaccggacaatggttagctaatatcttggttaactt
cctgtttaaggttgccgatggttctccagcattgaatcagactttcaa
ccacggtttctcctatctcgttttcgcagcattaagtatcttaggtgg
cttgattgttgctcgcttcgtgccggaaaccaaaggtcggagcctgga
tgaaatcgaggagatgtggcgctcccagaagtag
SEQ ID no: 26
Description: Zymomonas mobilis
glucose transport (facilitator) (glf)
GenBank: AAA27691.1
Protein sequence
mssessqglvtrlaliaaiggllfgydsaviaaigtpvdihfiaprhl
sataaaslsgmvvvavlvgcvtgsllsgwigirfgrrggllmssicfv
aagfgaalteklfgtggsalqifcffrflaglgigvvstltptyiaei
rppdkrgqmvsgqqmaivtgaltgyiftwllahfgsidwvnasgwcws
pasegligiafllllltapdtphwlvmkgrhseaskilarlepqadpn
ltiqkikagfdkamdkssaglfafgitvvfagvsvaafqqlvginavl
yyapqmfqnlgfgadtallqtisigvvnfiftmiasrwdrfgrkplli
wgalgmaaramavlgccfwfkvggvlplasvllyiavfgmswgpvcwv
vlsemfpssikgaampiavtgqwlanilvnfIfkvadgspalnqtfnh
gfsylvfaalsilgglivarfvpetkgrsldeieemwrsqk
SEQ ID no: 27
Description: Zymomonas mobilis
glucose-6-phosphate 1-dehydrogenase (zwf)
NCBI Ref.: NC_013355.1
(997079 . . . 998536)
Comp. DNA sequence
atgacaaataccgtttcgacgatgatattgtttggctcgactggcgac
ctttcacagcgtatgctgttgccgtcgctttatggtcttgatgccgat
ggtttgcttgcagatgatctgcgtatcgtctgcacctctcgtagcgaa
tacgacacagatggtttccgtgattttgcagaaaaagctttagatcgc
tttgtcgcttctgaccggttaaatgatgacgctaaagctaaattcctt
aacaagcttttctacgcgacggtcgatattacggatccgacccaattc
ggaaaattagctgacctttgtggcccggtcgaaaaaggtatcgccatt
tatctttcgactgcgccttctttgtttgaaggggcaatcgctggcctg
aaacaggctggtctggctggtccaacttctcgcctggcgcttgaaaaa
cctttaggtcaggatcttgcttcttccgatcatattaatgatgcggtt
ttgaaagttttctctgaaaagcaagtttatcgtattgaccattatctg
ggtaaagaaacggttcagaaccttctgaccctgcgctttggtaatgct
ttgtttgaaccgctttggaattcaaaaggcattgaccacgttcagatc
agcgttgctgaaacggttggtcttgaaggtcgtatcggttatttcgac
ggttctggcagcttgcgcgatatggttcaaagccatatccttcagttg
gtcgctttggttgcaatggaaccgccggctcatatggaagccaacgct
gttcgtgacgaaaaggtaaaagttttccgcgctctgcgtccgatcaat
aacgacaccgtctttacgcataccgttaccggtcaatatggtgccggt
gtttctggtggtaaagaagttgccggttacattgacgaactgggtcag
ccttccgataccgaaacctttgttgctatcaaagcgcatgttgataac
tggcgttggcagggtgttccgttctatatccgcactggtaagcgttta
cctgcacgtcgttctgaaatcgtggttcagtttaaacctgttccgcat
tcgattttctcttcttcaggtggtatcttgcagccgaacaagctgcgt
attgtcttacagcctgatgaaaccatccagatttctatgatggtgaaa
gaaccgggtcttgaccgtaacggtgcgcatatgcgtgaagtttggctg
gatctttccctcacggatgtgtttaaagaccgtaaacgtcgtatcgct
tatgaacgcctgatgcttgatcttatcgaaggcgatgctactttattt
gtgcgtcgtgacgaagttgaggcgcagtgggtttggattgacggaatt
cgtgaaggctggaaagccaacagtatgaagccaaaaacctatgtctct
ggtacatgggggccttcaactgctatagctctggccgaacgtgatgga
gtaacttggtatgactga
SEQ ID no: 28
Description: Zymomonas mobilis
glucose-6-phosphate 1-dehydrogenase(zwf)
NCBI Ref: Yp_003226003.1
Protein sequence
MTNTVSTMILFGSTGDLSQRMLLPSLYGLDADGLLADDLRIVCTSRSE
YDTDGFRDFAEKALDRFVASDRLNDDAKAKFLNKLFYATVDITDPTQF
GKLADLCGPVEKGIAIYLSTAPSLFEGAIAGLKQAGLAGPTSRLALEK
PLGQDLASSDHINDAVLKVFSEKQVYRIDHYLGKETVQNLLTLRFGNA
LFEPLWNSKGIDHVQISVAETVGLEGRIGYFDGSGSLRDMVQSHILQL
VALVAMEPPAHMEANAVRDEKVKVFRALRPINNDTVFTHTVTGQYGAG
VSGGKEVAGYIDELGQPSDTETFVAIKAHVDNWRWQGVPFYIRTGKRL
PARRSEIVVQFKPVPHSIFSSSGGILQPNKLRIVLQPDETIQISMMVK
EPGLDRNGAHMREVWLDLSLTDVFKDRKRRIAYERLMLDLIEGDATLF
VRRDEVEAQWVWIDGIREGWKANSMKPKTYVSGTWGPSTAIALAERDG
VTWYD
SEQ ID no: 29
Description: Zymomonas mobilis
6-phosphgluconate dehydratase (edd)
NCBI Ref.: NC_013355.1
(995263 . . . 997086)
Complement
DNA sequence
atgactgatctgcattcaacggtagaaaaggttaccgcgcgcgttatt
gaacgctcgcgggaaacccgtaaggcttatctggatttgatccagtat
gagcgggaaaaaggcgtagaccgtccaaacctgtcctgtagtaacctt
gctcatggctttgcggctatgaatggtgacaagccagctttgcgcgac
ttcaaccgcatgaatatcggcgtcgtgacttcctacaacgatatgttg
tcggctcatgaaccatattatcgctatccggagcagatgaaagtattt
gctcgcgaagttggcgcaacggttcaggtcgccggtggcgtgcctgct
atgtgcgatggtgtgacccaaggtcagccgggcatggaagaatccctg
tttagccgcgatgttatcgctttggctaccagcgtttctttgtctcat
ggtatgtttgaaggggctgcccttctcggtatctgtgacaagattgtc
cctggtctgttgatgggcgctctgcgcttcggccacctgccgaccatt
ctggtcccatcaggcccgatgacgaccggtatcccgaacaaagaaaaa
atccgtatccgtcagctctatgctcagggtaaaatcggccagaaagaa
cttctggatatggaagcggcttgctaccatgctgaaggtacctgcacc
ttctatggtacggcaaacaccaaccagatggttatggaagtcctcggt
cttcatatgccaggttcggcatttgttaccccgggtaccccgctccgt
caggctctgacccgtgctgctgtgcatcgcgttgctgaattgggttgg
aagggcgacgattatcgtccgcttggtaagatcattgacgaaaaatca
atcgtcaatgccattgttggtctgttggcaaccggtggttccaccaac
cataccatgcatattccggctattgctcgtgctgctggtgttatcgtt
aactggaatgacttccatgatctttctgaagttgttccgttgattgcc
cgcatttacccgaatggcccgcgcgacatcaatgaattccagaatgca
ggcggcatggcttatgtcatcaaagaactgctttctgctaatctgttg
aaccgtgatgtcacgaccattgccaagggcggtatcgaagaatacgcc
aaggctccggcattaaatgacgctggcgaattggtatggaagccagct
ggcgaacctggtgatgacaccattctgcgtccggtttctaatcctttc
gcaaaagatggcggtctgcgtctcttggaaggtaaccttggacgtgca
atgtacaaagccagtgcagttgatcctaaattctggaccattgaagca
ccggttcgcgtcttctctgaccaagacgatgttcagaaagccttcaag
gctggcgaattgaacaaagacgttatcgttgttgttcgtttccagggc
ccgcgcgcaaacggtatgcctgaattgcataagctgaccccggctttg
ggtgttctgcaggataatggctacaaagttgctttggtaactgatggt
cgtatgtccggtgctaccggtaaagttccggttgctttgcatgtcagc
ccagaagctcttggcggtggtgccatcggtaaattacgtgatggcgat
atcgtccgtatctcggttgaagaaggcaaacttgaagctttggttcca
gctgatgagtggaatgctcgtccgcatgctgaaaaaccggctttccgt
ccgggaaccggacgcgaattgtttgatatcttccgtcagaacgctgct
aaagctgaagacggtgcagtcgcaatatatgcaggtgccggtatctaa
SEQ ID no: 30
Description: Zymomonas mobilis
6-phosphgluconate dehydratase (edd)
NCBI Ref: YP_003226002.1
Protein sequence
MTDLHSTVEKVTARVIERSRETRKAYLDLIQYEREKGVDRPNLSCSNL
AHGFAAMNGDKPALRDFNRMNIGVVTSYNDMLSAHEPYYRYPEQMKVF
AREVGATVQVAGGVPAMCDGVTQGQPGMEESLFSRDVIALATSVSLSH
GMFEGAALLGICDKIVPGLLMGALRFGHLPTILVPSGPMTTGIPNKEK
IRIRQLYAQGKIGQKELLDMEAACYHAEGTCTFYGTANTNQMVMEVLG
LHMPGSAFVTPGTPLRQALTRAAVHRVAELGWKGDDYRPLGKIIDEKS
IVNAIVGLLATGGSTNHTMHIPAIARAAGVIVNWNDFHDLSEVVPLIA
RIYPNGPRDINEFQNAGGMAYVIKELLSANLLNRDVTTIAKGGIEEYA
KAPALNDAGELVWKPAGEPGDDTILRPVSNPFAKDGGLRLLEGNLGRA
MYKASAVDPKFWTIEAPVRVFSDQDDVQKAFKAGELNKDVIVVVRFQG
PRANGMPELHKLTPALGVLQDNGYKVALVTDGRMSGATGKVPVALHVS
PEALGGGAIGKLRDGDIVRISVEEGKLEALVPADEWNARPHAEKPAFR
PGTGRELFDIFRQNAAKAEDGAVAIYAGAGI
SEQ ID no: 31
Description: Bacillus subtilis
phosphotransferase system (PTS)
glucose-specific enzyme IICBA
component (ptsG)
NCBI Ref: NC_000964.3
(1457187 . . . 1459286)
DNA sequence
atgtttaaagcattattcggcgttcttcaaaaaattgggcgtgcgctt
atgcttccagttgcgatccttccggctgcgggtattttgcttgcgatc
gggaatgcgatgcaaaataaggacatgattcaggtcctgcatttcttg
agcaatgacaatgttcagcttgtagcaggtgtgatggaaagtgctggg
cagattgttttcgataaccttccgcttcttttcgcagtaggtgtagcc
atcgggcttgccaatggtgatggagttgcagggattgcagcaattatc
ggttatcttgtaatgaatgtatccatgagtgcggttcttcttgcaaac
ggaaccattccttcggattcagttgaaagagccaagttctttacggaa
aaccatcctgcatatgtaaacatgcttggtatacctaccttggcgaca
ggggtgttcggcggtattatcgtcggtgtgttagctgcattattgttt
aacagattttacacaattgaactgccgcaataccttggtttctttgcg
ggtaaacgtttcgttccaattgttacgtcaatttctgcactgattctg
ggtcttattatgttagtgatctggcctccaatccagcatggattgaat
gccttttcaacaggattagtggaagcgaatccaacccttgctgcattt
atcttcggggtgattgaacgttcgcttatcccattcggattgcaccat
attttctattcaccgttctggtatgaattcttcagctataagagtgca
gcaggagaaatcatccgcggggatcagcgtatctttatggcgcagatt
aaagacggcgtacagttaacggcaggtacgttcatgacaggtaaatat
ccatttatgatgttcggtctgcctgctgcggcgcttgccatttatcat
gaagcaaaaccgcaaaacaaaaaactcgttgcaggtattatgggttca
gcggccttgacatctttcttaacggggatcacagagccattggaattt
tctttcttattcgttgctccagtcctgtttgcgattcactgtttgttt
gcgggactttcattcatggtcatgcagctgttgaatgttaagattggt
atgacattctccggcggtttaattgactacttcctattcggtatttta
ccaaaccggacggcatggtggcttgtcatccctgtcggcttagggtta
gcggtcatttactactttggattccgatttgccatccgcaaatttaat
ctgaaaacacctggacgcgaggatgctgcggaagaaacagcagcacct
gggaaaacaggtgaagcaggagatcttccttatgagattctgcaggca
atgggtgaccaggaaaacatcaaacaccttgatgcttgtatcactcgt
ctgcgtgtgactgtaaacgatcagaaaaaggttgataaagaccgtctg
aaacagcttggcgcttccggagtgctggaagtcggcaacaacattcag
gctattttcggaccgcgttctgacgggttaaaaacacaaatgcaagac
attattgcgggacgcaagcctagacctgagccgaaaacatctgctcaa
gaggaagtaggccagcaggttgaggaagtgattgcagaaccgctgcaa
aatgaaatcggcgaggaagttttcgtttctccgattaccggggaaatt
cacccaattacggatgttcctgaccaagtcttctcagggaaaatgatg
ggtgacggttttgcgattctcccttctgaaggaattgtcgtatcaccg
gttcgcggaaaaattctcaatgtgttcccgacaaaacatgcgatcggc
ctgcaatccgacggcggaagagaaattttaatccactttggtattgat
accgtcagcctgaagggcgaaggatttacgtctttcgtatcagaagga
gaccgcgttgagcctggacaaaaacttcttgaagttgatctggatgca
gtcaaaccgaatgtaccatctctcatgacaccgattgtatttacaaac
cttgctgaaggagaaacagtcagcattaaagcaagcggttcagtcaac
agagaacaagaagatattgtgaagattgaaaaataa
SEQ ID no: 32
Description: Bacillus subtilis
phosphotransferase system (PTS)
glucose-specific enzyme IICBA
component (ptsG)
NCBI Ref.: NP_389272.1
Protein sequence
MFKALFGVLQKIGRALMLPVAILPAAGILLAIGNAMQNKDMIQVLHFL
SNDNVQLVAGVMESAGQIVFDNLPLLFAVGVAIGLANGDGVAGIAAII
GYLVMNVSMSAVLLANGTIPSDSVERAKFFTENHPAYVNMLGIPTLAT
GVFGGIIVGVLAALLFNRFYTIELPQYLGFFAGKRFVPIVTSISALIL
GLIMLVIWPPIQHGLNAFSTGLVEANPTLAAFIFGVIERSLIPFGLHH
IFYSPFWYEFFSYKSAAGEIIRGDQRIFMAQIKDGVQLTAGTFMTGKY
PFMMFGLPAAALAIYHEAKPQNKKLVAGIMGSAALTSFLTGITEPLEF
SFLFVAPVLFAIHCLFAGLSFMVMQLLNVKIGMTFSGGLIDYFLFGIL
PNRTAWWLVIPVGLGLAVIYYFGFRFAIRKFNLKTPGREDAAEETAAP
GKTGEAGDLPYEILQAMGDQENIKHLDACITRLRVTVNDQKKVDKDRL
KQLGASGVLEVGNNIQAIFGPRSDGLKTQMQDIIAGRKPRPEPKTSAQ
EEVGQQVEEVIAEPLQNEIGEEVFVSPITGEIHPITDVPDQVFSGKMM
GDGFAILPSEGIVVSPVRGKILNVFPTKHAIGLQSDGGREILIHFGID
TVSLKGEGFTSFVSEGDRVEPGQKLLEVDLDAVKPNVPSLMTPIVFTN
LAEGETVSIKASGSVNREQEDIVKIKK
SEQ ID no: 33
Description: Bacillus subtilis
glucose/mannose:H+ symporter (glcP)
NCBI Ref.: NC_000964.3
(1125123 . . . 1126328)
Complement
DNA sequence
atgttaagagggacatatttatttggatatgctttcttttttacagta
ggtattatccatatatcaacagggagtttgacaccatttttattagag
gcttttaacaagacaacagatgatatttcggtcataatcttcttccag
tttaccggatttctaagcggagtattaatcgcacctttaatgattaag
aaatacagtcattttaggacacttactttagctttgacaataatgctt
gtagcgttaagtatcttttttctaaccaaggattggtattatattatt
gtaatggcttttctcttaggatatggagcaggcacattagaaacgaca
gttggttcatttgttattgctaatttcgaaagtaatgcagaaaaaatg
agtaagctggaagttctctttggattaggcgctttatctttcccatta
ttaattaattccttcatagatatcaataactggtttttaccatattac
tgtatattcacctttttattcgtcctattcgtagggtggttaattttc
ttgtctaagaaccgagagtacgctaagaatgctaaccaacaagtgacc
tttccagatggaggagcatttcaatactttataggagatagaaaaaaa
tcaaagcaattaggcttttttgtatttttcgctttcctatatgctgga
attgaaacaaattttgccaactttttaccttcaatcatgataaaccaa
gacaatgaacaaattagtcttataagtgtctcctttttctgggtaggg
atcatcataggaagaatattgattggtttcgtaagtagaaggcttgat
ttttccaaataccttctttttagctgtagttgtttaattgttttgttg
attgccttctcttatataagtaacccaatacttcaattgagtggtaca
tttttgattggcctaagtatagcggggatatttcccattgctttaaca
ctagcatcaatcattattcagaagtacgttgacgaagttacaagttta
tttattgcctcggcaagtttcggaggagcgatcatctctttcttaatt
ggatggagtttaaaccaggatacgatcttattaaccatgggaatattt
acaactatggcggtcattctagtaggtatttctgtaaagattaggaga
actaaaacagaagaccctatttcacttgaaaacaaagcatcaaaaaca
cagtag
SEQ ID no: 34
Description: Bacillus subtilis
glucose/mannose: H+ symporter (glcP)
NCBI Ref.: NP_388933.1
DNA/Protein sequence
MLRGTYLFGYAFFFTVGIIHISTGSLTPFLLEAFNKTTDDISVIIFFQ
FTGFLSGVLIAPLMIKKYSHFRTLTLALTIMLVALSIFFLTKDWYYII
VMAFLLGYGAGTLETTVGSFVIANFESNAEKMSKLEVLFGLGALSFPL
LINSFIDINNWFLPYYCIFTFLFVLFVGWLIFLSKNREYAKNANQQVT
FPDGGAFQYFIGDRKKSKQLGFFVFFAFLYAGIETNFANFLPSIMINQ
DNEQISLISVSFFWVGIIIGRILIGFVSRRLDFSKYLLFSCSCLIVLL
IAFSYISNPILQLSGTFLIGLSIAGIFPIALTLASIIIQKYVDEVTSL
FIASASFGGAIISFLIGWSLNQDTILLTMGIFTTMAVILVGISVKIRR
TKTEDPISLENKASKTQ
SEQ ID no: 35
Description: Bacillus subtilis
squalene-hopene cyclase (sqhC)
NCBI Ref.: NC_000964.3
(2102168 . . . 2104066)
DNA sequence
atgggcacacttcaggagaaagtgaggcgttttcaaaagaaaaccatt
accgagttaagagacaggcaaaatgctgatggttcatggacattttgc
tttgaaggaccaatcatgacaaattccttttttattttgctccttacc
tcactagatgaaggcgaaaatgaaaaagaactgatatcatcccttgca
gccggcattcatgcaaaacagcagccagacggcacatttatcaactat
cccgatgaaacgcgcggaaatctaacggctaccgtccaaggatatgtc
gggatgctggcttcaggatgttttcacagaactgagccgcacatgaag
aaagctgaacaatttatcatctcacatggcggtttgagacatgttcat
tttatgacaaaatggatgcttgccgcgaacgggctttatccttggcct
gctttgtatttaccattatcactcatggcgctccccccaacattgccg
attcatttctatcagttcagctcatatgcccgtattcattttgctcct
atggctgtaacactcaatcagcgatttgtccttattaaccgcaatatt
tcatctcttcaccatctcgatccgcacatgacaaaaaatcctttcact
tggcttcggtctgatgctttcgaagaaagagatctcacgtctattttg
ttacattggaaacgcgtttttcatgcaccatttgcttttcagcagctg
ggcctacagacagctaaaacgtatatgctggaccggattgaaaaagat
ggaacattatacagctatgcgagcgcaaccatatatatggtttacagc
cttctgtcacttggtgtgtcacgctattctcctattatcaggagggcg
attaccggcattaaatcactggtgactaaatgcaacgggattccttat
ctggaaaactctacttcaactgtttgggatacagctttaataagctat
gcccttcaaaaaaatggtgtgaccgaaacggatggctctgttacaaaa
gcagccgactttttgctagaacgccagcataccaaaatagcagattgg
tctgtcaaaaatccaaattcagttcctggcggctgggggttttcaaac
attaatacaaataaccctgactgtgacgacactacagccgttttaaag
gcgattccccgcaatcattctcctgcagcatgggagcggggggtatct
tggcttttatcgatgcaaaacaatgacggcggattttctgctttcgaa
aaaaatgtgaaccatccactgatccgccttctgccgcttgaatccgcc
gaggacgctgcagttgacccttcaaccgccgacctcaccggacgtgta
ctgcactttttaggcgagaaagttggcttcacagaaaaacatcaacat
attcaacgcgcagtgaagtggcttttcgaacatcaggaacaaaatggg
tcttggtacggcagatggggtgtttgctacatttacggcacttgggct
gctcttactggtatgcatgcatgcggggttgaccgaaagcatcccggt
atacaaaaggctctgcgttggctcaaatccatacaaaatgatgacgga
agctggggagaatcctgcaaaagcgccgaaatcaaaacatatgtaccg
cttcatagaggaaccattgtacaaacggcctgggctttagacgctttg
ctcacatatgaaaattccgaacatccgtctgttgtgaaaggcatgcaa
taccttaccgacagcagttcgcatagcgccgatagcctcgcgtatcca
gcagggatcggattgccgaagcaattttatattcgctatcacagttat
ccatatgtattctctttgctggctgtcgggaagtatttagattctatt
gaaaaggagacagcaaatgaaacgtga
SEQ ID no: 36
Description: Bacillus subtilis
squalene-hopene cyclase (sqhC)
NCBI Ref.: NP_389814.2
Protein sequence
MGTLQEKVRRFQKKTITELRDRQNADGSKTFCFEGPIMTNSFFILLLT
SLDEGENEKELISSLAAGIHAKQQPDGTFINYPDETRGNLTATVQGYV
GMLASGCFHRTEPHMKKAEQFIISHGGLRHVHFMTKWMLAANGLYPKP
ALYLPLSLMALPPTLPIHFYQFSSYARIHFAPMAVTLNQRFVLINRNI
SSLHHLDPHMTKNPFTWLRSDAFEERDLTSILLHWKRVFHAPFAFQQL
GLQTAKTYMLDRIEKDGTLYSYASATIYMVYSLLSLGVSRYSPIIRRA
ITGIKSLVTKCNGIPYLENSTSTVWDTALISYALQKNGVTETDGSVTK
AADFLLERQHTKIADWSVKNPNSVPGGWGFSNINTNNPDCDDTTAVLK
AIPRNHSPAAWERGVSWLLSMQNNDGGFSAFEKNVNHPLIRLLPLESA
EDAAVDPSTADLTGRVLHFLGEKVGFTEKHQHIQRAVKWLFEHQEQNG
SWYGRWGVCYIYGTWAALTGMHACGVDRKHPGIQKALRWLKSIQNDDG
SWGESCKSAEIKTYVPLHRGTIVQTAWALDALLTYENSEHPSVVKGMQ
YLTDSSSHSADSLAYPAGIGLPKQFYIRYHSYPYVFSLLAVGKYLDSI
EKETANET
SEQ ID no: 37
Description: Bacillus subtilis
expansin (yoaJ)
GenBank: AF027868.1
(12919 . . . 13617)
DNA sequence
ttattcaggaaactgaacatggcccggtactgtataggctttggacgt
tccgctttcaggcagctttggaatggtgtctttcacaacttttccgcg
gatgtcagtcattctgactttgagagagccagtacctaaattcgtact
cacaaaatggttatagtccattttctccatgttgatccacttaccatc
cttttcatattccattttcataacaggatacttgtgatttctgacttg
gattgctgcccaccacctgctgctgccttctttgatccggtacgtgaa
attgccggtgattggggctttgacaacacgccatttaatattgatttt
tccgtctttcatattgccgattttacggaaggcattaggtgacagatc
aagagctccccgagcgccttcgggataaagatcagtaacatatacggt
tgttttcccttttggcccttcaacttccaaataagagccggcaagtgc
cgcttttactcctccgtaattgagatccgccggatttattgcagtaat
ctccatatcggaaggaatgggatccagcaggaaagctcctcctgaata
gcctgaccctgtatacgttgcataaccttcatgcaggtcgtcatatgc
tgccgaagcttgcggggaaaaacagaagatcgtcaacaaaaccatacc
aacaaatgcactcatgatctttttcat
SEQ ID no: 38
Description: Bacillus subtilis
expansin (yoaJ)
GenBank: AAB84448.1
Protein sequence
MKKIMSAFVGMVLLTIFCFSPQASAAYDDLHEGYATYTGSGYSGGAFL
LDPIPSDMEITAINPADLNYGGVKAALAGSYLEVEGPKGKTTVYVTDL
YPEGARGALDLSPNAFRKIGNMKDGKINIKWRVVKAPITGNFTYRIKE
GSSRWWAAIQVRNHKYPVMKMEYEKDGKWINMEKMDYNHFVSTNLGTG
SLKVRMTDIRGKVVKDTIPKLPESGTSKAYTVPGHVQFPE
SEQ ID no: 39
Description: Bacillus subtilis
beta-galactosidase (lacA)
GenBank: EU585783.1
DNA sequence
gtgatgtcaaagcttgaaaaaacgcacgtaacaaaagcgaaatttatg
ctccatgggggagactacaaccccgatcagtggctggatcggcccgat
attttagctgacgatatcaaactgatgaagctttctcatacgaatacg
ttttctgtcggtatttttgcatggagcgcacttgagccggaggagggc
gtatatcaatttgaatggctggatgatatttttgagcggattcacagt
ataggcggccgggtcatattagcaacgccgagcggagcccgtccggcc
tggctgtcgcaaacctatccggaagttttgcgcgtcaatgcctcccgc
gtcaaacagctgcacggcggaaggcgcaaccactgcctcacatctaaa
gtctaccgagaaaagacacggcacatcaaccgcttattagcagaacga
tacggaaatcacccggggctgttaatgtggcacatttcaaacgaatac
gggggagattgccactgtgatctatgccagcatgcttttcgggagtgg
ctgaaatcgaaatatgacaacagcctcaaggcattgaaccaggcgtgg
tggacccctttttggagccatacgttcaatgactggtcacaaattgaa
agcccttcgccgatcggtgaaaatggcttgcatggcctgaatttagat
tggcgccggttcgtcaccgatcaaacgatttcgttttataaaaatgaa
atcattccgctgaaagaattgacgcctgatatccctatcacaacgaat
tttatggctgacacaccggatttgatcccgtatcagggcctcgactac
agcaaatttgcaaagcatgtcgatgtcatcagctgggacgcttatcct
gtctggcacaatgactgggaaagcacagctgatttggcgatgaaggtc
ggttttatcaacgatctgtaccgaagcttgaagcagcagtctttctta
ttaatggagtgtacgccaagcgcggtcaattggcataacgtcaacaag
gcaaagcgcccgggcatgaatctgctgtcatccatgcaaatgattgcc
cacggctcggacagcgtactctatttccaataccgcaaatcacggggg
tcatcagaaaaattacacggagcggttgtggatcatgacaatagccca
aagaaccgcgtctttcaagaagtggccaaggtaggcgagacattggaa
cggctgtccgaagttgtcggaacgaagaggccggctcaaaccgcgatt
ttatatgactgggaaaatcattgggcgttcggggatgctcaggggttt
gcgaaggcgacaaaacgttatccgcaaacgcttcagcagcattaccgc
acattctgggaacacgatatccctgtcgacgtcattacgaaagaacaa
gacttttcaccatataaactgctgatcgtcccgatgctgtatttaatc
agcgaggacaccatttcccgtttaaaagcgtttacggctgacggcggc
accttagtcatgacgtatatcagcggggttgtgaatgagcatgactta
acatacacaggcggatggcatccggaccttcaagctatatttggagtt
gagcctcttgaaacggacaccctgtatccgaaggatcgaaacgctgtc
agctaccgcagccaaatatacgaaatgaaggattatgcaaccgtgatt
gatgtaaagactgctccagtggaagcggtgtatcaagaggatttttac
gcccgtacgccagctgtcacaagccatcaatatcagcagggcaaggcg
tattttatcggcgcgcgtttggaggatcaatttcaccgtgatttctat
gagggtctgatcacagacctgtctctttcacctgtttttccggttcgg
catggaaaaggcgtctccgtacaagcgaggcaggatcaggacaatgat
tatatttttgtgatgaactttacggaagaaaaacagctggtcacgttt
gaccagagtgtgaaggacataatgacaggagacatattgtcaggcgac
ctgacgatggaaaagtatgaagtgagaattgtcgtaaacacacattaa
SEQ ID no: 40
Description: Bacillus subtilis
beta-galactosidase (lacA)
GenBank: ACB72733.1
Protein sequence
MMSKLEKTHVTKAKFMLHGGDYNPDQWLDRPDILADDIKLMKLSHTNT
FSVGIFAWSALEPEEGVYQFEWLDDIFERIHSIGGRVILATPSGARPA
WLSQTYPEVLRVNASRVKQLHGGRRNHCLTSKVYREKTRHINRLLAER
YGNHPGLLMWHISNEYGGDCHCDLCQHAFREWLKSKYDNSLKALNQAW
WTPFWSHTFNDWSQIESPSPIGENGLHGLNLDWRRFVTDQTISFYKNE
IIPLKELTPDIPITTNFMADTPDLIPYQGLDYSKFAKHVDVISWDAYP
VWHNDWESTADLAMKVGFINDLYRSLKQQSFLLMECTPSAVNWHNVNK
AKRPGMNLLSSMQMIAHGSDSVLYFQYRKSRGSSEKLHGAVVDHDNSP
KNRVFQEVAKVGETLERLSEVVGTKRPAQTAILYDWENHWAFGDAQGF
AKATKRYPQTLQQHYRTFWEHDIPVDVITKEQDFSPYKLLIVPMLYLI
SEDTISRLKAFTADGGTLVMTYISGVVNEHDLTYTGGWHPDLQAIFGV
EPLETDTLYPKDRNAVSYRSQIYEMKDYATVIDVKTAPVEAVYQEDFY
ARTPAVTSHQYQQGKAYFIGARLEDQFHRDFYEGLITDLSLSPVFPVR
HGKGVSVQARQDQDNDYIFVMNFTEEKQLVTFDQSVKDIMTGDILSGD
LTMEKYEVRIVVNTH
SEQ ID no: 41
Description: Pseudoalteromonas haloplanktis
cellulase, GH5 (celG)
GenBank: CAA76775.1
DNA sequence
taacttcaatttaaggaaatacgatgaataacagttcaaataatcaca
aaagaaaggattttaaagtggcgagcttatcgttagctttattattag
gatgctcaacaatggccaatgccgctgttgagaagttaacggtgagtg
ggaatcaaattcttgcgggtggagaaaacacaagctttgcaggaccta
gcctattttggagtaatacggggtggggcgctgaaaaattttatacag
cagaaacagtagcaaaggcaaaaactgaatttaatgcaacattaattc
gtgcagctattggtcatggtacgagtactggtggtagtttgaactttg
attgggagggcaatatgagccgtcttgatactgttgtaaacgcagcta
ttgctgaggatatgtacgttattattgattttcatagccatgaagcac
ataccgatcaggcgactgcagttcgcttttttgaagacgtagctacca
aatatgggcagtacgacaatgttatttatgaaatttataacgagccat
tacaaatctcgtgggttaacgatattaagccttacgcagaaacagtta
ttgataaaattagagcaatcgaccctgataacttaattgtggttggaa
cgcctacgtggtcgcaagatgttgatgtggcatcacaaaacccaattg
atcgtgccaatattgcttacactctgcatttttatgctggcacgcatg
gtcaatcgtatcgaaataaagcacaaacagcactcgataacggcattg
cactattcgccacagagtggggaacagttaatgctgatggaaatggtg
gtgttaatatcaatgaaaccgatgcatggatggcattttttaaaacaa
acaatattagccacgctaactgggctttaaacgataaaaacgaaggtg
catcgttatttactccaggcggtagttggaattcactaacatcgtcag
gctctaaagttaaagagatcattcaaggttggggtggtggtagtagca
atgttgatttagatagcgacggggatggcgtaagtgacagccttgatc
agtgcaataatactcccgcaggtacaacggttgatagtattggttgtg
cagtaactgacagcgatgccgatggtattagcgataatgttgatcaat
gtcctaatacaccagtaggtgaaactgttaataatgtaggttgcgttg
ttgaagtagttgagccacaaagcgatgcggataacgatggtgtgaatg
atgatatcgatcagtgcccagatacacccgctggtacaagtgttgata
caaacggatgcagtgttgtaagctcaacagattgtaacggtattaatg
cataccctaattgggtgaacaaagattactcaggtggtccgtttaccc
acaataacaccgacgataaaatgcaatatcaaggtaatgcatacagcg
caaattggtatacaaacagccttccaggaagtgatgcttcgtggacgc
ttctttatacttgtaattaagcacgttttataaaatatgcgaagaagg
taaataatacatttaccttctttttaaaagtattagcctttataaaca
ctttgg
SEQ ID no: 42
Description: Pseuderomonas haloplanktis
cellulase, GH5 (celG)
GenBank:
Protein sequence
MNNSSNNHKRKDFKVASLSLALLLGCSTMANAAVEKLTVSGNQILAGG
ENTSFAGPSLFWSNTGWGAEKFYTAETVAKAKTEFNATLIRAAIGHGT
STGGSLNFDWEGNMSRLDTVVNAAIAEDMYVIIDFHSHEAHTDQATAV
RFFEDVATKYGQYDNVIYEIYNEPLQISWVNDIKPYAETVIDKIRAID
PDNLIVVGTPTWSQDVDVASQNPIDRANIAYTLHFYAGTHGQSYRNKA
QTALDNGIALFATEWGTVNADGNGGVNINETDAWMAFFKTNNISHANW
ALNDKNEGASLFTPGGSWNSLTSSGSKVKEIIQGWGGGSSNVDLDSDG
DGVSDSLDQCNNTPAGTTVDSIGCAVTDSDADGISDNVDQCPNTPVGE
TVNNVGCVVEVVEPQSDADNDGVNDDIDQCPDTPAGTSVDTNGCSVVS
STDCNGINAYPNWVNKDYSGGPFTHNNTDDKMQYQGNAYSANWYTNSL
PGSDASKTLLYTCN
SEQ ID no: 43
Description: Clostridium cellulolyticum
nicotinate-nucleotide
pyrophosphorylase (Ccel_3478)
NCBI Ref: NC_011898.1
(4046259 . . . 4047098)
DNA sequence
ctattctatattcatacttatatcaatagaatttgcagagtgagtaag
tttacctatagatataatatcaactcctgttaacgctacattatatat
agtttcttcacttatattccccgaggcctccgcaagagctcttttatt
tataagcttgacagcctcagccatctgttcatttgacatattatcaag
cataattatatctgccttgcattcgagagcctcacgaacctcttccat
ggactctacttctacttcgatctttacagtatgaggaatactgtttct
tacacgttgaaccgcatttgttattcctccggcagcagcaatgtggtt
atcctttatgagaacaccgtcagaaagcgaaaatctgtgattggctcc
tcctcctgcacttactgcatatttctccagaagtctcagaccgggagt
agtttttcttgtatcagttacctttacaggtaacccctgaactttact
aacatatctgttagtcatagtagcaattgcagataacctttgcataaa
gttcaatgcagtcctttcaccttttaacaaagctcttgtcgaaccgct
tacctcggctataatatcacctttcgaaaccttgtctccatcttttac
aaaggccttaaaacatatgccgctatccagtacctcaaaaacatactt
cgcaacatcgagccctgcaataaccgcatcctgctttgccataaattc
ggctctggatgaatctccttctgaaagaatattgtctgttgtaatatc
acctagtggcatatcctcttttaatgcattcataactatttcatggat
ataaagattactgagtttcat
SEQ ID no: 44
Description: Clostridium cellulolyticum
nicotinate-nucleotide
pyrophosphorylase (Ccel_3478)
NCBI Ref: YP_002507746.1
Protein sequence
MKLSNLYIHEIVMNALKEDMPLGDITTDNILSEGDSSRAEFMAKQDAV
IAGLDVAKYVFEVLDSGICFKAFVKDGDKVSKGDIIAEVSGSTRALLK
GERTALNFMQRLSAIATMTNRYVSKVQGLPVKVTDTRKTTPGLRLLEK
YAVSAGGGANHRFSLSDGVLIKDNHIAAAGGITNAVQRVRNSIPHTVK
IEVEVESMEEVREALECKADIIMLDNMSNEQMAEAVKLINKRALAEAS
GNISEETIYNVALTGVDIISIGKLTHSANSIDISMNIE
SEQ ID no: 45
Description: Clostridium cellulolyticum
L-aspartate oxidase (Ccel_3479)
NCBI Ref: NC_011898.1
(4047107 . . . 4048711)
DNA sequence
ttaaaatggtgaagccatttttcccttctccaattccttaactatatt
ttttctccagttcgtatcatcagttttgtcgtagtctgttctataatg
agcacctctgctctcttttctttcaagagctgattctataacaagccc
cgctactgtaagcatattcaacacttccagctttacaagactgaatcc
tgtaaaatccgtgtacttcttataaatatctttaataatttgggcagc
cttttcaagaccttgttgacttctgattatacctacatactttgtcat
tgcagcctgtatctcttccttcatagatttaagagccgcatcattttc
tttattggatacataacagagccttgaattgacggctgaattattaca
aggtcttccttcggactcgatcttctttgcgattttcctgccgaaaac
cagtccttctagcaaagaattgcttgcgagcctgtttgcaccgtgaat
ccctgtacaagctacctctccacatgcatacagacccggaatatttgt
ctgcccgtcaacatctgtttttactccccccatacaataatgctctgc
gggagcaaccggaataaaatccttagaaatatcaataccgtaatccag
acatgttttaaagatattaggaaacctactttcgatatattccctacc
tttaaatgttatatccagaaatacatttttggaatcagtaagatacat
ttctttaaaaatcgctcttgaaacaatgtctctgggtgccagttcacc
caactcgtgatatttcttcataaaaggctcaccgttgctatttttaag
ttgagcaccctctcctctaaccgcctcagatattaggaaactcttgtc
ttttgggtggtatagtactgtaggatggaactgtataaactccatatc
catggcctgggcacccgctctcaaacacattccgactccgtcaccagt
tgcgacctcaggattagtagtatgtgcataaatctgtccaaaaccccc
agttgcaacaactaccgagccggatttaaatatcttaattttatcttc
aatttcgtcataaactattacacctttgcatttgccctcttcgatcac
aagatcgactgcaaagtgactctcaaaaatcgatatgttcttctttct
ccgggcaacctcaataagcttgtcacagacttccttaccagtcgtatc
tcctgagtgaataattctatttacactatgggccccttctctagtaag
ggatagatgttgtccgcttttatcaaagtttacccctaggctgcacaa
aattctaatattttcagcagcctcttctaccagaacccatacgctctt
ttgatcatttaatcctgcacctgcaaaaagagtatctttgaaatgtag
ttgtggagaatcattcttctcatcaagagatactgctattcccccttg
tgcgagaactgaattgcttatgtccagtgtctctttggtaattatccc
tatctggaaactgtcgggtatttccaatgcagtatatactccggctat
tccgctaccaatgatgacgacatccttgtgtatgacctcaacatcaac
cttattactatcctcttccat
SEQ ID no: 46
Description: Clostridium cellulolyticum
L-aspartate oxidase (Ccel_3479)
NCBI Ref: YP_002507747.1
Protein sequence
MEEDSNKVDVEVIHKDVVIIGSGIAGVYTALEIPDSFQIGIITKETLD
ISNSVLAQGGIAVSLDEKNDSPQLHFKDTLFAGAGLNDQKSVWVLVEE
AAENIRILCSLGVNFDKSGQHLSLTREGAHSVNRIIHSGDTTGKEVCD
KLIEVARRKKNISIFESHFAVDLVIEEGKCKGVIVYDEIEDKIKIFKS
GSVVVATGGFGQIYAHTTNPEVATGDGVGMCLRAGAQAMDHEFIQFHP
TVLYHPKDKSFLISEAVRGEGAQLKNSNGEPFMKKYHELGELAPRDIV
SRAIFKEMYLTDSKNVFLDITFKGREYIESRFPNIFKTCLDYGIDISK
DFIPVAPAEHYCMGGVKTDVDGQTNIPGLYACGEVACTGIHGANRLAS
NSLLEGLVFGRKIAKKIESEGRPCNNSAVNSRLCYVSNKENDAALKSM
KEEIQAAMTKYVGIIRSQQGLEKAAQIIKDIYKKYTDFTGFSLVKLEV
LNMLTVAGLVIESALERKESRGAHYRTDYDKTDDTNWRKNIVKELEKG
KMASPF
SEQ ID no: 47
Description: Clostridium cellulolyticum
quinolinate synthase (Ccel_3480)
NCBI Ref: NC_011898.1
(4048820 . . . 4049734)
DNA sequence
ctatttccctactgccagcattctattcaaactaccggatgcacgttc
tataataccgctatccaatgtaatttcgtattgcctcttagctaaggc
atcatgaacactctgtaatgatgttttcttcatattcggacaaatcag
ccctgttgacatcatataaaaagtcttgtttgggttctccttttttaa
ctggtaaagaacacccatctcagttccaataataaatttgtcatgctc
ggaatttcttgcataatctataatctgctttgtgcttcccacaaaatc
agcaagctcctgtatttcgggtcggcactccggatgtaccagcaaaat
agcatcaggatgaagtctctttgactctatgacagcatctttcttaat
cttatgatgtgtaatgcagtagccttcccaaaaaataatgtttttttc
aggaaccttttttgctacataactgccaagatttttatctggagcaaa
tataatatcctttttatcgatagatctgattactttctccgcatttga
agatgtacagcagatatcacactcggccttaacctcagcacttgagtt
tatataacatacaacagctgcgtgaggatactttttcttagcctcttt
cagagcctcagccgtaaccatatctgccattgggcaacctgcatttat
ttcaggcaacagaaccgttttttcaggcgatagaagcttcgcactttc
tgccataaagtgtaccccgcaaaaaactatagtatccgcctgactgga
ggcacaaaattgacttagagctaatgaatctcctgtaacgtcagcaat
ctcctgcacctcatcaacctgataactgtgagcaacaataactgcgtt
ctgctctttcttcatttttttaatgttactaatcaacaaatctttatc
cat
SEQ ID no: 48
Description: Clostridium cellulolyticum
quinolinate synthase (Ccel_3480)
NCBI Ref: YP_002507748.1
Protein sequence
MDKDLLISNIKKMKKEQNAVIVAHSYQVDEVQEIADVTGDSLALSQFC
ASSQADTIVFCGVHFMAESAKLLSPEKTVLLPEINAGCPMADMVTAEA
LKEAKKKYPHAAVVCYINSSAEVKAECDICCTSSNAEKVIRSIDKKDI
IFAPDKNLGSYVAKKVPEKNIIFWEGYCITHHKIKKDAVIESKRLHPD
AILLVHPECRPEIQELADFVGSTKQIIDYARNSEHDKFIIGTEMGVLY
QLKKENPNKTFYMMSTGLICPNMKKTSLQSVHDALAKRQYEITLDSGI
IERASGSLNRMLAVGK
SEQ ID no: 49
Description: Clostridium cellulolyticum
pyridoxal biosynthesis lyase PdxS (Ccel_ 858)
NCBI Ref: NC_011898.1
(2211367 . . . 2212245)
DNA sequence
atgaacgagagatatcaattaaacaaaaatcttgcccaaatgctaaag
ggcggagtaatcatggatgtagtaaatgccaaagaagcagaaattgca
caaaaagccggagccgttgcagtaatggctctcgaaagagttccttcc
gatataagaaaagccggaggagttgcaagaatgtccgatccaaaaatg
ataaaagatatacaaagtgccgtatcaattcctgttatggccaaagtt
agaataggacattttgttgaagcacaggttcttgaagccctttcaatt
gactatattgatgaaagcgaggttttaactccggcagacgaagaattt
cacatagataagcataccttcaaggttccatttgtatgcggtgcaaaa
aatctcggagaagctctcagaagaattagtgaaggtgcatccatgata
agaactaaaggtgaagccggtacaggaaatgttgttgaagccgtccga
catatgagaactgtaacaaatgaaatcagaaaggtgcagagtgcatcc
aagcaggaacttatgaccatagcaaaagaatttggtgctccatatgac
cttattttatatgttcacgaaaacggtaagcttcctgttataaacttt
gcagcaggcggaatcgcaactcccgccgatgcggcattaatgatgcag
cttggatgcgacggcgtatttgttggttcgggaatatttaaatcctca
gatccagccaaaagagcaaaggcaatcgtaaaggcaactacatactat
aatgatccgcaaatcattgcagaggtctctgaagagcttggtactgcc
atggattccatagatgtaagagagttaacaggcaacagtctgtatgcc
tctagaggatggtaa
SEQ ID no: 50
Description: Clostridium cellulolyticum
pyridoxal biosynthesis lyase PdxS (Ccel_1858)
NCBI Ref: YP_002506186.1
Protein sequence
MNERYQLNKNLAQMLKGGVIMDVVNAKEAEIAQKAGAVAVMALERVPS
DIRKAGGVARMSDPKMIKDIQSAVSIPVMAKVRIGHFVEAQVLEALSI
DYIDESEVLTPADEEFHIDKHTFKVPFVCGAKNLGEALRRISEGASMI
RTKGEAGTGNVVEAVRHMRTVTNEIRKVQSASKQELMTIAKEFGAPYD
LILYVHENGKLPVINFAAGGIATPADAALMMQLGCDGVFVGSGIFKSS
DPAKRAKAIVKATTYYNDPQIIAEVSEELGTAMDSIDVRELTGNSLYA
SRGW
SEQ ID no: 51
Description: Clostridium cellulolyticum
glutamine amidotransferase subunit
PdxT (Ccel_1859)
NCBI Ref: NC_011898.1
(2212266 . . . 2212835)
DNA sequence
atgaaaaaaataggtgtgttaggcttgcagggtgctatctcagaacat
ttggataaactatccaaaataccaaatgtagagccattcagcctaaaa
tataaagaagaaattgatacaatagacggacttatcatacccggcggt
gaaagtactgcaatcggcaggcttctctctgattttaacctgacagaa
ccactgaaaacaagggtaaatgccgggatgcctgtatggggaacctgt
gcaggcatgattatccttgcaaaaacgattactaatgaccgccgacgt
catctggaggttatggacataaatgttatgcggaacgggtatggaaga
cagttgaacagctttacaacagaggtttccctggctaaagtttcttct
gataaaatcccgttggtttttattagagcaccttatgtagtcgaggta
gctccgaatgttgaagttcttctgcgtgtagacgaaaacatagtcgcg
tgcaggcaggacaatatgctggccacctcctttcatccggagctgaca
gaagacctgagttttcacaggtactttgcagaaatgatataa
SEQ ID no: 52
Description: Clostridium cellulolyticum
glutamine amidotransferase subunit
PdxT (Ccel_1859)
NCBI Ref: YP_ 002506187.1
Protein sequence
MKKIGVLGLQGAISEHLDKLSKIPNVEPFSLKYKEEIDTIDGLIIPGG
ESTAIGRLLSDFNLTEPLKTRVNAGMPVWGTCAGMIILAKTITNDRRR
HLEVMDINVMRNGYGRQLNSFTTEVSLAKVSSDKIPLVFIRAPYVVEV
APNVEVLLRVDENIVACRQDNMLATSFHPELTEDLSFHRYFAEMI
SEQ ID no: 53
Description: Clostridium cellulolyticum
Dihydrofolate reductase (Ccel_1310)
NCBI Ref: NC_011898.1
(1615000 . . . 1615485)
DNA sequence
atgatttcaatgatatgggctatgggccgcaacaacgcccttggatgt
aaaaacagaatgccctggtacattcccgcagattttgcatatttcaaa
aaagttacaatgggaaaaccggtcattatggggagaaaaacttttgaa
tctatcggtaaacctttaccgggcagaaagaacatagtaattactcga
gacacaggatatgatccacaaggctgtattgtggttaattctatagaa
aaagccatggagtatacagaagaaaaggaagtctttataataggggga
gcagaaatatacaaagaatttcttcctattgcagacagactatatata
actctgatagaaaaagagtttgaagcggatgcatttttcccggaaata
gactatagtaagtggaagcagatatcctgcgaaacaggaatcaaggat
gaaaaaaatccatatgagtataagtggttggtatacgaaagagttaaa
caataa
SEQ ID no: 54
Description: Clostridium cellulolyticum
Dihydrofolate reductase (Ccel_1310)
NCBI Ref: YP_002505644.1
Protein sequence
MISMIWAMGRNNALGCKNRMPWYIPADFAYFKKVTMGKPVIMGRKTFE
SIGKPLPGRKNIVITRDTGYDPQGCIVVNSIEKAMEYTEEKEVFIIGG
AEIYKEFLPIADRLYITLIEKEFEADAFFPEIDYSKWKQISCETGIKD
EKNPYEYKWLVYERVKQ
SEQ ID no: 55
Description: Haematobia irritans
Transposase (Himar1)
GenBank: DQ236098.1
(365 . . . 1411)
DNA sequence
ttattcaacatagttcccttcaagagcgatacaacgattataacgacc
ttccaattttttgataccattttggtagtactccttcggttttgcctc
aaaataggcctcagtttcggcgatcacctcttcattgcagccaaattt
tttccctgcgagcatccttttgaggtctgagaacaagaaaaagtcgct
gggggccagatctggagaatacggtgggtggggaagcaattcgaagcc
caattcatgaatttttgccatcgttctcaatgacttgtggcacggtgc
gttgtcttggtggaacaacacttttttcttcttcatgtggggccgttt
tgccgcgatttcgaccttcaaacgctccaataacgccatataatagtc
actgttgatggtttttcccttctcaagataatcgataaaaattattcc
atgcgcatcccaaaaaacagaggccattactttgccagcggacttttg
agtctttccacgcttcggagacggttcaccggtcgctgtccactcagc
cgactgtcgattggactcaggagtgtagtgatggagccatgtttcatc
cattgtcacatatcgacggaaaaactcgggtgtattacgagttaacag
ctgcaaacaccgctcagaatcatcaacacgttgttgtttttggtcaaa
tgtgagctcgcgcggcacccattttgcacagagcttccgcatatccaa
atattgatgaatgatatgaccaacacgttcctttgatatctttaaggc
ctctgctatctcgatcaacttcattttacggtcattcaaaatcatttt
gtggatttttttgatgttttcgtcggtaaccacctctttcgggcgtcc
actgcgttcaccgtcctccgtgctcatttcaccacgcttgaattttgc
ataccaatcaattattgttgatttccctggggcagagtccggaaactc
attatcaagccaagtttttgcttccaccgtattttttcccttcagaaa
acagtattttatcaaaacacgaaattcctttttttccat
SEQ ID no: 56
Description: Haematobia irritans
Transposase (Himar1)
GenBank: ABB59013.1
MEKKEFRVLIKYCFLKGKNTVEAKTWLDNEFPDSAPGKSTIIDWYAKF
KRGEMSTEDGERSGRPKEVVTDENIKKIHKMILNDRKMKLIEIAEALK
ISKERVGHIIHQYLDMRKLCAKWVPRELTFDQKQQRVDDSERCLQLLT
RNTPEFFRRYVTMDETWLHHYTPESNRQSAEWTATGEPSPKRGKTQKS
AGKVMASVFWDAHGIIFIDYLEKGKTINSDYYMALLERLKVEIAAKRP
HMKKKKVLFHQDNAPCHKSLRTMAKIHELGFELLPHPPYSPDLAPSDF
FLFSDLKRMLAGKKFGCNEEVIAETEAYFEAKPKEYYQNGIKKLEGRY
NRCIALEGNYVE
Protein sequence
SEQ ID no: 57
Description: Escherichia coli
toxin, RNase (mazF)
GenBank: AERR01000023.1
(132931 . . . 133266)
DNA sequence
ctacccaatcagtacgttaattttggctttaatgagttgtaattcctc
tggggcaaccgttcctttcttcgttgctcctcttgcccgccaggcgat
actttttacctgatcagctaacgctacgccatcacgttcctgaccgga
taaaacaacttcgaacggatatccttttgattgcgttgtacaaggaac
acacagacacatacctgttttgttgttgtacatgaacggactcaggac
aacagccggacgatgtccggcttgctcgctaccttttgtcgggtcaaa
atcaacccaaatcagatcgcccatatcgggtacgtatcggcttaccat
SEQ ID no: 58
Description: Escherichia coli
toxin, RNase (mazF)
GenBank: EGD66739.1
Protein sequence
MVSRYVPDMGDLIWVDFDPTKGSEQAGHRPAVVLSPFMYNNKTGMCLC
VPCTTQSKGYPFEVVLSGQERDGVALADQVKSIAWRARGATKKGTVAP
EELQLIKAKINVLIG
SEQ ID no: 59
Description: Escherichia coli
antitoxin to mazF (mazE)
GenBank: AERR01000023.1
(133266 . . . 133514)
DNA sequence
ttaccagacttccttatctttcggctctccccagtcgatattctcgtg
gaggttttccggcgtgatgtcgttgaccagttcagcaagcgtaaatac
gggctctttacgcactggctcaataattaatttgccatccaccaggtc
aatcttcacttcatcatcaatattcagattgagcgcctgcattaacgt
agccgggatccgcaccgccggtgaatttccccaacgctttacgctact
gtggatcat
SEQ ID no: 60
Description: Escherichia coli
antitoxin to mazF (mazE)
GenBank: EGD66740.1
DNA/Protein sequence
MIHSSVKRWGNSPAVRIPATLMQALNLNIDDEVKIDLVDGKLIIEPVR
KEPVFTLAELVNDITPENLHENIDWGEPKDKEVW

In another embodiment, more effective biomass fermentation pathways can be created by transforming host cells with multiple copies of enzymes of a pathway and then combining the cells producing the individual enzymes. This approach allows for the combination of enzymes to more particularly match the biomass of interest by altering the relative ratios of the multiple-transformed strains. In one embodiment two times as many cells expressing the first enzyme of a pathway can be added to a mix where the first step of the reaction pathway is a limiting step of the overall reaction pathway.

In another embodiment, a biofuel plant or process disclosed herein is useful for producing biofuel with a microorganism engineered to knockout or reduce naturally-occurring lactate dehydrogenase (LDH knockout). An LDH knockout is useful for increasing yields of ethanol or other biofuels, or other chemical products from the hydrolysis of biomass in comparison to other mesophilic fermenting microorganisms. In one embodiment, a mesophilic LDH knockout can be used for reducing the amount of lactic acid in the yield of ethanol or other biofuels or fermentive end products.

In one embodiment, an LDH knockout construct can be expressed in a microorganism that does not express pyruvate carboxylase. In another embodiment, an LDH knockout construct can be expressed in a microorganism that does not produce ethanol as a primary product of its metabolic process. A microorganism that does not produce ethanol as a primary product can be a naturally occurring, or a genetically modified microorganism. For example, in a microorganism producing ethanol, lactic acid and acetic acid, the microorganism can be engineered to produce undetectable amount of lactic acid and acetic acid. The microorganism can further be engineered to express an acetic acid knockout and/or a formic acid knockout.

Methods and compositions described herein are useful for obtaining increased fermentive yields. In one embodiment, increased fermentive yield activity is obtained by transforming a microorganism with an LDH knockout construct. In another embodiment, the microorganism is selected from the group of Clostridia. In another embodiment, the microorganism is a strain selected from C. phytofermentans.

In another embodiment, a microorganism comprises a heterologous alcohol dehydrogenase gene and a pyruvate decarboxylase gene. In one embodiment, the pyruvated decarboxylase gene can be endogenous or heterologous. In a further embodiment, the expression of the heterologous genes results in the production of enzymes which redirect the metabolism to yield ethanol as a primary fermentation product. The heterologous genes may be obtained from microorganisms that typically undergo anaerobic fermentation, including Zymomonas species, including Zymomonas mobilis.

In another embodiment, the wild-type microorganism is mesophilic or thermophilic. In one embodiment, the microorganism is a Clostridium species. In another embodiment, the Clostridium species is C. phytofermentans, Clostridium sp. Q.D, Clostridium phytofermentans Q.8, Clostridium phytofermentans Q.12, Clostridium phytofermentans Q.13, or genetically-modified cells thereof. In a further embodiment, the microorganism is cellulolytic. In a further embodiment, the microorganism is xylanolytic. In some embodiments, the microorganism is gram negative or gram positive. In some embodiments, the microorganism is anaerobic.

Microorganisms selected for modification are said to be “wild-type” and are useful in the fermentation of carbonaceous biomass. In one example, the microorganisms can be mutants or strains of Clostridium sp. and are mesophilic, anaerobic, and C5/C6 saccharifying microorganisms. The microorganisms can be isolated from environmental samples expected to contain mesophiles. Isolated wild-type microorganisms will have the ability to produce ethanol but, unmodified, lactate is likely to be a fermentation product. The isolates are also selected for their ability to grow on hexose and/or pentose sugars, and oligomers thereof, at mesophilic (10° C. to 40° C.) temperatures.

In most instances, the microorganism described herein has characteristics that permit it to be used in a fermentation process. In addition, the microorganism should be stable to at least 6% ethanol and should have the ability to utilize C3, C5 and C6 sugars (or their oligomers) as a substrate, including cellobiose and starch. In one embodiment, the microorganism can saccharify C5 and C6 polysaccharides as well as ferment oligomers of these polysaccharides and monosaccharides. In one embodiment, the microorganism produces ethanol in a yield of at least 50 g/l over a 5-8 day fermentation.

In one embodiment, the microorganism is a spore-former. In another embodiment, the microorganism does not sporulate. The success of the fermentation process does not depend necessarily on the ability of the microorganism to sporulate, although in certain circumstances it may be preferable to have a sporulator, e.g. when it is desirable to use the microorganism as an animal feed-stock at the end of the fermentation process. This is due to the ability of sporulators to provide a good immune stimulation when used as an animal feed-stock. Spore-forming microorganisms also have the ability to settle out during fermentation, and therefore can be isolated without the need for centrifugation. Accordingly, the microorganisms can be used in an animal feed-stock without the need for complicated or expensive separation procedures.

In one embodiment, production of a fermentation end-product comprises: a carbonaceous biomass, a microorganism that is capable of direct hydrolysis and fermentation of the biomass to a fermentation end-product disclosed herein.

In another embodiment, a product for production of a biofuel comprises: a carbonaceous biomass, a microorganism that is capable of hydrolysis and fermentation of the biomass, wherein the microorganism is modified to provide enhanced production of a fermentation end-product disclosed herein.

In yet a further embodiment, a product for production of fermentation end-products comprises: (a) a fermentation vessel comprising a carbonaceous biomass; (b) and a modified microorganism that is capable of hydrolysis and fermentation of the biomass; wherein the fermentation vessel is adapted to provide suitable conditions for fermentation of one or more carbohydrates into fermentation end-products.

In one embodiment a microorganism utilized in products or processes described herein can be one that is capable of hydrolysis and fermentation of C5 and C6 carbohydrates (such as lignocellulose or hemicelluloses). In one embodiment, such a capability is achieved through modifying the microorganism to express one or more genes encoding proteins associated with C5 and C6 carbohydrate metabolism.

Microorganisms useful in compositions and methods of these embodiments include but are not limited to bacteria, yeast or fungi that can hydrolyze and ferment feedstock or biomass. In some embodiments, two or more different microorganisms can be utilized during saccharification and/or fermentation processes to produce an end-product. Microorganisms utilized in methods and compositions described herein can be recombinant.

In one embodiment, a microorganism utilized in compositions or methods described herein is a strain of Clostridia. In a further embodiment, the microorganism is Clostridium phytofermentans, C. sp. Q.D, or genetically modified variant thereof.

Organisms described herein can be modified to comprise one or more heterologous or exogenous polynucleotides that enhance enzyme function. In one embodiment, enzymatic function is increased for one or more cellulase enzymes.

A microorganism used in products and processes described herein can be capable of uptake of one or more complex carbohydrates from biomass (e.g., biomass comprises a higher concentration of oligomeric carbohydrates relative to monomeric carbohydrates).

In some embodiments, one or more enzymes are utilized in products and processes in these embodiments, which are added externally (e.g., enzymes provided in purified form, cell extracts, culture medium or commercially available source).

Enzyme activity can also be enhanced by modifying conditions in a reaction vessel, including but not limited to time, pH of a culture medium, temperature, concentration of nutrients and/or catalyst, or a combination thereof. A reaction vessel can also be configured to separate one or more desired end-products.

Products or processes described in these embodiments provide for hydrolysis of biomass resulting in a greater concentration of cellobiose relative to monomeric carbohydrates. Such monomeric carbohydrates can comprise xylose and arabinose.

In some embodiments, batch fermentation with a microorganism described herein and of a mixture of hexose and pentose saccharides using methods and processes disclosed herein provides uptake rates of about 0.1, 0.2, 0.4, 0.5, 0.6, 0.7, 0.8, 1, 2, 3, 4, 5, or about 6 g/L/h or more of hexose (e.g. glucose, cellulose, cellobiose etc.), and about 0.1, 0.2, 0.4, 0.5, 0.6 0.7, 0.8, 1, 2, 3, 4, 5, or about 6 g/L/h or more of pentose (xylose, xylan, hemicellulose etc.). For example, C. phytofermentans, Clostridium sp. Q.D. or variants thereof are capable of hydrolysis and fermentation of C5 and C6 sugars.

### Biofuel Plant and Process of Producing Biofuel

In one aspect, provided herein is a fuel plant that includes a hydrolysis unit configured to hydrolyze a biomass material comprising a high molecular weight carbohydrate, and a fermentor configured to house a medium and one or more species of microorganisms. In one embodiment the microorganism is Clostridium phytofermentans. In another embodiment, the microorganism is Clostridium sp. Q.D.

In another embodiment, the microorganism is Clostridium phytofermentans Q.12. In another embodiment, the microorganism is Clostridium phytofermentans Q.12. In another embodiment, the microorganism is Clostridium phytofermentans Q.13.

In another aspect, provided herein are methods of making a fuel or chemical end-product that includes combining a microorganism (such as Clostridium phytofermentans, Clostridium sp. Q.D, Clostridium phytofermentans Q.12, Clostridium phytofermentans Q.13 or a similar species of Clostridium that hydrolyzes and ferments C5/C6 carbohydrates) and a lignocellulosic material (and/or other biomass material) in a medium, and fermenting the lignocellulosic material under conditions and for a time sufficient to produce a fermentation end-product, (e.g., ethanol, propanol, methane, or hydrogen).

In some embodiments, a process is provided for producing a fermentation end-product from biomass using acid hydrolysis pretreatment. In some embodiments, a process is provided for producing a fermentation end-product from biomass using enzymatic hydrolysis pretreatment. In another embodiment a process is provided for producing a fermentation end-product from biomass using biomass that has not been enzymatically pretreated. In another embodiment a process is provided for producing a fermentation end-product from biomass using biomass that has not been chemically or enzymatically pretreated, but is optionally steam treated.

In another aspect, provided herein are end-products made by any of the processes described herein. Those skilled in the art will appreciate that a number of genetic modifications can be made to the methods exemplified herein. For example, a variety of promoters can be utilized to drive expression of the heterologous genes in a recombinant microorganism (such as Clostridium phytofermentans, Clostridium sp. Q.D, Clostridium phytofermentans Q.12 or Clostridium phytofermentans Q.13). The skilled artisan, having the benefit of the instant disclosure, will be able to readily choose and utilize any one of the various promoters available for this purpose. Similarly, skilled artisans, as a matter of routine preference, can utilize a higher copy number plasmid. In another embodiment, constructs can be prepared for chromosomal integration of the desired genes. Chromosomal integration of foreign genes can offer several advantages over plasmid-based constructions, the latter having certain limitations for commercial processes. Ethanologenic genes have been integrated chromosomally in E. coli B; see Ohta et al. (1991) Appl. Environ. Microbiol. 57:893-900. In general, this is accomplished by purification of a DNA fragment containing (1) the desired genes upstream from an antibiotic resistance gene and (2) a fragment of homologous DNA from the target microorganism. This DNA can be ligated to form circles without replicons and used for transformation. Thus, the gene of interest can be introduced in a heterologous host such as E. coli, and short, random fragments can be isolated and ligated in Clostridium phytofermentans, Clostridium sp. Q.D. Clostridium phytofermentans Q.8, Clostridium phytofermentans Q.12, Clostridium phytofermentans Q.13, or variants thereof, to promote homologous recombination.

Large Scale Fermentation End-Product Production from Biomass

In one aspect a fermentation end-product (e.g., ethanol) from biomass is produced on a large scale utilizing a microorganism, such as C. phytofermentans, Clostridium sp. Q.D, Clostridium phytofermentans Q.8, Clostridium phytofermentans Q.12, Clostridium phytofermentans Q.13 or variants thereof. In one embodiment, a biomass that includes high molecular weight carbohydrates is hydrolyzed to lower molecular weight carbohydrates, which are then fermented using a microorganism to produce ethanol. In another embodiment, the biomass is fermented without chemical and/or enzymatic pretreatment. In one embodiment, hydrolysis can be accomplished using acids, e.g., Bronsted acids (e.g., sulfuric or hydrochloric acid), bases, e.g., sodium hydroxide, hydrothermal processes, steam explosion, ammonia fiber explosion processes (“AFEX”), lime processes, enzymes, or combination of these. Hydrogen, and other products of the fermentation can be captured and purified if desired, or disposed of, e.g., by burning. For example, the hydrogen gas can be flared, or used as an energy source in the process, e.g., to drive a steam boiler, e.g., by burning. Hydrolysis and/or steam treatment of the biomass can increase porosity and/or surface area of the biomass, often leaving the cellulosic materials more exposed to the microorganismal cells, which can increase fermentation rate and yield. In another embodiment removal of lignin can provide a combustible fuel for driving a boiler, and can also increase porosity and/or surface area of the biomass, often increasing fermentation rate and yield. In some embodiments, the initial concentration of the carbohydrates in the medium is greater than 20 mM, e.g., greater than 30 mM, 50 mM, 75 mM, 100 mM, 150 mM, 200 mM, or even greater than 500 mM.

In one aspect, these embodiments feature a fuel plant that comprises a hydrolysis unit configured to hydrolyze a biomass material that includes a high molecular weight carbohydrate; a fermentor configured to house a medium with a C5/C6 hydrolyzing and fermenting microorganism (e.g., Clostridium phytofermentans, Clostridium sp. Q.D, Clostridium phytofermentans Q.8, Clostridium phytofermentans Q.12, Clostridium phytofermentans Q.13, or variants thereof); and one or more product recovery system(s) to isolate a fermentation end-product or end-products and associated by-products and co-products.

In another aspect, these embodiments feature methods of making a fermentation end-product or end-products that include combining a C5/C6 hydrolyzing and fermenting microorganism (e.g., Clostridium phytofermentans, Clostridium sp. Q.D, Clostridium phytofermentans Q.8, Clostridium phytofermentans Q.12, Clostridium phytofermentans Q.13, or variants thereof) and a carbonaceous biomass in a medium, and fermenting the biomass material under conditions and for a time sufficient to produce a fermentation end-products (e.g. ethanol, propanol, hydrogen, lignin, terpenoids, and the like). In one embodiment the fermentation end-product is a biofuel or chemical product.

In another aspect, these embodiments feature one or more fermentation end-products made by any of the processes described herein. In one embodiment one or more fermentation end-products can be produced from biomass on a large scale utilizing a C5/C6 hydrolyzing and fermenting microorganism (e.g., Clostridium phytofermentans, Clostridium sp. Q.D, Clostridium phytofermentans Q.8, Clostridium phytofermentans Q.12, Clostridium phytofermentans Q.13, or variants thereof). In one embodiment depending on the type of biomass and its physical manifestation, the process can comprise a milling of the carbonaceous material, via wet or dry milling, to reduce the material in size and increase the surface to volume ratio (physical modification).

In some embodiments, the treatment includes treatment of a biomass with acid. In some embodiments, the acid is dilute. In some embodiments, the acid treatment is carried out at elevated temperatures of between about 85 and 140° C. In some embodiments, the method further comprises the recovery of the acid treated biomass solids, for example by use of a sieve. In some embodiments, the sieve comprises openings of approximately 150-250 microns in diameter. In some embodiments, the method further comprises washing the acid treated biomass with water or other solvents. In some embodiments, the method further comprises neutralizing the acid with alkali. In some embodiments, the method further comprises drying the acid treated biomass. In some embodiments, the drying step is carried out at elevated temperatures between about 15-45° C. In some embodiments, the liquid portion of the separated material is further treated to remove toxic materials. In some embodiments, the liquid portion is separated from the solid and then fermented separately. In some embodiments, a slurry of solids and liquids are formed from acid treatment and then fermented together.

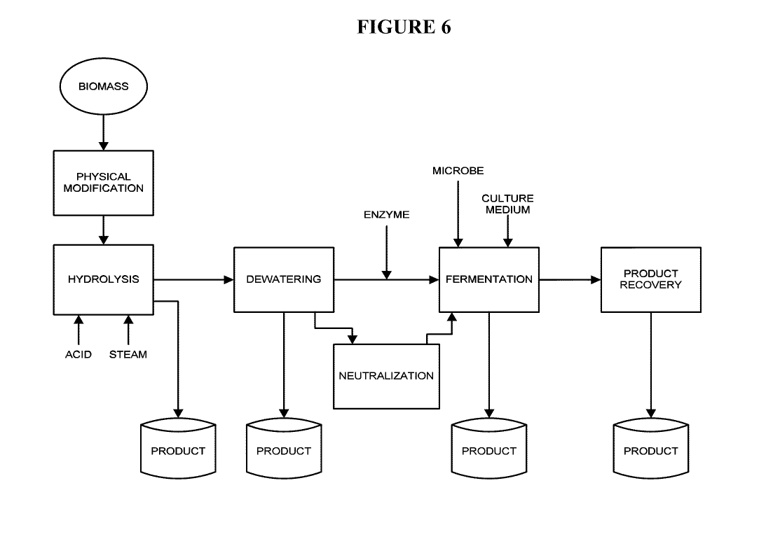

FIG. 6 illustrates an example of a method for producing a fermentation end-product from biomass by first treating biomass with an acid at elevated temperature and pressure in a hydrolysis unit. The biomass can first be heated by addition of hot water or steam. The biomass can be acidified by bubbling gaseous sulfur dioxide through the biomass that is suspended in water, or by adding a strong acid, e.g., sulfuric, hydrochloric, or nitric acid with or without preheating/presteaming/water addition. During the acidification, the pH is maintained at a low level, e.g., below about 5. The temperature and pressure can be elevated after acid addition. In addition to the acid already in the acidification unit, optionally, a metal salt such as ferrous sulfate, ferric sulfate, ferric chloride, aluminum sulfate, aluminum chloride, magnesium sulfate, or mixtures of these can be added to aid in the hydrolysis of the biomass. The acid-impregnated biomass is fed into the hydrolysis section of the pretreatment unit. Steam is injected into the hydrolysis portion of the pretreatment unit to directly contact and heat the biomass to the desired temperature. The temperature of the biomass after steam addition is, e.g., between about 130° C. and 220° C. The hydrolysate is then discharged into the flash tank portion of the pretreatment unit, and is held in the tank for a period of time to further hydrolyze the biomass, e.g., into oligosaccharides and monomeric sugars. Steam explosion can also be used to further break down biomass. Alternatively, the biomass can be subject to discharge through a pressure lock for any high-pressure pretreatment process. Hydrolysate is then discharged from the pretreatment reactor, with or without the addition of water, e.g., at solids concentrations between about 15% and 60%.

In some embodiments, after pretreatment, the biomass can be dewatered and/or washed with a quantity of water, e.g. by squeezing or by centrifugation, or by filtration using, e.g. a countercurrent extractor, wash press, filter press, pressure filter, a screw conveyor extractor, or a vacuum belt extractor to remove acidified fluid. The acidified fluid, with or without further treatment, e.g. addition of alkali (e.g. lime) and or ammonia (e.g. ammonium phosphate), can be re-used, e.g., in the acidification portion of the pretreatment unit, or added to the fermentation, or collected for other use/treatment. Products can be derived from treatment of the acidified fluid, e.g., gypsum or ammonium phosphate. Enzymes or a mixture of enzymes can be added during pretreatment to assist, e.g. endoglucanases, exoglucanases, cellobiohydrolases (CBH), beta-glucosidases, glycoside hydrolases, glycosyltransferases, lyases, and esterases active against components of cellulose, hemicelluloses, pectin, and starch, in the hydrolysis of high molecular weight components.

In one embodiment the fermentor is fed with hydrolyzed biomass; any liquid fraction from biomass pretreatment; an active seed culture of Clostridium phytofermentans, Clostridium sp. Q.D, Clostridium phytofermentans Q.8, Clostridium phytofermentans Q.12, Clostridium phytofermentans Q.13, a mutagenized or genetically-modified variant thereof, optionally a co-fermenting microorganism (e.g., yeast or E. coli) and, as needed, nutrients to promote growth of the Clostridium cells or other microorganisms. In another embodiment the pretreated biomass or liquid fraction can be split into multiple fermentors, each containing a different strain of Clostridium phytofermentans, Clostridium sp. Q.D, Clostridium phytofermentans Q.12. Clostridium phytofermentans Q.13, a mutagenized or genetically-modified variant thereof and/or other microorganisms; with each fermentor operating under specific physical conditions. Fermentation is allowed to proceed for a period of time, e.g., between about 15 and 150 hours, while maintaining a temperature of, e.g., between about 25° C. and 50° C. Gas produced during the fermentation is swept from fermentor and is discharged, collected, or flared with or without additional processing, e.g. hydrogen gas can be collected and used as a power source or purified as a co-product.

After fermentation, the contents of the fermentor are transferred to product recovery. Products are extracted, e.g., ethanol is recovered through distillation and rectification. Methods and compositions described herein can include extracting or separating fermentation end-products, such as ethanol, from biomass. Depending on the product formed, different methods and processes of recovery can be provided.

In one embodiment, a method for extraction of lactic acid from a fermentation broth uses freezing and thawing of the broth followed by centrifugation, filtration, and evaporation. (Omar, et al. 2009 African J. Biotech. 8:5807-5813) Other methods that can be utilized are membrane filtration, resin adsorption, and crystallization. (See, e.g., Huh, et al. 2006 Process Biochemistry).

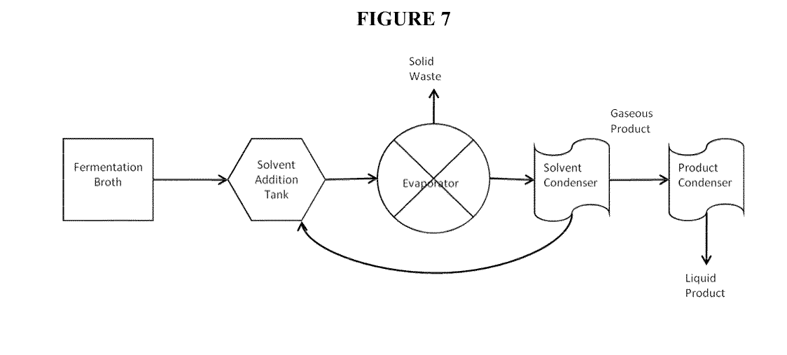

In another embodiment for solvent extraction of a variety of organic acids (such as ethyl lactate, ethyl acetate, formic, butyric, lactic, acetic, succinic), the process can take advantage of preferential partitioning of the product into one phase or the other. In some cases the product might be carried in the aqueous phase rather than the solvent phase. In other embodiments, the pH is manipulated to produce more or less acid from the salt synthesized from the microorganism. The acid phase is then extracted by vaporization, distillation, or other methods. (See FIG. 7).

In yet a further embodiment, a system for production of fermentation end-products comprises: (a) a fermentation vessel comprising a carbonaceous biomass; (b) and a microorganism that is capable of hydrolysis and fermentation of the biomass; wherein the fermentation vessel is adapted to provide suitable conditions for fermentation of one or more carbohydrates into fermentation end-products. In one embodiment the microorganism is genetically modified. In another embodiment the microorganism is not genetically modified.

Chemical Production from Biomass

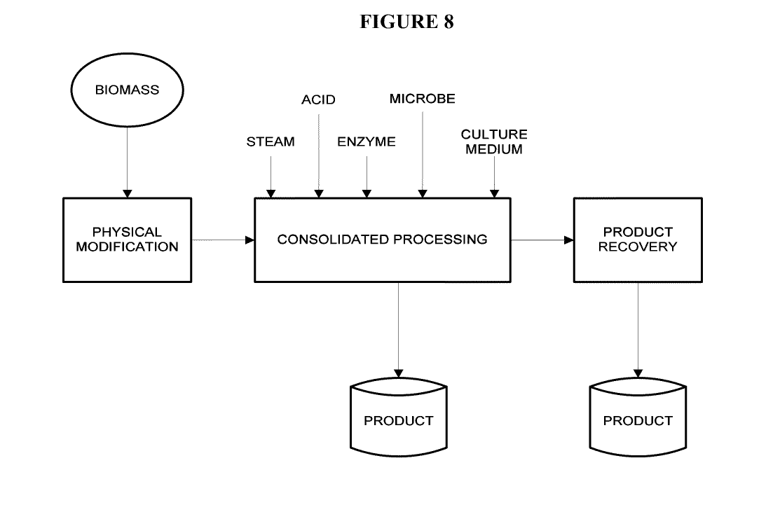

FIG. 8 depicts a method for producing chemicals from biomass by charging biomass to a fermentation vessel. The biomass can be allowed to soak for a period of time, with or without addition of heat, water, enzymes, or acid/alkali. The pressure in the processing vessel can be maintained at or above atmospheric pressure. Acid or alkali can be added at the end of the pretreatment period for neutralization. At the end of the pretreatment period, or at the same time as pretreatment begins, an active seed culture of a C5/C6 hydrolyzing and fermenting microorganism (e.g., Clostridium phytofermentans, Clostridium sp. Q.D, Clostridium phytofermentans Q.8, Clostridium phytofermentans Q.12, Clostridium phytofermentans Q.13 or variant thereof) and, if desired, a co-fermenting microorganism, e.g., yeast or E. coli, and, if required, nutrients to promote growth of a C5/C6 hydrolyzing and fermenting microorganism (e.g., Clostridium phytofermentans, Clostridium sp. Q.D, Clostridium phytofermentans Q.8, Clostridium phytofermentans Q.12, Clostridium phytofermentans Q.13, or mutagenized or genetically-modified cells thereof are added. Fermentation is allowed to proceed as described above. After fermentation, the contents of the fermentor are transferred to product recovery as described above. Any combination of the chemical production methods and/or features can be utilized to make a hybrid production method. In any of the methods described herein, products can be removed, added, or combined at any step. A C5/C6 hydrolyzing and fermenting microorganism (e.g., Clostridium phytofermentans, Clostridium sp. Q.D, Clostridium phytofermentans Q.8, Clostridium phytofermentans Q.12, or Clostridium phytofermentans Q.13) can be used alone or synergistically in combination with one or more other microorganisms (e.g. yeasts, fungi, or other bacteria). In some embodiments different methods can be used within a single plant to produce different end-products.

In another aspect, these embodiments feature a fuel plant that includes a hydrolysis unit configured to hydrolyze a biomass material that includes a high molecular weight carbohydrate, a fermentor configured to house a medium and contains a C5/C6 hydrolyzing and fermenting microorganism (e.g., Clostridium phytofermentans, Clostridium sp. Q.D, Clostridium phylofermentans Q.8, Clostridium phytofermentans Q.12, Clostridium phytofermentans Q.13, or mutagenized or genetically-modified cells thereof).

In another aspect, the invention features a chemical production plant that includes a hydrolysis unit configured to hydrolyze a biomass material that includes a high molecular weight carbohydrate, a fermentor configured to house a medium and contains a C5/C6 hydrolyzing and fermenting microorganism (e.g., Clostridium phytofermentans, Clostridium sp. Q.D, Clostridium phytofermentans Q.8, Clostridium phytofermentans Q.12, Clostridium phytofermentans Q.13, or mutagenized or genetically-modified cells thereof).

In another aspect, these embodiments feature methods of making a chemical(s) or fuel(s) that include combining a C5/C6 hydrolyzing and fermenting microorganism (e.g., Clostridium phytofermentans, Clostridium sp. Q.D, Clostridium phytofermentans Q.8, Clostridium phytofermentans Q.12, Clostridium phytofermentans Q.13, or mutagenized or genetically-modified cells thereof), and a lignocellulosic material (and/or other biomass material) in a medium, and fermenting the lignocellulosic material under conditions and for a time sufficient to produce a chemical(s) or fuel(s), e.g., ethanol, propanol and/or hydrogen or another chemical compound.

In some embodiments, a process is provided for producing ethanol and hydrogen from biomass using acid hydrolysis pretreatment. In some embodiments, a process is provided for producing ethanol and hydrogen from biomass using enzymatic hydrolysis pretreatment. Other embodiments provide a process for producing ethanol and hydrogen from biomass using biomass that has not been enzymatically pretreated. Still other embodiments disclose a process for producing ethanol and hydrogen from biomass using biomass that has not been chemically or enzymatically pretreated, but is optionally steam treated.

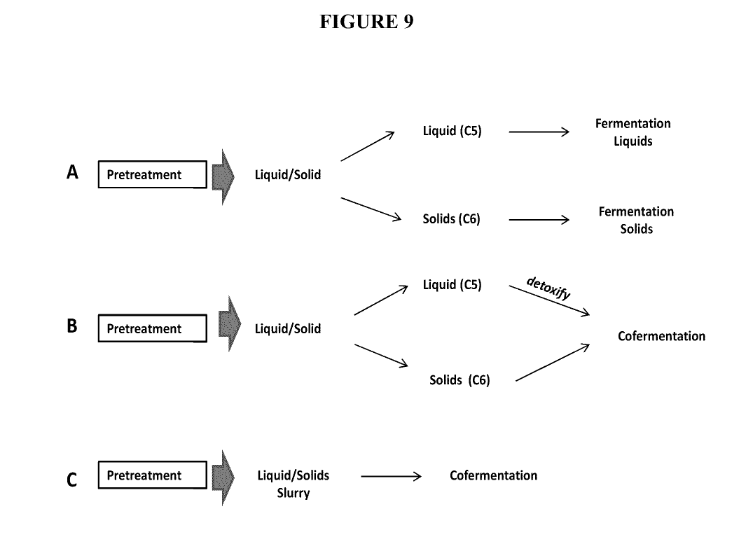

FIG. 9 discloses pretreatments that produce hexose or pentose saccharides or oligomers that are then unprocessed or processed further and either, fermented separately or together. FIG. 9A depicts a process (e.g., acid pretreatment) that produces a solids phase and a liquid phase which are then fermented separately. FIG. 9B depicts a similar pretreatment that produces a solids phase and liquids phase. The liquids phase is separated from the solids and elements that are toxic to the fermenting microorganism are removed prior to fermentation. At initiation of fermentation, the two phases are recombined and cofermented together. This is a more cost-effective process than fermenting the phases separately. The third process (FIG. 9C) is the least costly. The pretreatment results in a slurry of liquids or solids that are then cofermented. There is little loss of saccharides component and minimal equipment required.

### EXAMPLES

### Recombinant Bioenergetic Pathways

Glycolysis is the metabolic pathway that converts glucose, C₆H₁₂O₆, into pyruvate, CH₃COCOO⁻+H⁺. The free energy released in this process is used to form the high energy compounds, ATP (adenosine triphosphate) and NADH (reduced nicotinamide adenine dinucleotide). Glucose enters the glycolysis pathway by conversion to glucose-6-phosphate. Early in this pathway, the hexose, fructose-6-bisphosphate, is split into two triose sugars, dihydroxyacetone phosphate, a ketone, and glyceraldehyde 3-phosphate, an aldehyde, thus two molecules of pyruvate are generated for each glucose molecule that is metabolized.

Anaerobic organisms lack a respiratory chain. They must reoxidize NADH produced in glycolysis through some other reaction, because NAD is needed for the glyceraldehydes-3-phosphate dehydrogenases reaction (FIG. 2). Usually NADH is reoxidized as pyruvate is converted to a more reduced compound. For example, lactate dehydrogenase catalyzes the reduction of the keto group in pyruvate to a hydroxyl, yielding lactate, as NADH is oxidized to NAD⁺. In C. phytofermentans or Q.D, very little lactate dehydrogenase is synthesized however. These cellulolytic species metabolize pyruvate to ethanol as a primary product, which is excreted as a waste product. NADH is converted to NAD in the reaction catalyzed by alcohol dehydrogenase. In Clostridium sp Q.D., the organism also converts an intermediate, acetyl-CoA, to acetic acid as an end product.

### Example 1

### Increase in Ethanol Tolerance

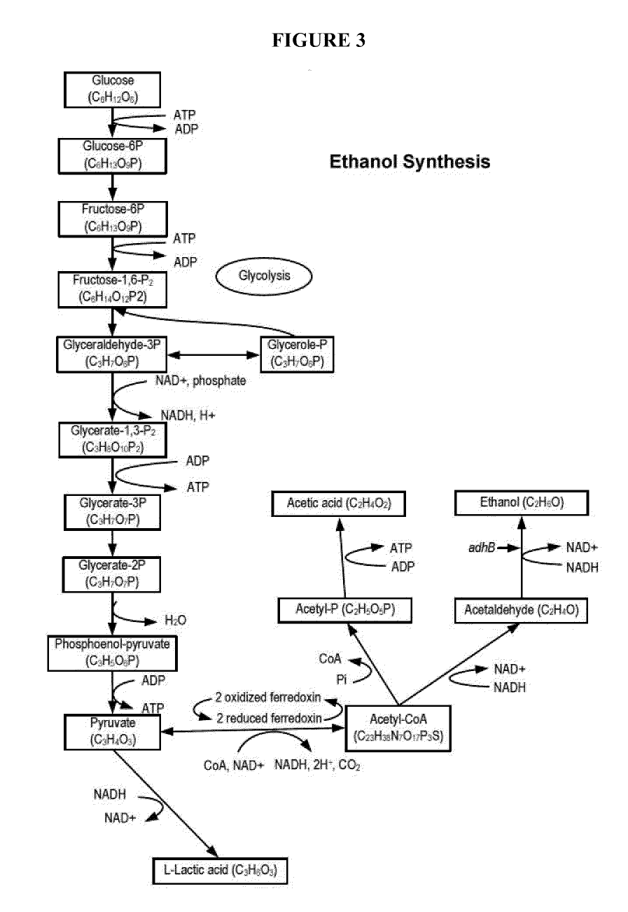

In addition to the endogenous alcohol dehydrogenases that reduces acetaldehyde to ethanol in C. phytofermentans and Q.D, a heterologous alcohol dehydrogenase that does not exhibit end-product inhibition at ethanol concentrations below 60 g/L can be expressed to function in these organisms. In one embodiment, an example of such and alcohol dehydrogenase (ADH) is adhB, from Zymomonas mobilis (FIG. 3). This would prevent the eventual accumulation and toxic effects of acetaldehyde observed at ethanol concentrations greater than 35 g/L and allow ethanol titers to increase beyond the current limit in C. phytofermentans or Clostridium sp Q.D. A potential corollary effect would be an extended growth phase due to reduce toxicity of fermentation intermediates (e.g. acetaldehyde). Introduction and expression of adhB from Z. mobilis can be in conjunction with the expression of C. phytofermentans or Q.D's native ADH's or by replacement of one or more by gene knockout.

### Example 2

### Increase in Ethanol Production Through High Glycolytic Flux

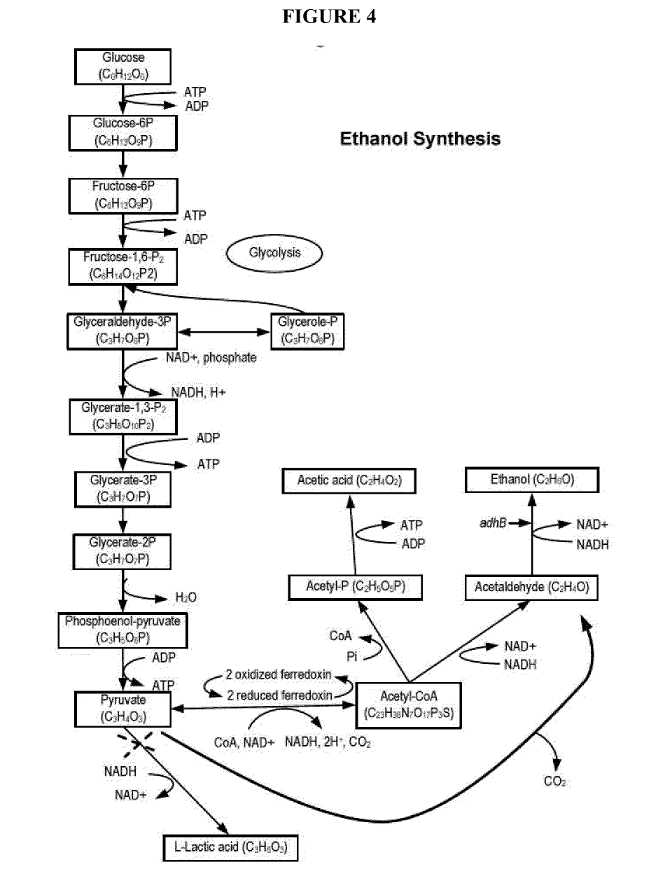

Introduction of a pyruvate decarboxylase (either in conjunction with an alcohol dehydrogenase that doesn't exhibit end product inhibition, or alone with C. phytofermentans or Q.D's own alcohol dehydrogenases), would allow a direct conversion of pyruvate to acetaldehyde (then directly to ethanol from ADH) without the requirement to make Acetyl CoA (FIG. 4). This can facilitate ethanol production through high glycolytic flux (i.e. where redox balance requirements results in a shift of carbon flux from pyruvate to organic acid (e.g. Lactic acid) instead of pyruvate to Acetyl CoA as is usual in C. phytofermentans or Q.D) resulting quicker fermentation rates with high sugar concentrations. Introduction of pyruvate decarboxylase can facilitate the production of ethanol without the requirement for cell division or anabolism by bypassing the acetyl CoA step. This would alleviate the need for a rich growth supporting medium, and allow for growth to an acceptable density then keep the ethanol production rate per unit dry cell weight high. The pyruvate decarboxylase (pdc) gene (e.g. Saccharomyces, Zymomonas) can be added to complement the pyruvate synthase (pyruvate to Acetyl CoA) to facilitate acceptable cell density and then “turned on” by a regulatory element at the right stage of growth. Pyruvate decarboxylase can be used to replace one the several LDH's in C. phytofermentans. or Q.D, or the activity of two or more LDH's can be disrupted along with pyruvate decarboxylase introduction, or pyruvate decarboxylase can be added in addition to C. phytofermentans or Q.D's own pathway.

### Example 3

### Expression of Acetyl CoA Synthetase

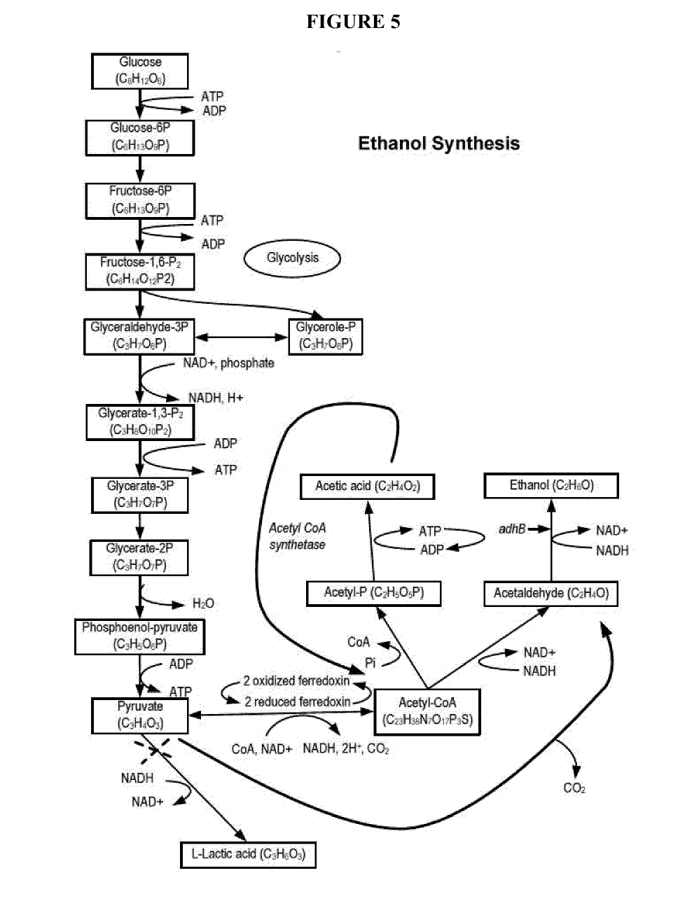

To prevent the buildup of acetic acid and to maintain a high pool of acetyl-CoA (required for fatty acid synthesis), expression of acetyl-CoA synthetase would keep the yield of ethanol high, especially in Q.D (FIG. 5). Another advantage of recycling acetic acid is that the pH of the fermentation media would not drop as fast. Because the conversion of acetic acid to acetyl-CoA requires ATP, it is an energy-neutral step.

### Example 4

### Disruption of LDH gene

Because C. phytofermentans and Clostridium sp. Q.D generate very small amounts of lactic acid (lactate), disruption of any their endogenous lactate dehydrogenase genes will increase ethanol production but will not result in the increased ethanol yields expected through the means described supra. However, such a knockout will prevent any diversion of product to lactic acid. Methods and knockouts for Clostridium phytofermentans are described in U.S. application Ser. No. 12/729,037 and PCT application Serial No. PCT/US11/29102, both of which are herein incorporated by reference in its entirety. The same methods and genes are used to disrupt LDH in Clostridium sp. Q.D.

The wild-type strain of C. phytofermentans and eight lactate dehydrogenase derivative strains (LDH knockout strains) were deposited in the AGRICULTURAL RESEARCH SERVICE CULTURE COLLECTION(NRRL)(International Depositary Authority), National Center for Agricultural Utilization Research, Agricultural Research Service, U.S. Department of Agriculture, 1815 North University Street, Peoria, Ill. 61604 U.S.A. on Mar. 9, 2010 in accordance with and under the provisions of the Budapest Treaty for the International Recognition of the Deposit of Microorganisms for the Purpose of Patent Procedure, i.e., they will be stored with all the care necessary to keep them viable and uncontaminated for a period of at least five years after the most recent request for the furnishing of a sample of the deposits, and in any case, for a period of at least 30 (thirty) years after the date of deposit or for the enforceable life of any patent which may issue disclosing the cultures plus five years after the last request for a sample from the deposit. The strains were tested by the NRRL and determined to be viable. The NRRL has assigned the following NRRL deposit accession numbers to strains: C. phytofermentans Q8 (NRRL B-50351), C. phytofermentans 1117-1 (NRRL B-50352), C. phytofermentans 1117-2 (NRRL B-50353), C. phytofermentans 1117-3 (NRRL B-50354), C. phytofermentans 1117-4 (NRRL B-50355), C. phytofermentans 1232-1 (NRRL B-50356), C. phytofermentans 1232-4 (NRRL B-50357), C. phytofermentans 1232-5 (NRRL B-50358), and C. phytofermentans 1232-6 (NRRL B-50359).

Additional C. phytofermentans strains and derivatives were deposited in the NRRL in accordance with and under the provisions of the Budapest treaty. The NRRL has assigned the following NRRL deposit accession numbers to strains: Clostridium sp. Q.D (NRRL B-50361), Clostridium sp. Q.D-5 (NRRL B-50362), Clostridium sp. Q.D-7 (NRRL B-50363), Clostridium phytofermentans Q.7D (NRRL B-50364), all of which were deposited on Apr. 9, 2010; Clostridium phytofermentans Q.12 (NRRL B-50436) and Clostridium phytofermentans Q.13 (NRRL B-50437), deposited on Nov. 3, 2010.

The depositor acknowledges the duty to replace the deposits should the depository be unable to furnish a sample when requested, due to the condition of the deposits. All restrictions on the availability to the public of the subject culture deposits will be irrevocably removed upon the granting of a patent disclosing them. The deposits are available as required by foreign patent laws in countries wherein counterparts of the subject application, or its progeny, are filed. However, it should be understood that the availability of a deposit does not constitute a license to practice the subject matter disclosed herein in derogation of patent rights granted by governmental action.

### Example 5

### Expression of PDC and adhB

In order to improve glycolytic flux and ethanol production in Clostridium phytofermentans, several genes from other organisms were cloned and expressed in C. phytofermentans. Of particular interest were fungal species such as Zymomonas mobilis.

C. phytofermentans converts pyruvate to acetyl-coA via pyruvate ferredoxin oxidoreductase (pfor). The acetyl-coA is then converted to ethanol in two steps by the bi-function acetaldehyde-alcohol dehydrogenase (Cphy_{—}3925). However, acetyl-coA can be converted to a number of other products such as acetic acid and lactic acid. Production of these species diverts carbon from ethanol production. (FIGS. 1 & 2). One approach to optimizing the level of ethanol production (“titer”) is to bypass the production of acetyl-coA by expressing a fungal glycolytic enzyme such as pyruvate decarboxylase (PDC) in C. phytofermentans (FIG. 4). This enzyme converts pyruvate directly into acetaldehyde which can then be converted to ethanol by endogenous alcohol dehydrogenases (i.e. Cphy_{—}1029).

The predominant alcohol dehydrogenase (adh) in C. phytofermentans (Cphy_{—}3925) is bi-functional and prefers the substrate acetyl-coA. Other adh gene products exist but may not be expressed at sufficient levels to reduce all the acetaldehyde to ethanol. This could pose serious metabolic consequences for C. phytofermentans as acetaldehyde is toxic and the microorganism may not be able to further process the excess acetaldehyde produced by heterologous expression of PDC.

To compensate for a possible lack of increased alcohol dehydrogenase activity in C. phytofermentans, a heterologous adh was expressed. The adhB gene from Zymomonas mobilis was selected for its ability to produce higher titers of ethanol.

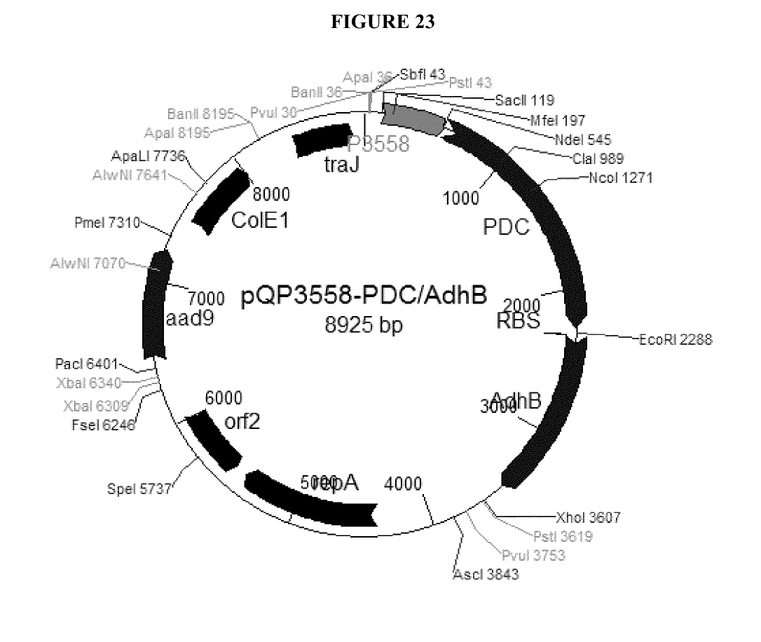

The two genes described above, PDC and adhB, were cloned from Zymomonas mobilis ATCC 10988 by PCR amplification. The primers used were designed to add appropriate restriction enzyme recognition sequences to the ends of the PCR products so as to facilitate cloning into the pMTL82351 plasmid. In addition, the upstream primer for adhB included an optimized ribosome-binding site (RBS) to ensure proper translation of the AdhB mRNA. The promoter sequence for the C. phytofermentans pfor (pyruvate formate oxidative reductase, Cphy_{—}3558) was similarly cloned using PCR. These three modules were ligated into the pMTL82351 in a sequential manner to generate the plasmids pMTL82351-P3558-PDC and pMTL82351-P3558-PDC-AdhB (see FIGS. 23 & 24). These plasmids also bear several functional modules including a gram-positive replication origin (repA) for replication in C. phytofermentans; a gram-negative replication origin (colE1) for replication in E. coli; the aad9 gene that confers resistance to spectinomycin; and the traJ origin for conjugal transfer. The three cloned modules P3558, PDC and AdhB were also cloned into the pMTL82251 vector using the same restriction sites. pMTL82251 is identical to pMTL82351 except that the aad9 spectinomycin-resistance marker is replaced with the ErmB erythromycin-resistance marker.

This embodiment outlines the cloning and expression of Z. mobilis PDC and AdhB in C. phytofermentans but other glycolytic genes from C. phytofermentans or from other organisms can be expressed or overexpressed in C. phytofermentans in order to improve glycolytic flux and ethanol titer using this system. Among these are facilitated glucose transporters from Bacillus subtilis and Z. mobilis; Z. mobilis glucokinase; C. phytofermentans pfor; and glyceraldehydes-3-phosphate dehydrogenase from B. subtilis or Z. mobilis. Other examples can be found in Table 6. This list represents only a sub-set of all possible candidate genes for improving glycolytic flux and ethanol titer in C. phytofermentans and is not exhaustive or intended to be limiting.

### Plasmid Construction

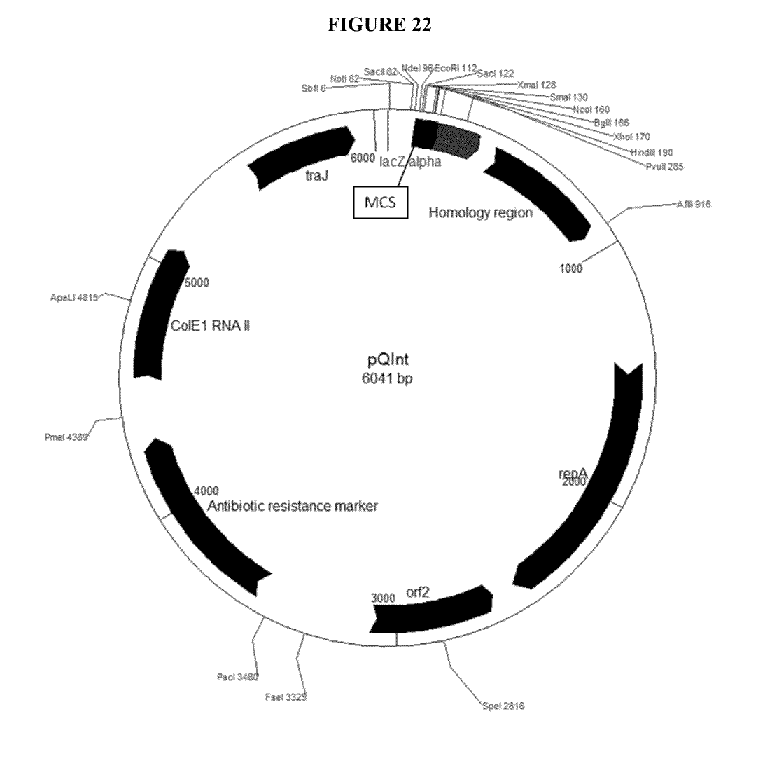

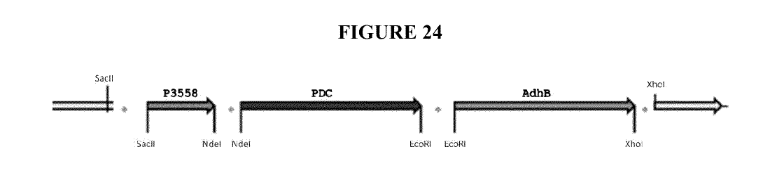

The general form of the plasmid backbone selected is illustrated in FIG. 22. These plasmids consist of five key elements. 1) A gram-negative origin of replication for propagation of the plasmid in E. coli or other gram-negative host(s). 2) A gram-positive replication origin for propagation of the plasmid in gram-positive organisms. In C. phytofermentans, this origin allows for suitable levels of replication prior to integration. 3) A selectable marker; typically a gene encoding antibiotic resistance. 4) An optional integration sequence (homology region); a sequence of DNA at least 400 base pairs in length and identical to a locus in the host chromosome. This represents the preferred site of integration. 5) A multi-cloning site (“MCS”) with or without a heterologous gene expression cassette cloned. An additional element for conjugal transfer of plasmid DNA (traJ) is an optional element described in certain embodiments. Plasmids containing the optional integration sequence are designated pQint. Those lacking this module are designated pQ. The promoter region from the C. phytofermentans pfor gene was amplified from the chromosome by PCR. This element, designated P3558, was amplified using primers designed to add specific restriction sites to the ends of the PCR product. The restriction sites chosen were SacII on the upstream primer and NdeI on the downstream primer. The choice of these primers in this particular embodiment is not particular or limiting. The P3558 element is illustrated in FIG. 24. The PCR product was digested with SacII and NdeI and ligated into the pQ plasmid also digested with the same enzymes. Ligation products were transformed into E. coli and screened both by colony PCR and by restriction analysis of purified plasmid. A clone verified to contain the correct insert was designated pQP3558. The pyruvate decarboxylase gene (PDC) was amplified by PCR from the Zymomonas mobilis, strain Zml (ATCC 10988). The primers were designed to add specific restriction sites to the ends of the PCR product. The restriction sites used were NdeI and EcoRI but the choice of these sites is not limiting. The resulting PDC element (operon) is also illustrated in FIG. 24. This element and the pQP3558 plasmid were both digested with NdeI and EcoRI. The digested PDC element was ligated to the digested pQP3558 plasmid and ligation products were transformed into E. coli. Candidate clones were screened by colony PCR and restriction digestion of purified plasmid. A clone verified to contain the correct PDC insert was designated pQP3558-PDC. The alcohol dehydrogenase II gene (AdhB) was also amplified from Zymomonas mobilis, strain Zml (ATCC 10988) by PCR. The primers used were designed to add specific restriction sites to the ends of the product. The restriction sites used were EcoRI and XhoI but the choice of these sites is not meant to be limiting. The upstream primer was further designed to add an optimized ribosome-binding site (RBS) to the PCR product. The resulting AdhB element (FIG. 24) and the pQP3558-PDC plasmid were both digested with EcoRI and XhoI. The digested AdhB element was ligated to the pQP3558-PDC plasmid and ligation products were transformed into E. coli. Candidate clones were screened by colony PCR and restriction digestion of purified plasmid. A clone verified to contain the correct PDC insert was designated pQP3558-PDC/AdhB. FIG. 24 illustrates all three of these elements and the orientation of the elements within the MCS of the pQ1 plasmid. FIG. 23 shows the complete pQP3558-PDC/AdhB plasmid. This figure further illustrates the use of the aad9 spectinomycin-resistance marker for selection of transformants in both E. coli and C. phytofermentans. The choice of this marker is not exclusive of other markers.

Expression of PDC and AdhB in C. phytofermentans

The plasmids pQ1 (identical to pQint shown in FIG. 22 but lacking the homology region and containing the aad9 spectinomycin-resistance marker), pQP3558-PDC and pQP3558-PDC/AdhB were transferred into C. phytofermentans using electroporation (described supra). Transformants were selected on BM agar plates containing 150 m/ml spectinomycin. Transformants were validated by restreaking on fresh BM plates with spectinomycin and by colony PCR (“cPCR”) to amplify plasmid sequences. cPCR was also performed with primers that amplify specific chromosomal loci to serve as a control to verify the PCR and that the clones were C. phytofermentans. Validated transformants were fermented in FM medium with 80-100 g/L cellobiose as a carbon source. The transformants were grown to mid-exponential growth phase prior to inoculation into the experimental shake flasks at 10% v/v. Fermentations were carried out at 35° C. for 5 to 6 days. Samples were collected twice a day and tested for pH. The pH of the fermentations was then adjusted with sodium hydroxide to keep the pH at 6.8. The samples were then analyzed for ethanol, lactic, acetic acid and residual sugars by high pressure liquid chromatography. All fermentations were conducted with the addition of 150 m/ml spectinomycin to maintain segregational stability of the plasmids.

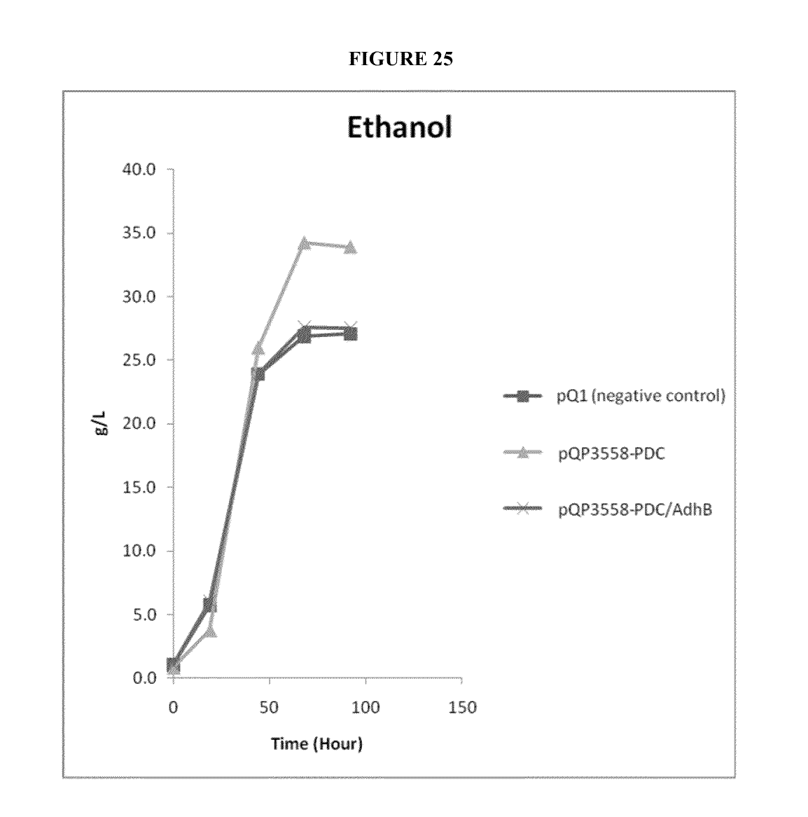

The expression of the PDC gene lead to a consistent 8-10 g/L increase in final ethanol titer over the control regardless of the specific strain of C. phytofermentans tested (FIG. 25). The expression of the adhB gene in conjuction with PDC abrogated the increase in titer seen with PDC alone, demonstrating that C. phytofermentans adh gene expressed products were sufficient to convert any excess acetaldehyde to ethanol and, in fact, showed improved activity over Z. mobilis adhB.

### Example 6

### Expression of Heterologous Genes in C. phytofermentans and Clostridium sp Q.D.

### Propagation Media (QM1) and Culture

| | g/L: |
|---|---|
| QM Base Media: | |
| KH₂PO₄ | 1.92 |
| K₂HPO₄ | 10.60 |
| Ammonium sulfate | 4.60 |
| Sodium citrate tribasic * 2H₂O | 3.00 |
| Bacto yeast extract | 6.00 |
| Cysteine | 2.00 |
| 20x Substrate Stock | |
| Maltose | 400.00 |
| 100X QM Salts solution: | |
| MgCl₂•6H₂O | 100 |
| CaCl₂•2H₂O | 15 |
| FeSO₄•7H₂O | 0.125 |

The seed propagation media was prepared according to the protocol above. Base media, salts and substrates were degassed with nitrogen prior to autoclave sterilization. Following sterilization, 94 ml of base media was combined with 1 ml of 100× salts and 5 mls of 20× substrate to achieve final concentrations of 1× for each. All additions were prepared anaerobically and aseptically.

Clostridium phytofermentans or Clostridium sp. Q.D. was propagated in QM media 24 hrs to an active cell density of 2×10⁹ cells per ml. The cells were concentrated by centrifugation and then transferred into the QM media bottles to achieve an initial cell density of 2×10⁹ cells per ml for the start of fermentation.

Cultures were then incubated at pH 6.5 and at 35° C. for 120 hr or until fermentations were complete. Product formation was determined by HPLC analysis using refractive index detection. Compositional analysis for the NaOH-treated corn stover was obtained via NREL standard methods using two-stage acid hydrolysis procedures.

### Microorganism Modification

Constitutive Expression of pIMPCphy

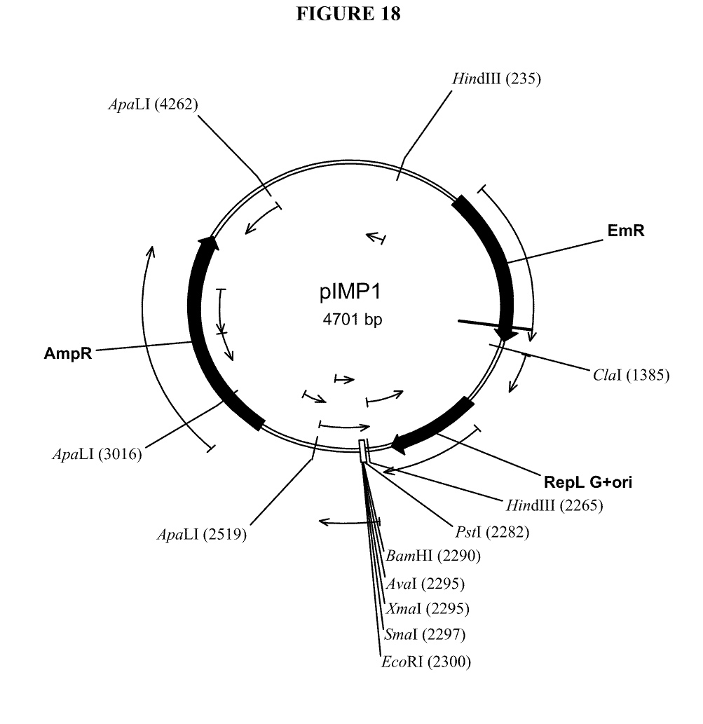

Plasmids suitable for use in Clostridium phytofermentans were constructed using portions of plasmids obtained from bacterial culture collections (Deutsche Sammlung von Mikroorganismen and Zellkulturen GmbH, Inhoffenstraβe 7 B, 38124 Braunschweig, Germany, hereinafter “DSMZ”). Plasmid pIMP1 is a non-conjugal shuttle vector that can replicate in Escherichia coli and C. phytofermentans; additionally, pIMP1 (FIG. 18) encodes for resistance to erythromycin (Em^{R}). The origin of transfer for the RK2 conjugal system was obtained from plasmid pRK29O (DSMZ) as DSM 3928, and the other conjugation functions of RK2 were obtained from pRK2013 (DSMZ) as DSM 5599. The polymerase chain reaction (PCR) was used to amplify the 112 base pair origin of transfer region (oriT) from pRK29O using primers that added ClaI restriction sites flanking the oriT region. This DNA fragment was inserted into the ClaI site on pIMP1 to yield plasmid pIMPT. pIMPT was shown to able to be transferred from one strain of E. coli to another when pRK2013 was also present to supply other conjugation functions. PCR was used to amplify the promoter of the alcohol dehydrogenase (Adh) gene Cphy_{—}1029 from the C. phytofermentans chromosome and it was used to replace the promoter of the erythromycin gene in pIMPT to create pIMPTCphy.

The successful transfer of pIMPTCphy into C. phytofermentans via electroporation was demonstrated by the ability to grow in the presence of 10 μg/mL erythromycin. In addition to phenotypic proof of electroporation provided by the growth on erythromycin, successive plasmid isolations from C. phytofermentans confirmed that the same plasmid was isolated from Clostridium phytofermentans and transferred into E. coli and recovered.

The method of conjugal transfer of pIMPTCphy from E. coli to C. phytofermentans involved constructing an E. coli strain (DHSalpha) that contains both pIMPTCphy and pRK2013. Fresh cells E. coli culture and fresh cells of the C. phytofermentans recipient culture were obtained by growth to mid-log phase using appropriate growth media (L broth and QM1 media respectively). The two bacterial cultures were then centrifuged to yield cell pellets and the pellets resuspended in the same media to obtain cell suspensions that were concentrated about ten-fold having cell densities of about 10¹⁰ cells per ml. These concentrated cell suspensions were then mixed to achieve a donor-to-recipient ratio of five-to-one, after which the cell suspension was spotted onto QM1 agar plates and incubated anaerobically at 30° C. for 24 hours. The cell mixture was removed from the QM1 plate and placed on solid or in liquid QM1 media containing antibiotics that allow the survival of C. phytofermentans recipient cells expressing erythromycin resistance. This was accomplished by using a combination of antibiotics consisting of trimethoprim (20 μg/ml), cycloserine (250 μg/ml), and erythromycin (10 μg/ml). The E. coli donor was unable to survive exposure to these concentrations of trimethoprim and cycloserine, while the C. phytofermentans recipient was unable to survive exposure to this concentration of erythromycin (but could tolerate trimethoprim and cycloserine at these concentrations). Accordingly, after anaerobic incubation on antibiotic-containing plates or liquid media for 5 to 7 days at 30° C., derivatives of C. phytofermentans were obtained that were erythromycin resistant and these C. phytofermentans derivatives were subsequently shown to contain pIMPCphy as demonstrated by PCR analyses.

The vector pIMPCphy was constructed as a shuttle vector for C. phytofermentans and Clostridium. sp. Q.D. It has an Ampicillin-resistance cassette and an Origin of Replication (ori) for selection and replication in E. coli. It contains a Gram-positive origin of replication that allows the replication of the plasmid in C. phytofermentans. In order to select for the presence of the plasmid, the pIMPCphy carries an erythromycin resistance gene under the control of the C. phytofermentans promoter of the gene Cphy1029. This plasmid can be transferred to C. phytofermentans by electroporation or by transconjugation with an E. coli strain that has a mobilizing plasmid, for example pRK2030. A plasmid map of pIMPCphy is depicted in FIG. 19. The DNA sequence of pIMPCphy was identified supra as SEQ ID NO: 1. pIMPCphy is an effective replicative vector system for all microbes, including all gram⁺ and gram⁻ bacteria, and fungi (including yeasts).

### Constitutive Promoter

In a first step, several promoters from C. phytofermentans were chosen that show high expression of their corresponding genes in all growth stages as well as on different substrates. These promoters also work well in Clostridium sp Q.D. A promoter element can be selected by selecting key genes that would necessarily be involved in constitutive pathways (e.g., ribosomal genes, or for ethanol production, alcohol dehydrogenase genes). Examples of promoters from such genes include but are not limited to:

Cphy_{—}1029: iron-containing alcohol dehydrogenase

Cphy_{—}3510: Ig domain-containing protein

Cphy_{—}3925: bifunctional acetaldehyde-CoA/alcohol dehydrogenase

### Cloning of Promoter

The different promoters in the upstream regions of the genes were amplified by PCR. The primers for this PCR reaction were chosen in a way that they include the promoter region but do not include the ribosome binding sites of the downstream gene. The primers were engineered to introduce restriction sites at the end of the promoter fragments that are present in the multiple cloning site of pIMPCphy but are otherwise not present in the promoter region itself, for example SalI, BamHI, XmaI, SmaI, EcoRI.

The PCR reaction was performed with a commercially available PCR Kit, e.g. GoTaq® Green Master

Mix (Promega Corporation, 2800 Woods Hollow Road, Madison, Wis. 53711 USA), according to the manufacturer's conditions. The reaction is run in a thermal cycler, e.g. Gene Amp System 2400 (PerkinElmer, 940 Winter St., Waltham Mass. 02451 USA). The PCR products were purified with the GenElute™ PCR Clean-Up Kit (Sigma-Aldrich Corp., St. Louis, Mo., USA). Both the purified PCR products as well as the plasmid pIMPCphy were then digested with the corresponding enzymes with the appropriate amounts according to the manufacturer's conditions (restriction enzymes from New England Biolabs, 240 County Road, Ipswich, Mass. 01938 USA and Promega). The PCR products and the plasmid were then analyzed and gel-purified on a Recovery FlashGel (Lonza Biologics, Inc., 101 International Drive, Portsmouth, N.H.03801 USA). The PCR products were subsequently ligated to the plasmid with the Quick Ligation Kit (New England Biolabs) and competent cells of E. coli (DH5α) are transformed with the ligation mixtures and plated on LB plates with 100 μg/ml ampicillin. The plates are incubated overnight at 37° C.

Ampicillin resistant E. coli colonies were picked from the plates and restreaked on new selective plates. After growth at 37° C., liquid LB medium with 100 μg/ml ampicillin was inoculated with a single colony and grown overnight at 37° C. Plasmids were isolated from the liquid culture with the Gene Elute™ Plasmid isolation kit.

### Mintprep Kit (Sigma-Aldrich).

Plasmids were checked for the right insert by PCR reaction and restriction digest with the appropriate primers and by restriction enzymes respectively. To ensure the sequence integrity, the insert is sequenced at this step.

### Cloning of Genes

One or more genes disclosed in Table 2, which can include each gene's own ribosome binding sites, were amplified via PCR and subsequently digested with the appropriate enzymes as described previously under Cloning of Promoter. Resulting plasmids were also treated with the corresponding restriction enzymes and the amplified genes are mobilized into plasmids through standard ligation. E. coli were transformed with the plasmids and correct inserts were verified from transformants selected on selection plates.

### Transconjugation

E. coli DH5α along with the helper plasmid pRK2030, were transformed with the different plasmids discussed above. E. coli colonies with both of the foregoing plasmids were selected on LB plates with 100 μg/ml ampicillin and 50 μg/ml kanamycin after growing overnight at 37° C. Single colonies were obtained after re-streaking on selective plates at 37° C. Growth media for E. coli (e.g. LB or LB supplemented with 1% glucose and 1% cellobiose) was inoculated with a single colony and either grown aerobically at 37° C. or anaerobically at 35° C. overnight. Fresh growth media was inoculated 1:100 with the overnight culture and grown until mid log phase. A C. phytofermentans strain was also grown in the same media until mid log.

The two different cultures, C. phytofermentans and E. coli with pRK2030 and one of the plasmids, were then mixed in different ratios, e.g. 1:1000, 1:100, 1:10, 1:1, 10:1, 100:1, 1000:1. The mating was performed in either liquid media, on plates or on 25 mm Nucleopore Track-Etch Membrane (Whatman, Inc., 800 Centennial Avenue, Piscataway, N.J. 08854 USA) at 35° C. The time was varied between 2 h and 24 h, and the mating media was the same growth media in which the culture was grown prior to the mating. After the mating procedure, the bacteria mixture was either spread directly onto plates or first grown on liquid media for 6 h to 18 h and then plated. The plates contain 10 μg/ml erythromycin as selective agent for C. phytofermentans and 10 μg/ml Trimethoprim, 150 μg/ml Cyclosporin and 100 μg/ml Nalidixic acid as counter selectable media for E. coli.

After 3 to 5 days incubation at 35° C., erythromycin-resistant colonies were picked from the plates and restreaked on fresh selective plates. Single colonies were picked and the presence of the plasmid is confirmed by PCR reaction.

### Gene Expression

The expression of the genes on the different plasmids is then tested under conditions where there is little to no expression of the corresponding genes from the chromosomal locus. Positive candidates show constitutive expression of the cloned genes.

### Constitutive Expression of a Cellulase

pCphyP3510-1163

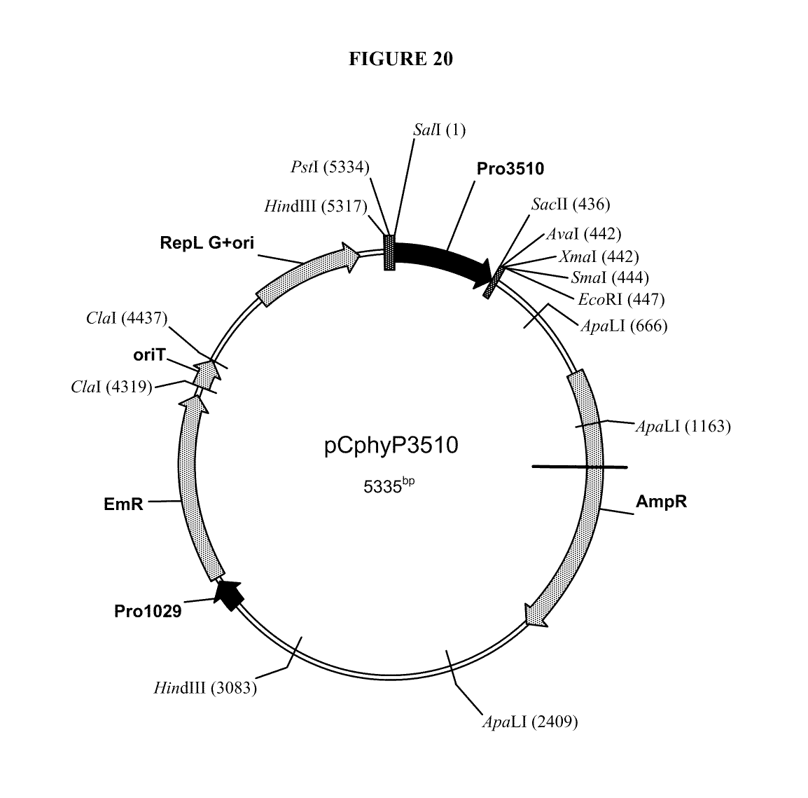

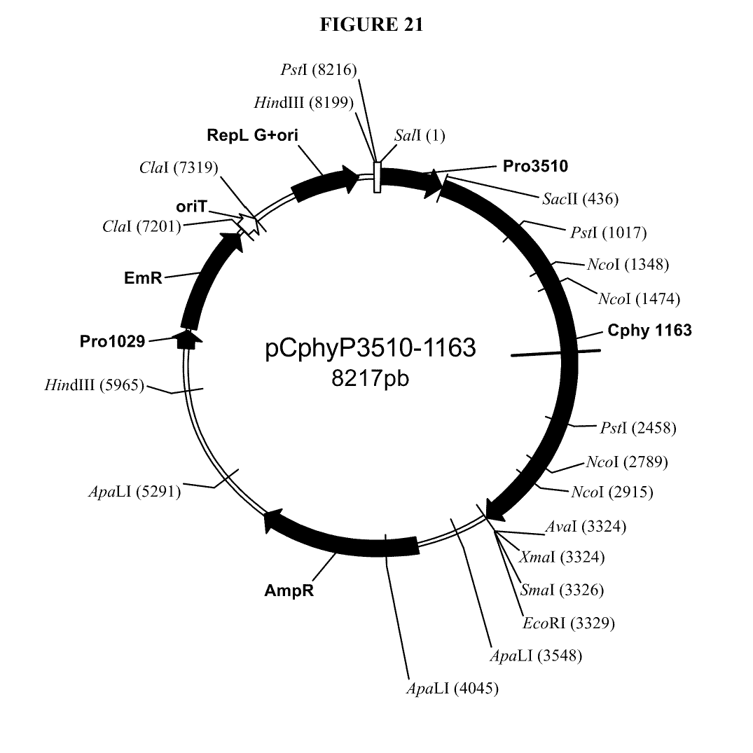

Two primers were chosen to amplify Cphy_{—}1163 using C. phytofermentans genomic DNA as template. The two primers were: cphy_{—}1163F: 5′-CCG CGG AGG AGG GTT TTG TAT GAG TAA AAT CAG AAG AAT AGT TTC-3 (SEQ ID NO: 2), which contained a SacII restriction enzyme site and ribosomal site; and cphy_{—}1163R: CCC GGG TTA GTG GTG GTG GTG GTG GTG TTT TCC ATA ATA TTG CCC TAA TGA (SEQ ID NO: 3), which containing a XmaI site and His-tag. The amplified gene was cloned into Topo-TA first, then digested with SacII and XmaI, the cphy_{—}1163 fragment was gel purified and ligated with pCPHY3510 (FIG. 20) digested with SacII and XmaI, respectively. The plasmid was transformed into E. coli, purified and then transformed into C. phytofermentans by electroporation. The plasmid map is shown in FIG. 21.

Using the methods above genes encoding Cphy_{—}3367, Cphy_{—}3368, Cphy_{—}3202 and Cphy_{—}2058 were cloned into pCphy3510 to produce pCphy3510_{—}3367, pCphy3510_{—}3368, pCphy3510_{—}3202, and pCphy3510_{—}2058 respectively. These vectors were transformed into C. phytofermentans via electroporation as described infra. In addition, genes encoding the heat shock chaperonin proteins, Cphy_{—}3289 and Cphy_{—}3290 were incorporated into pCphy3510. In another embodiment, an endogenous or exogenous gene can be cloned into this vector and used to transform C. phytofermentans, C. sp. Q.D, or another bacteria or fungal cell.

Electroporation Conditions for Clostridium sp. Q.D

No electroporation protocol existed for Clostridium Q.D; therefore a new protocol was established to transfer plasmids into this organism. Based on kill curve experiments, it was noted that cell suspensions containing Clostridium sp. Q.D. will arch at the following condition: 3000V, 600 ohms, and 25 uF. However, the ideal electroporation condition was noted at 2000-2250 V, 600 ohms, and 25 uF; the experimental values for time constants range from 3.2-5.1 ms (average) over the course of 23 independent electroporation procedures. Additionally, the experimental voltage for 2500 V fluctuates from 2400-2500 V based on the freshness of the electroporation buffer.

### Example 7

### Microorganism Modification and Vector Construction

### Plasmid Construction

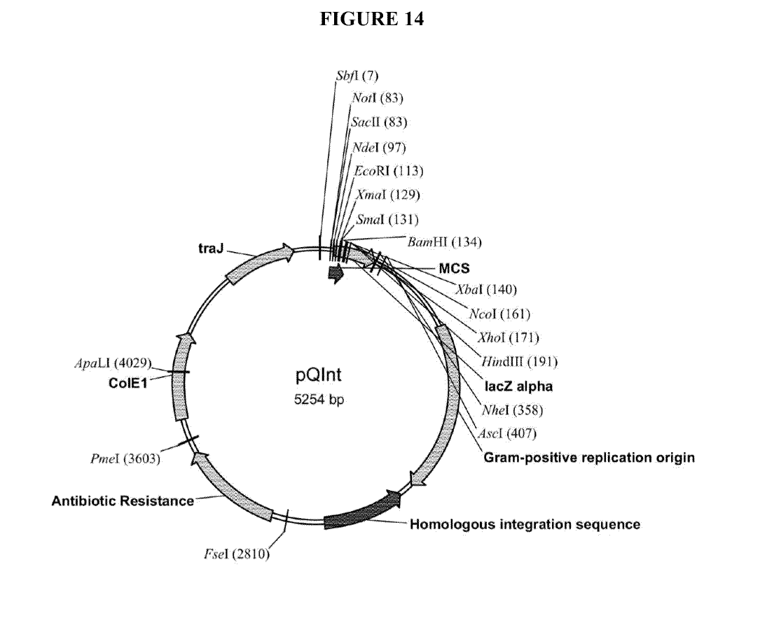

A general illustration of an integrating replicative plasmid, pQInt, is shown in FIG. 14. Identified elements include a Multi-cloning site (MCS) with a LacZ-α reporter for use in E. coli; a gram-positive replication origin; the homologous integration sequence; an antibiotic-resistance cassette; the ColE1 gram-negative replication origin and the traJ origin for conjugal transfer. Several unique restriction sites are indicated but are not meant to be limiting on any embodiment. The arrangement of the elements can be modified.

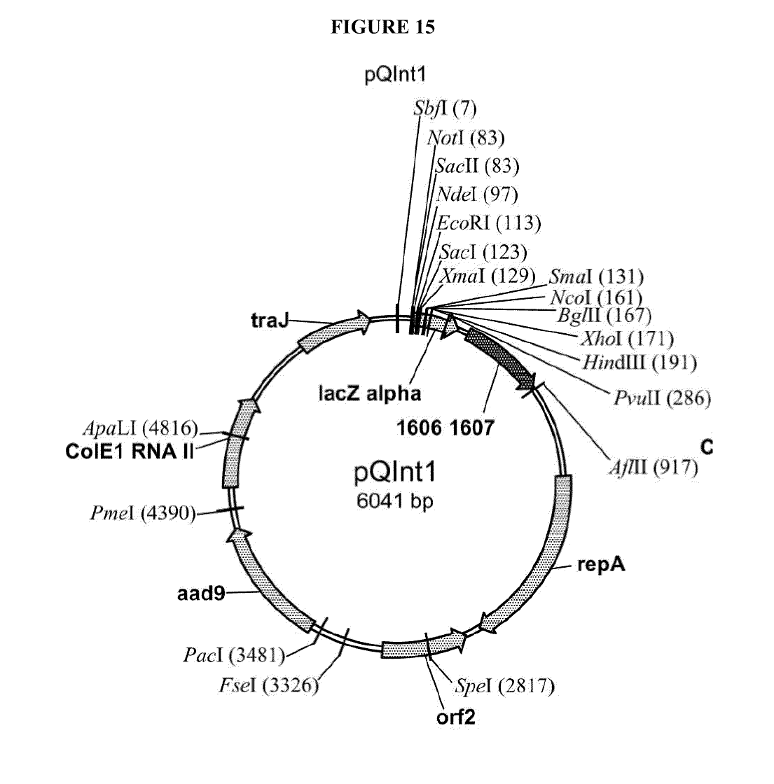

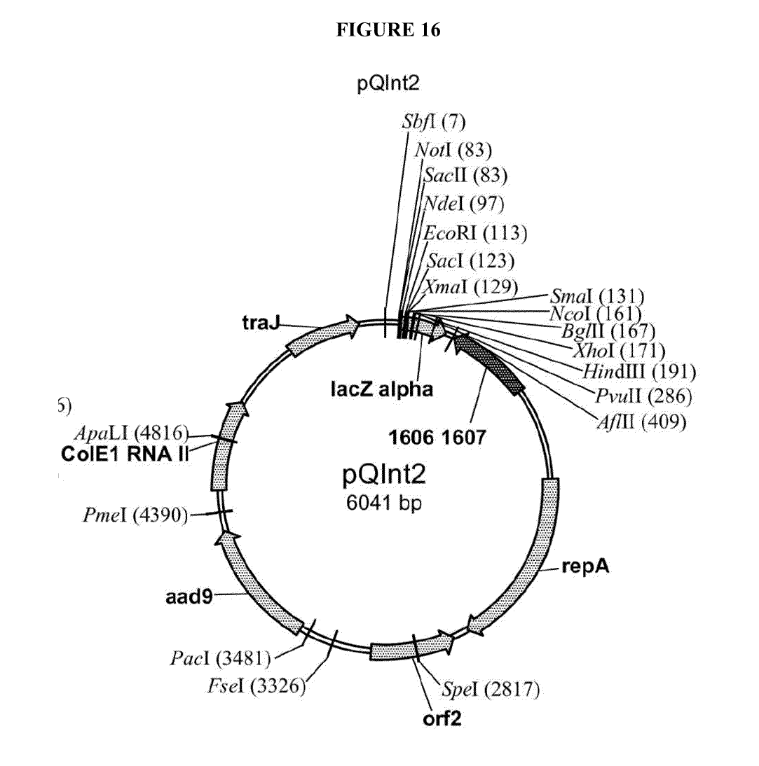

Another embodiment, depicted in FIG. 15 and FIG. 16, is a map of the plasmids pQInt1 and pQInt2. These plasmids contain gram-negative (ColE1) and gram-positive (repA/Orf2) replication origins; the bi-functional aad9 spectinomycin-resistance gene; traJ origin for conjugal transfer; LacZ-α/MCS and the 1606-1607 region of chromosomal homology. Since the 1606-1607 region of homology is cloned into a single AscI site, it can be obtained in two different orientations in a single cloning step. Plasmid pQInt2 is identical to pQInt1 except the orientation of the homology region is reversed.

These plasmids consist of five key elements. 1) A gram-negative origin of replication for propagation of the plasmid in E. coli or other gram-negative host(s). 2) A gram-positive replication origin for propagation of the plasmid in gram-positive organisms. In C. phytofermentans, this origin allows for suitable levels of replication prior to integration. 3) A selectable marker; typically a gene encoding antibiotic resistance. 4) An integration sequence; a sequence of DNA at least 400 base pairs in length and identical to a locus in the host chromosome. This represents the preferred site of integration. 5) A multi-cloning site (“MCS”) with or without a heterologous gene expression cassette cloned. An additional element for conjugal transfer of plasmid DNA is an optional element described in certain embodiments.

### Plasmid Utilization

The plasmid is digested with suitable restriction enzyme(s) to allow a heterologous gene expression cassette (“insert”) to be ligated in the MCS. Ligation products are transformed into a suitable cloning host, typically E. coli. Antibiotic resistant transformants are screened to verify the presence of the desired insert. The plasmid is then transformed into C. phytofermentans or other suitable expression host strain. Transformants are selected based on resistance to the appropriate antibiotic. Resistant colonies are propagated in the presence of antibiotic to allow for homologous recombination integration of the plasmid. Integration is verified by a “junction PCR” protocol. This protocol uses either a preparation of host chromosomal DNA or a sample of transformed cells. The junction PCR utilizes one primer that hybridizes to the plasmid backbone flanking the MCS and a second primer that hybridizes to the chromosome flanking the site of integration. The primers must be designed so they are unique. That is, the plasmid primer cannot hybridize to chromosomal sequences and the chromosomal primer cannot hybridize to the plasmid. The ability to amplify a PCR product demonstrates integration at the correct site (see FIGS. 14-16).

Standard gene expression systems use autonomously replicating plasmids (“episomes” or “episomal plasmids”). Such plasmids are not suitable for use in C. phytofermentans, Clostridium sp. Q.D. and most other Clostridia due to segregational instability. The use of homologous sequences to allow for integration of a replicative gene expression in C. phytofermentans is not usual for transformation.

Use of a series of plasmids each containing a different antibiotic resistance gene, allows for versatility in cases where certain antibiotics are not suitable for specific organisms. The embodiments use an “integration sequence” which is easily cloned from the chromosome by PCR using primers with tails that encode the appropriate restriction enzyme recognition sequences. This allows for the targeted integration of the entire plasmid at a chosen locus. The inclusion of a gram-negative replication origin allows for cloning and the easy propagation of the plasmid in a host such as E. coli. The gram-positive replication origin allows for a level of replication of the plasmid in C. phytofermentans after transformation and prior to integration. This contrasts with true suicide integration which utilizes non-replicating plasmids. In true suicide integration, the only way to obtain an antibiotic resistant transformant is to have the plasmid integrate immediately after transformation. This is a low probability event. Replication from the gram-positive origin after transformation results in a greater number of transformed cells which makes the integration event statistically more likely.

The integrated plasmid is stable indefinitely. The transformed strain can be indefinitely propagated without loss of plasmid DNA. The transformant can be evaluated for heterologous gene expression under any suitable conditions. Stability of the integrated DNA can be ensured by continuous culture in the presence of the appropriate antibiotic. It is also possible to remove the antibiotic if so desired.

### Constitutive Expression of Cellulases I

Plasmids suitable for use in Clostridium phytofermentans were constructed using pQInt with the promoter from the C. phytofermentans pyruvate ferredoxin oxidase reductase gene Cphy_{—}3558 and the C. phytofermentans cellulase gene Cphy_{—}3202. The sequence of this vector (pMTL82351-P3558-3202) inserted DNA (SEQ ID NO: 61) is as follows:

SEQ ID NO: 61:
CCTGCAGGATAAAAAAATTGTAGATAAATTTTATAAAATAGTTTTATC
TACAATTTTTTTATCAGGAAACAGCTATGACCGCGGGGATTTTACACG
TTTCATTAATAATTTCTTATATTTCTTTATTTGTTTGTAAAATTTACT
TAAATTTCGCCAGAAAACAAAAGAAAGCCTTTACTAATTAATAGTTTA
GTGATACTCTTTTATGTAGGTATTTTTTAAAATACATTAAACCTAGGT
AATTGAGGAAAGTTACAATTACCATTATATAAGGAGGATATTCATATG
AAAAGAAAACTGAAACAAAGATGTGCTGTTTTAGTGGCAGTTGCAACG
ATGATAGCTTCGTTGCAATGGGGGAGAGTGCCAGTACAAGCAGTAACA
GCAGACGGTCTTACCTCTCAACAGTATGTTGAGGCAATGGGCGAAGGC
TGGAACTTAGGAAATTCCTTTGATGGTTTTGATTCTGATACTTCAAAA
CCAGATCAAGGCGAGACCGCTTGGGGAAATCCTAAGGTTACAAAAGAG
CTAATCCATGCAGTCAAACAAAAAGGCTATAGTAGTATCCGCATACCA
ATGACCCTATATCGTAGATATACGGAGAGCAATGGTGTATGCACTATC
GATAGCGCATGGATAGCACGTTACAAAGAAGTAGTAGATTATGCAGTT
GCAGAAGGTTTATACGTTATGATAAACATTCACCATGATTCCTGGATA
TGGTTATCTTCATGGGATGGAAATAAGAGTTCTGTGCAATATGTAAGA
TTTACTCAGATGTGGGATCAACTTGCGAAGGCATTTAAAGATTATCCG
TTACAAGTATGTTTTGAAACGATAAATGAGCCGAACTTTCAAAACTCT
GGAAACGTTACTGCACAGAATAAATTAGATATGCTTAACCAAGCGGCT
TACAATATAATTCGTGCCTCTGGTGGATCAAATGCAAAGAGAATGATT
GTTTTACCATCACTAAATACGAACCATGATAATAGTGTACCATTAGCT
GATTTCATAACTAAATTGAATGATTCTAATATCATTGCAACCGTTCAT
TATTATAGTGAATGGGTATTTAGTGCTAACCTTGGTAAGACAAGCTTT
GATGAAGATTTATGGGGAAATGGTGATTACACTCCTCGTGATGCGGTA
AATAAGGCGTTTGATACCATTTCCAATGCATTTACAGCAAAAAAAATC
GGTGTTGTTATCGGAGAATTTGGTCTTTTAGGTTATGACTCTGATTTT
GAAAATAATCAACCAGGCGAAGAATTAAAATATTATGAGTATATGAAT
TATGTAGCTAGACAAAAGAAAATGTGCCTTATGTTTTGGGATAACGGA
TCTGGAATTAATCGTAACGACTCTAAGTATAGTTGGAAAAAACCTATA
GTTGGAAAGATGTTAGAAGTATCTATGACAGGACGTTCCTCTTATGCA
ACAGGCCTTGATACCATTTACCTAAACGGCAGCTCATTTAATGATATT
AATATCCCGCTTACTCTAAACGGTAACACCTTTGTTGGAGTTACAGGA
TTAACCAGTGGTACCGATTTTACGTATAACCAATCCAATGCAACACTA
ACATTAAAATCATCCTACGTGAAGAAGGTTTATGATGCAATGGGAAGT
AATTATGGTACGGTAGCTGATTTGGTACTTAAGTTTTCAAGTGGAGCT
GATTGGCATGAGTATTTAGTGAAATACAAAGCACCAGTATTTCAAAAT
GCGAATGGAACTGTTTCCAATGGAATTAATATTCCAGTTCAATTTAAC
GGAAGTAAACTCCGTCGTTCTACAGCTTATATAGGTTCTAATCGAGTT
GGCCCGAATCAAAGCTGGTGGATGTATTTAGAGTATGGTGCAACTTTT
GTGGCGAACTATACGAACAATATTTTAACCATTAAGCCTGATTTCTTT
AAGGATGGTTCTGTTTATGATGGAAATATATCATTTGAGATGGAGTTT
TATGATGGACAAAAGTTAAAATATAATCTTAATAAATCAAATGGTAAC
ATAACAGGAACTGCAGCAGCAGTAACCCCTACACCAACACCAACGGCG
ACACCAACACCAACAGCGACGCCAACACCAACCGTAACACCAAAACCA
ACAATAACCCCAACAGTAACGCCGACACCAACAGTAACGCCAAAACCA
ACAATAACACCGACAGTAACACCAACTCCTACTCCAATCCCAGGAACA
GGTCCAGTTACATTAAAATACGAAGTAACGAATACTTGGGATAAGCAT
ACACAGGCGAATATTACATTAACCAATACCTCTAATACAGCACTAAAG
AATTTTGTTGTATCATTTACTTATAAAGGGTATATAGACCAAATGTGG
AGTGCAGATTTGGTTAGTCAAAATTCGGGTACCATTACAGTGAAGGGA
CCAGCATGGGCTACGAATCTAGATCCAGGGCAAAGTATAACATTTGGT
TTTATTGCTTCACATGATACACCGTCTGTTGATCCACCATCAAATGTT
ACTTTAGTTAGTTCAAATTAAAATTGTATTCAAATCTCGAGGCCTGCA
GACATGCAAGCTTGGCACTGGCCGTCGTTTTACAACGTCGTGACTGGG
AAAACCCTGGCGTTACCCAACTTAATCGCCTTGCAGCACATCCCCCTT
TCGCCAGCTGGCGTAATAGCGAAGAGGCCCGCACCGATCGCCCTTCCC
AACAGTTGCGCAGCCTGAATGGCGAATGGCGCTAGCATAAAAATAAGA
AGCCTGCATTTGCAGGCTTCTTATTTTTATGGCGCGCCGTTCTGAATC
CTTAGCTAATGGTTCAACAGGTAACTATGACGAAGATAGCACCCTGGA
TAAGTCTGTAATGGATTCTAAGGCATTTAATGAAGACGTGTATATAAA
ATGTGCTAATGAAAAAGAAAATGCGTTAAAAGAGCCTAAAATGAGTTC
AAATGGTTTTGAAATTGATTGGTAGTTTAATTTAATATATTTTTTCTA
TTGGCTATCTCGATACCTATAGAATCTTCTGTTCACTTTTGTTTTTGA
AATATAAAAAGGGGCTTTTTAGCCCCTTTTTTTTAAAACTCCGGAGGA
GTTTCTTCATTCTTGATACTATACGTAACTATTTTCGATTTGACTTCA
TTGTCAATTAAGCTAGTAAAATCAATGGTTAAAAAACAAAAAACTTGC
ATTTTTCTACCTAGTAATTTATAATTTTAAGTGTCGAGTTTAAAAGTA
TAATTTACCAGGAAAGGAGCAAGTTTTTTAATAAGGAAAAATTTTTCC
TTTTAAAATTCTATTTCGTTATATGACTAATTATAATCAAAAAAATGA
AAATAAACAAGAGGTAAAAACTGCTTTAGAGAAATGTACTGATAAAAA
AAGAAAAAATCCTAGATTTACGTCATACATAGCACCTTTAACTACTAA
GAAAAATATTGAAAGGACTTCCACTTGTGGAGATTATTTGTTTATGTT
GAGTGATGCAGACTTAGAACATTTTAAATTACATAAAGGTAATTTTTG
CGGTAATAGATTTTGTCCAATGTGTAGTTGGCGACTTGCTTGTAAGGA
TAGTTTAGAAATATCTATTCTTATGGAGCATTTAAGAAAAGAAGAAAA
TAAAGAGTTTATATTTTTAACTCTTACAACTCCAAATGTAAAAAGTTA
TGATCTTAATTATTCTATTAAACAATATAATAAATCTTTTAAAAAATT
AATGGAGCGTAAGGAAGTTAAGGATATAACTAAAGGTTATATAAGAAA
ATTAGAAGTAACTTACCAAAAGGAAAAATACATAACAAAGGATTTATG
GAAAATAAAAAAAGATTATTATCAAAAAAAAGGACTTGAAATTGGTGA
TTTAGAACCTAATTTTGATACTTATAATCCTCATTTTCATGTAGTTAT
TGCAGTTAATAAAAGTTATTTTACAGATAAAAATTATTATATAAATCG
AGAAAGATGGTTGGAATTATGGAAGTTTGCTACTAAGGATGATTCTAT
AACTCAAGTTGATGTTAGAAAAGCAAAAATTAATGATTATAAAGAGGT
TTACGAACTTGCGAAATATTCAGCTAAAGACACTGATTATTTAATATC
GAGGCCAGTATTTGAAATTTTTTATAAAGCATTAAAAGGCAAGCAGGT
ATTAGTTTTTAGTGGATTTTTTAAAGATGCACACAAATTGTACAAGCA
AGGAAAACTTGATGTTTATAAAAAGAAAGATGAAATTAAATATGTCTA
TATAGTTTATTATAATTGGTGCAAAAAACAATATGAAAAAACTAGAAT
AAGGGAACTTACGGAAGATGAAAAAGAAGAATTAAATCAAGATTTAAT
AGATGAAATAGAAATAGATTAAAGTGTAACTATACTTTATATATATAT
GATTAAAAAAATAAAAAACAACAGCCTATTAGGTTGTTGTTTTTTATT
TTCTTTATTAATTTTTTTAATTTTTAGTTTTTAGTTCTTTTTTAAAAT
AAGTTTCAGCCTCTTTTTCAATATTTTTTAAAGAAGGAGTATTTGCAT
GAATTGCCTTTTTTCTAACAGACTTAGGAAATATTTTAACAGTATCTT
CTTGCGCCGGTGATTTTGGAACTTCATAACTTACTAATTTATAATTAT
TATTTTCTTTTTTAATTGTAACAGTTGCAAAAGAAGCTGAACCTGTTC
CTTCAACTAGTTTATCATCTTCAATATAATATTCTTGACCTATATAGT
ATAAATATATTTTTATTATATTTTTACTTTTTTCTGAATCTATTATTT
TATAATCATAAAAAGTTTTACCACCAAAAGAAGGTTGTACTCCTTCTG
GTCCAACATATTTTTTTACTATATTATCTAAATAATTTTTGGGAACTG
GTGTTGTAATTTGATTAATCGAACAACCAGTTATACTTAAAGGAATTA
TAACTATAAAAATATATAGGATTATCTTTTTAAATTTCATTATTGGCC
TCCTTTTTATTAAATTTATGTTACCATAAAAAGGACATAACGGGAATA
TGTAGAATATTTTTAATGTAGACAAAATTTTACATAAATATAAAGAAA
GGAAGTGTTTGTTTAAATTTTATAGCAAACTATCAAAAATTAGGGGGA
TAAAAATTTATGAAAAAAAGGTTTTCGATGTTATTTTTATGTTTAACT
TTAATAGTTTGTGGTTTATTTACAAATTCGGCCGGCCCAATGAATAGG
TTTACACTTACTTTAGTTTTATGGAAATGAAAGATCATATCATATATA
ATCTAGAATAAAATTAACTAAAATAATTATTATCTAGATAAAAAATTT
AGAAGCCAATGAAATCTATAAATAAACTAAATTAAGTTTATTTAATTA
ACAACTATGGATATAAAATAGGTACTAATCAAAATAGTGAGGAGGATA
TATTTGAATACATACGAACAAATTAATAAAGTGAAAAAAATACTTCGG
AAACATTTAAAAAATAACCTTATTGGTACTTACATGTTTGGATCAGGA
GTTGAGAGTGGACTAAAACCAAATAGTGATCTTGACTTTTTAGTCGTC
GTATCTGAACCATTGACAGATCAAAGTAAAGAAATACTTATACAAAAA
ATTAGACCTATTTCAAAGAAAATAGGAGATAAAAGCAACTTACGATAT
ATTGAATTAACAATTATTATTCAGCAAGAAATGGTACCGTGGAATCAT
CCTCCCAAACAAGAATTTATTTATGGAGAATGGTTACAAGAGCTTTAT
GAACAAGGATACATTCCTCAGAAGGAATTAAATTCAGATTTAACCATA
ATGCTTTACCAAGCAAAACGAAAAAATAAAAGAATATACGGAAATTAT
GACTTAGAGGAATTACTACCTGATATTCCATTTTCTGATGTGAGAAGA
GCCATTATGGATTCGTCAGAGGAATTAATAGATAATTATCAGGATGAT
GAAACCAACTCTATATTAACTTTATGCCGTATGATTTTAACTATGGAC
ACGGGTAAAATCATACCAAAAGATATTGCGGGAAATGCAGTGGCTGAA
TCTTCTCCATTAGAACATAGGGAGAGAATTTTGTTAGCAGTTCGTAGT
TATCTTGGAGAGAATATTGAATGGACTAATGAAAATGTAAATTTAACT
ATAAACTATTTAAATAACAGATTAAAAAAATTATAAAAAAATTGAAAA
AATGGTGGAAACACTTTTTTCAATTTTTTTGTTTTATTATTTAATATT
TGGGAAATATTCATTCTAATTGGTAATCAGATTTTAGAAGTTTAAACT
CCTTTTTGATAATCTCATGACCAAAATCCCTTAACGTGAGTTTTCGTT
CCACTGAGCGTCAGACCCCGTAGAAAAGATCAAAGGATCTTCTTGAGA
TCCTTTTTTTCTGCGCGTAATCTGCTGCTTGCAAACAAAAAAACCACC
GCTACCAGCGGTGGTTTGTTTGCCGGATCAAGAGCTACCAACTCTTTT
TCCGAAGGTAACTGGCTTCAGCAGAGCGCAGATACCAAATACTGTTCT
TCTAGTGTAGCCGTAGTTAGGCCACCACTTCAAGAACTCTGTAGCACC
GCCTACATACCTCGCTCTGCTAATCCTGTTACCAGTGGCTGCTGCCAG
TGGCGATAAGTCGTGTCTTACCGGGTTGGACTCAAGACGATAGTTACC
GGATAAGGCGCAGCGGTCGGGCTGAACGGGGGGTTCGTGCACACAGCC
CAGCTTGGAGCGAACGACCTACACCGAACTGAGATACCTACAGCGTGA
GCTATGAGAAAGCGCCACGCTTCCCGAAGGGAGAAAGGCGGACAGGTA
TCCGGTAAGCGGCAGGGTCGGAACAGGAGAGCGCACGAGGGAGCTTCC
AGGGGGAAACGCCTGGTATCTTTATAGTCCTGTCGGGTTTCGCCACCT
CTGACTTGAGCGTCGATTTTTGTGATGCTCGTCAGGGGGGCGGAGCCT
ATGGAAAAACGCCAGCAACGCGGCCTTTTTACGGTTCCTGGCCTTTTG
CTGGCCTTTTGCTCACATGTTCTTTCCTGCGTTATCCCCTGATTCTGT
GGATAACCGTATTACCGCCTTTGAGTGAGCTGATACCGCTCGCCGCAG
CCGAACGACCGAGCGCAGCGAGTCAGTGAGCGAGGAAGCGGAAGAGCG
CCCAATACGCAGGGCCCCCTGCTTCGGGGTCATTATAGCGATTTTTTC
GGTATATCCATCCTTTTTCGCACGATATACAGGATTTTGCCAAAGGGT
TCGTGTAGACTTTCCTTGGTGTATCCAACGGCGTCAGCCGGGCAGGAT
AGGTGAAGTAGGCCCACCCGCGAGCGGGTGTTCCTTCTTCACTGTCCC
TTATTCGCACCTGGCGGTGCTCAACGGGAATCCTGCTCTGCGAGGCTG
GCCGGCTACCGCCGGCGTAACAGATGAGGGCAAGCGGATGGCTGATGA
AACCAAGCCAACCAGGAAGGGCAGCCCACCTATCAAGGTGTACTGCCT
TCCAGACGAACGAAGAGCGATTGAGGAAAAGGCGGCGGCGGCCGGCAT
GAGCCTGTCGGCCTACCTGCTGGCCGTCGGCCAGGGCTACAAAATCAC
GGGCGTCGTGGACTATGAGCACGTCCGCGAGCTGGCCCGCATCAATGG
CGACCTGGGCCGCCTGGGCGGCCTGCTGAAACTCTGGCTCACCGACGA
CCCGCGCACGGCGCGGTTCGGTGATGCCACGATCCTCGCCCTGCTGGC
GAAGATCGAAGAGAAGCAGGACGAGCTTGGCAAGGTCATGATGGGCGT
GGTCCGCCCGAGGGCAGAGCCATGACTTTTTTAGCCGCTAAAACGGCC
GGGGGGTGCGCGTGATTGCCAAGCACGTCCCCATGCGCTCCATCAAGA
AGAGCGACTTCGCGGAGCTGGTGAAGTACATCACCGACGAGCAAGGCA
AGACCGATCGGGCCC

The successful transfer of pMTL82351-P3558-3202 into C. phytofermentans strain Q.13 via electroporation was demonstrated by the ability to grow in the presence of 10 μg/mL erythromycin. The plasmid has been serially propagated in this transformant for over four months.

### Constitutive Promoter

Several other promoters from C. phytofermentans were chosen for vector use that show high expression of their corresponding genes in all growth stages as well as on different substrates. A promoter element can be selected by selecting key genes that would necessarily be involved in constitutive pathways (e.g., ribosomal genes, or for ethanol production, alcohol dehydrogenase genes). Examples of promoters from such genes include but are not limited to:

Cphy_{—}1029: iron-containing alcohol dehydrogenase

Cphy_{—}3510: Ig domain-containing protein

Cphy_{—}3925: bifunctional acetaldehyde-CoA/alcohol dehydrogenase

### Cloning of Cellulase Genes

One or more genes disclosed (see Table 2), which can include each gene's own ribosome binding sites, were amplified via PCR and subsequently digested with the appropriate enzymes as described previously under Cloning of Promoter. Resulting plasmids were also treated with the corresponding restriction enzymes and the amplified genes are mobilized into plasmids through standard ligation. E. coli were transformed with the plasmids and correct inserts were verified from transformants selected on selection plates.

### Example 8

### Transconjugation

E. coli DH5α along with the helper plasmid pRK2030, were transformed with the different plasmids discussed above. E. coli colonies with both of the foregoing plasmids were selected on LB plates with 100 μg/ml ampicillin and 50 μg/ml kanamycin after growing overnight at 37° C. Single colonies were obtained after re-streaking on selective plates at 37° C. Growth media for E. coli (e.g. LB or LB supplemented with 1% glucose and 1% cellobiose) was inoculated with a single colony and either grown aerobically at 37° C. or anaerobically at 35° C. overnight. Fresh growth media was inoculated 1:100 with the overnight culture and grown until mid log phase. A C. phytofermentans strain was also grown in the same media until mid log.

The two different cultures, C. phytofermentans and E. coli with pRK2030 and one of the plasmids, were then mixed in different ratios, e.g. 1:1000, 1:100, 1:10, 1:1, 10:1, 100:1, 1000:1. The mating was performed in either liquid media, on plates or on 25 mm Nucleopore Track-Etch Membrane (Whatman, Inc., 800 Centennial Avenue, Piscataway, N.J. 08854 USA) at 35° C. The time was varied between 2 h and 24 h, and the mating media was the same growth media in which the culture was grown prior to the mating. After the mating procedure, the bacteria mixture was either spread directly onto plates or first grown on liquid media for 6 h to 18 h and then plated. The plates contain 10 μg/ml erythromycin as selective agent for C. phytofermentans and 10 μg/ml Trimethoprim, 150 μg/ml Cyclosporin and 100 μg/ml Nalidixic acid as counter selectable media for E. coli.

After 3 to 5 days incubation at 35° C., erythromycin-resistant colonies were picked from the plates and restreaked on fresh selective plates. Single colonies were picked and the presence of the plasmid is confirmed by PCR reaction.

### Cellulase Gene Expression

The expression of the cellulase genes on the different plasmids was then tested under conditions where there is little to no expression of the corresponding genes from the chromosomal locus. Positive candidates showed constitutive expression of the cloned cellulases.

### Example 9

### Electroporation Procedure

All procedures were conducted anaerobically except centrifugation wherein the centrifuge tubes were sealed from the atmosphere.

Inoculated with C. phytofermentans, 50 mL of culture broth (QM) was grown at 37° C. overnight to an OD660=0.850. The entire culture was transferred to a 50 mL Falcon tube which was spun at 8,500 RPM (˜18,000 g) for 10 minutes. The supernatant was discarded and the pellet resuspended with 2.0 mL of Electroporation Buffer (EPB: 250 mM sucrose, 5 mM sodium phosphate, 2 mM MgSO₄). The suspension was again spun at 8,500 RPM (˜18,000 g) for 10 minutes. The supernatant was discarded and the pellet resuspended with 2.0 mL EPB wherein the sample was placed on ice.

575 μL of competent C. phytofermentans cells were transferred into a 0.4 cm electroporation cuvette (BioRad, Inc., 1000 Alfred Nobel Drive, Hercules, Calif. 94547), and the cuvettes kept on ice. 25 μL of DNA (˜1.0 μg) was added to each cuvette on ice. The solution was mixed by gently circulating the pipette tip. It was not mixed by pipetting or vortexing. The cells were incubated on ice for 4 minutes.

When ready for electroporation, the metal contacts of the electroporation cuvette were cleaned with a Kimwipe or other adsorbent material to ensure no trace of moisture was present. Electroporation was conducted using a Gene Pulser Xcell™ apparatus (BioRad, Inc.) at 1500 V to 2500 V, 25 μF, and 600 ohms. The ideal time constant was in the interval of 0.8 ms to 1.8 ms.

Immediately, the contents of the cuvette were diluted with 1 mL of prewarmed (37° C.) QM media. The entire solution was poured into a 10 mL QM tube and incubated anaerobically at 37° C. Following 150 minutes incubation, 2 μg/mL of erythromycin was added and the cells allowed to grow for two additional generations. A dilution series was then performed on the transformed C. phytofermentans with selective media.

### Example 10

### Assays

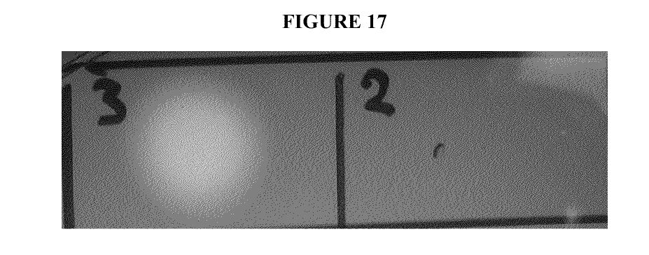

The transformants from the QM plate, which contained 20 μg/ml of erythromycin, were transformed into QM liquid medium, which contained 2% cellobiose and 20 μg/ml of erythromycin. The enzyme activities from the supernatant of overnight culture were assayed by CMC-congo red plate assay and Cellazyme T assay kit (Megazyme International Ireland, Ltd., Bray Business Park, Bray, Co., Wicklow, Ireland). The CMC-congo plate and the Cellazyme T assays indicated the transformant of another vector C. phytofermentans pCphy3510_{—}1163 showed increased activity than that of the control strain (FIG. 17). The CEL-T assay showed the transformant had an activity level of 54.5 mU/ml (left box “3”) whereas the control activity was only 3.7 mU/ml (right box “2”).

Using the methods above, other pQInt vectors, as listed below, have been constructed and different genes electroporated into C. phytofermentans strains. Several are listed below in Table 7.

TABLE 7

| Vector backbone | Promoter | Gene(s) |
|---|---|---|
| pMTL82351 | P3558 | Cpy_3202 |
| pMTL82351 | P3558 | Zymomonas PDC |
| pMTL82351 | P3558 | Zm PDC/AdhB |
| pMTL82351 | P3510 | glcP (B. subtilis glf)/Zm glk |
| pMTL82351 | P1029 | Ccel_3478-3479-3480 (NAD) |
| pMTL82351 | P1029 | Ccel_1310 (DHFR) |
| pMTL82351 | P1029 | B. sub LacA (beta-galactosidase) |
| pMTL82351 | P1029 | ermB (erythromycin-resistance) |
| pMTL82351 | P3925 | Q13_3925 (Adh) |
| pMTL82351 | None | Δpta (internal fragment) |
| pMTL82351 | None | Δpfl (double crossover) |
| pMTL82351 | None | Cpy_1163 |
| pMTL82251 | P3558 | Zm PDC |
| pMTL82251 | P3558 | Zm PDC/AdhB |
| pMTL82254 | P3668 | Himar1 (transposase) + Tn(spec) |
| pMTL82351 | P3668 | Himar1 (transposase) + Tn(catP) |
| pMTL82151 | P3558 | Zm PDC |
| pMTL82151 | P3558 | Zm PDC/AdhB |
| pMTL82151 | None | None |
| pMTL82251 | None | None |
| pMTL82351 | None | None |
| pMTL82351 | P1029 | None |

While preferred embodiments have been shown and described herein, it will be obvious to those skilled in the art that such embodiments are provided by way of example only. Numerous variations, changes, and substitutions will now occur to those skilled in the art without departing from the scope of invention. It should be understood that various alternatives to the embodiments described herein may be employed in practicing the invention. It is intended that the following claims define the scope of the invention and that methods and structures within the scope of these claims and their equivalents be covered thereby.

What is claimed is:

1. A genetically modified microorganism that expresses a pyruvate decarboxylase protein, wherein said genetically modified microorganism can hydrolyze and ferment cellulosic and/or lignocellulosic material.

2. The genetically modified microorganism of claim 1, further comprising a genetic modification that expresses a heterologous alcohol dehydrogenase protein.

3. The genetically modified microorganism of claim 1, further comprising a genetic modification that expresses a heterologous acetyl-CoA synthetase protein.

4. The genetically modified microorganism of claim 1, further comprising a genetic modification that inactivates an endogenous lactate dehydrogenase gene.

5. The genetically modified microorganism of claim 1, wherein said genetically modified microorganism produces an increased yield of a fermentation end-product as compared to a non-genetically modified microorganism.

6. The genetically modified microorganism of claim 5, wherein said fermentation end-product is an alcohol.

7. The genetically modified microorganism of claim 1, wherein said genetically modified microorganism is a genetically modified Clostridium bacterium.

8. The genetically modified microorganism of claim 1, wherein said genetically modified microorganism is a genetically modified C. phytofermentans.

9. A method of producing a fermentation end-product comprising:
a) contacting a carbonaceous biomass with a microorganism genetically modified to express a pyruvate decarboxylase protein, wherein said genetically modified microorganism can hydrolyze and ferment cellulosic and/or lignocellulosic material; and,
b) allowing sufficient time for hydrolysis and fermentation to produce said fermentation end-product.

10. The method of claim 9, wherein said microorganism further comprises a genetic modification that expresses a heterologous alcohol dehydrogenase protein.

11. The method of claim 9, wherein said genetically modified microorganism produces an increased yield of said fermentation end-product as compared to a non-genetically modified microorganism.

12. The method of claim 9, wherein said genetically modified microorganism is a genetically modified Clostridium bacterium.

13. The method of claim 9, wherein said genetically modified microorganism is genetically modified C. phytofermentans.

14. The method of claim 9, wherein said fermentation end-product is an alcohol.

15. The method of claim 14, wherein said alcohol is ethanol.

16. The method of claim 9, wherein said biomass comprises cellulosic or lignocellulosic materials.

17. The method of claim 9, wherein said biomass comprises woody plant matter, non-woody plant matter, cellulosic material, lignocellulosic material, hemicellulosic material, carbohydrates, pectin, starch, inulin, fructans, glucans, corn, corn stover, sugar cane, grasses, switch grass, sorghum, bamboo, distillers grains, Distillers Dried Solubles (DDS), Distillers Dried Grains (DDG), Condensed Distillers Solubles (CDS), Distillers Wet Grains (DWG), Distillers Dried Grains with Solubles (DDGS), peels, citrus peels, bagasse, poplar, or algae.

18. A system for producing a fermentation end-product comprising:
a) a fermentation vessel;
b) a carbonaceous biomass;
c) A genetically modified microorganism that expresses a pyruvate decarboxylase protein, wherein said genetically modified microorganism can hydrolyze and ferment cellulosic and/or lignocellulosic material; and,
d) a medium.

19. The system for producing a fermentation end-product of claim 16, wherein said fermentation vessel is configured to house said medium and said microorganism, and wherein said carbonaceous biomass comprises a cellulosic and/or lignocellulosic material.

20. The system of claim 16, wherein said microorganism further comprises a genetic modification that expresses a heterologous alcohol dehydrogenase protein.

21. The system of claim 16, wherein said genetically modified microorganism produces an increased yield of said fermentation end-product as compared to a non-genetically modified microorganism.

22. The system of claim 16, wherein said genetically modified microorganism is a genetically modified Clostridium bacterium.

23. The system of claim 16, wherein said genetically modified microorganism is a genetically modified C. phytofermentans.

24. The system of claim 16, wherein said fermentation end-product is an alcohol.

25. The system of claim 16, wherein said alcohol is ethanol.

26. The system of claim 16, wherein said biomass comprises woody plant matter, non-woody plant matter, cellulosic material, lignocellulosic material, hemicellulosic material, carbohydrates, pectin, starch, inulin, fructans, glucans, corn, corn stover, sugar cane, grasses, switch grass, sorghum, bamboo, distillers grains, Distillers Dried Solubles (DDS), Distillers Dried Grains (DDG), Condensed Distillers Solubles (CDS), Distillers Wet Grains (DWG), Distillers Dried Grains with Solubles (DDGS), peels, citrus peels, bagasse, poplar, or algae.


SEQUENCE LISTING (63 sequences)

SEQ ID NO: 1 - DNA, 4904 bp, Artificial Sequence
  Description of Artificial Sequence Synthetic polynucleotide
gcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaaagctttggctaacacacacgccattccaaccaatagttttctcggcataaagccatgctctgacgcttaaatgcactaatgccttaaaaaaacattaaagtctaacacactagacttatttacttcgtaattaagtcgttaaaccgtgtgctctacgaccaaaagtataaaacctttaagaactttcttttttcttgtaaaaaaagaaactagataaatctctcatatcttttattcaataatcgcatcagattgcagtataaatttaacgatcactcatcatgttcatatttatcagagctccttatattttatttcgatttatttgttatttatttaacatttttctattgacctcatcttttctatgtgttattcttttgttaattgtttacaaataatctacgatacatagaaggaggaaaaactagtatactagtatgaacgagaaaaatataaaacacagtcaaaactttattacttcaaaacataatatagataaaataatgacaaatataagattaaatgaacatgataatatctttgaaatcggctcaggaaaagggcattttacccttgaattagtacagaggtgtaatttcgtaactgccattgaaatagaccataaattatgcaaaactacagaaaataaacttgttgatcacgataatttccaagttttaaacaaggatatattgcagtttaaatttcctaaaaaccaatcctataaaatatttggtaatataccttataacataagtacggatataatacgcaaaattgtttttgatagtatagctgatgagatttatttaatcgtggaatacgggtttgctaaaagattattaaatacaaaacgctcattggcattatttttaatggcagaagttgatatttctatattaagtatggttccaagagaatattttcatcctaaacctaaagtgaatagctcacttatcagattaaatagaaaaaaatcaagaatatcacacaaagataaacagaagtataattatttcgttatgaaatgggttaacaaagaatacaagaaaatatttacaaaaaatcaatttaacaattccttaaaacatgcaggaattgacgatttaaacaatattagctttgaacaattcttatctcttttcaatagctataaattatttaataagtaagttaagggatgcataaactgcatcccttaacttgtttttcgtgtacctattttttgtgaatcgatccggccagcctcgcagagcaggattcccgttgagcaccgccaggtgcgaataagggacagtgaagaaggaacacccgctcgcgggtgggcctacttcacctatcctgcccggatcgattatgtcttttgcgcattcacttcttttctatataaatatgagcgaagcgaataagcgtcggaaaagcagcaaaaagtttcctttttgctgttggagcatgggggttcagggggtgcagtatctgacgtcaatgccgagcgaaagcgagccgaagggtagcatttacgttagataaccccctgatatgctccgacgctttatatagaaaagaagattcaactaggtaaaatcttaatataggttgagatgataaggtttataaggaatttgtttgttctaatttttcactcattttgttctaatttcttttaacaaatgttcttttttttttagaacagttatgatatagttagaatagtttaaaataaggagtgagaaaaagatgaaagaaagatatggaacagtctataaaggctctcagaggctcatagacgaagaaagtggagaagtcatagaggtagacaagttataccgtaaacaaacgtctggtaacttcgtaaaggcatatatagtgcaattaataagtatgttagatatgattggcggaaaaaaacttaaaatcgttaactatatcctagataatgtccacttaagtaacaatacaatgatagctacaacaagagaaatagcaaaagctacaggaacaagtctacaaacagtaataacaacacttaaaatcttagaagaaggaaatattataaaaagaaaaactggagtattaatgttaaaccctgaactactaatgagaggcgacgaccaaaaacaaaaatacctcttactcgaatttgggaactttgagcaagaggcaaatgaaatagattgacctcccaataacaccacgtagttattgggaggtcaatctatgaaatgcgattaagcttagcttggctgcaggtcgacggatccccgggaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgccgagcgcagcgagtcagtgagcgaggaagcggaaga

SEQ ID NO: 2 - DNA, 3255 bp, Artificial Sequence
  Description of Artificial Sequence Synthetic polynucleotide
accaagctatacaatatttcacaatgatactgaaacattttccagcctttggactgagtgtaagtctgactttaaatcatttttagcagattatgaaagtgatacgcaacggtatggaaacaatcatagaatggaaggaaagccaaatgctccggaaaacatttttaatgtatctatgataccgtggtcaaccttcgatggctttaatctgaatttgcagaaaggatatgattatttgattcctatttttactatggggaaatattataaagaagataacaaaattatacttcctttggcaattcaagttcatcacgcagtatgtgacggatttcacatttgccgttttgtaaacgaattgcaggaattgataaatagttaacttcaggtttgtctgtaactaaaaacaagtatttaagcaaaaacatcgtagaaatacggtgttttttgttaccctaaaatctacaattttatacataaccacgaattcggcgcgccctgggcctcatgggccttcctttcactgcccgctttccagtcgggaaacctgtcgtgccagctgcattaacatggtcatagctgtttccttgcgtattgggcgctctccgcttcctcgctcactgactcgctgcgctcggtcgttcgggtaaagcctggggtgcctaatgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttattagaaaaattcatccagcagacgataaaacgcaatacgctggctatccggtgccgcaatgccatacagcaccagaaaacgatccgcccattcgccgcccagttcttccgcaatatcacgggtggccagcgcaatatcctgataacgatccgccacgcccagacggccgcaatcaataaagccgctaaaacggccattttccaccataatgttcggcaggcacgcatcaccatgggtcaccaccagatcttcgccatccggcatgctcgctttcagacgcgcaaacagctctgccggtgccaggccctgatgttcttcatccagatcatcctgatccaccaggcccgcttccatacgggtacgcgcacgttcaatacgatgtttcgcctgatgatcaaacggacaggtcgccgggtccagggtatgcagacgacgcatggcatccgccataatgctcactttttctgccggcgccagatggctagacagcagatcctgacccggcacttcgcccagcagcagccaatcacggcccgcttcggtcaccacatccagcaccgccgcacacggaacaccggtggtggccagccagctcagacgcgccgcttcatcctgcagctcgttcagcgcaccgctcagatcggttttcacaaacagcaccggacgaccctgcgcgctcagacgaaacaccgccgcatcagagcagccaatggtctgctgcgcccaatcatagccaaacagacgttccacccacgctgccgggctacccgcatgcaggccatcctgttcaatcatactcttcctttttcaatattattgaagcatttatcagggttattgtctcatgagcggatacatatttgaatgtatttagaaaaataaacaaataggggttccgcgcacatttccccgaaaagtgccacctaaattgtaagcgttaatattttgttaaaattcgcgttaaatttttgttaaatcagctcattttttaaccaataggccgaaatcggcaaaatcccttataaatcaaaagaatagaccgagatagggttgagtggccgctacagggcgctcccattcgccattcaggctgcgcaactgttgggaagggcgtttcggtgcgggcctcttcgctattacgccagctggcgaaagggggatgtgctgcaaggcgattaagttgggtaacgccagggttttcccagtcacgacgttgtaaaacgacggccagtgagcgcgacgtaatacgactcactatagggcgaattgaaggaaggccgtcaaggccgcatttaattaaggatccggcagtttttctttttcggcaagtgttcaagaagttattaagtcgggagtgcagtcgaagtgggcaagttgaaaaattcacaaaaatgtggtataatatctttgttcattagagcgataaacttgaatttgagagggaacttagatggtatttgaaaaaattgataaaaatagttggaacagaaaagagtattttgaccactactttgcaagtgtaccttgtacatacagcatgaccgttaaagtggatatcacacaaataaaggaaaagggaatgaaactatatcctgcaatgctttattatattgcaatgattgtaaaccgccattcagagtttaggacggcaatcaatcaagatggtgaattggggatatatgatgagatgataccaagctatacaatatttcacaatgatactgaaacattttccagcctttggactgagtgtaagtctgactttaaatca

SEQ ID NO: 3 - DNA, 960 bp, Clostridium phytofermentans
atggcaaaaccaagaaaagtcattattatcggagcaggtcacgtaggatctcatgctggatatgcactggcagagcaggggcttgcagaagaaattatctttattgatattgatagagaaaaagcgaaagcacaagcactggatatctacgatgctacagtatacctaccacacagagttaaggtaaaatcgggtgattatagtgatgcagctgatgcagatctcatggtgattgcagtaggaaccaatccagataaaaataagggtgaaacaagaatgagtacccttacgaatactgctctaattattaaagaggtagcttggcatatcaaaaattcaggttttgatggtatgattgttagcatttcaaatccagcagatgtaataacacattatttacagcatttacttcagtactcatccaataaaattatttcaacaagtacggtactagactctgccagacttagaagagcaattgcagatgctgttgaaattgatcaaaaatcaatctatggatttgttcttggagaacacggagaaagccagatggttgcatggtcaacggtatctatagctggaaaaccaattttggaactaatcaaggaaaaacctgaaaaatatgggcagattgatctttctaagctttctgatgaagctagagcagggggatggcatatcctaactggaaaaggctcaacggaatttggtattggtgcatcactagctgaggttacacgagccattttctcagatgagaagaaggtattaccagtatctactctcttaaatggtgagtatggccagcatgatgtctatgcatctgttcctacggtacttggaattcatggtgtagaagaaatcattgagctaaatttgacacctgaagaaaagggaaaattcgatgcttcttgtagaacaatgaaagaaaattttcagtatgcattgacgctatcataa

SEQ ID NO: 4 - protein, 319 aa, Clostridium phytofermentans
MAKPRKVIIIGAGHVGSHAGYALAEQGLAEEIIFIDIDREKAKAQALDIYDATVYLPHRVKVKSGDYSDAADADLMVIAVGTNPDKNKGETRMSTLTNTALIIKEVAWHIKNSGFDGMIVSISNPADVITHYLQHLLQYSSNKIISTSTVLDSARLRRAIADAVEIDQKSIYGFVLGEHGESQMVAWSTVSIAGKPILELIKEKPEKYGQIDLSKLSDEARAGGWHILTGKGSTEFGIGASLAEVTRAIFSDEKKVLPVSTLLNGEYGQHDVYASVPTVLGIHGVEEIIELNLTPEEKGKFDASCRTMKENFQYALTLS

SEQ ID NO: 5 - DNA, 978 bp, Clostridium phytofermentans
atggcgattacaataaaccgaagtaaagttattgttgtgggtgcaggtttagttggtacttcaacggcgtttagtctaattacgcaaagtgtttgtgatgaggttatgttgatagatatcaatcgtgctaaggcgcatggggaagtaatggatttgtgtcatagtatcgagtatttaaatcgaaatgttttggtaacggaaggagattatacagactgtaaggacgctgatattgttgtaataactgcagggcctccgccaaaaccaggacagtcgcggcttgatactcttgggttatccgcagatattgtgagcacgattgtggaacctgtcatgaagagtgggttcaatggaatattcttagtcgtgacgaatccggtggattcgattgctcaatatgtttatcaattatcggggcttccaaagcaacaagttcttggaactggaacagcgattgactctgcaagattaaaacactttattggagatattttacatgtagatcctagaagcatacaggcttatacgatgggagagcatggagattctcaaatgtgtccttggtcgcttgttacggttggcggtaaaaatattatggacatcgtacgggataacaaagagtattccgatattgactttaatgaaatcttatataaggttaccagggtaggttttgatattttatcagtgaagggtactacttgttatggaatagcgtcagcagctgtggggattataaaagcaattctttatgatgagaattccatccttccggtctctaccttattggagggggaatatggtgagtttgatgtatatgcaggggtaccatgcattctaaatcgtttcggcgtgaaggatgtagtggaagtaaatatgacagaagtagagttaaatcaattccgagcctctgttcacgttgtgagggaagctattgaaaacttaaaagacagagataaaaaggcattatttttataa

SEQ ID NO: 6 - protein, 325 aa, Clostridium phytofermentans
MAITINRSKVIVVGAGLVGTSTAFSLITQSVCDEVMLIDINRAKAHGEVMDLCHSIEYLNRNVLVTEGDYTDCKDADIVVITAGPPPKPGQSRLDTLGLSADIVSTIVEPVMKSGFNGIFLVVTNPVDSIAQYVYQLSGLPKQQVLGTGTAIDSARLKHFIGDILHVDPRSIQAYTMGEHGDSQMCPWSLVTVGGKNIMDIVRDNKEYSDIDFNEILYKVTRVGFDILSVKGTTCYGIASAAVGIIKAILYDENSILPVSTLLEGEYGEFDVYAGVPCILNRFGVKDVVEVNMTEVELNQFRASVHVVREAIENLKDRDKKALFL

SEQ ID NO: 7 - DNA, 27 bp, Artificial Sequence
  Description of Artificial Sequence Synthetic primer
gtatgattgttagcatttcaaatccag

SEQ ID NO: 8 - DNA, 25 bp, Artificial Sequence
  Description of Artificial Sequence Synthetic primer
ttgagccttttccagttaggatatg

SEQ ID NO: 9 - DNA, 23 bp, Artificial Sequence
  Description of Artificial Sequence Synthetic primer
gtttatcaattatcggggcttcc

SEQ ID NO: 10 - DNA, 29 bp, Artificial Sequence
  Description of Artificial Sequence Synthetic primer
ccataacaagtagtacccttcactgataa

SEQ ID NO: 11 - DNA, 996 bp, Clostridium phytofermentans
atgggatttattgatgacatcaaggcaagagctaaacaaagtattaagactattgttttacctgagagtatggacagaagaacaattgaggcagctgctaagactttagaagagggcaatgctaacgtaattattatcggtagtgaggaagaagttaagaagaattcagaaggtcttgacatttcgggagctacaatcgttgaccctaagacatcggacaagcttccagcttacattaacaagcttgtagaacttagacaggcaaaaggcatgacccctgaaaaagcaaaagagcttttaacaacagactacattacatacggtgtaatgatggttaagatgggcgatgcagatggtttagtatctggtgcttgtcactctacagcagataccttaagaccatgtcttcagattttaaaaactgctccaaatactaagttagtttctgctttcttcgtaatggtagtacctaattgtgatatgggcgcaaatggaactttccttttctctgatgctggtttaaatcagaatccaaatgctgaagagttagcagcaatcgctggttccacagcgaagagttttgaacaattagttggctctgaacctatcgtagctatgctttctcattcaacaaagggaagcgcaaagcatgcagatgttgataaggttgtagaagcaactaagattgcaaatgaattatacccagaatataagatcgacggcgagttccagttagatgcagcaatcgttcctagtgtaggtgcttcaaaagctcctggtagtgatattgctggaaaagctaacgtattaatcttcccagaccttgatgctggtaacattggatataagttaacacagcgtcttgcaaaggcagaagcttatggaccattaactcagggtattgcagctccagtaaatgatttatcaagaggttgttcttctgatgatatcgttggtgttgttgcaatcactgctgttcaggcacagagtaaataa

SEQ ID NO: 12 - protein, 331 aa, Clostridium phytofermentans
MGFIDDIKARAKQSIKTIVLPESMDRRTIEAAAKTLEEGNANVIIIGSEEEVKKNSEGLDISGATIVDPKTSDKLPAYINKLVELRQAKGMTPEKAKELLTTDYITYGVMMVKMGDADGLVSGACHSTADTLRPCLQILKTAPNTKLVSAFFVMVVPNCDMGANGTFLFSDAGLNQNPNAEELAAIAGSTAKSFEQLVGSEPIVAMLSHSTKGSAKHADVDKVVEATKIANELYPEYKIDGEFQLDAAIVPSVGASKAPGSDIAGKANVLIFPDLDAGNIGYKLTQRLAKAEAYGPLTQGIAAPVNDLSRGCSSDDIVGVVAITAVQAQSK

SEQ ID NO: 13 - protein, 395 aa, Clostridium phytofermentans
MKVLVINCGSSSLKYQLIDSVTEQALAVGLCERIGIDGRLTHKSADGEKVVLEDALPNHEVAIKNVIAALMNENYGVIKSLDEINAVGHRVVHGGEKFAHSVVINDEVLNAIEECNDLAPLHNPANLIGINACKSIMPNVPMVAVFDTAFHQTMPKEAYLYGIPFEYYDKYKVRRYGFHGTSHSYVSKRATTLAGLDVNNSKVIVCHLGNGASISAVKNGESVDTSMGLTPLEGLIMGTRSGDLDPAIIDFVAKKENLSLDEVMNILNKKSGVLGMSGVSSDFRDIEAAANEGNEHAKEALAVFAYRVAKYVGSYIVAMNGVDAVVFTAGLGENDKNIRAAVSSHLEFLGVSLDAEKNSQRGKELIISNPDSKVKIMVIPTNEELAICREVVELV

SEQ ID NO: 14 - DNA, 1188 bp, Clostridium phytofermentans
atgaaagttttagttattaattgcggaagttcttcccttaaatatcagttaatcgactctgtgacagagcaagcattagcagtaggtctttgtgaaagaatcggtattgatggccgtcttactcacaagtcagctgacggtgagaaggtagttcttgaggatgcacttccaaaccatgaggttgctattaaaaatgtaatcgctgctcttatgaatgaaaattatggtgtgattaagtccttagatgaaatcaacgctgttggacatagagtagtacatggtggtgagaaatttgctcattccgtagtaatcaatgatgaagtcttaaatgcaattgaagagtgtaatgatcttgcacctttacacaacccagcaaaccttattggtatcaacgcttgtaaatcaattatgccaaatgtaccaatggtagctgtttttgatactgcattccatcagacaatgccaaaagaagcttacctttatggtattccatttgagtactatgataaatataaggtaagaagatatggtttccacggaacaagtcacagctatgtttctaaaagagcaaccacgcttgctggcttagatgtaaataactcaaaagttatcgtttgtcaccttggtaatggcgcatccatttccgcagttaaaaacggtgagtctgtagatacaagtatgggtcttacaccacttgaaggtttaatcatgggaacaagaagtggtgatcttgatccagcaatcattgatttcgttgctaagaaagaaaacttatccttagatgaagtaatgaatatcttaaataagaaatctggtgtattaggtatgtccggagtatcttctgactttagagatatcgaagcagcagcaaacgaaggcaatgagcatgcaaaagaagctttagcagtttttgcataccgtgttgctaaatatgtaggttcttatatcgtagctatgaatggtgtagatgctgttgtatttacagcaggacttggtgagaatgataagaacatcagagcagcagtaagttcacaccttgagttccttggtgtatctttagatgctgagaagaattctcaaagaggtaaagaattaatcatctctaacccagattctaaggttaagattatggttatcccaactaacgaagagcttgcaatctgtagagaagttgttgaattagtgtag

SEQ ID NO: 15 - protein, 867 aa, Clostridium phytofermentans
MMAEPKKGYEKSPRIQKLMDALYEKMPEIESKRAVLITESYQQTEGEPIISRRSKAFEHIVKNLPVVIRENELIVGSATVAERGCQTFPEFSFDWLIAELDTVATRTADPFYISEEAKKELRKVHSYWKGKTTSELADYYMAPETKLAMEHNVFTPGNYFYNGVGHITVQYDKVIAIGYEGIKDEVLSRKKELHLGDADYASRLTFYDAVIRSCDSAILYAKRYAAEAKRLALSCQDEKRRQELLMISSNCERVPAKGANTFYEACQAFWFVQLLLQIEASGHSISPGRFDQYLYSYYKADREAGRITGEQAQEIIDCIFVKLNDINKCRDAASAEGFAGYGMFQNMIVGGQDSNGRDATNELSFMILEASIHTMLPQPSLSIRVWNGSPHDLLIKAAEVTRTGIGLPAYYNDEVIIPAMMNKGATLEEARNYNIIGCVEPQVPGKTDGWHDAAFFNMCRPLEMVFSSGYENGKLVGAPTGSVENFTTFEAFYDAYKTQMEYFISLLVNADNSIDIAHAKLCPLPFESSMVEDCIGRGLCVQEGGAKYNFTGPQGFGIANMTDSLYAIKKLVYEEGKVSITELKEALLHNFGMTTKNAGLKESSHLSIDIILAQQITVQIVKELKERGKEPSEKEIEQILKTVLEAKKENTESPISTRVSENTSNHSRYQEILQMIEVLPKYGNDILEIDEFAREIAYTYTKPLQKYKNPRGGVFQAGLYPVSANVPLGEQTGATPDGRLANTPIADGVGPAPGRDTKGPTAAANSVARLDHMDATNGTLYNQKFHPSALQGRGGLEKFVALIRAFFDQKGMHVQFNVVSRETLLDAQKHPENYKHLVVRVAGYSALFTTLSRSLQDDIINRTTQGF

SEQ ID NO: 16 - DNA, 2604 bp, Clostridium phytofermentans
atgatggctgaacccaaaaaaggatatgaaaaatcacctcgtatacaaaagcttatggatgctttatacgagaaaatgccagagattgaatcaaaacgtgcagttttaatcacggaatcgtatcagcagacggaaggagagcctatcattagtagacgctccaaggcttttgaacatatagtaaagaatcttccagtagtaattcgagagaatgaattaattgtaggaagcgcaaccgttgcagaaagaggatgtcaaacctttccggaattctcttttgattggttaattgctgaacttgataccgtagcaactagaactgctgatccgttttatatctcagaggaagcaaaaaaagagttaagaaaagtacatagctattggaagggaaaaacaacaagtgaattagcagattattacatggctccagaaacgaaacttgcgatggagcacaatgtatttacaccaggtaactatttttataacggtgtagggcacattacagtgcagtatgataaggtaattgcgatcggttatgaaggaattaaagatgaagtcttaagcagaaaaaaagaattacatctaggtgatgctgattatgcaagtcgccttactttctatgacgctgtaatcagaagttgtgactcggctattttgtatgctaagagatatgcagcggaagcaaaaagacttgcactttcttgtcaggatgagaagagaagacaagaacttttaatgatttcatctaattgtgagagagtcccagcaaagggtgcgaatacattttatgaagcatgtcaggcattttggtttgtacaacttttattacagattgaagctagtggacattcgatttcaccaggtagatttgaccaatatttatattcatattataaagcagatcgtgaagcaggcagaatcactggtgaacaggcacaagaaatcatcgattgtatttttgtgaaattaaatgatattaacaaatgccgtgatgctgcttctgcggaaggttttgcaggctatggtatgttccagaacatgattgttggcggacaggatagtaacggaagggatgctacgaatgaacttagttttatgatattagaggcatccatacacaccatgcttccacagccttccttaagtatccgtgtatggaatggttctccgcatgatttactaattaaagctgcggaagttaccagaactggtatcggtttacctgcttattacaacgatgaagttattatcccagctatgatgaataagggtgcaactttagaggaagcgagaaactataatattatcggttgcgtggaacctcaagtacctggtaagaccgacggatggcatgacgcagcattctttaatatgtgtcgcccattggaaatggtattttctagtggatatgaaaatggaaaattagttggtgctccaacaggttcggttgaaaacttcactacatttgaggcattttatgatgcttataaaactcagatggaatactttatctctttactagtcaatgcggataattcaatcgatattgcgcatgcaaaactttgcccattaccatttgaatcctctatggtagaagattgtatcggacgtgggttatgtgttcaagaaggtggagcaaaatataattttaccggaccacaagggtttggtatcgccaatatgacagactccttatatgcgattaagaaacttgtatacgaagaaggcaaggtttctattactgaattaaaagaagcacttctacataatttcggaatgacaacgaagaacgctggcttaaaggaaagctctcatctgtccatagatatcatattagcgcagcaaatcacagtgcagattgtaaaagaattgaaagagcgtggaaaagagccttcagagaaggaaatagaacaaatattaaagacagttcttgaagcaaagaaagaaaacacagagagtccaatatctacaagagtgtcagagaacacaagtaatcattcaagatatcaagaaattctacagatgattgaagtgttaccaaagtacggaaatgatatcctagagattgatgaattcgccagggagattgcttatacctatacaaagccattacaaaaatataaaaatccaagaggtggtgtattccaagctggtttatatccggtttccgcaaatgtaccgttaggtgaacaaacaggggctactccagatggaagacttgcgaataccccaattgcagatggtgttggcccagcgccaggacgtgataccaaaggaccaacagcggcagctaattccgtagcacgccttgatcatatggatgcaacaaatggtaccttatacaatcaaaaattccatccatctgcgttacagggtcgtggtggactagagaagtttgtagcgttaatccgtgccttctttgatcaaaagggtatgcatgtacagtttaatgtagtaagtagagaaactttattagacgcacaaaagcacccagaaaactataaacatttggtggtacgtgttgctggttacagtgccctatttactacattatccaggtccttacaggatgatattattaatcgaacaacacaagggttctag

SEQ ID NO: 17 - DNA, 1822 bp, Zymomonas mobilis
gatctgataaaactgatagacatattgcttttgcgctgcccgattgctgaaaatgcgtaaaattggtgattttactcgttttcaggaaaaactttgagaaaacgtctcgaaaacgggattaaaacgcaaaaacaatagaaagcgatttcgcgaaaatggttgttttcgggttgttgctttaaactagtatgtagggtgaggttatagctatggcttcttcaactttttatattcctttcgtcaacgaaatgggcgaaggttcgcttgaaaaagcaatcaaggatcttaacggcagcggctttaaaaatgccctgatcgtttctgatgctttcatgaacaaatccggtgttgtgaagcaggttgctgacctgttgaaaacacagggtattaattctgctgtttatgatggcgttatgccgaacccgactgttaccgcagttctggaaggccttaagatcctgaaggataacaattcagacttcgtcatctccctcggtggtggttctccccatgactgcgccaaagccatcgctctggtcgcaaccaatggtggtgaagtcaaagactacgaaggtatcgacaaatctaagaaacctgccctgcctttgatgtcaatcaacacgacggctggtacggcttctgaaatgacgcgtttctgcatcatcactgatgaagtccgtcacgttaagatggccattgttgaccgtcacgttaccccgatggtttccgtcaacgatcctctgttgatggttggtatgccaaaaggcctgaccgccgccaccggtatggatgctctgacccacgcatttgaagcttattcttcaacggcagctactccgatcaccgatgcttgcgctttgaaagcagcttccatgatcgctaagaatctgaagaccgcttgcgacaacggtaaggatatgccagctcgtgaagctatggcttatgcccaattcctcgctggtatggccttcaacaacgcttcgcttggttatgtccatgctatggctcaccagttgggcggttactacaacctgccgcatggtgtctgcaacgctgttctgcttccgcatgttctggcttataacgcctctgtcgttgctggtcgtctgaaagacgttggtgttgctatgggtctcgatatcgccaatctcggcgataaagaaggcgcagaagccaccattcaggctgttcgcgatctggctgcttccattggtattccagcaaatctgaccgagctgggtgctaagaaagaagatgtgccgcttcttgctgaccacgctctgaaagatgcttgtgctctgaccaacccgcgtcagggtgatcagaaagaagttgaagaactcttcctgagcgctttctaatttcaaaacaggaaaacggttttccgtcctgtcttgattttcaagcaaacaatgcctccgatttctaatcggaggcatttgtttttgtttattgcaaaaacaaaaaatattgttacaaatttttacaggctattaagcctaccgtcataaataatttgccatttaaagcctattatcaggattttcgccccgatttcagccatggcagaaatcttttcggtttaatagcgggaaattctttgatagctggccttttgctcgcttgctttattatttttacatccaggcggtgaaagtgtacagaaaagccgcgtttgccttatgaaggcgacgaaatatttttcagataaagtctttaccttgttaaaaccgcttttcgttttatcgggtaaatgcctaatgcagagtttgatttcaggcctatgtttccgaataaaaagacgccgttgttagacaagatc

SEQ ID NO: 18 - protein, 383 aa, Zymomonas mobilis
MASSTFYIPFVNEMGEGSLEKAIKDLNGSGFKNALIVSDAFMNKSGVVKQVADLLKTQGINSAVYDGVMPNPTVTAVLEGLKILKDNNSDFVISLGGGSPHDCAKAIALVATNGGEVKDYEGIDKSKKPALPLMSINTTAGTASEMTRFCIITDEVRHVKMAIVDRHVTPMVSVNDPLLMVGMPKGLTAATGMDALTHAFEAYSSTAATPITDACALKAASMIAKNLKTACDNGKDMPAREAMAYAQFLAGMAFNNASLGYVHAMAHQLGGYYNLPHGVCNAVLLPHVLAYNASVVAGRLKDVGVAMGLDIANLGDKEGAEATIQAVRDLAASIGIPANLTELGAKKEDVPLLADHALKDACALTNPRQGDQKEVEELFLSAF

SEQ ID NO: 19 - DNA, 2223 bp, Zymomonas mobilis
ggatcctgtaacagctcattgataaagccggtcgctcgcctcgggcagttttggattgatcctgccctgtcttgtttggaattgatgaggccgttcatgacaacagccggaaaaattttaaaacaggcgtcttcggctgctttaggtctcggctacgtttctacatctggttctgattcccggtttacctttttcaaggtgtcccgttcctttttcccctttttggaggttggttatgtcctataatcacttaatccagaaacgggcgtttagctttgtccatcatggttgtttatcgctcatgatcgcggcatgttctgatatttttcctctaaaaaagataaaaagtcttttcgcttcggcagaagaggttcatcatgaacaaaaattcggcatttttaaaaatgcctatagctaaatccggaacgacactttagaggtttctgggtcatcctgattcagacatagtgttttgaatatatggagtaagcaatgagttatactgtcggtacctatttagcggagcggcttgtccaaattggtctcaagcatcacttcgcagtcgcgggcgactacaacctcgtccttcttgacaacctgcttttaaacaaaaacatggagcaggtttattgctgtaacgaactgaactgcggtttcagtgcagaaggttatgctcgtgccaaaggcgcagcagcagccgtcgttacctacagcgtcggtgcgctttccgcattcgatgctatcggtggcgcctatgcagaaaaccttccggttatcctgatctccggtgctccgaacaacaatgaccacgctgctggtcacgtgttgcatcatgctcttggcaaaaccgactatcactatcagttggaaatggccaagaacatcacggccgccgctgaagcgatttataccccggaagaagctccggctaaaatcgatcacgtgattaaaactgctcttcgtgagaagaagccggtttatctcgaaatcgcttgcaacattgcttccatgccctgcgccgctcctggaccggcaagcgcattgttcaatgacgaagccagcgacgaagcttctttgaatgcagcggttgaagaaaccctgaaattcatcgccgaccgcgacaaagttgccgtcctcgtcggcagcaagctgcgcgcagctggtgctgaagaagctgctgtcaaatttgctgatgctcttggtggcgcagttgctaccatggctgctgcaaaaagcttcttcccagaagaaaacccgcattacatcggtacctcatggggtgaagtcagctatccgggcgttgaaaagacgatgaaagaagccgatgcggttatcgctctggctcctgtctttaacgactactccaccactggttggacggatattcctgatcctaagaaactggttctcgctgaaccgcgttctgtcgtcgttaacggcattcgcttccccagcgtccacctgaaagactatctgacccgtttggctcagaaagtttccaagaaaaccggtgctttggacttcttcaaatccctcaatgcaggtgaactgaagaaagccgctccggctgatccgagtgctccgttggtcaacgcagaaatcgcccgtcaggtcgaagctcttctgaccccgaacacgacggttattgctgaaaccggtgactcttggttcaatgctcagcgcataaagctcccgaacggtgctcgcgttgaatatgaaatgcagtggggtcacattggttggtccgttcctgccgccttcggttatgccgtcggtgctccggaacgtcgcaacatcctcatggttggtgatggttccttccagctgacggctcaggaagtcgctcagatggttcgcctgaaaccgccggttatcatcttcttgatcaataactatggttacaccatcgaagttatgatccatgatggtccgtacaacaacatcaagaactgggattatgccggtctgatggaagtgttcaacggtaacggtggttatgacagcggtgctggtaaaggccttaaagctaaaaccggtggcgaactggcagaagctatcaaggttgctctggcaaacaccgacggcccaaccctgatcgaatgcttcatcggtcgggaagactgcactgaagaattggtcaaatggggtaagcgcgttgctgccgccaacagccgtaagcctgttaacaagctcctctagtttttaaataaacttagagaattc

SEQ ID NO: 20 - protein, 568 aa, Zymomonas mobilis
MSYTVGTYLAERLVQIGLKHHFAVAGDYNLVLLDNLLLNKNMEQVYCCNELNCGFSAEGYARAKGAAAAVVTYSVGALSAFDAIGGAYAENLPVILISGAPNNNDHAAGHVLHHALGKTDYHYQLEMAKNITAAAEAIYTPEEAPAKIDHVIKTALREKKPVYLEIACNIASMPCAAPGPASALFNDEASDEASLNAAVEETLKFIADRDKVAVLVGSKLRAAGAEEAAVKFADALGGAVATMAAAKSFFPEENPHYIGTSWGEVSYPGVEKTMKEADAVIALAPVFNDYSTTGWTDIPDPKKLVLAEPRSVVVNGIRFPSVHLKDYLTRLAQKVSKKTGALDFFKSLNAGELKKAAPADPSAPLVNAEIARQVEALLTPNTTVIAETGDSWFNAQRIKLPNGARVEYEMQWGHIGWSVPAAFGYAVGAPERRNILMVGDGSFQLTAQEVAQMVRLKPPVIIFLINNYGYTIEVMIHDGPYNNIKNWDYAGLMEVFNGNGGYDSGAGKGLKAKTGGELAEAIKVALANTDGPTLIECFIGREDCTEELVKWGKRVAAANSRKPVNKLL

SEQ ID NO: 21 - DNA, 1959 bp, Escherichia coli
atgagtcaaattcacaaacacaccattcctgccaacatcgcagaccgttgcctgataaaccctcagcagtacgaggcgatgtatcaacaatctattaacgcacctgataccttctggggcgaacagggaaaaattctcgactggatcaaaccgtaccagaaggtgaaaaacacctcctttgcccccggtaatgtgtccattaaatggtacgaggacggcacgctgaatctggcggcaaactgccttgaccgccatctgcaagaaaacggcgatcgtaccgccatcatctgggaaggcgacgacgccagccagagcaaacatatcagctataaagagctgcaccgcgacgtctgccgcttcgccaataccctgctcaagctgggcattaaaaaaggtgatgtggtggcgatttatatgccgatggtgccggaagccgcggttgcgatgctggcctgcgcccgtattggcgcggtgcattcggtaattttcggtggcttctcgccggaagcggttgccgggcgcattatcgattccaactcacgactggtgatcacttccgacgaaggcgtgcgcgccgggcgtagtattccgctgaagaaaaacgttgatgacgcactaaaaaacccgaacgtcaccagcgtagagcatgtggtggtactgaagcgtactggcgggaaaattgactggcaggaagggcgcgacctgtggtggcacgaccaggttgagcaagccagcgatcagcaccaggcggaagagatgaacgccgaagatccgctgtttattctctatacctccggttctaccggaaaaccaaaaggcgtactgcacactaccggcggttatctggtgtacgcggcgctgacctttaaatatgtctttgattatcatccgggcgatatctactggtgcaccgccgatgtgggctgggtgaccggacacagttatttgctgtacggcccgctggcctgcggcgcgaccacgctgatgtttgaaggcgtaccgaactggccgacgcctgcccgtatggcacaggtggtggacaagcatcaggtcaatattctctataccgcgcccacggcgattcgcgcgctgatggcggaaggcgataaagcgatcgaaggcaccgaccgttcgtcgctgcgcattctcggttccgtgggcgagccaattaacccggaagcgtgggagtggtactggaaaaaaatcggcaacgagaaatgtccggtggtcgatacctggtggcagaccgaaaccggcggtttcatgatcaccccgctgcctggcgctaccgagctgaaagccggttcggcaacacgtccgttcttcggcgtgcaaccggcgctggtcgataacgaaggtaacccgctggaaggggctaccgaaggtagcctggtgatcaccgactcctggccgggtcaggcgcgtacgctgtttggcgatcacgaacgttttgagcagacctatttttccaccttcaaaaatatgtatttcagcggcgacggcgcgcgtcgtgatgaagatagctattactggatcaccgggcgtgtggacgatgtgctgaacgtctccggtcaccgtctgggaacggcggagattgagtcggcgctggtggcgcatccgaaaatcgccgaagccgctgtcgtcggtattccgcacaatattaaaggtcaggcgatctacgcctacgtcacgcttaatcacggggaggaaccgtcaccagaactgtacgcagaagtccgcaactgggtgcgtaaagagattggcccgctggcgacgccagacgtgctgcactggaccgactccctgcctaaaacccgctccggcaaaattatgcgccgtattctgcgcaaaattgcggcgggcgataccagcaacctgggcgatacctcgacgcttgccgatcctggcgtagtcgagaagctgcttgaagagaagcaggctatcgcgatgccatcgtaa

SEQ ID NO: 22 - protein, 652 aa, Escherichia coli
MSQIHKHTIPANIADRCLINPQQYEAMYQQSINAPDTFWGEQGKILDWIKPYQKVKNTSFAPGNVSIKWYEDGTLNLAANCLDRHLQENGDRTAIIWEGDDASQSKHISYKELHRDVCRFANTLLKLGIKKGDVVAIYMPMVPEAAVAMLACARIGAVHSVIFGGFSPEAVAGRIIDSNSRLVITSDEGVRAGRSIPLKKNVDDALKNPNVTSVEHVVVLKRTGGKIDWQEGRDLWWHDQVEQASDQHQAEEMNAEDPLFILYTSGSTGKPKGVLHTTGGYLVYAALTFKYVFDYHPGDIYWCTADVGWVTGHSYLLYGPLACGATTLMFEGVPNWPTPARMAQVVDKHQVNILYTAPTAIRALMAEGDKAIEGTDRSSLRILGSVGEPINPEAWEWYWKKIGNEKCPVVDTWWQTETGGFMITPLPGATELKAGSATRPFFGVQPALVDNEGNPLEGATEGSLVITDSWPGQARTLFGDHERFEQTYFSTFKNMYFSGDGARRDEDSYYWITGRVDDVLNVSGHRLGTAEIESALVAHPKIAEAAVVGIPHNIKGQAIYAYVTLNHGEEPSPELYAEVRNWVRKEIGPLATPDVLHWTDSLPKTRSGKIMRRILRKIAAGDTSNLGDTSTLADPGVVEKLLEEKQAIAMPS

SEQ ID NO: 23 - DNA, 975 bp, Zymomonas mobilis
atggaaattgttgcgattgacatcggtggaacgcatgcgcgtttctctattgcggaagtaagcaatggtcgggttctttctcttggagaagaaacgacttttaaaacggcagaacatgctagcttacagttagcttgggaacgtttcggtgaaaaactgggtcgtcctctgccacgtgccgcagctattgcatgggctggcccggttcatggtgaagttttaaaacttaccaataacccttgggtattaagaccagctactctgaatgaaaagctggacatcgatacgcatgttctgatcaatgacttcggtgcggttgcccacgcggttgcgcatatggattcttcttatctggatcatatttgtggtcctgatgaagcgcttcctagcgatggtgttatcactattcttggtccgggaacgggcttgggtgttgcccatctgttgcggactgaaggccgttatttcgtcatcgaaactgaaggcggtcatatcgactttgctccgcttgacagacttgaagacaaaattctggcacgtttacgtgaacgtttccgccgcgtttctatcgaacgcattatttctggcccgggtcttggtaatatctacgaagcactggctgccattgaaggcgttccgttcagcttgctggatgatattaaattatggcagatggctttggaaggtaaagacaaccttgctgaagccgctttggatcgcttctgcttgagccttggcgctatcgctggtgatcttgctttggcacagggtgcaaccagtgttgttattggcggtggtgtcggtcttcgtatcgcttcccatttgccggaatctggcttccgtcagcgctttgtttcaaaaggacgctttgaacgcgtcatgtccaagattccggttaagttgattacttatccgcagcctggactgctgggtgcggcagctgcctatgccaacaaatattctgaagttgaataa

SEQ ID NO: 24 - protein, 324 aa, Zymomonas mobilis
MEIVAIDIGGTHARFSIAEVSNGRVLSLGEETTFKTAEHASLQLAWERFGEKLGRPLPRAAAIAWAGPVHGEVLKLTNNPWVLRPATLNEKLDIDTHVLINDFGAVAHAVAHMDSSYLDHICGPDEALPSDGVITILGPGTGLGVAHLLRTEGRYFVIETEGGHIDFAPLDRLEDKILARLRERFRRVSIERIISGPGLGNIYEALAAIEGVPFSLLDDIKLWQMALEGKDNLAEAALDRFCLSLGAIAGDLALAQGATSVVIGGGVGLRIASHLPESGFRQRFVSKGRFERVMSKIPVKLITYPQPGLLGAAAAYANKYSEVE

SEQ ID NO: 25 - DNA, 1426 bp, Zymomonas mobilis
cgccatgagttctgaaagtagtcagggtctagtcacgcgactagccctaatcgctgctataggcggcttgcttttcggttacgattcagcggttatcgctgcaatcggtacaccggttgatatccattttattgcccctcgtcacctgtctgctacggctgcggcttccctttctgggatggtcgttgttgctgttttggtcggttgtgttaccggttctttgctgtctggctggattggtattcgcttcggtcgtcgcggcggattgttgatgagttccatttgtttcgtcgccgccggttttggtgctgcgttaaccgaaaaattatttggaaccggtggttcggctttacaaattttttgctttttccggtttcttgccggtttaggtatcggtgtcgtttcaaccttgaccccaacctatattgctgaaattcgtccgccagacaaacgtggtcagatggtttctggtcagcagatggccattgtgacgggtgctttaaccggttatatctttacctggttactggctcatttcggttctatcgattgggttaatgccagtggttggtgctggtctccggcttcagaaggcctgatcggtattgccttcttattgctgctgttaaccgcaccggatacgccgcattggttggtgatgaagggacgtcattccgaggctagcaaaatccttgctcgtctggaaccgcaagccgatcctaatctgacgattcaaaagattaaagctggctttgataaagccatggacaaaagcagcgcaggtttgtttgcttttggtatcaccgttgtttttgccggtgtatccgttgctgccttccagcagttagtcggtattaacgccgtgctgtattatgcaccgcagatgttccagaatttaggttttggagctgatacggcattattgcagaccatctctatcggtgttgtgaacttcatcttcaccatgattgcttcccgtgttgttgaccgcttcggccgtaaacctctgcttatttggggtgctctcggtatggctgcaatgatggctgttttaggctgctgtttctggttcaaagtcggtggtgttttgcctttggcttctgtgcttctttatattgcagtctttggtatgtcatggggccctgtctgctgggttgttctgtcagaaatgttcccgagttccatcaagggcgcagctatgcctatcgctgttaccggacaatggttagctaatatcttggttaacttcctgtttaaggttgccgatggttctccagcattgaatcagactttcaaccacggtttctcctatctcgttttcgcagcattaagtatcttaggtggcttgattgttgctcgcttcgtgccggaaaccaaaggtcggagcctggatgaaatcgaggagatgtggcgctcccagaagtag

SEQ ID NO: 26 - protein, 473 aa, Zymomonas mobilis
MSSESSQGLVTRLALIAAIGGLLFGYDSAVIAAIGTPVDIHFIAPRHLSATAAASLSGMVVVAVLVGCVTGSLLSGWIGIRFGRRGGLLMSSICFVAAGFGAALTEKLFGTGGSALQIFCFFRFLAGLGIGVVSTLTPTYIAEIRPPDKRGQMVSGQQMAIVTGALTGYIFTWLLAHFGSIDWVNASGWCWSPASEGLIGIAFLLLLLTAPDTPHWLVMKGRHSEASKILARLEPQADPNLTIQKIKAGFDKAMDKSSAGLFAFGITVVFAGVSVAAFQQLVGINAVLYYAPQMFQNLGFGADTALLQTISIGVVNFIFTMIASRVVDRFGRKPLLIWGALGMAAMMAVLGCCFWFKVGGVLPLASVLLYIAVFGMSWGPVCWVVLSEMFPSSIKGAAMPIAVTGQWLANILVNFLFKVADGSPALNQTFNHGFSYLVFAALSILGGLIVARFVPETKGRSLDEIEEMWRSQK

SEQ ID NO: 27 - DNA, 1458 bp, Zymomonas mobilis
atgacaaataccgtttcgacgatgatattgtttggctcgactggcgacctttcacagcgtatgctgttgccgtcgctttatggtcttgatgccgatggtttgcttgcagatgatctgcgtatcgtctgcacctctcgtagcgaatacgacacagatggtttccgtgattttgcagaaaaagctttagatcgctttgtcgcttctgaccggttaaatgatgacgctaaagctaaattccttaacaagcttttctacgcgacggtcgatattacggatccgacccaattcggaaaattagctgacctttgtggcccggtcgaaaaaggtatcgccatttatctttcgactgcgccttctttgtttgaaggggcaatcgctggcctgaaacaggctggtctggctggtccaacttctcgcctggcgcttgaaaaacctttaggtcaggatcttgcttcttccgatcatattaatgatgcggttttgaaagttttctctgaaaagcaagtttatcgtattgaccattatctgggtaaagaaacggttcagaaccttctgaccctgcgctttggtaatgctttgtttgaaccgctttggaattcaaaaggcattgaccacgttcagatcagcgttgctgaaacggttggtcttgaaggtcgtatcggttatttcgacggttctggcagcttgcgcgatatggttcaaagccatatccttcagttggtcgctttggttgcaatggaaccgccggctcatatggaagccaacgctgttcgtgacgaaaaggtaaaagttttccgcgctctgcgtccgatcaataacgacaccgtctttacgcataccgttaccggtcaatatggtgccggtgtttctggtggtaaagaagttgccggttacattgacgaactgggtcagccttccgataccgaaacctttgttgctatcaaagcgcatgttgataactggcgttggcagggtgttccgttctatatccgcactggtaagcgtttacctgcacgtcgttctgaaatcgtggttcagtttaaacctgttccgcattcgattttctcttcttcaggtggtatcttgcagccgaacaagctgcgtattgtcttacagcctgatgaaaccatccagatttctatgatggtgaaagaaccgggtcttgaccgtaacggtgcgcatatgcgtgaagtttggctggatctttccctcacggatgtgtttaaagaccgtaaacgtcgtatcgcttatgaacgcctgatgcttgatcttatcgaaggcgatgctactttatttgtgcgtcgtgacgaagttgaggcgcagtgggtttggattgacggaattcgtgaaggctggaaagccaacagtatgaagccaaaaacctatgtctctggtacatgggggccttcaactgctatagctctggccgaacgtgatggagtaacttggtatgactga

SEQ ID NO: 28 - protein, 485 aa, Zymomonas mobilis
MTNTVSTMILFGSTGDLSQRMLLPSLYGLDADGLLADDLRIVCTSRSEYDTDGFRDFAEKALDRFVASDRLNDDAKAKFLNKLFYATVDITDPTQFGKLADLCGPVEKGIAIYLSTAPSLFEGAIAGLKQAGLAGPTSRLALEKPLGQDLASSDHINDAVLKVFSEKQVYRIDHYLGKETVQNLLTLRFGNALFEPLWNSKGIDHVQISVAETVGLEGRIGYFDGSGSLRDMVQSHILQLVALVAMEPPAHMEANAVRDEKVKVFRALRPINNDTVFTHTVTGQYGAGVSGGKEVAGYIDELGQPSDTETFVAIKAHVDNWRWQGVPFYIRTGKRLPARRSEIVVQFKPVPHSIFSSSGGILQPNKLRIVLQPDETIQISMMVKEPGLDRNGAHMREVWLDLSLTDVFKDRKRRIAYERLMLDLIEGDATLFVRRDEVEAQWVWIDGIREGWKANSMKPKTYVSGTWGPSTAIALAERDGVTWYD

SEQ ID NO: 29 - DNA, 1824 bp, Zymomonas mobilis
atgactgatctgcattcaacggtagaaaaggttaccgcgcgcgttattgaacgctcgcgggaaacccgtaaggcttatctggatttgatccagtatgagcgggaaaaaggcgtagaccgtccaaacctgtcctgtagtaaccttgctcatggctttgcggctatgaatggtgacaagccagctttgcgcgacttcaaccgcatgaatatcggcgtcgtgacttcctacaacgatatgttgtcggctcatgaaccatattatcgctatccggagcagatgaaagtatttgctcgcgaagttggcgcaacggttcaggtcgccggtggcgtgcctgctatgtgcgatggtgtgacccaaggtcagccgggcatggaagaatccctgtttagccgcgatgttatcgctttggctaccagcgtttctttgtctcatggtatgtttgaaggggctgcccttctcggtatctgtgacaagattgtccctggtctgttgatgggcgctctgcgcttcggccacctgccgaccattctggtcccatcaggcccgatgacgaccggtatcccgaacaaagaaaaaatccgtatccgtcagctctatgctcagggtaaaatcggccagaaagaacttctggatatggaagcggcttgctaccatgctgaaggtacctgcaccttctatggtacggcaaacaccaaccagatggttatggaagtcctcggtcttcatatgccaggttcggcatttgttaccccgggtaccccgctccgtcaggctctgacccgtgctgctgtgcatcgcgttgctgaattgggttggaagggcgacgattatcgtccgcttggtaagatcattgacgaaaaatcaatcgtcaatgccattgttggtctgttggcaaccggtggttccaccaaccataccatgcatattccggctattgctcgtgctgctggtgttatcgttaactggaatgacttccatgatctttctgaagttgttccgttgattgcccgcatttacccgaatggcccgcgcgacatcaatgaattccagaatgcaggcggcatggcttatgtcatcaaagaactgctttctgctaatctgttgaaccgtgatgtcacgaccattgccaagggcggtatcgaagaatacgccaaggctccggcattaaatgacgctggcgaattggtatggaagccagctggcgaacctggtgatgacaccattctgcgtccggtttctaatcctttcgcaaaagatggcggtctgcgtctcttggaaggtaaccttggacgtgcaatgtacaaagccagtgcagttgatcctaaattctggaccattgaagcaccggttcgcgtcttctctgaccaagacgatgttcagaaagccttcaaggctggcgaattgaacaaagacgttatcgttgttgttcgtttccagggcccgcgcgcaaacggtatgcctgaattgcataagctgaccccggctttgggtgttctgcaggataatggctacaaagttgctttggtaactgatggtcgtatgtccggtgctaccggtaaagttccggttgctttgcatgtcagcccagaagctcttggcggtggtgccatcggtaaattacgtgatggcgatatcgtccgtatctcggttgaagaaggcaaacttgaagctttggttccagctgatgagtggaatgctcgtccgcatgctgaaaaaccggctttccgtccgggaaccggacgcgaattgtttgatatcttccgtcagaacgctgctaaagctgaagacggtgcagtcgcaatatatgcaggtgccggtatctaa

SEQ ID NO: 30 - protein, 607 aa, Zymomonas mobilis
MTDLHSTVEKVTARVIERSRETRKAYLDLIQYEREKGVDRPNLSCSNLAHGFAAMNGDKPALRDFNRMNIGVVTSYNDMLSAHEPYYRYPEQMKVFAREVGATVQVAGGVPAMCDGVTQGQPGMEESLFSRDVIALATSVSLSHGMFEGAALLGICDKIVPGLLMGALRFGHLPTILVPSGPMTTGIPNKEKIRIRQLYAQGKIGQKELLDMEAACYHAEGTCTFYGTANTNQMVMEVLGLHMPGSAFVTPGTPLRQALTRAAVHRVAELGWKGDDYRPLGKIIDEKSIVNAIVGLLATGGSTNHTMHIPAIARAAGVIVNWNDFHDLSEVVPLIARIYPNGPRDINEFQNAGGMAYVIKELLSANLLNRDVTTIAKGGIEEYAKAPALNDAGELVWKPAGEPGDDTILRPVSNPFAKDGGLRLLEGNLGRAMYKASAVDPKFWTIEAPVRVFSDQDDVQKAFKAGELNKDVIVVVRFQGPRANGMPELHKLTPALGVLQDNGYKVALVTDGRMSGATGKVPVALHVSPEALGGGAIGKLRDGDIVRISVEEGKLEALVPADEWNARPHAEKPAFRPGTGRELFDIFRQNAAKAEDGAVAIYAGAGI

SEQ ID NO: 31 - DNA, 2100 bp, Bacillus subtilis
atgtttaaagcattattcggcgttcttcaaaaaattgggcgtgcgcttatgcttccagttgcgatccttccggctgcgggtattttgcttgcgatcgggaatgcgatgcaaaataaggacatgattcaggtcctgcatttcttgagcaatgacaatgttcagcttgtagcaggtgtgatggaaagtgctgggcagattgttttcgataaccttccgcttcttttcgcagtaggtgtagccatcgggcttgccaatggtgatggagttgcagggattgcagcaattatcggttatcttgtaatgaatgtatccatgagtgcggttcttcttgcaaacggaaccattccttcggattcagttgaaagagccaagttctttacggaaaaccatcctgcatatgtaaacatgcttggtatacctaccttggcgacaggggtgttcggcggtattatcgtcggtgtgttagctgcattattgtttaacagattttacacaattgaactgccgcaataccttggtttctttgcgggtaaacgtttcgttccaattgttacgtcaatttctgcactgattctgggtcttattatgttagtgatctggcctccaatccagcatggattgaatgccttttcaacaggattagtggaagcgaatccaacccttgctgcatttatcttcggggtgattgaacgttcgcttatcccattcggattgcaccatattttctattcaccgttctggtatgaattcttcagctataagagtgcagcaggagaaatcatccgcggggatcagcgtatctttatggcgcagattaaagacggcgtacagttaacggcaggtacgttcatgacaggtaaatatccatttatgatgttcggtctgcctgctgcggcgcttgccatttatcatgaagcaaaaccgcaaaacaaaaaactcgttgcaggtattatgggttcagcggccttgacatctttcttaacggggatcacagagccattggaattttctttcttattcgttgctccagtcctgtttgcgattcactgtttgtttgcgggactttcattcatggtcatgcagctgttgaatgttaagattggtatgacattctccggcggtttaattgactacttcctattcggtattttaccaaaccggacggcatggtggcttgtcatccctgtcggcttagggttagcggtcatttactactttggattccgatttgccatccgcaaatttaatctgaaaacacctggacgcgaggatgctgcggaagaaacagcagcacctgggaaaacaggtgaagcaggagatcttccttatgagattctgcaggcaatgggtgaccaggaaaacatcaaacaccttgatgcttgtatcactcgtctgcgtgtgactgtaaacgatcagaaaaaggttgataaagaccgtctgaaacagcttggcgcttccggagtgctggaagtcggcaacaacattcaggctattttcggaccgcgttctgacgggttaaaaacacaaatgcaagacattattgcgggacgcaagcctagacctgagccgaaaacatctgctcaagaggaagtaggccagcaggttgaggaagtgattgcagaaccgctgcaaaatgaaatcggcgaggaagttttcgtttctccgattaccggggaaattcacccaattacggatgttcctgaccaagtcttctcagggaaaatgatgggtgacggttttgcgattctcccttctgaaggaattgtcgtatcaccggttcgcggaaaaattctcaatgtgttcccgacaaaacatgcgatcggcctgcaatccgacggcggaagagaaattttaatccactttggtattgataccgtcagcctgaagggcgaaggatttacgtctttcgtatcagaaggagaccgcgttgagcctggacaaaaacttcttgaagttgatctggatgcagtcaaaccgaatgtaccatctctcatgacaccgattgtatttacaaaccttgctgaaggagaaacagtcagcattaaagcaagcggttcagtcaacagagaacaagaagatattgtgaagattgaaaaataa

SEQ ID NO: 32 - protein, 699 aa, Bacillus subtilis
MFKALFGVLQKIGRALMLPVAILPAAGILLAIGNAMQNKDMIQVLHFLSNDNVQLVAGVMESAGQIVFDNLPLLFAVGVAIGLANGDGVAGIAAIIGYLVMNVSMSAVLLANGTIPSDSVERAKFFTENHPAYVNMLGIPTLATGVFGGIIVGVLAALLFNRFYTIELPQYLGFFAGKRFVPIVTSISALILGLIMLVIWPPIQHGLNAFSTGLVEANPTLAAFIFGVIERSLIPFGLHHIFYSPFWYEFFSYKSAAGEIIRGDQRIFMAQIKDGVQLTAGTFMTGKYPFMMFGLPAAALAIYHEAKPQNKKLVAGIMGSAALTSFLTGITEPLEFSFLFVAPVLFAIHCLFAGLSFMVMQLLNVKIGMTFSGGLIDYFLFGILPNRTAWWLVIPVGLGLAVIYYFGFRFAIRKFNLKTPGREDAAEETAAPGKTGEAGDLPYEILQAMGDQENIKHLDACITRLRVTVNDQKKVDKDRLKQLGASGVLEVGNNIQAIFGPRSDGLKTQMQDIIAGRKPRPEPKTSAQEEVGQQVEEVIAEPLQNEIGEEVFVSPITGEIHPITDVPDQVFSGKMMGDGFAILPSEGIVVSPVRGKILNVFPTKHAIGLQSDGGREILIHFGIDTVSLKGEGFTSFVSEGDRVEPGQKLLEVDLDAVKPNVPSLMTPIVFTNLAEGETVSIKASGSVNREQEDIVKIEK

SEQ ID NO: 33 - DNA, 1206 bp, Bacillus subtilis
atgttaagagggacatatttatttggatatgctttcttttttacagtaggtattatccatatatcaacagggagtttgacaccatttttattagaggcttttaacaagacaacagatgatatttcggtcataatcttcttccagtttaccggatttctaagcggagtattaatcgcacctttaatgattaagaaatacagtcattttaggacacttactttagctttgacaataatgcttgtagcgttaagtatcttttttctaaccaaggattggtattatattattgtaatggcttttctcttaggatatggagcaggcacattagaaacgacagttggttcatttgttattgctaatttcgaaagtaatgcagaaaaaatgagtaagctggaagttctctttggattaggcgctttatctttcccattattaattaattccttcatagatatcaataactggtttttaccatattactgtatattcacctttttattcgtcctattcgtagggtggttaattttcttgtctaagaaccgagagtacgctaagaatgctaaccaacaagtgacctttccagatggaggagcatttcaatactttataggagatagaaaaaaatcaaagcaattaggcttttttgtatttttcgctttcctatatgctggaattgaaacaaattttgccaactttttaccttcaatcatgataaaccaagacaatgaacaaattagtcttataagtgtctcctttttctgggtagggatcatcataggaagaatattgattggtttcgtaagtagaaggcttgatttttccaaataccttctttttagctgtagttgtttaattgttttgttgattgccttctcttatataagtaacccaatacttcaattgagtggtacatttttgattggcctaagtatagcggggatatttcccattgctttaacactagcatcaatcattattcagaagtacgttgacgaagttacaagtttatttattgcctcggcaagtttcggaggagcgatcatctctttcttaattggatggagtttaaaccaggatacgatcttattaaccatgggaatatttacaactatggcggtcattctagtaggtatttctgtaaagattaggagaactaaaacagaagaccctatttcacttgaaaacaaagcatcaaaaacacagtag

SEQ ID NO: 34 - protein, 401 aa, Bacillus subtilis
MLRGTYLFGYAFFFTVGIIHISTGSLTPFLLEAFNKTTDDISVIIFFQFTGFLSGVLIAPLMIKKYSHFRTLTLALTIMLVALSIFFLTKDWYYIIVMAFLLGYGAGTLETTVGSFVIANFESNAEKMSKLEVLFGLGALSFPLLINSFIDINNWFLPYYCIFTFLFVLFVGWLIFLSKNREYAKNANQQVTFPDGGAFQYFIGDRKKSKQLGFFVFFAFLYAGIETNFANFLPSIMINQDNEQISLISVSFFWVGIIIGRILIGFVSRRLDFSKYLLFSCSCLIVLLIAFSYISNPILQLSGTFLIGLSIAGIFPIALTLASIIIQKYVDEVTSLFIASASFGGAIISFLIGWSLNQDTILLTMGIFTTMAVILVGISVKIRRTKTEDPISLENKASKTQ

SEQ ID NO: 35 - DNA, 1899 bp, Bacillus subtilis
atgggcacacttcaggagaaagtgaggcgttttcaaaagaaaaccattaccgagttaagagacaggcaaaatgctgatggttcatggacattttgctttgaaggaccaatcatgacaaattccttttttattttgctccttacctcactagatgaaggcgaaaatgaaaaagaactgatatcatcccttgcagccggcattcatgcaaaacagcagccagacggcacatttatcaactatcccgatgaaacgcgcggaaatctaacggctaccgtccaaggatatgtcgggatgctggcttcaggatgttttcacagaactgagccgcacatgaagaaagctgaacaatttatcatctcacatggcggtttgagacatgttcattttatgacaaaatggatgcttgccgcgaacgggctttatccttggcctgctttgtatttaccattatcactcatggcgctccccccaacattgccgattcatttctatcagttcagctcatatgcccgtattcattttgctcctatggctgtaacactcaatcagcgatttgtccttattaaccgcaatatttcatctcttcaccatctcgatccgcacatgacaaaaaatcctttcacttggcttcggtctgatgctttcgaagaaagagatctcacgtctattttgttacattggaaacgcgtttttcatgcaccatttgcttttcagcagctgggcctacagacagctaaaacgtatatgctggaccggattgaaaaagatggaacattatacagctatgcgagcgcaaccatatatatggtttacagccttctgtcacttggtgtgtcacgctattctcctattatcaggagggcgattaccggcattaaatcactggtgactaaatgcaacgggattccttatctggaaaactctacttcaactgtttgggatacagctttaataagctatgcccttcaaaaaaatggtgtgaccgaaacggatggctctgttacaaaagcagccgactttttgctagaacgccagcataccaaaatagcagattggtctgtcaaaaatccaaattcagttcctggcggctgggggttttcaaacattaatacaaataaccctgactgtgacgacactacagccgttttaaaggcgattccccgcaatcattctcctgcagcatgggagcggggggtatcttggcttttatcgatgcaaaacaatgacggcggattttctgctttcgaaaaaaatgtgaaccatccactgatccgccttctgccgcttgaatccgccgaggacgctgcagttgacccttcaaccgccgacctcaccggacgtgtactgcactttttaggcgagaaagttggcttcacagaaaaacatcaacatattcaacgcgcagtgaagtggcttttcgaacatcaggaacaaaatgggtcttggtacggcagatggggtgtttgctacatttacggcacttgggctgctcttactggtatgcatgcatgcggggttgaccgaaagcatcccggtatacaaaaggctctgcgttggctcaaatccatacaaaatgatgacggaagctggggagaatcctgcaaaagcgccgaaatcaaaacatatgtaccgcttcatagaggaaccattgtacaaacggcctgggctttagacgctttgctcacatatgaaaattccgaacatccgtctgttgtgaaaggcatgcaataccttaccgacagcagttcgcatagcgccgatagcctcgcgtatccagcagggatcggattgccgaagcaattttatattcgctatcacagttatccatatgtattctctttgctggctgtcgggaagtatttagattctattgaaaaggagacagcaaatgaaacgtga

SEQ ID NO: 36 - protein, 632 aa, Bacillus subtilis
MGTLQEKVRRFQKKTITELRDRQNADGSWTFCFEGPIMTNSFFILLLTSLDEGENEKELISSLAAGIHAKQQPDGTFINYPDETRGNLTATVQGYVGMLASGCFHRTEPHMKKAEQFIISHGGLRHVHFMTKWMLAANGLYPWPALYLPLSLMALPPTLPIHFYQFSSYARIHFAPMAVTLNQRFVLINRNISSLHHLDPHMTKNPFTWLRSDAFEERDLTSILLHWKRVFHAPFAFQQLGLQTAKTYMLDRIEKDGTLYSYASATIYMVYSLLSLGVSRYSPIIRRAITGIKSLVTKCNGIPYLENSTSTVWDTALISYALQKNGVTETDGSVTKAADFLLERQHTKIADWSVKNPNSVPGGWGFSNINTNNPDCDDTTAVLKAIPRNHSPAAWERGVSWLLSMQNNDGGFSAFEKNVNHPLIRLLPLESAEDAAVDPSTADLTGRVLHFLGEKVGFTEKHQHIQRAVKWLFEHQEQNGSWYGRWGVCYIYGTWAALTGMHACGVDRKHPGIQKALRWLKSIQNDDGSWGESCKSAEIKTYVPLHRGTIVQTAWALDALLTYENSEHPSVVKGMQYLTDSSSHSADSLAYPAGIGLPKQFYIRYHSYPYVFSLLAVGKYLDSIEKETANET

SEQ ID NO: 37 - DNA, 699 bp, Bacillus subtilis
ttattcaggaaactgaacatggcccggtactgtataggctttggacgttccgctttcaggcagctttggaatggtgtctttcacaacttttccgcggatgtcagtcattctgactttgagagagccagtacctaaattcgtactcacaaaatggttatagtccattttctccatgttgatccacttaccatccttttcatattccattttcataacaggatacttgtgatttctgacttggattgctgcccaccacctgctgctgccttctttgatccggtacgtgaaattgccggtgattggggctttgacaacacgccatttaatattgatttttccgtctttcatattgccgattttacggaaggcattaggtgacagatcaagagctccccgagcgccttcgggataaagatcagtaacatatacggttgttttcccttttggcccttcaacttccaaataagagccggcaagtgccgcttttactcctccgtaattgagatccgccggatttattgcagtaatctccatatcggaaggaatgggatccagcaggaaagctcctcctgaatagcctgaccctgtatacgttgcataaccttcatgcaggtcgtcatatgctgccgaagcttgcggggaaaaacagaagatcgtcaacaaaaccataccaacaaatgcactcatgatctttttcat

SEQ ID NO: 38 - protein, 232 aa, Bacillus subtilis
MKKIMSAFVGMVLLTIFCFSPQASAAYDDLHEGYATYTGSGYSGGAFLLDPIPSDMEITAINPADLNYGGVKAALAGSYLEVEGPKGKTTVYVTDLYPEGARGALDLSPNAFRKIGNMKDGKINIKWRVVKAPITGNFTYRIKEGSSRWWAAIQVRNHKYPVMKMEYEKDGKWINMEKMDYNHFVSTNLGTGSLKVRMTDIRGKVVKDTIPKLPESGTSKAYTVPGHVQFPE

SEQ ID NO: 39 - DNA, 2064 bp, Bacillus subtilis
gtgatgtcaaagcttgaaaaaacgcacgtaacaaaagcgaaatttatgctccatgggggagactacaaccccgatcagtggctggatcggcccgatattttagctgacgatatcaaactgatgaagctttctcatacgaatacgttttctgtcggtatttttgcatggagcgcacttgagccggaggagggcgtatatcaatttgaatggctggatgatatttttgagcggattcacagtataggcggccgggtcatattagcaacgccgagcggagcccgtccggcctggctgtcgcaaacctatccggaagttttgcgcgtcaatgcctcccgcgtcaaacagctgcacggcggaaggcgcaaccactgcctcacatctaaagtctaccgagaaaagacacggcacatcaaccgcttattagcagaacgatacggaaatcacccggggctgttaatgtggcacatttcaaacgaatacgggggagattgccactgtgatctatgccagcatgcttttcgggagtggctgaaatcgaaatatgacaacagcctcaaggcattgaaccaggcgtggtggacccctttttggagccatacgttcaatgactggtcacaaattgaaagcccttcgccgatcggtgaaaatggcttgcatggcctgaatttagattggcgccggttcgtcaccgatcaaacgatttcgttttataaaaatgaaatcattccgctgaaagaattgacgcctgatatccctatcacaacgaattttatggctgacacaccggatttgatcccgtatcagggcctcgactacagcaaatttgcaaagcatgtcgatgtcatcagctgggacgcttatcctgtctggcacaatgactgggaaagcacagctgatttggcgatgaaggtcggttttatcaacgatctgtaccgaagcttgaagcagcagtctttcttattaatggagtgtacgccaagcgcggtcaattggcataacgtcaacaaggcaaagcgcccgggcatgaatctgctgtcatccatgcaaatgattgcccacggctcggacagcgtactctatttccaataccgcaaatcacgggggtcatcagaaaaattacacggagcggttgtggatcatgacaatagcccaaagaaccgcgtctttcaagaagtggccaaggtaggcgagacattggaacggctgtccgaagttgtcggaacgaagaggccggctcaaaccgcgattttatatgactgggaaaatcattgggcgttcggggatgctcaggggtttgcgaaggcgacaaaacgttatccgcaaacgcttcagcagcattaccgcacattctgggaacacgatatccctgtcgacgtcattacgaaagaacaagacttttcaccatataaactgctgatcgtcccgatgctgtatttaatcagcgaggacaccatttcccgtttaaaagcgtttacggctgacggcggcaccttagtcatgacgtatatcagcggggttgtgaatgagcatgacttaacatacacaggcggatggcatccggaccttcaagctatatttggagttgagcctcttgaaacggacaccctgtatccgaaggatcgaaacgctgtcagctaccgcagccaaatatacgaaatgaaggattatgcaaccgtgattgatgtaaagactgctccagtggaagcggtgtatcaagaggatttttacgcccgtacgccagctgtcacaagccatcaatatcagcagggcaaggcgtattttatcggcgcgcgtttggaggatcaatttcaccgtgatttctatgagggtctgatcacagacctgtctctttcacctgtttttccggttcggcatggaaaaggcgtctccgtacaagcgaggcaggatcaggacaatgattatatttttgtgatgaactttacggaagaaaaacagctggtcacgtttgaccagagtgtgaaggacataatgacaggagacatattgtcaggcgacctgacgatggaaaagtatgaagtgagaattgtcgtaaacacacattaa

SEQ ID NO: 40 - protein, 687 aa, Bacillus subtilis
MMSKLEKTHVTKAKFMLHGGDYNPDQWLDRPDILADDIKLMKLSHTNTFSVGIFAWSALEPEEGVYQFEWLDDIFERIHSIGGRVILATPSGARPAWLSQTYPEVLRVNASRVKQLHGGRRNHCLTSKVYREKTRHINRLLAERYGNHPGLLMWHISNEYGGDCHCDLCQHAFREWLKSKYDNSLKALNQAWWTPFWSHTFNDWSQIESPSPIGENGLHGLNLDWRRFVTDQTISFYKNEIIPLKELTPDIPITTNFMADTPDLIPYQGLDYSKFAKHVDVISWDAYPVWHNDWESTADLAMKVGFINDLYRSLKQQSFLLMECTPSAVNWHNVNKAKRPGMNLLSSMQMIAHGSDSVLYFQYRKSRGSSEKLHGAVVDHDNSPKNRVFQEVAKVGETLERLSEVVGTKRPAQTAILYDWENHWAFGDAQGFAKATKRYPQTLQQHYRTFWEHDIPVDVITKEQDFSPYKLLIVPMLYLISEDTISRLKAFTADGGTLVMTYISGVVNEHDLTYTGGWHPDLQAIFGVEPLETDTLYPKDRNAVSYRSQIYEMKDYATVIDVKTAPVEAVYQEDFYARTPAVTSHQYQQGKAYFIGARLEDQFHRDFYEGLITDLSLSPVFPVRHGKGVSVQARQDQDNDYIFVMNFTEEKQLVTFDQSVKDIMTGDILSGDLTMEKYEVRIVVNTH

SEQ ID NO: 41 - DNA, 1590 bp, Pseudoalteromonas haloplanktis
taacttcaatttaaggaaatacgatgaataacagttcaaataatcacaaaagaaaggattttaaagtggcgagcttatcgttagctttattattaggatgctcaacaatggccaatgccgctgttgagaagttaacggtgagtgggaatcaaattcttgcgggtggagaaaacacaagctttgcaggacctagcctattttggagtaatacggggtggggcgctgaaaaattttatacagcagaaacagtagcaaaggcaaaaactgaatttaatgcaacattaattcgtgcagctattggtcatggtacgagtactggtggtagtttgaactttgattgggagggcaatatgagccgtcttgatactgttgtaaacgcagctattgctgaggatatgtacgttattattgattttcatagccatgaagcacataccgatcaggcgactgcagttcgcttttttgaagacgtagctaccaaatatgggcagtacgacaatgttatttatgaaatttataacgagccattacaaatctcgtgggttaacgatattaagccttacgcagaaacagttattgataaaattagagcaatcgaccctgataacttaattgtggttggaacgcctacgtggtcgcaagatgttgatgtggcatcacaaaacccaattgatcgtgccaatattgcttacactctgcatttttatgctggcacgcatggtcaatcgtatcgaaataaagcacaaacagcactcgataacggcattgcactattcgccacagagtggggaacagttaatgctgatggaaatggtggtgttaatatcaatgaaaccgatgcatggatggcattttttaaaacaaacaatattagccacgctaactgggctttaaacgataaaaacgaaggtgcatcgttatttactccaggcggtagttggaattcactaacatcgtcaggctctaaagttaaagagatcattcaaggttggggtggtggtagtagcaatgttgatttagatagcgacggggatggcgtaagtgacagccttgatcagtgcaataatactcccgcaggtacaacggttgatagtattggttgtgcagtaactgacagcgatgccgatggtattagcgataatgttgatcaatgtcctaatacaccagtaggtgaaactgttaataatgtaggttgcgttgttgaagtagttgagccacaaagcgatgcggataacgatggtgtgaatgatgatatcgatcagtgcccagatacacccgctggtacaagtgttgatacaaacggatgcagtgttgtaagctcaacagattgtaacggtattaatgcataccctaattgggtgaacaaagattactcaggtggtccgtttacccacaataacaccgacgataaaatgcaatatcaaggtaatgcatacagcgcaaattggtatacaaacagccttccaggaagtgatgcttcgtggacgcttctttatacttgtaattaagcacgttttataaaatatgcgaagaaggtaaataatacatttaccttctttttaaaagtattagcctttataaacactttgg

SEQ ID NO: 42 - protein, 494 aa, Pseudoalteromonas haloplanktis
MNNSSNNHKRKDFKVASLSLALLLGCSTMANAAVEKLTVSGNQILAGGENTSFAGPSLFWSNTGWGAEKFYTAETVAKAKTEFNATLIRAAIGHGTSTGGSLNFDWEGNMSRLDTVVNAAIAEDMYVIIDFHSHEAHTDQATAVRFFEDVATKYGQYDNVIYEIYNEPLQISWVNDIKPYAETVIDKIRAIDPDNLIVVGTPTWSQDVDVASQNPIDRANIAYTLHFYAGTHGQSYRNKAQTALDNGIALFATEWGTVNADGNGGVNINETDAWMAFFKTNNISHANWALNDKNEGASLFTPGGSWNSLTSSGSKVKEIIQGWGGGSSNVDLDSDGDGVSDSLDQCNNTPAGTTVDSIGCAVTDSDADGISDNVDQCPNTPVGETVNNVGCVVEVVEPQSDADNDGVNDDIDQCPDTPAGTSVDTNGCSVVSSTDCNGINAYPNWVNKDYSGGPFTHNNTDDKMQYQGNAYSANWYTNSLPGSDASWTLLYTCN

SEQ ID NO: 43 - DNA, 837 bp, Clostridium cellulolyticum
ctattctatattcatacttatatcaatagaatttgcagagtgagtaagtttacctatagatataatatcaactcctgttaacgctacattatatatagtttcttcacttatattccccgaggcctccgcaagagctcttttatttataagcttgacagcctcagccatctgttcatttgacatattatcaagcataattatatctgccttgcattcgagagcctcacgaacctcttccatggactctacttctacttcgatctttacagtatgaggaatactgtttcttacacgttgaaccgcatttgttattcctccggcagcagcaatgtggttatcctttatgagaacaccgtcagaaagcgaaaatctgtgattggctcctcctcctgcacttactgcatatttctccagaagtctcagaccgggagtagtttttcttgtatcagttacctttacaggtaacccctgaactttactaacatatctgttagtcatagtagcaattgcagataacctttgcataaagttcaatgcagtcctttcaccttttaacaaagctcttgtcgaaccgcttacctcggctataatatcacctttcgaaaccttgtctccatcttttacaaaggccttaaaacatatgccgctatccagtacctcaaaaacatacttcgcaacatcgagccctgcaataaccgcatcctgctttgccataaattcggctctggatgaatctccttctgaaagaatattgtctgttgtaatatcacctagtggcatatcctcttttaatgcattcataactatttcatggatataaagattactgagtttcat

SEQ ID NO: 44 - protein, 278 aa, Clostridium cellulolyticum
MKLSNLYIHEIVMNALKEDMPLGDITTDNILSEGDSSRAEFMAKQDAVIAGLDVAKYVFEVLDSGICFKAFVKDGDKVSKGDIIAEVSGSTRALLKGERTALNFMQRLSAIATMTNRYVSKVQGLPVKVTDTRKTTPGLRLLEKYAVSAGGGANHRFSLSDGVLIKDNHIAAAGGITNAVQRVRNSIPHTVKIEVEVESMEEVREALECKADIIMLDNMSNEQMAEAVKLINKRALAEASGNISEETIYNVALTGVDIISIGKLTHSANSIDISMNIE

SEQ ID NO: 45 - DNA, 1605 bp, Clostridium cellulolyticum
ttaaaatggtgaagccatttttcccttctccaattccttaactatattttttctccagttcgtatcatcagttttgtcgtagtctgttctataatgagcacctctgctctcttttctttcaagagctgattctataacaagccccgctactgtaagcatattcaacacttccagctttacaagactgaatcctgtaaaatccgtgtacttcttataaatatctttaataatttgggcagccttttcaagaccttgttgacttctgattatacctacatactttgtcattgcagcctgtatctcttccttcatagatttaagagccgcatcattttctttattggatacataacagagccttgaattgacggctgaattattacaaggtcttccttcggactcgatcttctttgcgattttcctgccgaaaaccagtccttctagcaaagaattgcttgcgagcctgtttgcaccgtgaatccctgtacaagctacctctccacatgcatacagacccggaatatttgtctgcccgtcaacatctgtttttactccccccatacaataatgctctgcgggagcaaccggaataaaatccttagaaatatcaataccgtaatccagacatgttttaaagatattaggaaacctactttcgatatattccctacctttaaatgttatatccagaaatacatttttggaatcagtaagatacatttctttaaaaatcgctcttgaaacaatgtctctgggtgccagttcacccaactcgtgatatttcttcataaaaggctcaccgttgctatttttaagttgagcaccctctcctctaaccgcctcagatattaggaaactcttgtcttttgggtggtatagtactgtaggatggaactgtataaactccatatccatggcctgggcacccgctctcaaacacattccgactccgtcaccagttgcgacctcaggattagtagtatgtgcataaatctgtccaaaacccccagttgcaacaactaccgagccggatttaaatatcttaattttatcttcaatttcgtcataaactattacacctttgcatttgccctcttcgatcacaagatcgactgcaaagtgactctcaaaaatcgatatgttcttctttctccgggcaacctcaataagcttgtcacagacttccttaccagtcgtatctcctgagtgaataattctatttacactatgggccccttctctagtaagggatagatgttgtccgcttttatcaaagtttacccctaggctgcacaaaattctaatattttcagcagcctcttctaccagaacccatacgctcttttgatcatttaatcctgcacctgcaaaaagagtatctttgaaatgtagttgtggagaatcattcttctcatcaagagatactgctattcccccttgtgcgagaactgaattgcttatgtccagtgtctctttggtaattatccctatctggaaactgtcgggtatttccaatgcagtatatactccggctattccgctaccaatgatgacgacatccttgtgtatgacctcaacatcaaccttattactatcctcttccat

SEQ ID NO: 46 - protein, 534 aa, Clostridium cellulolyticum
MEEDSNKVDVEVIHKDVVIIGSGIAGVYTALEIPDSFQIGIITKETLDISNSVLAQGGIAVSLDEKNDSPQLHFKDTLFAGAGLNDQKSVWVLVEEAAENIRILCSLGVNFDKSGQHLSLTREGAHSVNRIIHSGDTTGKEVCDKLIEVARRKKNISIFESHFAVDLVIEEGKCKGVIVYDEIEDKIKIFKSGSVVVATGGFGQIYAHTTNPEVATGDGVGMCLRAGAQAMDMEFIQFHPTVLYHPKDKSFLISEAVRGEGAQLKNSNGEPFMKKYHELGELAPRDIVSRAIFKEMYLTDSKNVFLDITFKGREYIESRFPNIFKTCLDYGIDISKDFIPVAPAEHYCMGGVKTDVDGQTNIPGLYACGEVACTGIHGANRLASNSLLEGLVFGRKIAKKIESEGRPCNNSAVNSRLCYVSNKENDAALKSMKEEIQAAMTKYVGIIRSQQGLEKAAQIIKDIYKKYTDFTGFSLVKLEVLNMLTVAGLVIESALERKESRGAHYRTDYDKTDDTNWRKNIVKELEKGKMASPF

SEQ ID NO: 47 - DNA, 915 bp, Clostridium cellulolyticum
ctatttccctactgccagcattctattcaaactaccggatgcacgttctataataccgctatccaatgtaatttcgtattgcctcttagctaaggcatcatgaacactctgtaatgatgttttcttcatattcggacaaatcagccctgttgacatcatataaaaagtcttgtttgggttctccttttttaactggtaaagaacacccatctcagttccaataataaatttgtcatgctcggaatttcttgcataatctataatctgctttgtgcttcccacaaaatcagcaagctcctgtatttcgggtcggcactccggatgtaccagcaaaatagcatcaggatgaagtctctttgactctatgacagcatctttcttaatcttatgatgtgtaatgcagtagccttcccaaaaaataatgtttttttcaggaaccttttttgctacataactgccaagatttttatctggagcaaatataatatcctttttatcgatagatctgattactttctccgcatttgaagatgtacagcagatatcacactcggccttaacctcagcacttgagtttatataacatacaacagctgcgtgaggatactttttcttagcctctttcagagcctcagccgtaaccatatctgccattgggcaacctgcatttatttcaggcaacagaaccgttttttcaggcgatagaagcttcgcactttctgccataaagtgtaccccgcaaaaaactatagtatccgcctgactggaggcacaaaattgacttagagctaatgaatctcctgtaacgtcagcaatctcctgcacctcatcaacctgataactgtgagcaacaataactgcgttctgctctttcttcatttttttaatgttactaatcaacaaatctttatccat

SEQ ID NO: 48 - protein, 304 aa, Clostridium cellulolyticum
MDKDLLISNIKKMKKEQNAVIVAHSYQVDEVQEIADVTGDSLALSQFCASSQADTIVFCGVHFMAESAKLLSPEKTVLLPEINAGCPMADMVTAEALKEAKKKYPHAAVVCYINSSAEVKAECDICCTSSNAEKVIRSIDKKDIIFAPDKNLGSYVAKKVPEKNIIFWEGYCITHHKIKKDAVIESKRLHPDAILLVHPECRPEIQELADFVGSTKQIIDYARNSEHDKFIIGTEMGVLYQLKKENPNKTFYMMSTGLICPNMKKTSLQSVHDALAKRQYEITLDSGIIERASGSLNRMLAVGK

SEQ ID NO: 49 - DNA, 879 bp, Clostridium cellulolyticum
atgaacgagagatatcaattaaacaaaaatcttgcccaaatgctaaagggcggagtaatcatggatgtagtaaatgccaaagaagcagaaattgcacaaaaagccggagccgttgcagtaatggctctcgaaagagttccttccgatataagaaaagccggaggagttgcaagaatgtccgatccaaaaatgataaaagatatacaaagtgccgtatcaattcctgttatggccaaagttagaataggacattttgttgaagcacaggttcttgaagccctttcaattgactatattgatgaaagcgaggttttaactccggcagacgaagaatttcacatagataagcataccttcaaggttccatttgtatgcggtgcaaaaaatctcggagaagctctcagaagaattagtgaaggtgcatccatgataagaactaaaggtgaagccggtacaggaaatgttgttgaagccgtccgacatatgagaactgtaacaaatgaaatcagaaaggtgcagagtgcatccaagcaggaacttatgaccatagcaaaagaatttggtgctccatatgaccttattttatatgttcacgaaaacggtaagcttcctgttataaactttgcagcaggcggaatcgcaactcccgccgatgcggcattaatgatgcagcttggatgcgacggcgtatttgttggttcgggaatatttaaatcctcagatccagccaaaagagcaaaggcaatcgtaaaggcaactacatactataatgatccgcaaatcattgcagaggtctctgaagagcttggtactgccatggattccatagatgtaagagagttaacaggcaacagtctgtatgcctctagaggatggtaa

SEQ ID NO: 50 - protein, 292 aa, Clostridium cellulolyticum
MNERYQLNKNLAQMLKGGVIMDVVNAKEAEIAQKAGAVAVMALERVPSDIRKAGGVARMSDPKMIKDIQSAVSIPVMAKVRIGHFVEAQVLEALSIDYIDESEVLTPADEEFHIDKHTFKVPFVCGAKNLGEALRRISEGASMIRTKGEAGTGNVVEAVRHMRTVTNEIRKVQSASKQELMTIAKEFGAPYDLILYVHENGKLPVINFAAGGIATPADAALMMQLGCDGVFVGSGIFKSSDPAKRAKAIVKATTYYNDPQIIAEVSEELGTAMDSIDVRELTGNSLYASRGW

SEQ ID NO: 51 - DNA, 570 bp, Clostridium cellulolyticum
atgaaaaaaataggtgtgttaggcttgcagggtgctatctcagaacatttggataaactatccaaaataccaaatgtagagccattcagcctaaaatataaagaagaaattgatacaatagacggacttatcatacccggcggtgaaagtactgcaatcggcaggcttctctctgattttaacctgacagaaccactgaaaacaagggtaaatgccgggatgcctgtatggggaacctgtgcaggcatgattatccttgcaaaaacgattactaatgaccgccgacgtcatctggaggttatggacataaatgttatgcggaacgggtatggaagacagttgaacagctttacaacagaggtttccctggctaaagtttcttctgataaaatcccgttggtttttattagagcaccttatgtagtcgaggtagctccgaatgttgaagttcttctgcgtgtagacgaaaacatagtcgcgtgcaggcaggacaatatgctggccacctcctttcatccggagctgacagaagacctgagttttcacaggtactttgcagaaatgatataa

SEQ ID NO: 52 - protein, 189 aa, Clostridium cellulolyticum
MKKIGVLGLQGAISEHLDKLSKIPNVEPFSLKYKEEIDTIDGLIIPGGESTAIGRLLSDFNLTEPLKTRVNAGMPVWGTCAGMIILAKTITNDRRRHLEVMDINVMRNGYGRQLNSFTTEVSLAKVSSDKIPLVFIRAPYVVEVAPNVEVLLRVDENIVACRQDNMLATSFHPELTEDLSFHRYFAEMI

SEQ ID NO: 53 - DNA, 486 bp, Clostridium cellulolyticum
atgatttcaatgatatgggctatgggccgcaacaacgcccttggatgtaaaaacagaatgccctggtacattcccgcagattttgcatatttcaaaaaagttacaatgggaaaaccggtcattatggggagaaaaacttttgaatctatcggtaaacctttaccgggcagaaagaacatagtaattactcgagacacaggatatgatccacaaggctgtattgtggttaattctatagaaaaagccatggagtatacagaagaaaaggaagtctttataatagggggagcagaaatatacaaagaatttcttcctattgcagacagactatatataactctgatagaaaaagagtttgaagcggatgcatttttcccggaaatagactatagtaagtggaagcagatatcctgcgaaacaggaatcaaggatgaaaaaaatccatatgagtataagtggttggtatacgaaagagttaaacaataa

SEQ ID NO: 54 - protein, 161 aa, Clostridium cellulolyticum
MISMIWAMGRNNALGCKNRMPWYIPADFAYFKKVTMGKPVIMGRKTFESIGKPLPGRKNIVITRDTGYDPQGCIVVNSIEKAMEYTEEKEVFIIGGAEIYKEFLPIADRLYITLIEKEFEADAFFPEIDYSKWKQISCETGIKDEKNPYEYKWLVYERVKQ

SEQ ID NO: 55 - DNA, 1047 bp, Haematobia irritans
ttattcaacatagttcccttcaagagcgatacaacgattataacgaccttccaattttttgataccattttggtagtactccttcggttttgcctcaaaataggcctcagtttcggcgatcacctcttcattgcagccaaattttttccctgcgagcatccttttgaggtctgagaacaagaaaaagtcgctgggggccagatctggagaatacggtgggtggggaagcaattcgaagcccaattcatgaatttttgccatcgttctcaatgacttgtggcacggtgcgttgtcttggtggaacaacacttttttcttcttcatgtggggccgttttgccgcgatttcgaccttcaaacgctccaataacgccatataatagtcactgttgatggtttttcccttctcaagataatcgataaaaattattccatgcgcatcccaaaaaacagaggccattactttgccagcggacttttgagtctttccacgcttcggagacggttcaccggtcgctgtccactcagccgactgtcgattggactcaggagtgtagtgatggagccatgtttcatccattgtcacatatcgacggaaaaactcgggtgtattacgagttaacagctgcaaacaccgctcagaatcatcaacacgttgttgtttttggtcaaatgtgagctcgcgcggcacccattttgcacagagcttccgcatatccaaatattgatgaatgatatgaccaacacgttcctttgatatctttaaggcctctgctatctcgatcaacttcattttacggtcattcaaaatcattttgtggatttttttgatgttttcgtcggtaaccacctctttcgggcgtccactgcgttcaccgtcctccgtgctcatttcaccacgcttgaattttgcataccaatcaattattgttgatttccctggggcagagtccggaaactcattatcaagccaagtttttgcttccaccgtattttttcccttcagaaaacagtattttatcaaaacacgaaattcctttttttccat

SEQ ID NO: 56 - protein, 348 aa, Haematobia irritans
MEKKEFRVLIKYCFLKGKNTVEAKTWLDNEFPDSAPGKSTIIDWYAKFKRGEMSTEDGERSGRPKEVVTDENIKKIHKMILNDRKMKLIEIAEALKISKERVGHIIHQYLDMRKLCAKWVPRELTFDQKQQRVDDSERCLQLLTRNTPEFFRRYVTMDETWLHHYTPESNRQSAEWTATGEPSPKRGKTQKSAGKVMASVFWDAHGIIFIDYLEKGKTINSDYYMALLERLKVEIAAKRPHMKKKKVLFHQDNAPCHKSLRTMAKIHELGFELLPHPPYSPDLAPSDFFLFSDLKRMLAGKKFGCNEEVIAETEAYFEAKPKEYYQNGIKKLEGRYNRCIALEGNYVE

SEQ ID NO: 57 - DNA, 336 bp, Escherichia coli
ctacccaatcagtacgttaattttggctttaatgagttgtaattcctctggggcaaccgttcctttcttcgttgctcctcttgcccgccaggcgatactttttacctgatcagctaacgctacgccatcacgttcctgaccggataaaacaacttcgaacggatatccttttgattgcgttgtacaaggaacacacagacacatacctgttttgttgttgtacatgaacggactcaggacaacagccggacgatgtccggcttgctcgctaccttttgtcgggtcaaaatcaacccaaatcagatcgcccatatcgggtacgtatcggcttaccat

SEQ ID NO: 58 - protein, 111 aa, Escherichia coli
MVSRYVPDMGDLIWVDFDPTKGSEQAGHRPAVVLSPFMYNNKTGMCLCVPCTTQSKGYPFEVVLSGQERDGVALADQVKSIAWRARGATKKGTVAPEELQLIKAKINVLIG

SEQ ID NO: 59 - DNA, 249 bp, Escherichia coli
ttaccagacttccttatctttcggctctccccagtcgatattctcgtggaggttttccggcgtgatgtcgttgaccagttcagcaagcgtaaatacgggctctttacgcactggctcaataattaatttgccatccaccaggtcaatcttcacttcatcatcaatattcagattgagcgcctgcattaacgtagccgggatccgcaccgccggtgaatttccccaacgctttacgctactgtggatcat

SEQ ID NO: 60 - protein, 82 aa, Escherichia coli
MIHSSVKRWGNSPAVRIPATLMQALNLNIDDEVKIDLVDGKLIIEPVRKEPVFTLAELVNDITPENLHENIDWGEPKDKEVW

SEQ ID NO: 61 - DNA, 7887 bp, Artificial Sequence
  Description of Artificial Sequence Synthetic polynucleotide
cctgcaggataaaaaaattgtagataaattttataaaatagttttatctacaatttttttatcaggaaacagctatgaccgcggggattttacacgtttcattaataatttcttatatttctttatttgtttgtaaaatttacttaaatttcgccagaaaacaaaagaaagcctttactaattaatagtttagtgatactcttttatgtaggtattttttaaaatacattaaacctaggtaattgaggaaagttacaattaccattatataaggaggatattcatatgaaaagaaaactgaaacaaagatgtgctgttttagtggcagttgcaacgatgatagcttcgttgcaatgggggagagtgccagtacaagcagtaacagcagacggtcttacctctcaacagtatgttgaggcaatgggcgaaggctggaacttaggaaattcctttgatggttttgattctgatacttcaaaaccagatcaaggcgagaccgcttggggaaatcctaaggttacaaaagagctaatccatgcagtcaaacaaaaaggctatagtagtatccgcataccaatgaccctatatcgtagatatacggagagcaatggtgtatgcactatcgatagcgcatggatagcacgttacaaagaagtagtagattatgcagttgcagaaggtttatacgttatgataaacattcaccatgattcctggatatggttatcttcatgggatggaaataagagttctgtgcaatatgtaagatttactcagatgtgggatcaacttgcgaaggcatttaaagattatccgttacaagtatgttttgaaacgataaatgagccgaactttcaaaactctggaaacgttactgcacagaataaattagatatgcttaaccaagcggcttacaatataattcgtgcctctggtggatcaaatgcaaagagaatgattgttttaccatcactaaatacgaaccatgataatagtgtaccattagctgatttcataactaaattgaatgattctaatatcattgcaaccgttcattattatagtgaatgggtatttagtgctaaccttggtaagacaagctttgatgaagatttatggggaaatggtgattacactcctcgtgatgcggtaaataaggcgtttgataccatttccaatgcatttacagcaaaaaaaatcggtgttgttatcggagaatttggtcttttaggttatgactctgattttgaaaataatcaaccaggcgaagaattaaaatattatgagtatatgaattatgtagctagacaaaagaaaatgtgccttatgttttgggataacggatctggaattaatcgtaacgactctaagtatagttggaaaaaacctatagttggaaagatgttagaagtatctatgacaggacgttcctcttatgcaacaggccttgataccatttacctaaacggcagctcatttaatgatattaatatcccgcttactctaaacggtaacacctttgttggagttacaggattaaccagtggtaccgattttacgtataaccaatccaatgcaacactaacattaaaatcatcctacgtgaagaaggtttatgatgcaatgggaagtaattatggtacggtagctgatttggtacttaagttttcaagtggagctgattggcatgagtatttagtgaaatacaaagcaccagtatttcaaaatgcgaatggaactgtttccaatggaattaatattccagttcaatttaacggaagtaaactccgtcgttctacagcttatataggttctaatcgagttggcccgaatcaaagctggtggatgtatttagagtatggtgcaacttttgtggcgaactatacgaacaatattttaaccattaagcctgatttctttaaggatggttctgtttatgatggaaatatatcatttgagatggagttttatgatggacaaaagttaaaatataatcttaataaatcaaatggtaacataacaggaactgcagcagcagtaacccctacaccaacaccaacggcgacaccaacaccaacagcgacgccaacaccaaccgtaacaccaaaaccaacaataaccccaacagtaacgccgacaccaacagtaacgccaaaaccaacaataacaccgacagtaacaccaactcctactccaatcccaggaacaggtccagttacattaaaatacgaagtaacgaatacttgggataagcatacacaggcgaatattacattaaccaatacctctaatacagcactaaagaattttgttgtatcatttacttataaagggtatatagaccaaatgtggagtgcagatttggttagtcaaaattcgggtaccattacagtgaagggaccagcatgggctacgaatctagatccagggcaaagtataacatttggttttattgcttcacatgatacaccgtctgttgatccaccatcaaatgttactttagttagttcaaattaaaattgtattcaaatctcgaggcctgcagacatgcaagcttggcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgctagcataaaaataagaagcctgcatttgcaggcttcttatttttatggcgcgccgttctgaatccttagctaatggttcaacaggtaactatgacgaagatagcaccctggataagtctgtaatggattctaaggcatttaatgaagacgtgtatataaaatgtgctaatgaaaaagaaaatgcgttaaaagagcctaaaatgagttcaaatggttttgaaattgattggtagtttaatttaatatattttttctattggctatctcgatacctatagaatcttctgttcacttttgtttttgaaatataaaaaggggctttttagccccttttttttaaaactccggaggagtttcttcattcttgatactatacgtaactattttcgatttgacttcattgtcaattaagctagtaaaatcaatggttaaaaaacaaaaaacttgcatttttctacctagtaatttataattttaagtgtcgagtttaaaagtataatttaccaggaaaggagcaagttttttaataaggaaaaatttttccttttaaaattctatttcgttatatgactaattataatcaaaaaaatgaaaataaacaagaggtaaaaactgctttagagaaatgtactgataaaaaaagaaaaaatcctagatttacgtcatacatagcacctttaactactaagaaaaatattgaaaggacttccacttgtggagattatttgtttatgttgagtgatgcagacttagaacattttaaattacataaaggtaatttttgcggtaatagattttgtccaatgtgtagttggcgacttgcttgtaaggatagtttagaaatatctattcttatggagcatttaagaaaagaagaaaataaagagtttatatttttaactcttacaactccaaatgtaaaaagttatgatcttaattattctattaaacaatataataaatcttttaaaaaattaatggagcgtaaggaagttaaggatataactaaaggttatataagaaaattagaagtaacttaccaaaaggaaaaatacataacaaaggatttatggaaaataaaaaaagattattatcaaaaaaaaggacttgaaattggtgatttagaacctaattttgatacttataatcctcattttcatgtagttattgcagttaataaaagttattttacagataaaaattattatataaatcgagaaagatggttggaattatggaagtttgctactaaggatgattctataactcaagttgatgttagaaaagcaaaaattaatgattataaagaggtttacgaacttgcgaaatattcagctaaagacactgattatttaatatcgaggccagtatttgaaattttttataaagcattaaaaggcaagcaggtattagtttttagtggattttttaaagatgcacacaaattgtacaagcaaggaaaacttgatgtttataaaaagaaagatgaaattaaatatgtctatatagtttattataattggtgcaaaaaacaatatgaaaaaactagaataagggaacttacggaagatgaaaaagaagaattaaatcaagatttaatagatgaaatagaaatagattaaagtgtaactatactttatatatatatgattaaaaaaataaaaaacaacagcctattaggttgttgttttttattttctttattaatttttttaatttttagtttttagttcttttttaaaataagtttcagcctctttttcaatattttttaaagaaggagtatttgcatgaattgccttttttctaacagacttaggaaatattttaacagtatcttcttgcgccggtgattttggaacttcataacttactaatttataattattattttcttttttaattgtaacagttgcaaaagaagctgaacctgttccttcaactagtttatcatcttcaatataatattcttgacctatatagtataaatatatttttattatatttttacttttttctgaatctattattttataatcataaaaagttttaccaccaaaagaaggttgtactccttctggtccaacatatttttttactatattatctaaataatttttgggaactggtgttgtaatttgattaatcgaacaaccagttatacttaaaggaattataactataaaaatatataggattatctttttaaatttcattattggcctcctttttattaaatttatgttaccataaaaaggacataacgggaatatgtagaatatttttaatgtagacaaaattttacataaatataaagaaaggaagtgtttgtttaaattttatagcaaactatcaaaaattagggggataaaaatttatgaaaaaaaggttttcgatgttatttttatgtttaactttaatagtttgtggtttatttacaaattcggccggcccaatgaataggtttacacttactttagttttatggaaatgaaagatcatatcatatataatctagaataaaattaactaaaataattattatctagataaaaaatttagaagccaatgaaatctataaataaactaaattaagtttatttaattaacaactatggatataaaataggtactaatcaaaatagtgaggaggatatatttgaatacatacgaacaaattaataaagtgaaaaaaatacttcggaaacatttaaaaaataaccttattggtacttacatgtttggatcaggagttgagagtggactaaaaccaaatagtgatcttgactttttagtcgtcgtatctgaaccattgacagatcaaagtaaagaaatacttatacaaaaaattagacctatttcaaagaaaataggagataaaagcaacttacgatatattgaattaacaattattattcagcaagaaatggtaccgtggaatcatcctcccaaacaagaatttatttatggagaatggttacaagagctttatgaacaaggatacattcctcagaaggaattaaattcagatttaaccataatgctttaccaagcaaaacgaaaaaataaaagaatatacggaaattatgacttagaggaattactacctgatattccattttctgatgtgagaagagccattatggattcgtcagaggaattaatagataattatcaggatgatgaaaccaactctatattaactttatgccgtatgattttaactatggacacgggtaaaatcataccaaaagatattgcgggaaatgcagtggctgaatcttctccattagaacatagggagagaattttgttagcagttcgtagttatcttggagagaatattgaatggactaatgaaaatgtaaatttaactataaactatttaaataacagattaaaaaaattataaaaaaattgaaaaaatggtggaaacacttttttcaatttttttgttttattatttaatatttgggaaatattcattctaattggtaatcagattttagaagtttaaactcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcagggccccctgcttcggggtcattatagcgattttttcggtatatccatcctttttcgcacgatatacaggattttgccaaagggttcgtgtagactttccttggtgtatccaacggcgtcagccgggcaggataggtgaagtaggcccacccgcgagcgggtgttccttcttcactgtcccttattcgcacctggcggtgctcaacgggaatcctgctctgcgaggctggccggctaccgccggcgtaacagatgagggcaagcggatggctgatgaaaccaagccaaccaggaagggcagcccacctatcaaggtgtactgccttccagacgaacgaagagcgattgaggaaaaggcggcggcggccggcatgagcctgtcggcctacctgctggccgtcggccagggctacaaaatcacgggcgtcgtggactatgagcacgtccgcgagctggcccgcatcaatggcgacctgggccgcctgggcggcctgctgaaactctggctcaccgacgacccgcgcacggcgcggttcggtgatgccacgatcctcgccctgctggcgaagatcgaagagaagcaggacgagcttggcaaggtcatgatgggcgtggtccgcccgagggcagagccatgacttttttagccgctaaaacggccggggggtgcgcgtgattgccaagcacgtccccatgcgctccatcaagaagagcgacttcgcggagctggtgaagtacatcaccgacgagcaaggcaagaccgatcgggccc

SEQ ID NO: 62 - DNA, 45 bp, Artificial Sequence
  Description of Artificial Sequence Synthetic primer
ccgcggaggagggttttgtatgagtaaaatcagaagaatagtttc

SEQ ID NO: 63 - DNA, 51 bp, Artificial Sequence
  Description of Artificial Sequence Synthetic primer
cccgggttagtggtggtggtggtggtgttttccataatattgccctaatga